SEQUENCE LISTING (183 sequences)

SEQ ID NO: 1 - DNA, 391 bp, Artificial sequencce
  Probe No. 1
ttccagggaagcattatcttgaccagctgaaccacattttgggntattcttggatccccatcacaagaagacctgaattgtataataaatttaaaagctaggaactatttgctttctcttccacacaaaaataaggtgccatggaacaggctgttcccaaatgctgactccaaagctctggacttattggacaaaatgttgacattcaacccacacaagaggattgaagtagaacaggctctggcccacccatatctggagcagtattacgacccgagtgacgagcccatcgccgaagcaccattcaagttcgacatggaattggatgacttgcctaaggaaaagctcaaagaactaatttttgaagagactgctagattccagccaggat

SEQ ID NO: 2 - DNA, 452 bp, Artificial sequence
  Probe No. 2
ggaagctactttcaagcgacctctttgaggagtggatgggtgctctggagatgcaggacgaggaggacagaatcgaggccctgaaacaggttgcagataagctcccccggcccaacctcctgctactcaagcacttggtctatgtgctgcacctcatcagcaagaactctgaggtgaacaggatggactccagcaatctggccatctgcattggacccaacatgctcaccctggagaatgaccagagcctgtcatttgaagcccagaaggacctgaacaacaaggtttgttctgcttactgatgagaaatccccaacttatgatctcaccatctgtttgccaagtccaggcaataaaatgcttcagaatgtctccaactgactcagaatcacagtgaaaatataaaatgcaaaatgcttttgtcatagttccactctctcagatacatgtat

SEQ ID NO: 3 - DNA, 556 bp, Artificial sequence
  Probe No. 3
aagaaacggcgcaccacaagactatatcccacacctggctcagagggtcctacgcccacggaatctcgctgattgctagcacagcagtctgagatcaaactgcaaggcggcaacgaggctgggggaggggcgcccgccattgcccaggcttgcttaggtaaacaaagcagcctggaagctcgaactgggtggagcccaccacagctcaaggaggcctgcctgcctctgtaggctccacctctgggggcagggcacagacaaacaaaaagacagcagtaacctctgcagacttaagtgtccctgtctgacagctttgaagagagcagtggttctcccagcacgcagatggagatctgagaacgggcagactgcctcctcaagtgggtccctgacccctgacccccaagcagcctaactgggaggcaccccccaacaggggcacactgacacctcacacggcagggtattccaacagacctacagctgagggtcctgtctgttagaaggaaaactaacaaccagaaaggacatctacaccgaaaacccatctgtacat

SEQ ID NO: 4 - DNA, 519 bp, Artificial sequence
  Probe No. 4
aacagtacagtcctcaccctgatgaccttgaccccagaggggtcggagctacacatcatcctgggcctgttcggcctcctgctgttgctcacctgcctctgtggaactgcctggctctgttgcagccccaacaggaagaatcccctctggccaagtgtcccagacccagctcacagcagcctgggctcctgggtgcccacaatcatggaggaggatgccttccagctgcccggccttggcacgccacccatcaccaagctcacagtgctggaggaggatgaaaagaagccggtgccctgggagtcccataacagctcagagaccnnnnnnntccccactctggtccagacctatgtgctccagggggacccaagagcagtttccacccagccccaatcccagtctggcaccagcgatcaggctgggcctcccaggcgatctgcatactttaaggaccagatcatgctccatccagccccacccaatggccttttgtgcttgtttcctataacttcagta

SEQ ID NO: 5 - DNA, 448 bp, Artificial sequence
  Probe No. 5
caagggacgcttggacaacgggcaagttggcctatacccggcaaattatgtggaggcgatccagtgatgagtcggggacaggccagcggggggacggaggcggcgggcccaggagcctcagccagccacgtgggcatccactccttttcctgcaagagatgatggttccattgctcttggcttcatggtgttcctggaaggcagatgagctggtcatttcgcctgggactcggcacctttccgagtgcagctggnagggatctgagcgcaggaagacgcagaacaacagaaatagccgcccctccccgcccactgtgcctgttggcctatcatagatctctantgttcttgacttngtnctctcctttccgagtcaatggtgggnnnnacnnnnnnntngttccactgattactctctctgacgagtccatcacctgcaacttaaatg

SEQ ID NO: 6 - DNA, 394 bp, Artificial sequence
  Probe No. 6
ttcacttgcttctcagtgcaacttgataggagaatccagcatcttaaagttgcatatgtgtagcactaatgtttctttttaaatagttgggggaaaatgacctagaaaaccaaattgcagtttggtagccaaaattaactcttggtttatttgtcctttgtgtgtgaaaagtcctactattccgtgcgtcagacttcctcacagaactgttgactggttttggttcttagtactattgagatctttcgcgtcgatcccaacggccttagcggcggcagactggaataacaccttacacctttctggcctgcatttctgtagacttcactctcaagggaggagttttcttttcttacgttttgacttttgcacaccatatgcactagggattctg

SEQ ID NO: 7 - DNA, 387 bp, Artificial sequence
  Probe No. 7
ctcaatgttggctctttggcaggaatggctgctttaaatggtggcctgggcagcagtggcctttccaatggcaccgggagcaccatggaggccctcactcaggcctactcgggtatccagcaatatgctgctgctgcgctccccactctgtacaaccagaatcttctgacacagcagagtattggtgctgctggaagccagaaggaaggtccagagggagccaacctgttcatctaccacctgccccaggagtttggtgatcaggacctgctgcagatgtttatgccctttgggaatgtcgtgtctgccaaggttttcatagacaagcagacaaacctgagcaagtgttttggttttgtaagttacgacaatcctgtttcggcccaa

SEQ ID NO: 8 - DNA, 446 bp, Artificial sequence
  Probe No. 8
ggatgtcgaagaacacagtgtcgtcggcccgcttccggaaggtggacgtggatgaatatgacgagaacaagttcgtggacgaagaagatgggggcgacggccaggccgggcccgacgagggcgaggtggactcctgcctgcggcaaggaaacatgacagctgccctacaggcagctctgaagaacccccctatcaacaccaagagtcaggcagtgaaggaccgggcaggcagcattgtcttgaaggtgctcatctcttttaaagctaatgatatagaaaaggcagttcaatctctggacaagaatggtgtggatctcctaatgaagtatatttataaaggatttgagagcccgtctgacaatagcagtgctatgttactgcaatggcatgaaaaggcacttgctgctggaggagtagggtccattgttcgtgtcttgactgcaaga

SEQ ID NO: 9 - DNA, 354 bp, Artificial sequence
  Probe No. 9
ccattctgagtacttctccgcaaaccctttgtttcattaaggactgttttacatgaagggtgcaaaagtaggataaaaatgagaaccctagggtgaaacacgtgacagaagaataaagactattgaatagtcctcttctctacccatggacnttggnatttttatattngattttaaggaaatataacttagtagtaaagagatgagcattcaagtcaggcagacctgaatttgggtcaaggctgcgccactcaaaagctatatgacctctatatgagcagcttattcaacctcttttaacctccattttgtcatctgtagaatgatgataaatgcctagctcagaaggattcc

SEQ ID NO: 10 - DNA, 220 bp, Artificial sequence
  Probe No. 10
taacttccaaggtcccaccaacagttcagaaacctaccacagtaaatgttccaactacagaagtctcaccaacttctcagaaaaccaccacaaaaaccaccacaccaaatgctcaagcaacacggagtacacctgtttccaggacaaccaagcattttcatgaaacaaccccaaataaaggaagtggaaccacttcaggtactacccgtcttctatctgg

SEQ ID NO: 11 - DNA, 439 bp, Artificial sequence
  Probe No. 11
caagtactggcgagaccaagcgcaagagacactgaaatatgccctggagcttcagaagctcaacaccaacgtggctaagaatgtcatcatgttcctgggagatgggatgggtgtctccacagtgacggctgcccgcatcctcaagggtcagctccaccacaaccctggggaggagaccaggctggagatggacaagttccccttcgtggccctctccaagacgtacaacaccaatgcccaggtccctgacagcgccggcaccgncaccgnctacctgtgtggggtgaaggccaatgagggcaccgtgggggtaagcgcanccactgancgttcccggtgcaacaccacccangggaacgaggtcacctncatcctgcgctngggccaaggacnctngggaaatctgtgggcattgtgaccaccacgagagtgaaccatg

SEQ ID NO: 12 - DNA, 305 bp, Artificial sequence
  Probe No. 12
ttgggaggcccttgaagaatcccctcccacagcactgacagacagagctggaagataggaaggatcaggaagtcagaggcgtagtctaggaaatctagcccctgataggagtttcagaaaaagaaaagtgtcccgggacccaaaggctttgcatttccaggttaaaaagtccaccagaaagtccgcccaagaatggaaattacccaaaccacaatgcatcgctgtgaaatttcacaattcctggattaaacaaaggccctgaaagcttccagagcaaacagatcacataaacagctcaccagact

SEQ ID NO: 13 - DNA, 155 bp, Artificial sequence
  Probe No. 13
gaccacttatcaaaggcatgtgtgtgcaggacaggcttcgccgaaggtttgaggagccagaggggtttacattgcaccttgagctggaaaagtaaaatattggcagtctccgtccttcaaaaacagcaggtattttgtaactcaggctcctgcct

SEQ ID NO: 14 - DNA, 92 bp, Artificial sequence
  Probe No. 14
ctgcacctacgggtcctaataaatcttcactgtctgactttagtctcccactaaaactgcatttcctttctacaatttcaatttctcccttt

SEQ ID NO: 15 - DNA, 564 bp, Artificial sequence
  Probe No. 15
aatgttaacttccatctgtgtgtgtggcgtgtgagcgcacctgtgcgtgtgcacagaagtgctgggctttgggaatctcttactagccaaagttgctccgtggtttcaacagttgggctgtgtttgctttaaaggcaatatggggaatactgaacacacccggctttcttctgagtttttttggttttccagtgacaagttagtcatacaattcatcccccaaattcccattctgagggtccctaacagctttgtaaacattctacaagaacacagttattttgagttcacttaacttttttatcaacttttgatgaccgtcttggtgagtttttcctgagtattcacgggcactaagtatctcacttattttttcaacctacaggaaagtcagtttaaatgaaacgcactgtgttcacagtattctgctaggtgctttaaggaattaaaaaaacaaaaccaaaacaaaacaaaacaaaaaaacacttgaagggtaaaagtcattttccatgacggacactgaataagggcagggaaatgcttttacacatgaagtgctgcaca

SEQ ID NO: 16 - DNA, 73 bp, Artificial sequence
  Probe No. 16
gcagagcagtcagccctacggacagcagagttacagtggttatagccagtccacggacacttcaggctatggc

SEQ ID NO: 17 - DNA, 70 bp, Artificial sequence
  Probe No. 17
ggatgtgttcaaggcaagaccgaattcagaaggatatcgacgtcgtgatccagaagtccagagctgagga

SEQ ID NO: 18 - DNA, 551 bp, Artificial sequence
  Probe No. 18
tttcccagtagagcatacacagacgctcaacttctgtcaaggaacactggcaaaccaacttcacttatttgcctaaaaatgtgagtgagagctgtgaatgtaatttcagcccgaaacttcccagaggggagaagaaaccggattaaagtcttacctgtggtgtgcccatttcgcttttgtggtgaagcttctgccgttgagcctccaggtactcctgaaatggcttctgcagagatggacctatgccggggacagcactggaagcagggtacagtagcccaaagaaaaagacacatttgggaagaaaagcaggaaaaacgttaaagaaaatgtacttaccacctggactcaaaaggcagggattggataggaagaggaataaaatataaaaatcagagaactgctgaaattctgtgacccctttttagttnnnnnnnnnnnnnnnnnnnnnnnnnnnnactccatctaacaaccccttaaaaaccaaaatctctcccaccaacatgtctgggagaaaccaagagctgtaaatcagaaaccgcttccagcaa

SEQ ID NO: 19 - DNA, 326 bp, Artificial sequence
  Probe No. 19
gtagatcatgttcactgcaatgctggacactacaggtatctgtccctgggccagcagggacctctgaagccttctttgtggccttttttttttttcatcctgtggtttttctaatggactttcaggaattttgtaatctcataactttccaagctccaccacttcctaaatcttaagaactttaattgacagtttcaattgaaggtgctgtttgtagacttaacacccagtgaaagcccagccatcatgacaaatccttgaatgttctcttaagaaaatgatgctggtcatcgcagcttcagcatctcctgttttttgatgcttgg

SEQ ID NO: 20 - DNA, 380 bp, Artificial sequence
  Probe No. 20
tgaaggtggccgggcaggacggctccgtggtgcagttcaagatcaagaggcacacgccgctgagcaagctgatgaaggcctactgcgagaggcagggcttgtcaatgaggcagatcagattcaggttcgacgggcagccaatcaatgaaactgacactccagcacagctggagaatggaggacgaggacaccatcgacgtgttccagcagcagacgggaggtgtgccggagagcagcctggcagggcacagtttctagagggcccgtccccagcccgggccgtccatcctcgcattgctgttgaatggtgagcacgtgaccatgccgaccacaaaggtgtctgcggaaactcgaggacattcaccacgatgattttcctc

SEQ ID NO: 21 - DNA, 166 bp, Artificial sequence
  Probe No. 21
gcggcgactggcaatgtttggcctcaaaagaaacgcggtaatcggactcaacctctactgtgggggggccggcttgggggccggcagcggcggcgccacccgcccgggagggcgacttttggctacggagaaggaggcctcggcccggcgagagatagggggaggg

SEQ ID NO: 22 - DNA, 443 bp, Artificial sequence
  Probe No. 22
ccccacttcccaattcattaggtatgactgtggaaatacagacaaggatcttagttgatattttgggcttggggcagtgagggcttaggacaccccaagtggtttgggaaaggaggaggggagtggtgggtttatagggggaggaggaggcaggtggtctaagtgctgactggctacgtagttcgggcaaatcctccaaaagggaaagggaggatttgcttagaaggatggcgctcccagtgactactttttgacttctgtttgtcttacgcttctctcagggaaaaacatgcagtcctctagtgtttcatgtacattctgtggggggtgaacaccttggttctggttaaacagctgtacttttgatagctgtgccaggaagggttaggaccaactacaaattaatgttggttgtcaaatgtagtgtgtttccctaactttct

SEQ ID NO: 23 - DNA, 501 bp, Artificial sequence
  Probe No. 23
acgaggagagcatcgactggaccaagatcgagcccagcgtcaacttcctcaagtcgcccaaagacaacgtggacgaccccacggggaacttccgcagcgggcccctgacggggtggcgggtgttcctgctgctgctgtgcgctctcctgggcatcgttgtctgcgccgtggtgggggccgtggtgttccagaagcggcaggagcggaacaagcgcttctactgagtggcgcctccggcggggcctgtccctgggcccaggagccaatgtgaacttttttttttaccgggattataaaagaacaacaagatgaccttatttcttaactgtttcaaataaatgattaaagtattttcatacattttgcttcttgcccagcagggacaggtggcagagccgaggcttagggtctggcaccccccacagctggagacggaggctctcctggggctggtgtctcaggagcaggggtctgtgtctacagatgggctgtggcccctgc

SEQ ID NO: 24 - DNA, 512 bp, Artificial sequence
  Probe No. 24
caacatcatctgtggcatcacctctgttgccttctcgcgcagcggacggctgctgctcgctggctacgacgacttcaactgcaacatctgggatgccatgaagggcgaccgtgcaggagtcctcgctggccacgacaaccgcgtgagctgcctcggggtcaccgacgatggcatggctgtggccacgggctcctgggactccttcctcaagatctggaactaatggccccacccccactgggcccaggccaggaggggccctgccatgcccacactacaggccagggctgcggggctggcgcaatcccagcccccttccccgggccacggccttgggtccctgccctcccacccaggtttggttcctcccggggcccccactgtggagataagaaggggatggaatgggggaagaggaggagcaggaggccctcatccttctgctgccctggggttggggcctcacccctctggagggccggacagggaggtggaaaccccaggggctggct

SEQ ID NO: 25 - DNA, 544 bp, Artificial sequence
  Probe No. 25
tgtgccttcattcatgggttaatggattaatgggttatcacaggaatgggactggtggctttataagaagaggaaaagagaactgagctagcatgcccagcccacagagagcctccactagagtgatgctaagtggaaatgtgaggtgcagctgccacagagggcccccaccangggaaatgtctagtgtctagtggatccaggccacaggagagagtgccttgtggagcgctgggagcaggacctgaccaccaccaggaccccagaactgtggagtcagtggcagcatgcagcgcccccttgggaaagctttaggcaccagcctgcaacccattcgagcagccacgtaggctgcacccagcaaagccacaggcacggggctacctgangccttgggggcccaatccctgctccagtgtgtccgtgaggcagcacacgaagtcaaaagagattattctcttcccacagataccttttctctcccatgaccctttaacagcatctgcttcattcccctcaccttcccaggctgatctgaggtaaa

SEQ ID NO: 26 - DNA, 289 bp, Artificial sequence
  Probe No. 26
atcccacctgtgagaatatgaacttctcttggaggaatgaatgcaaccagtgtaaggcccctaaaccagatggcccaggagggggaccaggtggctctcacatggggggtaactacggggatgatcgtcgtggtggcagaggaggctatgatcgaggcggctaccggggccgcggcggggaccgtggaggcttccgagggggccggggtggtggggacagaggtggctttggccctggcaagatggattccaggggtgagcacagacaggatcgcagggagaggccgta

SEQ ID NO: 27 - DNA, 521 bp, Artificial sequence
  Probe No. 27
gaactaagcgataacagagtctcagggggcctggaagtattggcagaaaagtgtccgaacctcacgcatctaaatttaagtggcaacaaaattaaagacctcagcacaatagagccactgaaaaagttagaaaacctcaagagcttagaccttttcaattgcgaggtaaccaacctgaacgactaccgagaaaatgtgttcaagctcctcccgcaactcacatatctcgacggctatgaccgggacgacaaggaggcccctgactcggatgctgagggctacgtggagggcctggatgatgaggaggaggatgaggatgaggaggagtatgatgaagatgctcaggtagtggaagacgaggaggacgaggatgaggaggaggaaggtgaagaggaggacgtgagtggagaggaggaggaggatgaagaaggttataacgatggagaggtagatgacgaggaagatgaagaagagcttggtgaagaagaaaggggtcagaagcgaaaacgagaacctgaaga

SEQ ID NO: 28 - DNA, 478 bp, Artificial sequence
  Probe No. 28
aatacatggcttgctgcctgttgtaccgtggtgacgtggttcccaaagatgtcaatgctgccattgccaccatcaaaaccaagcgcacgatccagtttgtggattggtgccccactggcttcaaggttggcatcaactaccagcctcccactgtggtgcctggtggagacctggccaaggtacagagagctgtgtgcatgctgagcaacaccacagccattgctgaggcctgggctcgcctggaccacaagtttgacctgatgtatgccaagcgtgcctttgttcactggtacgtgggtgaggggatggaggaaggcgagttttcagaggcccgtgaagatatggctgcccttgagaaggattatgaggaggttggtgtggattctgttgaaggagagggtgaggaagaaggagaggaatactaattatccattccttttggccctgcagcatgtcatgctcccagaatttcagcttc

SEQ ID NO: 29 - DNA, 449 bp, Artificial sequence
  Probe No. 29
aggacgacgctgtcggaggcagggagagcaaattaccacagcttcttggcccagttctgcccttctttgctttgggattgcactgggccatcagctcatgccaggctatgggggcagccagttggcattgctccccagactgaacagaaacctggccgccggatgggacctcctttggcacagacttgactgtgtaactgcataaactgcagtagcatcattgccctagatgccccaggagacctggcaccatgaggattacagacagtggaatcttactgtcatctggacagctgttttcctgtttggatggtaaaggaagttgagagtctttagacctgtgcacagccccgcaccaaggggtgctgtatgctctaggcatcccctcccccaggggattttttaagtagatggggggacacggtgaactggctgtgtccatctttgtc

SEQ ID NO: 30 - DNA, 528 bp, Artificial sequence
  Probe No. 30
aggtggcccagctcaaacagaaggtcatgacccacgtcagcaacggctgtcagctgctgcttggggtcaagggacacgccttctgaacgtcccctgcccctttacggacaccccctcgcttggacggctgggcacacgcctcccactggggtccagggagcaggcggtgggcacccaccctgggacctaggggcgccgcaaaccacactggactccggcccccctaccctgcgcccagtccttccacctcgacgtttacaagcccccccttccacttttttttgtatgttttttttctgctggaaacagactcgattcatattgaatataatatatttgtgtatttaacagggaggggaagagggggcgatcgcggcggagctggccccgccgcctggtactcaagcccgcggggacattgggaaggggacccccgccccctgccctcccctctctgcaccgtactgtggaaaagaaacacgcacttagtctctaaagagtttattttaagacgtgtttgtgtttgtg

SEQ ID NO: 31 - DNA, 458 bp, Artificial sequence
  Probe No. 31
atcaagtaatccccttttccagaatgcattaacccactcccctgacctcacgctggggcaggtccccaagtgtgcaagctcagtattcatgatggtgggggatggagtgtcttccgaggttcttgggggaaaaaaaattgtagcatatttaagggaggcaatgaaccctctcccccacctcttccctgcccaaatctgtctcctagaatcttatgtgctgtgaataataggccttcactgcccctccagtttttatagacctgaggttccagtgtctcctggtaactggaacctctcctgagggggaatcctggtgctcaaattaccctccaaaagcaagtagccaaagccgttgccaaaccccacccataaatcaatgggccctttatttatgacgactttatttattctaatatgattttatagtatttatatatattgggtcgtctgcttccctt

SEQ ID NO: 32 - DNA, 364 bp, Artificial sequence
  Probe No. 32
ctggctccgtagcagaacactgtaaaagtgcccgcgtctttgcagtagttgcagatttcagtcgtcgtgttacttgtgcacaaacagaagctgggtcttacccgcagcacgagtgtctcgggctgcccggagtcgcccgggagcaggtgctgcagccagagttacgcgggggccacgcgggccggcgggggtggggggaacgtgggggaacctgtgtttcacgtgactcagcagtgcccgccgccgtcaccagctatgcattcactccgtttccagtgagcagatgtcttgcttggaaagtggacctgtgtctgtgtctgtcctgagaacttaccagcagaaatcctcatttctgtgctacgga

SEQ ID NO: 33 - DNA, 438 bp, Artificial sequence
  Probe No. 33
ggctagactttgccatggctgtcaaaagggacagccgcaaagccctggttgcccaggtaatcaaagagaagctaaggctgaagtctgcaacaggctctgaggtccggggaaagctagaaactaaatcggacctgaacatgcaacagcaggaagaggaggagaaagcccggctcctcattggtttaagtgtgggcgacaagaaccctggcaagaagtccatctttggcaggcgcaaatgatttggcgattcgagtggctgcagtacaggatctgactctggctcaggctccagggacttgtggggtgggaggggcttcccgttatccacgaggatttgtgggtgtcagagcccataggcatcactcttcagcacctggtctgttcgctgcagggcatggtggacagtaatgctgagttctgtctcacactgatcaggct

SEQ ID NO: 34 - DNA, 504 bp, Artificial sequence
  Probe No. 34
gcagttctctccctgaaaacacagtacaggttgagtcaaatgaggtcatgggtgcaccagatgacaggaccagaactccccttgagccatccaactgttggagtgacttagatggtgggaaccacacagagaatgtgggagaggcagcagtgactcaggttgaagagcaggcaggcacagtggcctcgtgtcctttagggcatagtgatgacacagtttatcatgatgacaaatgtatggtagaggtcccccaagagttagagacaagcacagggcatagtttagagaaagaattcaccaaccaggaagcagctgagcccaaggaggttccagcgcacagtacagaagtaggtagggatcacaacgaagaagagggtgaagaaacaggattaagggacgagaaaccaatcaagacagaagttcctggttctccagcaggaactgagggcaactgtcaggaagcgacaggtccaagtacagtagacactcaaaatgaacccttag

SEQ ID NO: 35 - DNA, 494 bp, Artificial sequence
  Probe No. 35
acaagttgacctccacggtgatgctgtggctgcagaccaacaaatctggctctggcaccatgaacctcggaggcagccttaccagacagatggagaaggatgaaactgtgagtgactgctccccacacatagccaacatcgggcgcctggtagaggacatggaaaataaaatcagaagtacgctgaacgagatctactttggaaaaacaaaggatatcgtcaatgggctgaggtctgtgcagacttttgcagacaaatcaaaacaagaagctctgaagaatgacctggtggaggctttgaagagaaagcagcaatgctaaacctctgtttcatgctaaccagacacgccgtgcactcgttagattcctttcttagaaaactcgttttctgctcccttccctcgtcccttccctccccgacaggtcacataacagctgcatcattgaccgcacagcgccatctctccctgagaataaagccgatagccacctcct

SEQ ID NO: 36 - DNA, 527 bp, Artificial sequence
  Probe No. 36
cagacaacagcctggtggcagcgggccacgactgcttcccggtgctgttcacctatgacgccgccgcggggatgctgagcttcggcgggcggctggacgttcctaagcagagctcgcagcgtggcttgacggcccgcgagcgcttccagaacctggacaagaaggcgagctccgagggtggcacggctgcgggcgcgggcctagactcgctgcacaagaacagcgtcagccagatctcggtgctcagcggcggcaaggccaagtgctcgcagttctgcaccactggcatggatggcggcatgagtatctgggatgtgaagagcttggagtcagccttgaaggacctcaagatcaaatgacctgtgaggaatatgttgccttcatcctaactgctggggaagcggggagaggggtcagggaggctaatggttgctttgctgaatgtttctggggtaccaatacgagttcccataggggctgctccctcaaaaagggaggggacagatggggagcttttcttacctatt

SEQ ID NO: 37 - DNA, 438 bp, Artificial sequence
  Probe No. 37
gaggaactaaaggaactgctacccgaaattagagagaagatagaagatgcaaaggagtctcagcgtagtgggaatgtagctgaactggctctgaaagctactctggtggagagttctacttcaggtttcactcctggtggaggaggctcttcagtctccatgattgccagtagaaagccaacagacggtgcttcctcatcaaattgtgtgactgatatttcccaccttgtcagaaagaagaggaaaccagaggaagagagtccccggaaagatgatgcaaagaaagccaaacaagagccggaggtgaacggaggcagtggggatgctgtcccgagtggaaatgaagtttcggaaaacatggaggaggaggctgagaatcagctgaaacgcggagcagcagtggaggggacactggaggctggagctacagttgaaagc

SEQ ID NO: 38 - DNA, 441 bp, Artificial sequence
  Probe No. 38
gacatctcgaagtggaagccctctcagagcctacccaccacaaacagtggcgtgagtgctcaggatcggcagttgctgtgcttctactatgaccaatgtgagacccatttcatttcccttctcaacgccattgacgcactcttcagttgtgtcagctcagcccagcccccgcgaatcttcgtggcacacagcaagtttgtcatcctcagtgcacacaaactggtgttcattggagacacgctgacacggcaggtgactgcccaggacattcgcaacaaagtcatgaactccagcaaccagctctgcgagcagctcaagactatagtcatggcaaccaagatggccgccctccattaccccagcaccacggccctgcaggaaatggtgcaccaagtgacagacctttctagaaatgcccagctgttcaagcgctctttgctg

SEQ ID NO: 39 - DNA, 559 bp, Artificial sequence
  Probe No. 39
tgagcatggccgtggagagcaccgggactgccaaggcggaggccgagtcccgtgcggaggcagcccggattgagggagaagggtccgtgctgcaggccaagctaaaagcacaggccttggccattgaaacggaggctgagctccagagggtccagaaggtccgagagctggaactggtctatgcccgggcccagctggagctggaggtgagcaaggctcagcagctggctgaggtggaggtgaagaagttcaagcagatgacagaggccataggccccagcaccatcagggaccttgctgtggctgggcctgagatgcaggtaaaactgctccagtccctgggcctgaaatcaaccctcatcaccgatggctccactcccatcaacctcttcaacacagcctttgggctgctggggatggggcccgagggtcagcccctgggcagaagggtgccagtggcccagccctggggaggggatatccccccagtctgctcaggcccctcaagctcctggagacaaccacgtggtgcctgtactgcgctaactcctgattaata

SEQ ID NO: 40 - DNA, 433 bp, Artificial sequence
  Probe No. 40
gatcccggtgcagctgaatgccggccagctgcagtatatccgcttagcccagcctgtatcaggcactcaagttgtgcagggacagatccagacacttgccaccaatgctcaacagattacacagacagaggtccagcaaggacagcagcagttcagccagttcacagatggacagcagctctaccagatccagcaagtcaccatgcctgcgggccaggacctcgcccagcccatgttcatccagtcagccaaccagccctccgacgggcaggccccccaggtgaccggcgactgagggcctgagctggcaaggccaaggacacccaacacaatttttgccatacagccccaggcaatgggcacagccttcctccccagaggacccggccgacctcagcgcctcctgcaggctaggacactggtgcactacacc

SEQ ID NO: 41 - DNA, 475 bp, Artificial sequence
  Probe No. 41
tgaatcagccataacgcacacacacgccacccagcctcttgtttctagtatgtactttgaaatgctaactgagggtcttgatgcttgagcctttgactgataaaactcaaatagcagtccccagtgatttgcctcttaggttctttcttaaattgttggtggatgactgtacattttagtgatttgaaaaataactgacaaaccattgaaacagtttattttatgttggaagagatggcgcagatgtgtgtcagaagggagatcacggtgtgagtttcgtagctatttaagtgatacatacctctagtttttgtatgtcttttgagatcctgagttcatcccctgtgaatcagagtgcacaagcacctctcctgtgagtggctaatgagaagagggacagaccgaccaccagcacagtagggcagatctggacagcagaatgttataacgcaagttcatgtgttgctcccaactc

SEQ ID NO: 42 - DNA, 454 bp, Artificial sequence
  Probe No. 42
gtcgtctttctattttcaggtcagctgattagccaccttagttccatctgcaactttagttcccactggctgtgtaacctaacatagtcacaggctctggggactgtcacgtggacatctttgggaggccgttattctgcccaccgcaccctccgttcatcccctgccctgccgggcacctcgctctaccccaggaaaatgtgagctcgttttcctgctcggcatgtgctccccctaaggctctgctcctccctgggcctgaaagttccttctcagcctgagagggggcccttcgatctcaggcatgactcagcccggctgatgcctctgcagtgctgagtcaggatttggggccggctctcttgggtctgtccccttttcccaggtactgccttacaaagctgtggccaggaagtggccggtataaaggatgcccaaggtctttgtacgtgtg

SEQ ID NO: 43 - DNA, 502 bp, Artificial sequence
  Probe No. 43
acaggagtcagtgtctggctttttcctctgagcccagctgcctggagagggtctcgctgtcactggctggctcctaggggaacagaccagtgaccccagaaaagcataacaccaatcccagggctggctctgcactaagcgaaaattgcactaaatgaatctcgttccaaagaactaccccttttcagctgagccctggggactgttccaaagccagtgaatgtgaaggaaactcccctccttcggggcaatgctccctcagcctcagaggagctctaccctgctccctgctttggctgaggggcttgggaaaaaaacttggcactttttcgtgtggatcttgccacatttctgatcagaggtgtacactaacatttcccccgagctcttggcctttgcatttatttatacagtgccttgctcggggcccaccaccccctcaagccccagcagccctcaacaggcccagggagggaagtgtgagcgccttggtatgacttaa

SEQ ID NO: 44 - DNA, 513 bp, Artificial sequence
  Probe No. 44
gcaggagctcagcatagacccagctctctgggggatggtcacctggtgatttcaatgatggcatccaggaattagctgagccaacagaccatgtggacagctttggccagagctcccgtgtggcatctgggagccacagtgacccagccacctggctcaggctagttccaaattccaaaagattggcttgtaaaccttcgtctccctctcttttacccagagacagcacatacgtgtgcacacgcatgcacacacacattcagtattttaaaagaatgttttcttggtgccattttcattttattttattttttaattcttggagggggaaataagggaataaggccaaggaagatgtatagctttagctttagcctggcaacctggagaatccacataccttgtgtattgaaccccaggaaaaggaagaggtcgaaccaaccctgcggaaggagcatggtttcaggagtttattttaagactgctgggaaggaaacaggccccattttgtat

SEQ ID NO: 45 - DNA, 478 bp, Artificial sequence
  Probe No. 45
ctgtggccacagcagctttgtacacgaagaccatccatcctcccttcgtccaccactctactccctccaccctccctccctgatcccgtgtgccaccaggagggagtggcagctatagtctggcaccaaagtccaggacacccagtggggtggagtcggagccactggtcctgctgctggctgcctctctgctccaccttgtgacccagggtggggacaggggctggcccagggctgcaatgcagcatgttgccctggcacctgtggccagtactcgggacagactaagggcgcttgtcccatcctggacttttcctctcatgtctttgctgcagaactgaagagactaggcgctggggctcagcttccctcttaagctaagactgatgtcagaggccccatggcgaggccccttggggccactgcctgaggctcacggtacagaggcctgccctgcctggccgggcaggaggttctc

SEQ ID NO: 46 - DNA, 468 bp, Artificial sequence
  Probe No. 46
aaagcatcatttgcacctatgtgggaactttgcctgttgcaaagtattgtggccgagctgcagctgggagcctgctttctgccagtcttgaggttctgaagatcagctttgaaaggaaagtatgtcctagcttagccattcagaagagaaaaatggaatatcagagttacagttgtcagtgaaactactttggattttaacctcttagaggaagaaaaaaggttagggaagtgtcaactctggatgaaggtgatgtgtttgcctctcagtctttcattcatagcctgctagtgaaaaggaagtaaatgagattcttttgtgtgactttgtagtctctttgtattaccaaatagttggggtgttgactcctgtgtgttttgcaagaatgtgtggtaagcctgggtaaagagaaggaactgcggtgttgggagagtctttgtgttggggagtggcaggggatgatttgtt

SEQ ID NO: 47 - DNA, 510 bp, Artificial sequence
  Probe No. 47
actgcccaaggcatgttttgcccaccagatcatggcccacgtggaggcccacctgcctctgtctcactgaactagaagccgagcctagaaactaacacagccatcaagggaatgacttgggcggccttgggaaatcgatgagaaattgaacttcagggagggtggtcattgcctagaggtgctcattcatttaacagagcttccttaggttgatgctggaggcagaatcccggctgtcaaggggtgttcagttaaggggagcaacagaggacatgaaaaattgctatgactaaagcagggacaatttgctgccaaacacccatgcccagctgtatggctgggggctcctcgtatgcatggaacccccagaataaatatgctcagccaccctgtgggccgggcaatccagacagcaggcataaggcaccagttaccctgcatgttggcccagacctcaggtgctagggaaggcgggaaccttgggttgagtaatgctcgtctgtgtgtttt

SEQ ID NO: 48 - DNA, 199 bp, Artificial sequence
  Probe No. 48
agggcactaaggcacagtatctggcagccaaggccctaaagaagcagtcatggcgattccacaccaagtacatgatgtggttccagaggcacgaggagcccaagaccatcactgacgagtttgagcagggcacctacatctactttgactacgagaagtggggccagcggaagaaggaaggcttcacctttgagtaccg

SEQ ID NO: 49 - DNA, 422 bp, Artificial sequence
  Probe No. 49
gaagatggttggcggcattgcccagatcatcgcagcacaggaagaaatgcttcggaaggaacgagagctggaagaggcgcggaagaaactggcccagatccggcagcagcagtacaagtttctgccttcagagcttcgagatgagcactaaagaagcctcttctatttaatgcagacccggcccagagactgtgcgtgccactaccaaagccttctgggctgtcggggcccaacctgcccaaccccagcactccccaaagtgcctgccaaaccccagggcctggccccgcccagtcccgcagtacatcccctgtcccctccccaaccccaagtgccttcatgccctagggccccccaagtgcctgcccctccccagagtattaactctccaagagtattattaacgctgctgtacctcgatc

SEQ ID NO: 50 - DNA, 526 bp, Artificial sequence
  Probe No. 50
aaggactcagagccacacagaacttctgagaggggctgttagcattgcgcagcatcttcagttctccagtaaatgatattgcgttcgtgcctcagctttaagcacaagtagcagcagctcctgcttgagttctgagggcatcatggccctatgattaaccagagtgatctaacctagactaaaattgggaacttatttgcaatttttgaccctgaccactaactagtgattcttctccaaaattgagaaagacagcacccattgaaacagatatgtgtgtgaaagtatatttttcaattccagatttttaattttaaggctccaggaaagaaaggagagtagaacatttttcctcattttatcaaatcctctcttgccctccctcaattcccctgtaacattcctgaagctgttcccactcccagatggttttatcaatagcctagaggtaaagaactgtctttttctctgattctttaataaattatctttatagaatatgcacaagtttttctacactcagtgt

SEQ ID NO: 51 - DNA, 466 bp, Artificial sequence
  Probe No. 51
gaatatcacagcttaccttgggaatactactgacaatttctttaaaatttccaacctgaagatgggtcataattacacgttcaccgtccaagcaagatgcctttttggcaaccagatctgtggggagcctgccatcctgctgtacgatgagctggggtctggtgcagatgcatctgcaacgcaggctgccagatctacggatgttgctgctgtggtggtgcccatcttattcctgatactgctgagcctgggggtggggtttgccatcctgtacacgaagcaccggaggctgcagagcagcttcaccgccttcgccaacagccactacagctccaggctggggtccgcaatcttctcctctggggatgacctgggggaagatgatgaagatgcccctatgataactggattttcagatgacgtccccatggtgatagcctgaaagagctttcctcactagaaacca

SEQ ID NO: 52 - DNA, 541 bp, Artificial sequence
  Probe No. 52
ctatgtgctccagggggacccaagagcagtttccacccagccccaatcccagtctggcaccagcgatcaggtcctttatgggcagctgctgggcagccccacaagcccagggccagggcactatctccgctgtgactccactcagcccctcttggcgggcctcacccccagccccaagtcctatgagaacctctggttccaggccagccccttggggaccctggtaaccccagccccaagccaggaggacgactgtgtctttgggccactgctcaacttccccctcctgcaggggatccgggtccatgggatggaggcgctggggagcttctagggcttcctggggttcccttcttgggcctgcctcttaaaggcctgagctagctggagaagaggggagggtccataagcccatgactaaaaactaccccagcccaggctctcaccatctccagtcaccagcatctccctctcctcccaatctccataggctgggcctcccaggcgatctgcatactttaaggaccagatcatgctccat

SEQ ID NO: 53 - DNA, 437 bp, Artificial sequence
  Probe No. 53
ctaggaggtgcacgggccaccatagtcacactggcactgaaaagaaagcgttgccctggtgattctttcccccccgtttgtaatgttaactgatcaggaagtgcagtttgggtgggatgccgaatcgtcgtgctgacattgagtcacggatgaggaaggtacaagtcctttaagatcaaaactcaaacgggccgttctttctaaggtgtcggtatgtggggagtggtacaaaatggtctgatgctccttcaaaaacattcactttttacaacgtcaaggaattaagcataaaaaagattggttaaaagctttggtttctagtaaaggttagtgtgtgtggtttttttaagaagctgttttgctaaattatttttacttggaatgtttcaaacagatttcaggctgcaaacttgttttataatcgtttgcttctccaa

SEQ ID NO: 54 - DNA, 560 bp, Artificial sequence
  Probe No. 54
caagtatcggggctctgctatcaaggtgcaaagtccttcgtggatgcaacctcaaccatatattctacagcaccctggtgccgtgttaactccctcaatggagcacaccatgtcactacagcccgcatcaatgatcagccctctggcccagcagatgagtcatctgtcactaggcagcaccggaacatacatgcctgcaacgtcagctatgcaaggagcctacttgccacagtatgcacatatgcagacgacagcggttcctgttgaggaggcaagtggtcaacagcaggtggctgtcgagacgtctaatgaccattctccatatacctttcaacctaataagtaactgtgagatgtacagaaaggtgttcttacatgaagaagggtgtgaaggctgaacaatcatggatttttctgatcaattgtgctttaggaaattattgacagttttgcacaggttcttgaaaacgttatttataatgaaatcaactaaaactatttttgctataagttctataaggtgcataaaacccttaaattcatctagtagctgttccccc

SEQ ID NO: 55 - DNA, 420 bp, Artificial sequence
  Probe No. 55
cctctctgacacgcctttaggcgaaacatgccccaagacacagggaccgtttctcccctaggagcagcggtggggagcagggccaaggtcccctgaccactgctcagaggagccctaggccctggccgcagtgccttcagcgcccgacccgggcccccacctggtcagccctggcggggcccactcaggacagctgggggccggggcgtggcagggccctctctgtgcctctcctcctaagtaggaaggggctccgggtggctgctctgggactgggcacccacaagggctcagtgggcccaaacccttgaaatccgtgaaaccgggtggtcccaagagctagaaactcaggaaaccccaggtgctcagggccccgcgtctcgggggctccgtggggcagacccctgctaatatatgcaa

SEQ ID NO: 56 - DNA, 507 bp, Artificial sequence
  Probe No. 56
cgccatcacccgcaagagagaagctgaccaggccggccgcgtcccctgctgtgggcgagaagctgaaaaagaccgaacctgccgctggtaaagagacaggagctgccaaacccaagctgacgcctcaggagaagctgaaactgaggatgcagaaggcgctgaacaggcagttcaaggcggataagaaggcggcacaagaaaagatgatccagcaggagcatgagcggcaggagcgggaagacgagcttcgagccatggcccgcaagatccgcatgaaggagcgggaacgccgagagaaggagagagaagagtgggaacgccagtacagccggcagagccgctcaccctccccccgatacagtcgagaatacagctcttctcgaaggcgctcaaggtcccgatcccgaagcccccattaccgacattaggcagaagagtggggggtggggaggacaagggggtgggtaaggggctcaagctgtgatgctgctggttttatctctagtg

SEQ ID NO: 57 - DNA, 553 bp, Artificial sequence
  Probe No. 57
gatctacttgtgacttgttggccttcttcccacatctgcctcagactggggggggctcagctcctcgggtgatatctagcctgcttgtgagctctagcagggataaggagagctgagattggagggaattgtgttgctcctggaggaagcccaggcatcattaaacaagccagtaggtcacctggcttccgtggaccaattcatctttcagacaagctttagagaaatggactcagggaagagactcacatgctttggttagtatctgtgtttccggtgggtgtaataggggattagccccagaagggactgagctaaacagtgttattatgggaaaggaaatggcattgctgctttcaaccagcgactaatgcaatccattcctctcttgtttatagtaatctaagggttgagcagttaaaacggcttcaggatagaaagctgtttcccacctgtttcgttttaccattaaaagggaaacgtgcctctgccccacgggtagagggggtgcacgttcctcctggttccttcgcttgtgtttctgtacttacca

SEQ ID NO: 58 - DNA, 528 bp, Artificial sequence
  Probe No. 58
gccaaagattcggaacaccagccagcttgaccaccagagaccccgaggcgaaagtgggatttctgaaacctgtaggccccaagcccatcaacttgcccaaagaagattccaaacctacatttccctggcctcctggaaacaagccatctcttcacagtgtaaaccaagaccatgacttaaagccactaggcccgaaatctgggcctactcctccaacctcagaaaatgaacagaagcaagcgtttcccaaattgactggggttaaagggaaatttatgtcagcatcacaagatcttgaacccaagcccctcttccccaaacccgcctttggccagaagccgcccctaagtaccgagaactcccatgaagacgaaagccccatgaagaatgtgtcttcatcaaaagggtccccagctcccctgggagtcaggtccaaaagcggccctttaaaaccagcaagggaagactcagaaaataaagaccatgcaggggagatttcaagtttgccctttcctggagtggttttga

SEQ ID NO: 59 - DNA, 486 bp, Artificial sequence
  Probe No. 59
agcctgggaccctaacatttggagtgcaggaaaaacatatgaacaattaaaagaagagctgggagatgctgctgctatcatgcgaatcactgctgacatgaagaatgcaaccctctccctgaattctaatgacagtgagccaaaatattaccctatagcagttctgttgaaaaaccagaatcaggagctgcctgaggatgtaaaccctgccaaaaaggagaattacctctctgaacaggactttgtgtctgtgtttggcatcacaagagggcaatttgcagctctgcctggctggaaacagctccaaatgaagaaagaaaaggggcttttctaaagcaagaaggcctatacctattgcaaggccacagaaaagagcagatagtgccaatatcaggaaataatttatccaccaatttctgcctgacattcagctacttaatttagatataatagagtctgcaaatcacggcatgttctccatttttt

SEQ ID NO: 60 - DNA, 514 bp, Artificial sequence
  Probe No. 60
ataggaccaggtttacagagctttatatttgcactaggattttttttttttgtaattgtcacagaaaatgtaattgtgggtgtgtgtgcgtgcgtgtgtgtgtgtgtgtgtatcgtgtgtgtgtgttttgttttgatttgggggatattttgtacaaaaagaaaacccacgggaagatgtccgtggagaggcagagctttcatactgaattagatgtattttatgggaatttggtaaatttttctttgtattttttttttttacatataagtatatatacacttagagattgtcatatacttttaccacttgaattgatcttcttgccagcaatagatctcattttcaaaagcaattcttcggtctgtgtagctggcagaaagttctgtccagtaaacgcaggatggaattttcctgggactctacacccatcttaaggtggtataccttccaaatcctggttcagatggaagaaatagcaggagagaggacccattagctggcagacccaggg

SEQ ID NO: 61 - DNA, 534 bp, Artificial sequence
  Probe No. 61
ggagaacgcacacctggtggtcatcaactcctgggaggagcagaaattcattgtacaacacacgaaccccttcaatacctggataggtctcacggacagtgatggctcttggaaatgggtggatggcacagactataggcacaactacaagaactgggctgtcactcagccagataattggcacgggcacgagctgggtggaagtgaagactgtgttgaagtccagccggatggccgctggaacgatgacttctgcctgcaggtgtaccgctgggtgtgtgagaaaaggcggaatgccaccggcgaggtggcctgaccccagcacacctctggctaacccataccccacacctgcccagctctggcttctctgttgaggattttgaggaaaggaagaaacactgagacaggggtatggggaagagctgagcaaagagagaaaggaggtagtttaagagtccctgaccctggaggactgagatcccacctccttctgtaattcattgtaattattataatcgtcagcctcttcaa

SEQ ID NO: 62 - DNA, 527 bp, Artificial sequence
  Probe no. 62
ggccttgacacattacaagcctggaaaaaaacatcagaaataataaaaaatttcagagagaatcaagatacctttttttttcttttttttttcttttttttattatactctaagttttagggtacatgtgcacattgtgcaggttagttacatatgtatacatgtgccatgctggtgcgctgcacccactaatgtgtcatctagcattaggtatatctcccagtgctatccctcccccctcccccgaccccaccacagtccccagagtgtgatattccccttcctgtgtccatgtgatctcattgttcaattcccacctatgagtgagaatatgcggtgtttggttttttgttcttgcgatagtttactgagaatgatggtttccaatttcatccatgtccctacaaaggatatgaactcatcattttttatggctgcatagtattccatggtgtatatgtgccacattttcttaatccagtctatcattgttggacatttgggttggttccaagtctttgctattg

SEQ ID NO: 63 - DNA, 370 bp, Artificial sequence
  Probe No. 63
atgcagtagctgccatgtctaaagataaaaataacatgcaacatcgatatattgaactcttcttgaattctactcctggaggcggctctggcatgggaggttctggaatgggaggctacggaagagatggaatggataatcagggaggctatggatcagttggaagaatgggaatggggaacaattacagtggaggatatggtactcctgatggtttgggtggttatggccgtggtggtggaggcagtggaggttactatgggcaaggcggcatgagtggaggtggatggcgtgggatgtactgaaagcaaaaacaccaacatacaagtcttgacaacagcatctggtctactagactttcttacagatt

SEQ ID NO: 64 - DNA, 334 bp, Artificial sequence
  Probe No. 64
caagtatcggggctctgctatcaaggtgcaaagtccttcgtggatgcaacctcaaccatatattctacagcaccctggtgccgtgttaactccctcaatggagcacaccatgtcactacagcccgcatcaatgatcagccctctggcccagcagatgagtcatctgtcactaggcagcaccggaacatacatgcctgcaacgtcagctatgcaaggagcctacttgccacagtatgcacatatgcagacgacagcggttcctgttgaggaggcaagtggtcaacagcaggtggctgtcgagacgtctaatgaccattctccatatacctttcaa

SEQ ID NO: 65 - DNA, 434 bp, Artificial sequence
  Probe No. 65
gccattacgaactctattttgcccatcacaatctctttcatgaaggatctaataacttaatcctcatcttactggaacccattccacagaacagcattcccaacaagtaccacaagctgaaggctctcatgacgcagcggacttatttgcagtggcccaaggagaaaagcaaacgtgggctcttttgggctaacattagagccgcttttaatatgaaattaacactagtcactgaaaacaatgatgtgaaatcttaaaaaaatttaggaaattcaacttaagaaaccattatttacttggatgatggtgaatagtacagtcgtaagtnactgtctggaggtgcctccattatcctcatgccttcaggaaagacttaacaaaaacaatgtttcatctggggaactgagctaggcggtgaggttagcctgccagtt

SEQ ID NO: 66 - DNA, 470 bp, Artificial sequence
  Probe No. 66
gctctaaggaccgtcctgcgagatcgccttccaaccccacttttttctggaaaggaggggtcctgcaggggcaagcaggagctagcagccgcctacttggtgctaacccctcgatgtacatagcttttctcagctgcctgcgcgccgccgacagtcagcgctgtgcgcgcggagagaggtgcgccgtgggctcaagagcctgagtgggtggtttgcgaggatgagggacgctatgcctcatgcccgttttgggtgtcctcaccagcaaggctgctcgggggcccctggttcgtccctgagcctttttcacagtgcataagcagttttttttgtttttgttttgttttgttttgtttttaaatcaatcatgttacactaatagaaacttggcactcctgtgccctctgcctggacaagcacatagcaagctgaactgtcctaaggcaggggcgagcacggaacaatggggc

SEQ ID NO: 67 - DNA, 401 bp, Artificial sequence
  Probe No. 67
ggagtcacaagacactgttgcagagaatgatgatggcgggttcagtgaggaatgggaagcccagagggacagtcatctagggcctcatcgctctacacctgagtcacgagctgctgtccaggaactttccagcagtatcctcgctggtgaagacccagaggaaaggggagtaaaacttggattgggagatttcattttctacagtgttctggttggtaaagcctcagcaacagccagtggagactggaacacaaccatagcctgtttcgtagccatattaattggtttgtgccttacattattactccttgccattttcaagaaagcattgccagctcttccaatctccatcacctttgggcttgttttctactttgccacagattatcttgtacagcctt

SEQ ID NO: 68 - DNA, 471 bp, Artificial sequence
  Probe No. 68
gctgatggagatcgtcacctacggccggatcccttacccagggatgtcaaaccctgaagtgatccgagctctggagcgtggataccggatgcctcgcccagagaactgcccagaggagctctacaacatcatgatgcgctgctggaaaaaccgtccggaggagcggccgaccttcgaatacatccagagtgtgctggatgacttctacacggccacagagagccagtaccaacagcagccatgatagggaggaccagggcagggcagggggtgcccaggtggtggctcgaaggtggctccagcaccatccgccagggcccacacccccttcctactcccagacacccaccctcgcttcagccacagtttcctcatctgtccagtgggtaggttggactggaaaatctctttttgactcttgcaatccacaatctgacattctcaggaagcccccaagttgatatttctatt

SEQ ID NO: 69 - DNA, 556 bp, Artificial sequence
  Probe No. 69
tgaaaagtctcccatgtctacttctttctacacagacacggcaaccatccgatttctcaatcttttccccacctttcccccttttctattccacaaaaccgccattgtcatcatggcccgttctcaatgagctgttgggtacacctcccagacggggtggtggccgggcagaggggctcctcacttcccagtaggggcggccgggcagaggcgcccctcacctcctggacggggcggctggccgggcggggggctgacccccctacctccctcccagacagggcggctggccaggcagaggggctcctcacctcccagacggggcggcggggcagaggcgctcccatctcagacgatgggcggccgggcagagacgctcctcacttcctagatgggatggcggccgggcagagacactcctcactttccagactgggcagccaggcagaggggctcctcacatcccagacgatgggcggccaggcagagacgctcctcacttcccagacggggtagcggccgggcagaggctgcaatctcggcactttggggggcc

SEQ ID NO: 70 - DNA, 539 bp, Artificial sequence
  Probe No. 70
gaggacacgtatctctatatcttcctgggatgacaatcagctacatttgtgaccccggctacctgttagtgggaaagggcttcattttctgtacagaccagggaatctggagccaattggatcattattgcaaagaagtaaattgtagcttcccactgtttatgaatggaatctcgaaggagttagaaatgaaaaaagtatatcactatggagattatgtgactttgaagtgtgaagatgggtatactctggaaggcagtccctggagccagtgccaggcggatgacagatgggaccctcctctggccaaatgtacctctcgtgcacatgatgctctcatagttggcactttatctggtacgatcttctttattttactcatcattttcctctcttggataattctaaagcacagaaaaggcaataatgcacatgaaaaccctaaagaagtggctatccatttacattctcaaggaggcagcagcgttcatccccgaactctgcaaacaaatgaagaaaatagcagggtccttccttga

SEQ ID NO: 71 - DNA, 542 bp, Artificial sequence
  Probe No. 71
gtagcagtgctctcattggcctgctgntcatcgcagtggccattgccacggtcatcgtcatcagcctggtgatgctgaggaagaggcagtatggcaccatcagccacgggatcgtggaggttgatccaatgctcaccccagaagagcgtcacctgaacaagatgcagaaccatggctatgagaaccccacctacaaatacctggagcagatgcagatttaggtggcagggagcgcggcagccctggcggagggatgcaggtgggccggaagatcccacgattccgatcgactgccaagcagcagccgctgccaggggctgcgtctgacatcctgacctcctggactgtaggactatataaagtactactgtagaactgcaatttccattcttttaaatgggtgaaaaatggtaatataacaatatatgatatataaaccttaaatgaaaaaaatgatctattgcagatatttgatgtagttttcttttttaaattaatcagaaaccccacttccattgtattgtctgacacatgctctcaat

SEQ ID NO: 72 - DNA, 344 bp, Artificial sequence
  Probe No. 72
tcttacccccttgatggaagcagcttctggagggtatgcagaggttggaagagttcttcttgataaaggagcagatgttaatgctccccctgngccttcctcaagagatactgctttaacnatagcagcagacaaaggtcactacaaattttgtgaactcctgattcataggggagncccacattgatgttcgtaacaaaaagggaaatacgccactttggctggcatccaatggaggtcattttgatgttgtgcagttgctagtgcaagcaggtgctgatgtggatgcagcagataaccggaaaatcacacctcttatgtcagcatttcgcaagggtcatgta

SEQ ID NO: 73 - DNA, 321 bp, Artificial sequence
  Probe No. 73
ggacccattttcatttgacttctttgaagacccttttgaggacttctttgggaatcgaaggggtccccgaggaagcagaagccgagggacggggtcgtttttctctgcgttcagtggatttccgtcttttggaagtggattttcttcttttgatacaggatttacttcatttgggtcactaggtcacgggggcctcacttcattctcttccacgtcatttggtggtagtggcatgggcaacttcaaatcgatatcaacttcaactaaatggttaatggcagaaaaatcactacaaagagaattgtcgagaacggtcaagaa

SEQ ID NO: 74 - DNA, 514 bp, Artificial sequence
  Probe No. 74
gaagtaagcctcatcatcagagcctttcctcaaaactggagtcccaaatgtcatcaggttttgttttttttcagccactaagaacccctctgcttttaactctagaatttgggcttggaccagatctaacatcttgaatactctgccctctagagccttcagccttaatggaaggttggatccaaggaggtgtaatggaatcggaatcaagccactcggcaggcatggagctataactaagcatccttagggttctgcctctccaggcattagccctcacattagatctagttactgtggtatggctaatacctgtcaacatttggaggcaatcctaccttgcttttgcttctagagcttagcatatctgattgttgtcaggccatattatcaatgtttacttttttggtactataaaagctttctgccacccctaaactccaggggggacaatatgtgccaatcaatagcacccctactcacatacacacacacctagccagctgtcaagggc

SEQ ID NO: 75 - DNA, 512 bp, Artificial sequence
  Probe No. 75
gagtaactgctctctgagttttgcacacgaagttgccctcatctgctggagatcgataaggaaggcacaagacgttctcctctgcccgtgaggagcttcccgcagccgcctggcccagcctgggcacgttctccgaggcatgtgtctccctgctcaccctcgtctgggcacctcagcatctgtggacttgagcgtccaaaaaccctgagtgtgattctgggcagccggcctggcttgaagtccgccatgaccctgggcacaggggaagcccagccgtgggcttaggagagagggaccagcgcccagcgttagggctggaagacggcagtgttcagaattccagccgctcatctgaacacagaaggtgtgaactgacctctaaagcagcgtgagatgggaatgatctagaaaactttggatttttgaagtaaattttaatgtttcatattaatttcttgaaaatgtattaaatgtcattgaaagccttattacgcttttcagatcctttcaat

SEQ ID NO: 76 - DNA, 501 bp, Artificial sequence
  Probe No. 76
agatccaaagagccttcgctatgcctgcacccacgccttcctccagcccggtgcccaccctctctccagagcagcaggaaatgttgcaagcattctctacccagtctggcatgaacctcgagtggtcccagaagtgccttcaggacaacaactgggactacaccagatctgcccaggccttcactcatctcaaggccaagggcgagatcccagaagtggcattcatgaagtgatcgtagtcatgcctcagaagcagtcccccctgtaaatagtccttggatattaccgtctggttgtcgtctgtcatctcctcctgtctggcccgaggccgccccgtgactgtgaccgagggagggagggctgcctgatccctctcctcgcctgccttctggaagacttcagaagattgagcctcactggtgccaggaagccaaagcttactttgtagaactgacactaaactacccgaaggacttaggtgctttgtgtacttaaccccag

SEQ ID NO: 77 - DNA, 529 bp, Artificial sequence
  Probe No. 77
gaaacacattcctggtttttgcctacacttacgtgttagacaagaactatgatttttttttttaaagtactggtgtcaccctttgcctatatggtagagcaataatgctttttaaaaataaacttctgaaaacccaaggccaggtactgcattctgaatcagaatctcgcagtgtttctgtgaatagatttttttgtaaatatgacctttaagatattgtattatgtaaaatatgtatatacctttttttgtaggtcacaacaactcatttttacagagtttgtgaagctaaatatttaacattgttgatttcagtaagctgtgtggtgaggctaccagtggaagagacatcccttgacttttgtggcctgggggaggggtagtgctccacagcttttccttccccaccccccagccttagatgcctcgctcttttcaatctcttaatctaaatgctttttaaagagattatttgtttagatgtaggcattttaattttttaaaaattcctctaccagaactaagcact

SEQ ID NO: 78 - DNA, 514 bp, Artificial sequence
  Probe No. 78
tatctctccatgttcagttccaaggagtcccagcggggcatgggctacatgcccaaacgtggcctggaggtgaacaagtgtgagatcgccaggttctacaagctgcacgagcggaggtgtgagcccattgccatgacagtgcctcgaaagtcggacctgttccaggaggacctgtacccacccaccgcagggcccgaccctgccctcacggctgaggagtggctggggggtcgggatgctgggcccctcctcatctccctcaaggatggctacgtacccccaaagagccgggagctgagggtcaaccggggcctggacaccgggcgcaggagggcagcaccagaggccagtggcactcccagctcggatgccgtgtctcggctggaggaggagatgcggaagctccaggccacggtgcaggagctccagaagcgcttggacaggctggaggagacagtccaggccaagtagagccccgcagggcctccagcagggtcagacattcacacccatc

SEQ ID NO: 79 - DNA, 455 bp, Artificial sequence
  Probe No. 79
tgaacaccttctacagcaaactcttgcaagtccagtttcatccctgtaaggcaaatgtcttttcacgcagaaagtgccatatagacgagataaaggcagctaaaacgagggcagtagagagcacttacccgaccccaaggtgccagagatgccctgaggatggtggttaaggaaacaggagcaggaaatgtacacacagattcctgtccctttgccaactactccttccccatcaaagaaaaacacttgcacacagtaactaccagctccttctctcaaacttgtatttctcctggaaatgtatctcagaaatgacctcctctcccaaccacttcaacgattctttctttgggtttggggttcttgcagttctatcatctaaaataacctttggactgcaggtaaaatgcaattaggacaactaaccaagtagacgaaacaagttcccctaggca

SEQ ID NO: 80 - DNA, 352 bp, Artificial sequence
  Probe No. 80
ggaatgacacttacttacgacccaactacagctgctatacagaacggattttatccttcaccatacagtattgctacaaaccgaatgatcactcaaacttctattacaccctatattgcatctcctgtatctgcctaccaggtgcaaagtccttcgtggatgcaacctcaaccatatattctacagcaccctggtgccgtgttaactccctcaatggagcacaccatgtcactacagcccgcatcaatgatcagccctctggcccagcagatgagtcatctgtcactaggcagcaccggaacatacatgcctgcaacgtcagctatgcaaggagcctacttgccacagtatg

SEQ ID NO: 81 - DNA, 536 bp, Artificial sequence
  Probe No. 81
cagttctgaatatgctgctcatccccacctgtcttcaacagctccccattaccctcaggacaatgtctgaactctccagcttcgcgtgagaagtccccttccatcccagagggtgggcttcagggcgcacagcatgagagcctctgtgcccccatcaccctcgtttccagtgaattagtgtcatgtcagcatcagctcagggcttcatcgtggggctctcagttccgattccccaggctgaattgggagtgagatgcctgcatgctgggttctgcacagctggcctcccgcggttgggtcaacattgctggcctggaagggaggagcgccctctagggagggacatggccccggtgcggctgcagctcaccagccccaggggcagaagagacccaaccacttcctattttttgaggctatgaatatagtacctgaaaaaatgccaagcactagattatttttttaaaaagcgtactttaaatgtttgtgttaatacacattaaaacatcgcacaaaaacgatgcatctaccgctcc

SEQ ID NO: 82 - DNA, 499 bp, Artificial sequence
  Probe No. 82
gcatttcgcgggcgcagtacaacgcgctcaccctcaccttccagcgcgccatgcacgactacaaccaggccgagatgaagcagcgcgacaactgcaagatccgcatccagcgccagctggagatcatgggcaaggaagtctcgggcgaccagatcgaggacatgttcgagcagggtaagtgggacgtgttttccgagaacttgctggccgacgtgaagggcgcgcgggccgccctcaacgagatcgagagccgccaccgcgaactgctgcgcctggagagccgcatccgcgacgtacacgagctcttcttgcagatggcggtgctggtggagaagcaggccgacaccctgaacgtcatcgagctcaacgtacaaaagacggtcgactacaccggccaggccaaggcgcaggtgcggaaggccgtgcagtacgaggagaagaacccctgccggaccctctgctgcttctgctgtccctgcctcaagtagcaggccggccc

SEQ ID NO: 83 - DNA, 199 bp, Artificial sequence
  Probe No. 83
aaaactcttcagccatattacttctgccaggaaagctaggaaatatgaaatacctcatttcagacttaagaaggtggagaatattaaaatatggttatcactgcgttcctatctaaagagacgggggccacagcgttcagttgatgtggttgtatcctcggttttcctactgacactttcgattgctttcatttgttgt

SEQ ID NO: 84 - DNA, 260 bp, Artificial sequence
  Probe No. 84
agacggggacacggcagggatgcctggccctggtcacctgcggccgggcatgtccgggcaggacgaactcgccgtcggagtcaggggaagaactgggtccccgggctgggcaggagggacccggccgcgagggagcagagaggcggtccccctggctgccccgagcccgcgaagggagggaagttccagaatcgagagagggagggagtcaaggtggaacccatagagtgagcctcctgaagacacagagcggttgcctc

SEQ ID NO: 85 - DNA, 177 bp, Artificial sequence
  Probe No. 85
gggagtttgaccagagatgcaaggggtgaaggagcgcttcctaccgttagggaactctggggacagagcgccccggccgcctgatggccgaggcagggtgcgacccaggacccaggacggcgtcgggaaccataccatggcccggatccccaagaccctaaagttcgtcgtcgtcat

SEQ ID NO: 86 - DNA, 503 bp, Artificial sequence
  probe No. 86
tggaccgcagcggacactgcggctcagatctccaagcgcaagtgtgaggcggccaatgtggctgaacaaaggagagcctacctggagggcacgtgcgtggagtggctccacagatacctggagaacgggaaggagatgctgcagcgcgcggacccccccaagacacacgtgacccaccaccctgtctttgactatgaggccaccctgaggtgctgggccctgggcttctaccctgcggagatcatactgacctggcagcgggatggggaggaccagacccaggacgtggagctcgtggagaccaggcctgcaggggatggaaccttccagaagtgggcagctgtggtggtgccttctggagaggagcagagatacacgtgccatgtgcagcatgaggggctgccggagcccctcatgctgagatggaagcagtcttccctgcccaccatccccatcatgggtatcgttgctggcctggttgtccttgcagctgtagtcactgg

SEQ ID NO: 87 - DNA, 517 bp, Artificial sequence
  Probe No. 87
gagtgaggcgatttgacctgctcaaacgtatcttgaagatggacagaaaagctgtggagacccacctgctcaggaaccctcaccttgtttcggactatagagtgctgatggcagagattggtgaggatttggataaatctgatgtgtcctcattaattttcctcatgaaggattacatgggccgaggcaagataagcaaggagaagagtttcttggaccttgtggttgagttggagaaactaaatctggttgccccagatcaactggatttattagaaaaatgcctaaagaacatccacagaatagacctgaagacaaaaatccagaagtacaagcagtctgttcaaggagcagggacaagttacaggaatgttctccaagcagcaatccaaaagagtctcaaggatccttcaaataacttcaggatgataacaccctatgcccattgtcctgatctgaaaattcttggaaattgttccatgtgattaacatggaactgcctctacttaatcattct

SEQ ID NO: 88 - DNA, 473 bp, Artificial sequence
  Probe No. 88
cggctctcagcaggtctacctgaacgtctccctgcagagcaaagccacatcaggagtgactcagggggtggtcgggggagctggagccacagccctggtcttcctgtccttctgcgtcatcttcgttgtagtgaggtcctgcaggaagaaatcggcaaggccagcagcgggcgtgggagatacgggcatagaggatgcaaacgctgtcaggggttcagcctctcaggggcccctgactgaaccttgggcagaagacagtcccccagaccagcctcccccagcttctgcccgctcctcagtgggtgaaggagagctccagtatgcatccctcagcttccagatggtgaagccttgggactcgcggggacaggaggccactgacaccgagtactcggagatcaagatccacagatgagaaactgcagagactcaccctgattgagggatcacagcccctccaggcaagggagaag

SEQ ID NO: 89 - DNA, 525 bp, Artificial sequence
  Probe No. 89
tggacaatacctggctttcctaggcagaggtccctgcggccttccgcagtttttgtgtccctgggtacttgagattagggagtggtgatgactcttaaggagcatgctgccttcaagcatctgtttaacaaagcacatcttgcaccgcccttaatccattcaactctgtgacacagcacatgtttcagagagcacggggttgggggtaaggttatagattaacagaatctcaaggcagaagaatttttcttagtacagaacaaaatggagtctcctatgtctacttctttctacacagacacagtaacaatctgatctctcttgcttttccccacaggttgtcattacttaattatttaagattcaaaaatttaaaaattccacattgaagttgtgtgcagtggctcactactgtagtcccagttactagggaagctgaggcaggaagactgcttgagtccaggagatcgaggctccagtgagccatgatcacaccactaccctccagcctgagctacagagaga

SEQ ID NO: 90 - DNA, 539 bp, Artificial sequence
  Probe No. 90
aagggcagtttgctggatttcctgaagagcgatgaaggtggcaaagtgctgcttccaaagctcattgacttttctgctcagattgcagagggaatggcatacatcgagcggaagaactacattcaccgggacctgcgagcagctaatgttctggtctccgagtcactaatgtgcaaaattgcagattttggccttgctagagtaattgaagataatgagtacacagcaagggaaggtgctaagttccctattaagtggacggctccagaagcaatcaactttggatgtttcactattaagtctgatgtgtggtcctttggaatcctcctatacgaaattgtcacctatgggaaaattccctacccagggagaactaatgccgacgtgatgaccgccctgtcccagggctacaggatgccccgtgtggagaactgcccagatgagctctatgacattatgaaaatgtgctggaaagaaaaggcagaagagagaccaacgtttgactacttacagagcgtcctggatgatttctacacagc

SEQ ID NO: 91 - DNA, 493 bp, Artificial sequence
  Probe No. 90
ttcagaaccacgtcagatataccaagtgactgtgtgtggggtttgacaactgtggaaaggcgagcagaaaactccggcggtctgaggccatggaggtggttgctgcatttgagagggagtagggggctagatgtggctcctagtgcaaaccggaaaccatggcaccttccagagccgtggtctcaaggagtcagagcagggagctttgatgcaacttatttgtaagaaggatttttaaattttttatgggtagaattgtagtcaggaaaacagaaagggcttgaaatttaataagtgctgctggaggggattttccaagcctggaagggtattcagcagctgtggtggggaaagatttctcctgaaagactgaacgtgtttcttcatgacagctgctcaaagcaggtttctgagatagctgaccgagctctggtaaatctctttgtcaaattacgaaaacttcagggcgaaatcctatgcttccatgtacatt

SEQ ID NO: 92 - DNA, 489 bp, Artificial sequence
  Probe No. 92
ggacaagcctctggtcaaggtcacattcttccagaatggaaaatccaagaaattttcccgttcggatcccaacttctccatcccacaagcaaaccacagtcacagtggtgattaccactgcacaggaaacataggctacacgctgtactcatccaagcctgtgaccatcactgtccaagctcccagctcttcaccgatggggatcattgtggctgtggtcactgggattgctgtagcggccattgttgctgctgtagtggccttgatctactgcaggaaaaagcggatttcagccaattccactgatcctgtgaaggctgcccaatttgagatgctttcctgcagccacctggacgtcaaatgattgccatcagaaagagacaacctgaagaaaccaacaatgactatgaaacagctgacggcggctacatgactctgaaccccagggcacctactgacgatgataaaaacatctacctgactcttcct

SEQ ID NO: 93 - DNA, 511 bp, Artificial sequence
  Probe No. 93
atgtcaatgctgccattgccaccatcaaaaccaagcgcagcatccagtttgtggattggtgccccactggcttcaaggttggcatcaactaccagcctcccactgtggtgcctggtggagacctggccaaggtacagagagctgtgtgcatgctgagcaacaccacagccattgctgaggcctgggctcgcctggaccacaagtttgacctgatgtatgccaagcgtgcctttgttcactggtacgtgggtgaggggatggaggaaggcgagttttcagaggcccgtgaagatatggctgcccttgagaaggattatgaggaggttggtgtggattctgttgaaggagagggtgaggaagaaggagaggaatactaattatccattccttttggccctgcagcatgtcatgctcccagaatttcagcttcagcttaactgacagacgttaaagctttctggttagattgttttcacttggtgatcatgtcttttccatgtgtacctgtaat

SEQ ID NO: 94 - DNA, 481 bp, Artificial sequence
  Probe No. 94
ccaagcgcagcatccagtttgtggattggtgccccactggcttcaaggttggcatcaactaccagcctcccactgtggtgcctggtggagacctggccaaggtacagagagctgtgtgcatgctgagcaacaccacagccattgctgaggcctgggctcgcctggaccacaagtttgacctgatgtatgccaagcgtgcctttgttcactggtacgtgggtgaggggatggaggaaggcgagttttcagaggcccgtgaagatatggctgcccttgagaaggattatgaggaggttggtgtggattctgttgaaggagagggtgaggaagaaggagaggaatactaattatccattccttttggccctgcagcatgtcatgctcccagaatttcagcttcagcttaactgacagacgttaaagctttctggttagattgttttcacttggtgatcatgtcttttccatgtgtacctgtaat

SEQ ID NO: 95 - DNA, 463 bp, Artificial sequence
  Probe No. 95
gatcccggtgcagctgaatgccggccagctgcagtatatccgcttagcccagcctgtatcaggcactcaagttgtgcagggacagatccagacacttgccaccaatgctcaacagattacacagacagaggtccagcaaggacagcagcagttcagccagttcacagatggacagcagctctaccagatccagcaagtcaccatgcctgcgggccaggacctcgcccagcccatgttcatccagtcagccaaccagccctccgacgggcaggccccccaggtgaccggcgactgagggcctgagctggcaaggccaaggacacccaacacaatttttgccatacagccccaggcaatggcacagccttcctccccagaggacccggccgacctcagcgcctcctgcaggctaggacactggtgcactacaccccatgcctggggccgagattctccagcagaa

SEQ ID NO: 96 - DNA, 535 bp, Artificial sequence
  Probe No. 96
gtgcatctgactgtgctttctgagtggctggtgctccagacccctcacctggagttccaggagggagaaaccatcgtgctgaggtgccacagctggaaggacaagcctctggtcaaggtcatattcttccagaatggaaaatccaagaaattttcccgttcggatcccaacttctccatcccacaagcaaaccacagtcacagtggtgattaccactgcacaggaaacataggctacacgctgtactcatccaagcctgtgaccatcactgtccaagctcccagctcttcaccgatggggatcattgtggctgtggtcactgggattgctgtagcggccattgttgctgctgtagtggccttgatctactgcaggaaaaagcggatttcagccacctggacgtcaaatgattgccatcagaaagagacaacctgaagaaaccaacaatgactatgaaacagctgacggcggctacatgactctgaaccccagggcacctactgacgatgataaaaacatctacctgactcttcct

SEQ ID NO: 97 - DNA, 461 bp, Artificial sequence
  Probe No. 97
gtgtttgtgtccctgggtacttgagattagggagtggtgatgactcttaacgagcatgctgccttcaagcatctgtttaacaaagcacatcttgcaccacccttaatccattcaaccctgaggggacacagcacatgtttcagagagcacagggttgggggtaaggtcacagatcaacaggatcccaaggcagaagaatttttcttagtacagaacaaaatgaaaagtctcccatgtctacctctttctacacagacatggcaaccatccgatttctcaatcttttccccacctttcccccctttctattccacaaaaccgccattgtcatcatggcccgttctcaatgagctgttgagtacacctcccagacggggtggtggccgggcagatgggctcctcacttcccagtaggggcggctgggcagaggtgcccctcacctcctggacagggtggctgg

SEQ ID NO: 98 - DNA, 514 bp, Artificial sequence
  Probe No. 98
gcagtgagattcctggaggtggcacccagtgcactcttttgggccacagacatcattgctgttccccgttacctcgagctgactctagaggggaaggcagagctcaggagggtgggtgggagctgcagtgggctcagagtccagcaatgaggccccctggcctgggcacccagctgcaggcccctgccctacgtgcactacaggaaggggtgaggagagcagccagaggaaaaccagccccagaatgctgcaacttctcttctctctggacttcggtgtcctcggctctgaaggcgctctctgcacttagatgctgtcagccctcaacgtaggaggggccgtggggtccctaagtgattcttctccctggcaaggctcttccttttcagggatatctctgccaacccctcccctgtcctcgggttgggcttggccctctctgcctgagagagctcagcacacacacagctcagacccacggacaggaccccgggacagaaccccgggagcactg

SEQ ID NO: 99 - DNA, 561 bp, Artificial sequence
  Probe No. 99
acaagcatcctgtctcacgaagaacaaatgtttgttaatcgtgtgggccatgattatcagtggataggcctcaatgacaagatgtttgagcatgacttccgttggactgatggcagcacactgcaatacgagaattggagacccaaccagccagacagcttcttttctgctggagaagactgtgttgtaatcatttggcatgagaatggccagtggaatgatgttccctgcaattaccatctcacctatacgtgcaagaaaggaacagttgcttgcggccagccccctgttgtagaaaatgccaagacctttggaaagatgaaacctcgttatgaaatcaactccctgattagataccactgcaaagatggtttcattcaacgtcaccttccaactatccggtgcttaggaaatggaagatgggctatacctaaaattacctgcatgaacccatctgcataccaaaggacttattctatgaaatactttaaaaattcctcatcagcaaaggacaattcaataaatacatccaaacatgatcatcgttggagccggaggtgg

SEQ ID NO: 100 - DNA, 483 bp, Artificial sequence
  Probe No. 100
aaatgtgaccctcgccatggtaaatacatggcttgctgcctgttataccgtggtgacgtggttcccaaagatgtcaatgctgccattgccaccatcaaaaccaagcgtaccatccagtttgtggattggtgccccactggcttcaaggttggcattaattaccagcctcccactgtggtgcctggcggagacctggccaaggtacagagagctgtgtgcatgctgagcaataccacagctgttgccgaggcctgggctcgcctggaccacaagtttgacctgatgtatgccaagcgtgcctttgttcactggtacgtgggtgaggggatggaggaaggcgagttttcagaggcccgtgaggacatggctgcccttgagaaggattatgaggaggttggagcagatagtgctgacggagaggatgagggtgaagagtattaacctgtgtgctgtacttttacactcctttgtcttggaactgtc

SEQ ID NO: 101 - DNA, 415 bp, Artificial sequence
  Probe No. 101
tgtgacttgtatgaaaccctgaccatcacccaggcagtcatcttcatcaacacccggaggaaggtggactggctcaccgagaagatgcatgctcgagatttcactgtatccgccatgcatggagatatggaccaaaaggaacgagacgtgattatgagggagtttcgttctggctctagcagagttttgattaccactgacctgctgggaaaactatatccacagaatcggtcgaggtggacggtttggccgtaaaggtgtggctattaacatggtgacagaagaagacaagaggactcttcgagacattgagaccttctacaacacctccattgaggaaatgcccctcaatgttgctgacctcatctgaggggctgtcctgccacccagccccagccagggctcaatctctggg

SEQ ID NO: 102 - DNA, 152 bp, Artificial sequence
  Probe No. 102
atggctccacactacaggttcaagagaagagtaatacgtggtcctgggggattttgaagatgttaaagggaaaagatgacagaaagaaaagtatacgagagaaacctaaagtctctgactcagacaataatgaaggttcatctttccctgct

SEQ ID NO: 103 - DNA, 441 bp, Artificial sequence
  Probe No. 103
tccctgctcctcctaaacaattggacatgggagatgaagtttacgatgatgtggatacctctgatttccctgtttcatcagcagagatgagtcaaggaactaattttggaaaagctaagacagaagaaaaggaccttaagaagctaaaaaagcaggaaaaagaagaaaaagacttcaggaaaaaatttaaatatgatggtgaaattagagtcctatattcaactaaagttacaacttccataacttctaaaaagtggggaaccagagatctacaggtaaaacctggtgaatctctagaagttatacaaaccacagatgacacaaaagttctctgcagaaatgaagaagggaaatatggttatgtccttcggagttacctagcggacaatgatggagagatctatgatgatattgctgatggctgcatctatgacaatgact

SEQ ID NO: 104 - DNA, 496 bp, Artificial sequence
  Probe No. 104
agccagtccagtactatttcacgctggctcagcaacccaccgctctccaagtccagggccagcagcaaggccagcagaccaccagctccacgaccaccatccaggctgggcagatcatcatcgcacagcctcagcagggccagaccacacctgtgacaatgcaggttggagagggtcagcaggtgcagattgtccaggctcagccacagggtcaagcccaacaggcccagagtggcactggacagaccatgcaggtgatgcagcagatcatcactaacacaggagagatccagcagatcccggtccagctgaatgccggccaggtgcagtatatccgcttagcccagcctgtatcaggcactcaagttgtgcagggacagatccagacacttgtcaccaatgctcaacagattacacagacagaggtccagcaaggacagcagcagttcagccagttcacagatggacagcagctctaccagatccagcaagtcac

SEQ ID NO: 105 - DNA, 567 bp, Artificial sequence
  Probe no. 105
tcgagcgggatggacagccctactgtgaaaaggactaccacaacctcttctccccgcgctgctactactgcaacggccccatcctggataaagtggtgacagcccttgaccggacgtggcaccctgaacacttcttctgtgcacagtgtggagccttctttggtcccgaagggttccacgagaaggacggcaaggcctactgtcgcaaggactacttcgacatgttcgcacccaagtgtggcggctgcgcccgggccatcctggagaactatatctcagccctcaacacgctgtggcatcctgagtgctttgtgtgccgggaatgcttcacgccattcgtgaacggcagcttcttcgagcacgacgggcagccctactgtgaggtgcactaccacgagcggcgcggctcgctgtgttctggctgccagaagcccatcaccggccgctgcatcaccgccatggccaagaagttccaccccgagcacttcgtctgtgccttctgcctcaagcagctcaacaagggcaccttcaaggagcagaacgacaagccttactgtcagaactgct

SEQ ID NO: 106 - DNA, 470 bp, Artificial sequence
  Probe No. 106
gagagagtatgctctgtgtcgtcccagacatttctgcattccgagaaggttggagatgggtccggcaaccagtccaggttccagtaactttggtccgaaatgatggaatcatttattccaccagccttacctttacctacacaccagaaccagggccgcggccacattgcagtgcagcaggagcaatccttcgagccaattcaagccaggtgccccctaacgaatcaaacacaaacagcgagggaagttacacaaacgccagcacaaattcaaccagtgtcacatcatctacagccacagtggtatcctaactaccgtctttttgctaggacttaaactgacttgagtgtggcaaaaagttaacaaaaaaggagaaaaaatgaacaatcgtttgtggtttcttgggaaaacttttcataccaggtgatactattcaaaaaccccgttgtctccctgcaagtgctgatttg

SEQ ID NO: 107 - DNA, 361 bp, Artificial sequence
  Probe No. 107
aaaccacgcaaacccaagaggcagagggcggctgagatggaaccacctcccgaacccaagaggcggagggtcggtgacgtggaaccgtcacgcaaacccaagaggcggagggccgctgacgtggaaccatcatcacccgaacccaagaggcggagggtcggtgatgtggaaccgtcacgcaaacccaagaggcggagggccgctgacgtggaaccatcatcacccgaacccaagaggcggagggtcggtgacgtggaaccgtcacgcaaacccaagaggcggagggccgctgacgtggaaccatcattacccgaacccaagaggcggaggttgagctgagaagaggccagtgcactcaagc

SEQ ID NO: 108 - DNA, 463 bp, Artificial sequence
  Probe No. 108
gcagtagcagttctagtagcagttcaaccagtagcagcagtggaagtagttccagcagtggaagtagtagcagtcgcagtagttccagtagcagctccagtacaagtggcagcagcagcagagatagtagcagnnnnnntagcactagtagtagtagtgagagtagaagtcggagtaggggccggggnacataatagagatagaaagcacagaaggagcgtggatcggaagagaagggatacttcaggactagaaagaagtcacaaatcttcaaaaggtggtagtagtagagatacaaaaggatcaaaggataagaattcccggtccgacagaaagaggtctatatcagagagtagtcgatcaggcaaaagatcttcaagaagtgaaagagaccgaaaatcagacaggaaagacaaaaggcgttaatggaagaagccaggctttcttagccattctttgcagc

SEQ ID NO: 109 - DNA, 424 bp, Artificial sequence
  Probe No. 109
agagggccacgtaactgagagcttacagtgccaatgccgtttgtgttctggccagagtggagtgcgcagccctgactcccaggcgctgagattgttgcctggttacccaggaagctgctgttccggctgcccagcctttctctgagccagcggatgcacagtccgtggccttcttcaggcttattgatgatgctttttgcaaatgttgaatcatggttctgtttctaagttggatcttttttgttttctccttgccaccctaatttgacatcaaaattctctcttgtgcattgggccctgggtcattcaaacccaggtcacctcattccccttctctgttcacacctaatgtcttgaagagtaggtagcagcagtgtgggctgaacctaggccagcttgcttagcgggtcaccctgctgtgaag

SEQ ID NO: 110 - DNA, 510 bp, Artificial sequence
  Probe No. 110
gatcaccaatgcttgctttgagccagccaaccagatggtgaaatgtgaccctcgccatggtaaatacatggcttgctgcctgttntaccgtggtgacgtggttcccaaagatgtcaatgctgccattgccaccatcaaaaccaagcgcancatccagtttgtggattggtgccccactggcttcaaggttnggcatcaactaccancctcccactgtggtgcctggtggagacctggccaaggtacagagagctgtgtgcatgctgagcaacaccacanccattgctgaggcctgggcncnccnnggaccacaantttgacctgatgtatnccaagcgtgcctttgttcactggtacgtgggtgaggggatggaggaaggcnnnttnncagaggcccgtgaagatatggctgcccttgagaaggattatgaggaggttggtgtggannctgttgaaggagagggtgaggaagaaggagaggaatactaattatccattccttttggccct

SEQ ID NO: 111 - DNA, 344 bp, Artificial sequence
  Probe No. 111
aggagtggatcctggagcagctcacgcgcctctacgactgccaggaagaggagatcccagaactggagattgacgtggatgagctcctggacatggagagtgacgatgcccgggctgccagggtcaaggagctgctggttgactgttacaaacccacagaggccttcatttctggcctgctggacaagatccggggcatgcagaagctgagcacaccccagaagaagtgagggtccccgacccaggagaacggtggctcccacaggacaatcgctgccccccaacctcgtagcaacagcaataccgggggaccctgcggccaggcctggtgccatgagcagggc

SEQ ID NO: 112 - DNA, 451 bp, Artificial sequence
  Probe No. 112
gtgctcaatgtatttgcctgcttacaacactgggagatgtgtttgccagtaagttgctcatcacaagagcaccagacttgggggtgtaatctccggcaacttgcatgccctctgaaagaagggttttctgtgctgtgaaatgcatagaactatactttgccatgcacgactgttcctgcaattgatattgtgtgaaatctgggagggtggtctttgggtgttctcaggggccaatggtaatttttgggttggggagccagcttggggtggggaattttcacctgggcctccgctctttaactatataaacatttatctgtatatctatgtccctgtctggggggcaggaggaatctgccaaagaccaacagtcttactttatcttactatacttcacaaaggttctaaaatgtgaagagtttacttggattgcagtagcccattggttgtt

SEQ ID NO: 113 - DNA, 435 bp, Artificial sequence
  Probe No. 113
gtaacgaaatgcccatgacgcttccggagacaaccctggaaacactgaagcataaaatcaacccctcggcgggggaggcgttcccacaagcggtggacgtgctgctctacactccagggcatcttgacccagccgaaaaagttgaagatgctcaccccaagttatggtgtgctctgagcgaaggcaaggtgaccgtgttcaatgcttcttcatggaccatccaccagcactcctttaaagtgggcactgcaaaagtgaactgcatggtgatggccgaccagaaccaggtgtgggttggctcggaagactccgtcatctacatcatcaacgtccacagcatgtcctgcaacaagcagctcacagcccactgctccagtgtcacggatttgattgtgcaggacggacaggaggcacccagcaacgtgtactcgtgca

SEQ ID NO: 114 - DNA, 460 bp, Artificial sequence
  probe No. 114
gaaccagttcgaattgcctatgacaggncnnngnnncgtnccatgtccaaaaagaagaaacccaaggacttggacttcgcccagcagaagctgaccgataagaacctgggcttccagatgctgcagaagatgggctggaaggagggccatggcctgggctccctcggaaagggcatcagggagccggtcagcgtgggaaccccctcggaaggggaagggttgggtgctgacgggcaggagcacaaagaagacacattcgatgtgttccgacagaggatgatgcagatgtacagacacaagcgggccaacaaatagttnccagggtcacccccgagaggacaacaggcatctggaagtgctctctcgccactctgggtgctttactgtctctggcttgtttccatcactggaaatcacttagagaattgtagtgtttttggtccttgataaagcctagaag

SEQ ID NO: 115 - DNA, 443 bp, Artificial sequence
  Probe No. 115
gaataatgtggttcttatgcaggaaaagtgttccagcacagaatacagccagtgcaaatgccctgggatagcaatgcactaaacatgtttaaagaactgcaatgagacagtgtgaccaaaagagaacgtgagtggagaaaatggcagagacaaattaggagaagcagtgagaagccagatcgcgtggttctatgttatcacatggtaaacctttaaaaattattatctgagngagacaggaagcctttctagtgctttagaaaaaagaaataatatgctttaaattaggttttacaaagatcacattttggtgaatagactaagatgtcacagatggaagtcaagggaagtggcaagagcacatggaagaggatgcaagaagaatcatgatgagggatgggatgaaggcagcaaggggtaaccatcttgaccttctgtttttt

SEQ ID NO: 116 - DNA, 500 bp, Artificial sequence
  Probe No. 116
aaatgtgaccctcgccatggtaaatacatggcttgctgcctgttgtaccgtggtgacgtggttcccaaagatgtcaatgctgccattgccaccatcaaaaccaagcgcagcatccagtttgtggattggtgccccactggcttcaaggttggcatcaactaccagcctcccactgtggtgcctggtggagacctggccaaggtacagagagctgtgtgcatgctgagcaacaccacagccattgctgaggcctgggctcgcctggaccacaagtttgacctgatgtatgccaagcgtgcctttgttcactggtacgtgggtgaggggatggaggaaggcgagttttcagaggcccgtgaagatatggctgcccttgagaaggattatgaggaggttggtgtggattctgttgaaggagagggtgaggaagaaggagaggaatactaattatccattccttttggccctgcagcatgtcatgctcccagaatttcagcttc

SEQ ID NO: 117 - DNA, 200 bp, Artificial sequence
  Probe No. 117
cacatctaccaacaatcccggaaggacaactccaattccctgcaggtgaaaacgtgccacctggtcaggtactggatctccgccttcccagcggagtttgacttgaacccggagttggctgagcagatcaaggagctgaaggctnngnnnnnnnnagaagggaaccgacggcacagcagcctaatcgacatagacagcgt

SEQ ID NO: 118 - DNA, 500 bp, Artificial sequence
  Probe No. 118
gagcagattctagcacatcatggcagtgaccaagcgtggtcccgagaagggccagagcctggtagagactagggaagggaggtctcctctagactgactcacattgccttgagcttttcagttaagttgctgtaagcacctgggctgaggaggctgtttttgttccttcctgcgttatagcggggccttgtctcttcctctgcaggacacagatctggaggacgtggactggggtaggaaaccacctgagggtgttagtacctagtggtgaaatggatgaggtcatttctaaggtgtgttgcccgtggatctgggcacaatcattggaattccttggagccactgggattcatggctttgtatccaactgcatccaggcctgaggctgctgacgtttgacaccaggccagtagagagtgcccttttgtatcttaagccaagaagtgaggcctgggggtgggggagggggaaggggtgggagccaatactgagtgcctgca

SEQ ID NO: 119 - DNA, 128 bp, Artificial sequence
  probe No. 119
tggacttctcaaaccaacagtggcctctcagaaccagaaccttcctgttgccaaantcccacctagcaagttagtatctgatgacttggattcatctttagccaaccttgtgggcaatcttggcatcg

SEQ ID NO: 120 - DNA, 492 bp, Artificial sequence
  Probe No. 120
caaagaggggcaactcacacccatgccccgagagatggcaagatctttcaggagaaagtgcggtcaatcatgtacctgaggcattccagcagtggaggaaggtcccttatgagccctggatttatggtaataagcccatctggttttactgcttcaccatatgaaggagagaattcctctaatattattccacaacagatggccgcccatatgctgcgttctagaagcctaccagcattccctacttcttcactactaacgcaatcacaaaaactgactggaagtttgggttgtagtatcgacaggttacaaaatattgcagatacttatgttgccacccaatcaaagaaacaaaattctttggggagttccgacacactgaaaaaaggcaaagaggacgcattcatcagtagctgtgagtctgcaaaaactgtttgtgaaatggaagctgtcctctcagcccaggtctctgtcagtgatgtcccaaaggga

SEQ ID NO: 121 - DNA, 212 bp, Artificial sequence
  Probe No. 121
tctcacctatacgtgcaagaaaggaacagttgcttgcggccancccctgttgtagaaaatgccaagacctttggaaagatgaaacctcgttatgaaatcaactccctgattagataccacntgcaaagatggtttcattcaacgtcaccttccaactatccggtgcttaggaaatggaagatgggctatacctaaaattacctgcatgaacc

SEQ ID NO: 122 - DNA, 498 bp, Artificial sequence
  Probe No. 122
gcagatcgtgcggctgaagaccaaggacaggaagaagcaagtgggcatcaagatccccgagggctgcgtgcgccgggtgctgcaggagctgcggctgatggatgcggacgtgaagcgcaggcaggcgcccgccctgggctgccccgccccgcccgccccgcgcccgctggcgctgccttgcggccccggagaggtgctggacctcacctacagccccccggccgaggccttcccgccgcccccgcacttctctttcccggcgccgctgtccctggacgccggccccggcgtcgtgccgctgggcacccccgacgcccaggccgaccctgcggccctcgcgcaccagggctgcgacatcaacttcaaggaggtgctggaggacatgctgcgctcgctgcacgcggggccgccctccgagggcgcgctgggggagggcgcgggggcggggggcgcggcgggcggtggtcccgagcggcagagcgtgatccagttcagccc

SEQ ID NO: 123 - DNA, 439 bp, Artificial sequence
  Probe No. 123
gagaacccgctggtttattgcgacgggcacggctgcagcgtcgcggtgcatcaagcttgctatggcattgttcaagtacccactggaccgtggttttgcaggaaatgtgaatctcaggagagagcagccagagtggcaccccctgtaatggcctatcctgctactacaccaacaggcatgataggatatggaattcctccacaaatgggaagtgttcctgtaatgacgcaaccaaccttaatatacagcnagcctgtcatgagacctccaaacccctttggccctgtatcaggagcacagatacagtttatgtaacttgatggaagaaaatggaattactccaaaaagacaagtgctcaagcagcaaaatccttacttccagcaaaatccaaactgctgtctcttaaatctcttaaactctcttcttccattaaaatgt

SEQ ID NO: 124 - DNA, 421 bp, Artificial sequence
  Probe No. 124
gcgaaacctgcggagccagatttgtacaggtggcccacctccgtgcccatgtgcttatccacactggtgagaagccctatccctgtgaaatctgtggcacccgtttccggcaccttcagactctgaagagccacctgcgaatccacacaggagagaaaccttaccattgtgagaagtgtaacctgcatttccgtcacaaaagccagctgcgacttcacttgcgccagaagcatggcgccatcaccaacaccaaggtgcaataccgcgtgtcagccactgacctgcctccggagctccccaaagcctgctgaagcatggagtgttgatgctttcgtctccagccccttctcagaatctacccaaaggatactgtaacactttacaatgttcatcccatgatgtagtgcctctttcatccact

SEQ ID NO: 125 - DNA, 504 bp, Artificial sequence
  Probe No. 125
agagaccgagtgaacctaccttcatttcaggagggattggccgcttggcacatgacaactttgccagcttttcctcccttgggttctgatattgccgcactagaggatataggagaggaaaagtaaggtgcagttgccccaacctcagacttaccaggaagcagatacatntgagtgtggaagncngagggngtttatgtaagagcaccttcctcacttccatacagctctacgcggcaaattaacttgagttttatttatctnatcctctggtttaattacataaatatttattttttaagtgtaattttgccaaataataanancagnaaggaaattganantagagggaggtgtttaaagagaggttatagagtaaaagatttgatgctggagaggttaaggtgcaataagaattcagggagaaatgttgttcattattggagggtaaatgatgtggtgcctgaggtctgtacattacctcttaacaatttctgtccttca

SEQ ID NO: 126 - DNA, 474 bp, Artificial sequence
  Probe No. 126
gaaacaaagttgctcttgcagaggcctggtttgcagctttacttctccttctacatgggcagcaagaccctgcgaggcaggaacacatcctctgaataccaaatactaactgctagaagagaagactctgggttatactggtgcgaggctgccacagaggatggaaatgtccttaagcgcagccctgagttggagcttcaagtgcttggcctccagttaccaactcctgtctggtttcatgtccttttctatctggcagtgggaataatgtttttagtgaacactgttctctgggtgacaatacgtaaagaactgaaaagaaagaaaaagtgggatttagaaatctctttggattctggaggccaagcacttgaagctccaactcagggctgcgcttaaggacatttacatcctctgaataccaaatactaactgctagaagagaagactctgggttatactggtgcgaggctg

SEQ ID NO: 127 - DNA, 455 bp, Artificial sequence
  Probe No. 127
tgcaccgattccagagccaaaaaccaaggatgacctagagcagctcacgactgagattaagaaaagggccaacaacgtccggaacaaactgaagagcatggagaagcatattgaagaagatgaggtcaggtcatcggcagaccttcggattcggaaatcccagcactctgtcctttctcggaagtttgtggaggtgatgaccaaatacaatgaagctcaagtggacttccgagaacgcagcaaagggcgaatccagcggcagctcgaaattactggcaaaaagacaaccgatgaggagctggaggagatgttggagagtggcaacccggccatcttcacttctgggatcattgactcacagatttccaagcaagccctcagtgagattgagggacgacacaaggacattgtgaggctggagagcagcatcaaggagcttcacgacatgtttatgg

SEQ ID NO: 128 - DNA, 523 bp, Artificial sequence
  Probe No. 128
tacctggagggcacctgcatggagtggctccgcagacacctggagaacgggaaggagacgctgcagcgcgcggacccccccnaagacacacgtgacccaccnccctnnctctgaacatgaggcataacgaggtnctgggttctgggcttctaccctgcggagatcacattgacctggcagcgggatggggaggaccagacccaggacatggagctcgtggagaccaggcccacaggggatggaaccttccagaagtgggcggttgtggtagtgccttctggagaggaacagagatacacatgccatgtgcagcacaaggggcntgcccaagcccctcatcctgagatgggagccctctccccagcccaccatccccattgtgggtatcattgctggcctggttctccttggagctgtggtcactgnnnnnnnnnnnnnnnctgtgatgtggaggaagaagagctcagatagaaaaggagggagctactctcaggctgcaagcagccaaagtgcccagggctct

SEQ ID NO: 129 - DNA, 566 bp, Artificial sequence
  Probe No. 129
gcctcaacgtgaagcttttctgggagaagtttgttcccacagattgtcccccggccttcttcccgctggccgccatctgctgcagactggagcctgagagcagagccccccccggggccgcaggagagggcccgggctgcgcggatgatgagggcccagtgaggcgccaagggaaggtcaccatcaagtatgaccccaaggagctacggaagcacctcaacctagaggagtggatcctggagcagctcacgcgcctctacgactgccaggaagaggagatctcagaactagagattgacgtggatgagctcctggacatggagagtgacgatgcctgggcttccagggtcaaggagctgctggttgactgttacaaacccacagaggccttcatctctggcctgctggacaagatccgggccatgcagaagctgagcacaccccagaagaaaccagcattctcgaaattggaggactcctttgaggccctctccctgtacctgggggagctgggcatcccgctgcctgcagagctggaggagttggaccacactgtgagcatgcag

SEQ ID NO: 130 - DNA, 184 bp, Artificial sequence
  Probe No. 130
gcagggaatgggaaccgggaaggcacgaagtctctaaagcatccagaagacccctacaccagggtctggtccgctcctattcgccgcagcctttctgttccgcctgcaacccattttccagacagtaaaanggcggcgcacttctttctccgtcaggcaccaggtcataaggaacccaagagtc

SEQ ID NO: 131 - DNA, 250 bp, Artificial sequence
  Probe No. 131
gggaccgctataaggccagtcggactgcgacatagcccatcccctcgaccgctcgcgtcgcatttggccgcctccctaccgctccaagcccagccctcagccatggcatgccccctggatcaggccattggcctcctcgtggccatcttccacaagtactccggcagggagggtgacaagcacaccctgagcaagaaggagctgaaggagctgatccagaaggagctcaccattggctcgaagctgcagg

SEQ ID NO: 132 - DNA, 517 bp, Artificial sequence
  Probe No. 132
ttgcatgcgccaggcggtgggcagcgggggcctgtccagccctctcccgccatccttccccaagtgacgtccactgccttgtcaccagcgacctgcctgtcatgcccaccccctgaggaagcatggggaccctaacaccctggtgccctgcaccagacaggccgtggtcaggcccaggccaccggccgggttctgccacagcttcccacgtgcttgctgacatgcgtgtgcctgtgtgtggtgtctgttgctgtgtcgtgaaactgtgaccatcactcagtccaaacaagtgagtggccctcgaggccacagttatgcaactttcagtgtgtgtcataacgacgtcactgctttttaaactcgataactctttattttagtaaaatgcccaggagtcctggaagctacgcggacttgcagaggttttattttttggccttagaatctgcagaaattaggaggcaccgagcccagcgcagcagcctcggacccggattgcgtttgccttagcggatat

SEQ ID NO: 133 - DNA, 463 bp, Artificial sequence
  Probe No. 133
ggggtctcattagctttgcaacaggaaacatcctgttttattatggtagtggggtcaggaatgtaggaactggtatccattctgccaattcccacccattcagtttgcttatccctacagaacagtgactgaggttcctttttttttttttttttttttttttttcaaatttccatgtattttctgccatttttcagggtctaagattggtcatacattcccaatttactctcagttccagtcaagctggttgctctgaaagtaacccagcttgttgctctaaaatacctcagtagcctgagtgttatactagagatctaaagggttaacaggatagggtggaaaggttagagactcctagaaatctctggtcaccgtgatcttcggcctcattctaatacctgttctttggacagtctttttcctttggtgctctcttgcctttagctaccttctctaatat

SEQ ID NO: 134 - DNA, 503 bp, Artificial sequence
  Probe No. 134
tcgagactggtacccggaggagctgtctcaccaggagaccacgtcctggaagtgtccgggactcgcgggacctgtggctgcagaccccgccggcacgcaggcccagagctggcgcactcctgaggatgagactctgggggccctagccggggtccacgggagggctgtccttggggactctaggatggcttcgttctggcccggctcacttctggagctgtgagacccaagacaaaaggggctgagggatttctcattgacaagagttcgtgcgggaaaaccacctgacccctagggatttgtcatcttaagactcaaaaggcttaataccaggaaccaccttggcaagatatttacccaccggccatctctgtttactcatgaatgttaaatgttaaaacgcagcgctctaaccctgcatattatttacttgcaaatgtctgtaatctgtaattgtgatgcctctgatggaataaattatctttttcagtctcctctaaaaa

SEQ ID NO: 135 - DNA, 528 bp, Artificial sequence
  Probe No. 135
gtgcagagcttttaccagcaggagctggaaatggtggagtctttgctgtcccttgccaatcagcctgtgattcacagtgcctgctccgaccaagtgaattttaagaaggacaccacttccaaggcaattcatagtatatttaagaatgctatacaactgctgcaggaaaaaggacttgttttccagaaagatgatggttttgataacctatactatgtaaccagagaagacaaagacctgcacagaaagatccaccggatcattcagcaggactgccagaaaccaaatcacatggagaagggctgtcacttcctgcacatcttggcctgtgctcgcctgagcatccgcccgggcctgagcgaggctgtgctgcagcaagttctggagctcctggaggaccagagtgacattgtcagcacaatggagcactactacacagcgttctgagcagagacacgcagaccagctgaggaggacaaagataaggtggcattcacccccaggctctgactttcagcatcatgcagg

SEQ ID NO: 136 - DNA, 488 bp, Artificial sequence
  Probe No. 136
ggcaaggaagcaggtggatcccccagaaggaaccgcagctcgcgaggcacctctcctcgtccccacccacccccacatttgcacaccttccacagggtcaggatggagtagctgccttgcgaggtggtgagactcctgtctctggaggtctgcaagcactggtagtgatattgcagcagacaaggtctgggttgggcgtctgcaggaggagaccttgtgggacatctgaggacatccgcagattcttgtcagcctgtgaactaggccctgcctctgtcacctcattggtccatgaaggagcagccaggggtggtggaaggagcactgggctaggggtcaggggtcagagattctgtcctgctcctgggatgcactggctactcccttagtctactcccttcccctctctggtcctcagcttcctccatcatggaggagatgggatcatggattttctgggcataagtggctgttctgggagtcattcc

SEQ ID NO: 137 - DNA, 448 bp, Artificial sequence
  Probe No. 137
agatgacaggcatggccggggtcagctctttcagccgcgcttcagcgatgactccagtctgggtgtcccagcgagcccctgcagggacagtatggctgagggtcaggtgtgctgccagtaagtgagggaggggctggcaggaagggtggggtcctcacactccccgccctttgcagagctgggctctaccccaaaaggcttcaggccagctgccacagctggaagcagaggccttcgtaggtgatggcctgcatgttgtaactaccccgtcccgctgggctcaaggaacagctcagctaaagccctcgggttccatccgtttaaatctgtggcattttcagagcctcatctgtcagccttaatgtcagtggcaggaagtcataactccagctaaaaattacagagtaaagttccctgattcttaatgtgtaatgtctgccctatgtgt

SEQ ID NO: 138 - DNA, 551 bp, Artificial sequence
  Probe No. 138
gcaagtctcgggatcactcagatgcagccaagaaacacaggcatgaaaggggacatcatagggacaggcgtgaacgatctcgctcctttgagaggtcccataaaagcaagcaccatggtggcagtcgctcaggacatggcaggcacaggcgctgactttgtcttcctttgagcctgcatcagttcttggttttgcctatctaccagtgtgatgtatggactcaatcaaaaacattaaacgcaaaactgattaggatttgatttcttgaaaccctctaggtctctagaacactgaggacagtttcttttgaaaagaactatgttaatttttttgcacattaaaatgccctagcagtatctaattaaaaaccatggtcaggttcaattgtactttattatagttgtgtattgtttattgctataagaactggagcgtgaattctgtaaaaatgtatcttatttttatacagataaaattgcagacactgttctatttaagtggttatttgtttaaatgatggtgaatactttcttaacactggtttgtctgca

SEQ ID NO: 139 - DNA, 399 bp, Artificial sequence
  probe no. 139
gtcacagggtcgaatactactgcacagcaacgaatatgaatgaaaatatcgctatgcacagcaacatggataaatttcacagacatgaggtcaagcaaaagaggtcagagtcctcatcatcaagagagaattcattgtatgattctcttcctacaaaaagtacagaaataagcaaaactgatccatggtgttagaagccaggggaacagttaacaggggagggatactggggaggggcatcctggagtgctggtctacctcatctgggtgttgatttcacgagtattgtcagtttgtttccagactccctgttggagatgtggaaataaaaaccacctaaacaagagcagagaggccatttggtcaaagtttgcaaaggagtcagccatgattgcttgt

SEQ ID NO: 140 - DNA, 532 bp, Artificial sequence
  Probe No. 140
atcccgacagagtcatgctcgagccctgagtgaccccaccacgcctctgtgacctggagaagatccagaacttgcgtgcagcttctcctctcagcacactttgggctgggatggcagtggggcataatggagccctgggcgatcgctgaatttcttccctctgcttcctggacacagaggaggtctaacgaccagagtattgccctgccaccactatctctagtctccctagcttggtgccttctcctgcaggagtcagagcagccacattgcttgccttcataccctggaggtggggaagttatccctcttccggtgctttcccatcctgggccactgtatccaggacatcactcccatgccagccctccctggcagcccatgttctcctcttttctcaccccctgactttccctgagaagaatcatctctgccaggtcaactggagtccctggtgactccattctgaggtgtcacaagcaatgaagctatgcaaacaataggagggtgtgacaggggaaccgtagacttt

SEQ ID NO: 141 - DNA, 448 bp, Artificial sequence
  Probe No. 141
gcagatgtacaactcaccataccaccgggtgacagactgtgtacgggcagtgtggcaaaatgaaggggccggggccttttaccgcagctacaccacccagctgaccatgaacgttcctttccaagccattcacttcatgacctatgaattcctgcaggagcactttaacccccagagacggtacaacccaagctcccacgtcctctctggagcttgcgcaggagctgtagctgccgcagccacaaccccactggacgtttgcaaaacactgctcaacacccaggagtccttggctttgaactcacacattacaggacatatcacaggcatggctagtgccttcaggacggtatatcaagtaggtggggtgaccgcctatttccgaggggtgcaggccagagtaatttaccagatcccctccacagccatcgcatggtctgtgtatgag

SEQ ID NO: 142 - DNA, 494 bp, Artificial sequence
  Probe No. 142
gaaatacacccgtcagattctggagggtgtccattatttgcacagtaatatgattgtccatagagatatcaaaggcgcaaatatccttcgagattcaacaggcaacgtcaaactaggagattttggggccagcaaacggcttcagaccatctgtctctcagggacaggaatgaagtctgtcacgggcacaccatactggatgagccctgaagtcatcagtggacaaggctatggaagaaaagcagacatctggagtgttgcatgtaccgtggtagaaatgctaactgaaaagccgccttgggctgaatttgaagcaatggctgccatctttaaaatcgccactcagccaacaaacccaaagctgccacctcatgtctcagactatactcgagatttcctcaaacggatttttgtagaggccaaactgagaccttcagctgatgaactcttaaggcacatgtttgtgcattatcactagcagccagtaacctctc

SEQ ID NO: 143 - DNA, 514 bp, Artificial sequence
  Probe No. 143
tccactcagcacagttcatgttcagtagatgctgaacattcttagaaatactgtgtgtgaacttagaaaagtgcaagaagacaggcatgtctttgaccccaggaatgatcatttgctgaagatggtgtcaagtgaacctagattaacagccctccactccagatggatatccagtgattcctagaatgggatatagccagagaacaattctatgcaccctacactgacagactcccttaagcaacaccagatgctctactggtacttgaagtacatgactttgaagtcttgaccctccatgaatacctgaattatcagcaagcgggttttgaagctggtgcctcattgaggccatattagagcaacttgtacatttgacctcttgttatcagccatggtactctacttcgtgtgcaagagataactatgaaagccaaattcaaatactggcaacatttcctaaaggggctcaatatcttatcattcgtcttcttttccaaactacacatcactg

SEQ ID NO: 144 - DNA, 560 bp, Artificial sequence
  Probe No. 144
gttgtgaaggatgtggctctgccatgaaggatgtcctgttgcctttaaaatctggaagcgattcaagccaagctgaccaagaagccaaagaactggctaggcaaataagctttaaggcagaagtcaattcatctggaaagactatctctgagtcagacttaaaccactctttttcactaactgatttacaagatgatatacctacaacattccagggtgctacggccagtacatcgtacggagtccagaattcctcagcagcatcctttcatcaacctacccaacctgtagctaagaatacctccatgagccctcgacagcgccgggcccagcagcagagtcagagaaggttgtctacttcaccagatgtaatccagggccaccagccaagagacaaccacactgatcatggtgggtcagctgtactgattgtcatcctgactttggcattggcagctcttatattccgacgaatatatctggcaaacgaatacatatttgactttgagttataatatggttttgtgacttatgagctgtgactcaactgcttcattaaa

SEQ ID NO: 145 - DNA, 403 bp, Artificial sequence
  Probe No. 145
gagtgaagaaaacggctctttccttagtaaggattttgatgcccgaaaggcctacctggctggcttcatcaaagacattgtatctcagtttggaatgggaaactgttatcttacacacagcactgatgctaaagaaaagaattgtggtgtatcaccccaagatagaagcggtccaggagttcaccaggactctgcctgccctggtgtggcaccgacaggactggaccatccttcactcttacgtgcacctcaacgccgatgagctggaancctgcagatgtgcacaggttacgtcgctggatttgtagacttggaggtgagcaacagaccagacctctatgatgtgtttgtgaatctggcagagagtgagattaccattgctccccttgcaaaagaggccatg

SEQ ID NO: 146 - DNA, 450 bp, Artificial sequence
  Probe No. 146
cagcctcactgcggcttatacagtaccctaacctgctactaatcacagagaaaaatgtgaagaaggaggagaagaggaaggctagaagcctgagcaagtgagggtagaaccttttgggactggcctttgaagctctggccagggatggggtgggggccaaaaggacagagcctggtatgtcttcatagtcattgagaatgtggagataccagtttgggtggggggtgatcaccaggggacctagggagatccccttcccaccctctctgttggcctcagagtcactcctgccccctctccctgacttggtgctcacatgcacctcactagggtttgtgaccagggtctggatgagcttgaatttgaatgaattgagtttgtatttctagaaccctgggtttttacatgtttggtctttttttgttttggtttgtcaccctcgataaagga

SEQ ID NO: 147 - DNA, 540 bp, Artificial sequence
  Probe No. 147
aagcccaccctctaagagacattcaagctgaactatcacaattcttaatcagttacaatttacaaacagataagtttaaaataaacaatttacaaaatttttgaagcataccttaacatcttgttttgcagttaaacaatggaaaagtatttctcctacactaaaaaaaaacttgcttacacacaactgaaaatagaatcttacttgataatacaaaagctaccatcagaagaaatcccttcaggatcattaagccacttcctttgctctgcagtttctatagtagttttaaattattattaaatcacctgaaaaaaattccaaaagagaaccacacactaccatatccaaacaacttttgcatttcccataattgtagttaatgtcagcccagtaggccagaccaacccccagttcaatactttccttccccaaaagctctatactttgaaggaaaacagatacagtatcaaattatgacactttccttgcccaaattaatgcactggtacacccagtggctcatatttaacttccccc

SEQ ID NO: 148 - DNA, 303 bp, Artificial sequence
  Probe No. 148
cacctggccaagaataccacttttgaagttaatccttttgtgtgatacaggatgaacttgggatgtttgaaccctggacattccaaataaagaataggcccctgcctggctcctgggagataacctctaagccattagaatatcttgcctgataagagtgtttttgtttacctgtgggccttgggccatgcagtatcagcttgaccttgcaaggtcaagctgaggagactaagttagccatgtgggcagtgaagcatgccaatgtgatcaatccctagtaaaagccctggacacctaggcatg

SEQ ID NO: 149 - DNA, 402 bp, Artificial sequence
  Probe no. 149
acaccaaaaccccatctgtatgtcaccatcatcgaggaccaaaggtagataaaaccacaaagatggggaaaaaacagagcagaaaagctgaaaattataaaaatcagagcgcttctccccctccaaaggaccgcagctcctcaccagcaacggaacaaagctggacacagaatgactttgatgagttgagagaagaaggcttcagagaatcagacttctccgagctaaaggaggaagttcgaacccatcgcaaagaagctaaaaaccttgagaaaagattagacaaatggctaactagagtaaccagtgtagagaagtccttaaatgacctgatggagctgaaaaccatggcatgagaactacgtgacgaatgcacaagctttagtagctgattcaatcaac

SEQ ID NO: 150 - DNA, 443 bp, Artificial sequence
  Probe No. 150
agacagcctgtttcagagggttgttttgtttggggtgtgggtgttatcaagtgaattagtcacttgaaagatgggcgtcagacttgcatacgcagcagatcagcatccttcgctgccccttagcaacttaggtggttgatttgaaactgtgaaggtgtgattttttcaggagctggaagtcttagaaaagccttgtaaatgcctatattgtgggcttttaacgtatttaagggaccacttaagacgagattagatgggctcttctggatttgttcctcatttgtcacaggtgtcttgtgattgaaaatcatgagcgaagtgaaattgcattgaatttcaagggaatttagtatgtaaatcgtgccttagaaacacatctgttgtcttttctgtgtttggtcgatattaataatggcaaaatttttgcctatctagtatcttca

SEQ ID NO: 151 - DNA, 528 bp, Artificial sequence
  Probe No. 151
cccccagacggccacagagtgggccgagatcctggcgctgcagaagcaattccacagcgtggaggtgcacaagtggaggcagatcctgcgggcctccgtggagctcctggatgagatgaagttctcgctggagaagctgcaccaaggcatcacagtctcagaccctccctttgacacccagccccggcccgatgacagcttttcctgaggaccccggccacgcagctgttcccccacatggacagatggacacacagagcctcggcggccactgctggcacggtgtgagcgccaggcatctcccacccgcccctcccgacggcccaaccaggggctgtgcagacgtggggaccacggaaccgagatgcactttagaccagggagctggcccggcctctggcaggccccccactaacttattttgcccggctgaggttgtggggggcgcctcctggggtgcacgattccctcagctctgggtttaatgtattatatttatttggggccgacagtgccccaataaagggt

SEQ ID NO: 152 - DNA, 399 bp, Artificial sequence
  Probe No. 152
ggtgctggagacccatagagctgatgggagcagctggtgcctggccttcggntcctgcgtccccagaacccaagggaacgtcatggaggccacatggggccacccggctccctcgggatggctccgcctgcacttttgaaaccccggtttccttcaacntccacattccaggtgaccacacgtgtctcctcctcctcatcttagcttccaggttcaccctaaccctgtactaacctgcttggtggacttggaaaagacttggctctgtcgggaaaggagagacggggcctccatcacgcctgttaccagaggatccccgagagccacaccagctntggacatcaccgcccctggaactggggccaccagccctgggcacgagatttgctctgactttat

SEQ ID NO: 153 - DNA, 448 bp, Artificial sequence
  Probe No. 153
gagggacagcgatgtggccctctgtgttaagaataacgtgtcctgctttggcagagagaagaaaatagccactgcccgctttcaaggcaagatcgaccttttctgttttgttttgtttttctttctttttcctggccatgaggacaaaaattactgagtggcccttaaagagggaagtttgttttcagctgttctcttttgcccgtaggtgggagggtggggattgctgcgtcctagctagaggaatggctttgcttgaatgtgtagtgcacacgcacgggtgtttctgtgtgctagttgcttcttgctgctgcttcctgcttgtctgggactcacatacataacgtgnnatatatatatatatatataaatgtataaatatatattttatttttttttaaatccttggagcttctggttcctatcagttcctgttgttaatcgtaga

SEQ ID NO: 154 - DNA, 431 bp, Artificial sequence
  Probe No. 154
actttgcctctctagaagcagcttctcgcctttccctcccgagggctggcaggagtcactgtcagtcacccttcatggggtctgaaaggttgcatgggctcacctggtcttagacaaggacagttgcannttccgcaccaggcagacctgcagtttgagtggctggggataggtggagcggcggcctggccacagtgccctggacagcagcacatcacatcagttaacactgtttgccttccaactagttattacggtgtcctgtgggacaattagactggaggagccaaaggaatgtgcaggggcactgggtttctgtttggagagaggctctcggtaaacagagttcgcattcctccagattctgtgggaccagagtaaatatcattatgccacagccaggcttaagcaatctatctgcagtgccgttc

SEQ ID NO: 155 - DNA, 375 bp, Artificial sequence
  Probe No. 155
gggtgtaaggattcccactgtggctcttcacatgatggaactgtttgacacaactgtagagcagctgtatagcatcttcactgtaaaggagttaacaaataagaagatcatcatgaaatggagatgtgggaactggccagaagaacactatgccatggttgcactgaattttgtgcctactctagggcaaacagaattacaattgaaggagttcctatctatctgtaaagaagagaacatgaaattctgttggcagaagcagcattttgaagaaataaaaggttcactgcagctgacccccctaaatggttgaattaaaatttattataaggcattactttttgtaagcggaaaaagtgtcacatttacctcttc

SEQ ID NO: 156 - DNA, 352 bp, Artificial sequence
  Probe No. 156
caacagggtcagtgtcacagccacaacttcagaaagcagccatcccgcgtgtcgtccaaacagcagtgcctgcgtcccgctccacggagtcttccagaacacctccctntgaactaatcccggagcaccccacccccagcgagtcccacctggacctcttcaaagtcaaaactcttctctcgggacaaacaaatattcgggactttcgaagccctgaacttgagacaccaagtttgtctccatggaaacacgtagccgctgcagttggacagctgcgtgctggagttctgtccccaaaccaactgcagagaagaaagacgagcctcggcccaggacgcgatttctaacgaaa

SEQ ID NO: 157 - DNA, 396 bp, Artificial sequence
  Probe No. 157
attgactccactggatttgtgctctgttgagtcagaggctcccgggggttggggatcgaggggcgggggtcagctatgcagcccatcacgtgtgtttttcatctgggatgaaaaagcctggttctcttttgaaatgcttgattgtacttattgagctaaacaagtcttggtgactgttgttgatttgcctcaaaagttttaagtcctgggttttcagactactgtgtagcagctgtgtgtttaacatactgtagctttttctcccttgggggcacatacaaataggatgtgttgatgtggactctaaactgtaattttcctgtaactattttggaatgatgcatatttctaatgtttgttatacttgtacagagtattgctgttggttgctttttt

SEQ ID NO: 158 - DNA, 450 bp, Artificial sequence
  Probe No. 158
tgggaaggccgtctgtggcaagtgcagctccaagcgctcctccatccccctgatgggcttcgagtttgaagtgagggtctgtgacagctgccacgaggccatcacagatgaagaacgtgcacccacagccaccttccatgacagtaaacataacattgtgcatgtgcatttcgatgcaaccagaggatggttactgacttctggaactgacaaggttattaagttgtgggatatgaccccagtcgtgtcttgatgactctcccaggaatcagaaagatagtatttactaaagaaacggttgttttaacccaaatcattaccagagtggtaaagcagacatgtgagaagtaagaaagaaactaaagaccctgaatgaatttgcagattacccatgtgcacagtggggacctggccagtgagcactcgcaaggggactcttccaacttgttc

SEQ ID NO: 159 - DNA, 438 bp, Artificial sequence
  Probe No. 159
aagaccatcccaaaatgcttcaagtaaaaaataacaagtttaaggggttaagcacttttaaagtctgattaagggggtggggggaaaaaagagtaactaccagccatttctccaatggacatctcttccacagacctcaacgtgagaactgctctagtttctataaactgtaaacctgtggtggtctgattatcctgatattggattttcttgttttctgttacaccttgagtcatttgcctttaggattctagacagacctaagggaaaaagaactgaaaacatattttgcccccacccccacaaaaaaaaatactgaaaactcccccccgcctcagttacacatccaaactctacatttacaaaacgaattcagggtgaggaagtaaaaacaggtcatctattcacaaaactgaaatacttcattaccccaactaa

SEQ ID NO: 160 - DNA, 372 bp, Artificial sequence
  Probe No. 160
ctgccatagcactgatcagtgacaatttacaggaatgtagcagcgatggaattacctggaacagttttttgtttttgtttttgtttttgtttttgtgggggggggcaactaaacaaacacaaagtattctgtgtcaggtattgggctggacagggcagttgtgtgttggggtggtttttttctctatttttttgtttgtttcttgttttttaataatgtttacaatctgcctcaatcactctgtcttttataaagattccacctccagtcctctctcctcccccctactcaggcccttgaggctattaggagatgcttgaagaactcaacaaaatcccaatccaagtcaaactttgcacatatttatattta

SEQ ID NO: 161 - DNA, 454 bp, Artificial sequence
  Probe no. 161
actacctaagattattttcacagctaccaatttgtcagaaactttggtggtgttttgngggaggctgagattttttaaattaaaaatcaagtgaaaacacaaagactctaagacacgatcctgatttgtgtcatttctaagtatcagatttgttctcccttcatttgacaggtgttctcagtctctcctcctgtaagattctgtccttcccctgatgacaagccaatagttcttggtggggttgcatgtctcctagagccccaggacccctgtcgtgtcggggaaggggggagggcaggagggtgggcanagactcgggttgggggagggagttcaaagaaagagtgaaaatatgggtttatataaacatatttaaagcagtttagcaaaagctttctcgttgaacagctttaagaacaatgtgaatgaaatcttagcaacttggttagtaatc

SEQ ID NO: 162 - DNA, 410 bp, Artificial sequence
  Probe No. 162
aacatttccacttgccagtttaatttcttgaagactgttgcttgtttggaatgtttcttgtcactgattttaaggttgcatctggaaaagactaaaggcttcagtcccctcccaccaccagaaatgaacaaaaagcattttacctaaaaatacaccagcaaaatgtactcagcttcaatcacaaatacgactgcttaaaactgcagaaatttcctcaacactcagcctttatcactcagctggattttttccttcaacaatcactactccaagcattggggaacacaacttttaatcatactccagtcgtttcacaatgcattctaatagcagcgggatcagaacagtactgcatttacttgccaacagaacagacagacctgaagtcaagacaactgcattctctgtga

SEQ ID NO: 163 - DNA, 442 bp, Artificial sequence
  Probe No. 163
agatatttttggctgctgacctaggnnaatgattctactctatcacatctgnatactatgnntacttactgagtagactcagattttgagtaatcataccttgactgtagttgtncatatactctgaaagaaatttatatatcaacctgaaataattggttgaaaccctttgctcaggtactttttaaacttcgtaattatggaaatttgttttaagaaatgttcagtgctgatacatcatctttctgaaaggatgtaagttctgtctgtgatcaatgtgaagtaaaagagttacagccttttttgtaccattttatccctgaatacttacctgtattttaatctgaagtatgatcatttgtgccttctaaagcagattatttaactgattaaagagtcccttgaaattgatttttcaagcgttagaaggttagccatgtaa

SEQ ID NO: 164 - DNA, 282 bp, Artificial sequence
  Probe No. 164
ttctgccatttttcagggtctaagattggtcatacattcccaatttactctcagttccagtcaagctggttgctctgaaagtaacccagcttgttgctctaaaatacctcagtagcctgagtgttatactagagatctaaagggttaacaggatagggtggaaaggttagagactcctagaaatctctggtcaccgtgatcttcggcctcattctaatacctgttctttggacagtctttttcctttggtgctctcttgcctttagctaccttctctaatat

SEQ ID NO: 165 - DNA, 420 bp, Artificial sequence
  Probe No. 165
tctgcctttcttgacctaaactgtcatggtaaattcttcttantccnntgtcaccagccacagacagtcgccacaagccatgacaagtctgcgatttcatggtctttttttattttgatgatatttccccagtgtttgggggacagaagaatgcccacaatctataggtcttagtcacatggatggggacagaatatctgagagagagagaaggaacaagatctactggggctccttaaaattcttcttgaagaactcctttctcaccttggagtagggaagactttctgattatgactcaaaatttagaagctaagaaagaaaatacctttaaatctgaaaacataaaactcaaaaacttctgggtgtttttttgggggggttgnttttttttaaacagcacaagcaactcaaagggca

SEQ ID NO: 166 - DNA, 456 bp, Artificial sequence
  Probe No. 166
actgcagcaaactttctagcatctgatattggataagnatagcttgtgctanangntggagatnaatcnggtctgctgnctngcancnttagagnctgnatctnatggttggtgtnaggatgttgttgacagttctgaaagttagccatcaattcctgtgcagggtggagtcagacccagtgacttccttttcaatgtcagcaagagttttctcatgcctgctttggtcactttctcttggaaacttcacgcatttgacttgcagcttcttgacccgaggaatcaactgagctcccagtgctggctaccttgggcaaataaaatctgtggttgagagagttctctttgctgtgccacagtccctgtgatgtgccaaatggcaccagcatttgcagcaaagctcgttaaatctttttggaatgggcaaatattttttcaacttgagccctgctgagc

SEQ ID NO: 167 - DNA, 354 bp, Artificial sequence
  Probe No. 167
cttacctattgcctctgatatttacttgcttaaatttttttttattggaaatccagaaaaagtggatttagagaacaacactaactcccacctaatctatgacagagatnnnnnanagagtanctgtgaaaaatgtgaaagtatctgaaaaatgtaacctttggcagcctgagcatagtcaaccagaaaaactatctgaattaaaataattggtccataggtactattttatttggtccataaggattattttttcaactttttttnnaagtgtattattatgtcatttcccacgtaggttactgatacctgaagactttttccacctttaaccttactcgttgaggagctttg

SEQ ID NO: 168 - DNA, 536 bp, Artificial sequence
  Probe No. 168
cttgtctttggtttctgctaggttatccagtttgttggcctgtgatttttcatagtgccgtagtctaaccttgtctttggtttctgctaggttatccagtttgttggcctgtgatttttcatagtgccgtagtctaaccttgtctttggtttctgctaggttatccagtttgttggcctgtgatttttcatagtgccgtagtctaaccttgtctttggtttctgctaggttatccagtttgttggcctgtgatttttcatagtgccgtagtctaaccttgtctttggtttctgctaggttatccagtttgttggcctgtgatttttcatagtgccgtagtctaaccttgtctttggtttctgctaggttatccagtttgttggcctatgatttttcatagtgccgtagtctaaccttgtctttggtttctgctaggttatccagtttgttgacctatgatttttcatagtgccgtagtctaaccttgtctttggtttctgctaggttatctggtttgttggcctatgatttttcat

SEQ ID NO: 169 - DNA, 515 bp, Artificial sequence
  Probe No. 169
gatagggttactacttgagttgctatggctccagctgaaagaaagcccgtgcagtcatatcacgcgtaaacatttgctttatgctaaaaatatggtggacctggcattacagctattacaaatctcctaagatgtctcgggtagtgtattagttacttttcatactgctatgaagaaatactggaaactgggtaatttataaagaaaaagaggtttaatgtactcacagttccacaaggctggagaggcctcagaatcatggtggaaggcaaagaaggagcaaaaaggtatgtcttccatggcagcaggcaagagagcacgtgcagggaaactgccctttataaaaccatcagatttagtgagatgtattcactatcacgagaacagtatgggaaaaacctgcccccatgattcgattacctcctaccgggtccctcccacgacacatggggattatgggaactacaattcaagatgaaatttgggtggggacgcagccaaaccatatcgggtag

SEQ ID NO: 170 - DNA, 498 bp, Artificial sequence
  Probe No. 170
gacacttgagtttgaaatccagcccaaccatttactagttgtgtgacctctatcaagttatgcaacctctctgtgcctcagtttcctttttggcaaattggtgctaacacctcacaggactattgtaaggattaagttcatgtagataaagcacttaacacagcatccgacacatctcatcactcaataaattatcactgccctttgttactgtggttaacaaacacttgctgtaatttctctcttctaacttataggacaaacaaataatggaaatttttaacataagtatgtagagattgtggtaccacagtgataaactgatttttatgcttaagttaatgactctggtttatgtatgacaatttattcagtatgagaatctcagaaaactaatgtttttttctattccttaaccttcctaccctttttttggtaaagaaaattaccgaaaaaacaaaaaanaaaaaaaaaactacctctgtctatttattccat

SEQ ID NO: 171 - DNA, 529 bp, Artificial sequence
  Probe No. 171
gcatctagctcatagcaagtgcttaataaatgatctgaaataaagcaaaagagattttatcaagttgtgattgcccagtggcaatcacaaacagagaagtgatgccttctctgtttttttcaggtgcaggtgcacttttgtctatatgaccaagatcagtccttccattcagagtagcccctctttgagtagtgctgatacctaatttttgatgttgataatttttctgctctggagtntgctgcctaccacccttcagacatattatgttaattaaattatatcatattttaccctagcacaccccagagtaggccctctacagatatttgctaaatcagttagattttagcatctggaagatttggtagacttcttggtcttataagcaacttttctttttaaaccttatttccaaactatgggtatgtactttatttatacagctcttcatagattacactatgtactttgaatacatagtatttgttttcattcgatttactacgttggtttttttatcttgatg

SEQ ID NO: 172 - DNA, 508 bp, Artificial sequence
  probe No. 172
gccagcagattccattcattcgcttcagctggagactaagagaacagnangagagggcaagtatttcctgccatatatcagacagtgagaaagtgtnnccatgatttagtttagatgaattataatggcacacacaaagcacaagctgccttgattcaacaaagaagatggaaaacagaagccgctctgatcagtgaggaagaaaagaaaattacgaagacaagttatgtatttgctctgatttcagacactccgaatgtatttgttctgtggtgcagaatattaagaaaataacagacacgctcatgcattaaaagaataaccgcaatgtactcatgcataatctacaacatgtttttaaaggatggaaatcaaatctatctgtcatactttgccctcaagtattctgtgggctgttgacatcattttgaattacaaatgatgtatacagcttgtgctacaaagattttggaactagaaacagaatcagactcatgcattactttta

SEQ ID NO: 173 - DNA, 455 bp, Artificial sequence
  Probe No. 173
atgagtgaccctctgatagctactttacaggagcatcctaaacatttctcagcattcatctcatcacactttcgttcattctatgtaactgcagccaatcttatattccacagtcctttacaacaactctttgttttgagctttattatagtggaggtagacttttatgagtattttgtcaagagcgagccagggaacttccatattatcctttcaagagaattggcttccaaagtgacttttcaagagaaccagaagggtcatcccatgccttagtagtagagctaaactatcccagaatagaggctacttcaaacctaccctaaacacgtttaaaaagaagccatgaaaggatcaaacaaatctgattttccagtaacttaactgtcttctaacgaagccattctttaagggaacatggcaaagttcagatacttaactcaatgtccaccagc

SEQ ID NO: 174 - DNA, 516 bp, Artificial sequence
  Probe No. 174
aactgttcaaagccatacctgcacatgtttgaacttcaaaccctgtgggtgattcagtggcatctttctctaacccccagcctcccttcccacagaggccaccgtcatggccagttgctgcantttctttccagagaacctgtgtatgtgtaaagctgtacaggcgtgggtacaccacacagcctgtcttgcactgtggactgttgagttactagtacatctaggtaagcaccgcatatctgtattcatgtctgccttggtcttttcaacatctntgtgnnagnngngtttgaattaccnttcccttttngggnannnnccattanngttgttncagcaatttttactgtagataaggctataccgnatatctgtgtncatgggttttnatgnacatgggcannnatatctgngagagaaannnttcctcagnagnaattctgggcacagcatgtgtaaatttctaaatatgatggacacccccagcttccacctcaaggaggttggtcccattga

SEQ ID NO: 175 - DNA, 458 bp, Artificial sequence
  Probe No. 175
actagtgtaggctgacaacccaaattaaggaagcaggagagatcaaacagaactgctgctgggtggttgtcaggagctgctacacggagaaccctggactattcgatcaagcagcaaggctatatgttcacttatgcagaaatggacnattgcagatgctaanctttgttgtgcaagcgaaggctcacttggaaggaaatactcagcccctctctgggcagcatttgagttccttatggatgccgagtcgcgaaacaagttantttttttaatgtatccttctttatgaggagaatgctacccaaaaatgtattaaaggaatattaagtcgtccagagactgtcttgctaccaagaactgtgcaatggaattctttttaccaacattagactcctacactagagttagataacgttttctcacattgagtttagaagatctgccttgtcagggaagcc

SEQ ID NO: 176 - DNA, 423 bp, Artificial sequence
  Probe No. 176
ctcgtgggatgagcaaatgactctgaaacggtcccatgcgggaaatgtccatgaagtcctggattttatctaaaaagcccaggcaggggtggggcgggggcggcggggctacagttccacgctgagctncctcctggccgctcgtccccgccgcagtgcctnggcggcccgggcgcccgaccttggccgtggacaccttcgcggtgcgtgctgctcctccccatctgccactggaagatgctggggcgacccggctccaggtttagcaggacactgagaaaagggaatggctgcctttcggaggctgggtgagcccttctctgtgcctcacctgcccgccccacagcggccctgcacctcgtcccacggggcccattgccccggtaggatgcgcgcttttgttttgagggtcaggcatcttcc

SEQ ID NO: 177 - DNA, 493 bp, Artificial sequence
  Probe No. 177
tcagataaagcaaacccatgccctcaagaccccgcaggaatgccattagggggcaggaggcctgaatttggctgtaggaatttagagaatttggaggttctcttccattcaaattttttcctccaagtggtatatactttaccataattttcatcataagccaaaagtctgacattagcctgaaggactgtgtgaccattaaagttagtaaattaaactaacttattctttacaaaagagtaaatcattcttttatccaaagagtgactgaggtctacactgcactcagcactgaggggagcgccacggtggaagggaacacacgtgggtcacagtcaccgcacacaaggtacttatggtctagctggggatgcaacacagccacacgccaaccagggagtgaacaagacagcatgatacatagtaaaataatgttccaggttaaccaggaaagaagcaagtgcgactgtgcctttggttactatgtcttttc

SEQ ID NO: 178 - DNA, 485 bp, Artificial sequence
  Probe No. 178
ggcagtagcagtaatgatggaccggttaataagagactctgagtgtgatgtaaatagtatgtttattaaacaatagagcccattggaatttttttcttccacgtattgttagatgtaaaacctgatacactaccatacagacaggtgattgactagaccttgtttatgtagttctaatgtggaatacttagagttgttatgaattacgattatccataacttttgaatctaaaaagctggctacacaacgnattttgttnggaatctttttgtaataccaaatgtctacactctttgatctagagaatcacaaaattttagaaacatcactacncagnttaattgacttttttattgtttactgtccaaatacctgttacatattaatagtattctactttatatacctgtaataaactattcagggtttcttaannnggttgttgaaatgttttgtaatactgctcttcttaccttgttatagg

SEQ ID NO: 179 - DNA, 389 bp, Artificial sequence
  Probe No. 179
gtacatcagatacaacactcttctcatcttttttgtcattatttttcaatgttgttttaatctttccactgttgtttcatgtcatgcactttgttaatattctttaagttattcttagtttttancaatgatacttaagaaagacttaataggnctatagaataggtactccctattccccaaccctgggaacacaatataacatattagttcatttgtatttaagatctatcttgtttacncctcagtttagttattgctcttacctttcaaacagttaatattttgtatctagttctgtgaaagcaaaaagcaactttattctttcttacaattccccttttacttaatcggaacatttctttcctgtttgttcattagtcactctc

SEQ ID NO: 180 - DNA, 521 bp, Artificial sequence
  Probe No. 180
tagtgatggattgctttgtaatactttgggantctgggtttgncanagtgtgcccctatnaagactaagtgggactataatgattgngttttcttaaatcagaatcnggatgcataaccagatgaaggaagacatagtcgggagccatagttttantatctagattttggaattttagggtgatttactatagggacaaagtatttgaaattgggattggcggaacatcagtggaaccagcgattgctaagttaaatatatacagggacattagataatggatgatgaggaaatgggtaaaagaaagtgagggccttgtgagttgaaacaaaaataatcaaacctggaacactttccatttattgtattagatatcttcatttcaataaaattaatgtatatgacaaaattttattgtcctcctagttcaagtggtattctacttttatttccataaaaatatactttcaggatagggaaagggtaaacttgcattataagtttgtattttctcacgaagg

SEQ ID NO: 181 - DNA, 360 bp, Artificial sequence
  Probe No. 181
ctctcaagtagcttttctcatggacttaagtccccatcggtgacacggagggaaaagaacttcagaaccaactgttggccctgaaaagacctcaaagagggtgcaatccagttgatttttacattgaaaggtcacagttccccctctgagagtntgacggnttgtacaancccctnccccaggaancctgtatacacagtcanccancccatgaagcagtctcaggctaagaactntattatggaccgtagcccaatctgttcattttacagatgaggcaactgagtccacaggagagcagaacgtgacntgcgtagaacacaaagttagttagaggcagacctaggacccaggtaacca

SEQ ID NO: 182 - DNA, 390 bp, Artificial sequence
  Probe No. 182
gacctcacagctctttcagggaaagcacactttgtaagcaagataacatctagtaaaccttctgctgttgccagtgaaaaatttaaagaacaagttgatcttgcaaaaaccatgaccaatttatcaccaaccattcttggcaatgcagttcagttgatctcttcagtccccaaagggaaactgccaatcccaccctactcaagaatgaagacaatggaggtttacaaaatcaaatcagatgctaacattgcaggtttttctttaccaggacctaaggccgactgtgataagataccctccaccacagaaggctttaatgcagccaccaaggtggcaagcaggctacctgttccacaagtgtcacagcagagtgcctgtgaaagtgccttt

SEQ ID NO: 183 - DNA, 407 bp, Artificial sequence
  Probe No. 183
tgagactcccctaaaggccagaaaggtgcgcttctccgagaaggtcactgtccatttcctggctgtctgggncagggncggcccaggccgcccgccagggcccctgggagcagcttgctcgggatcgcagncnnttcgcacgccgcatcacccaggcccaggaggagctgagcccctgcctcancccnnnnnnnnnnnnnnnnnnnnnnggcacgcctcaggaacccacctttagcccccatccctgccctcacccagaccttgccttcctcctctgtcccttcgtccccagtccagaccacgcccttgagccaagctgtggccacaccttcccgctcgtctgctgctgcagcggctgccctggacctcagtgggaggcgtggctgagaccaactggtttgcctata

(19) United States
(12) Patent Application Publication
(10) Pub. No.: US 2011/0189680 A1
(43) Pub. Date: Aug. 4, 2011

(54) Methods of Diagnosing Rejection of a Kidney Allograft Using Genomic or Proteomic Expression Profiling

(75) Inventors: Paul Keown, Delta (CA); Andreas Scherer, Kontiolahti (FI); Oliver Gunther, Vancouver (CA); Robert Balshaw, Vancouver (CA); Raymond Ng, Vancouver (CA); Alice Mui, Burnaby (CA); Robert McMaster, Vancouver (CA); Bruce McManus, Vancouver (CA); Gabriela Cohen Freue, Vancouver (CA); Anna Meredith, Vancouver (CA)

(73) Assignee: The University of British Columbia, Vancouver, BC (CA)

(21) Appl. No.: 12/991,922

(22) PCT Filed: May 29, 2009
(86) PCT No.: PCT/CA2009/000744

§ 371 Date: Mar. 15, 2011

Related U.S. Application Data
(60) Provisional application No. 61/129,022, filed on May 30, 2008.

(51) Int. Cl. C12Q 1/68; G01N 33/53; G01N 33/68; C12Q 1/48; C12Q 1/00; C12Q 1/37; G01N 33/573; H01J 49/26

(52) U.S. Cl. 435/6.12; 436/94; 436/86; 436/501; 435/7.92; 435/15; 435/4; 435/23; 435/7.4; 250/282

(57) ABSTRACT

A method of determining the acute allograft rejection status of a subject, the method comprising the steps of: determining the nucleic acid expression profile of one or more than one nucleic acid markers, or one or more than one proteomic markers in a biological sample from the subject; comparing the expression profile of the one or more than one nucleic acid markers to a control profile; and determining whether the expression level of the one or more than one nucleic acid markers is increased relative to the control profile, wherein the increase of the one or more than one nucleic acid markers is indicative of the acute rejection status of the subject.

This application claims priority benefit of U.S. Provisional application 61/129,022, filed May 30, 2008, the contents of which is herein incorporated by reference.

### FIELD OF INVENTION

The present invention relates to methods of diagnosing rejection of a kidney allograft using genomic expression profiling or proteomic expression profiling.

### BACKGROUND OF THE INVENTION

Transplantation is considered the primary therapy for patients with end-stage vital organ failure. While the availability of immunosuppressants such as cyclosporine and tacrolimus has improved allograft recipient survival and wellbeing, identification of rejection of the allograft as early and as accurately as possible, and effective monitoring and adjusting immunosuppressive medication doses is still of primary importance to the continuing survival of the allograft recipient.

Rejection of an allograft results from a recipient's immune response to nonself antigens expressed by the donor tissues, and may occur with hours or days of receiving the allograft, or months to years later. Renal allograft rejection is characterized by features comprising oliguria, rapid deterioration of renal function and mild proteinuria. Renal allograft rejection can lead to nephropathy and kidney failure.

At present, invasive biopsies (e.g. endomyocardial, liver core, and renal fine-needle aspiration) are regarded as the gold standard for the surveillance and diagnosis of allograft rejections, but are invasive procedures which carry risks of their own (e.g. Mehra M R, et al. Curr. Opin. Cardiol. 2002 March; 17(2):131-136.). Biopsy results may also be subject to reproducibility and interpretation issues due to sampling errors and inter-observer variabilities, despite the availability of international guidelines such as the Banff schema for grading kidney and liver allograft rejection (Solez et al 2008 Am J Transplant 8: 753; Table 1) An allograft recipient may be exposed to the biopsy procedure multiple times in the first year following the transplant. Noninvasive surveillance techniques are currently used (the increase in blood creatinine levels), however serum creatinine levels are non-specifically reflective of kidney injury. The kidney injury can be from rejection, infection, or even recurrence of the original disease, thus, the test is not specific for rejection.

Indicators of allograft rejection may include a heightened and localized immune response as indicated by one or more of localized or systemic inflammation, tissue injury, allograft infiltration of immune cells, inflammatory cells which recognize donor-specific antigens on the graft, allospecific antibodies, cytotoxic T-cell activation, altered composition and concentration of tissue- and blood-derived proteins, differential oxygenation of allograft tissue, edema, infection, necrosis of the allograft and/or surrounding tissue, and the like.

Allograft rejection may be described as ‘acute’ or ‘chronic’. Acute rejection (also known as acute antibody-mediated rejection, AMR or active rejection) is generally considered to be rejection of a tissue or organ allograft within ˜6-12 months of the subject receiving the allograft. Rejection or acute rejection may be characterized by cellular and humoral insults on the donor tissue, leading to rapid graft dysfunction and failure of the tissue or organ. Rejection of a tissue or organ allograft beyond 6-12 months is generally considered to be chronic rejection, and may occur several years after receiving the allograft. Such late or chronic rejection may be the result of sub-clinical or not fully resolved acute rejection episodes. Later-onset or chronic rejection may be characterized by progressive tissue remodeling triggered by the alloimmune response may lead to gradual neointimal formation within arteries, contributing to obliterative vasculopathy, parenchymal fibrosis and consequently, failure and loss of the graft. Depending on the nature and severity of the rejection, there may be overlap in the indicators or clinical variables observed in a subject undergoing, or suspected of undergoing, allograft rejection—either chronic or acute.

The scientific and patent literature is blessed with reports of this marker or that being important for identification/diagnosis/prediction/treatment of every medical condition that can be named. Even within the field of allograft rejection, a myriad of markers are recited (frequently singly), and conflicting results may be presented. This conflict in the literature, added to the complexity of the genome (estimates range upwards of 30,000 transcriptional units), the variety of cell types (estimates range upwards of 200), organs and tissues, and expressed proteins or polypeptides (estimates range upwards of 80,000) in the human body, renders the number of possible nucleic acid sequences, genes, proteins, metabolites or combinations thereof useful for diagnosing acute organ rejection is staggering. Variation between individuals presents additional obstacles, as well as the dynamic range of protein concentration in plasma (ranging from 10⁻⁶ to 10³ μg/mL) with many of the proteins of potential interest existing at very low concentrations) and the overwhelming quantities of the few, most abundant plasma proteins (constituting ˜99% of the total protein mass.

PCT Publication WO 2006/125301 discloses nucleic acids that are differentially expressed in transplanted tissue, and methods and materials for detecting kidney tissue rejection.

U.S. Pat. No. 7,235,258 discloses methods of diagnosing or monitoring transplant rejection, including kidney transplant rejection in a subject, by detecting the expression level of one or more genes in the subject. Oligonucleotides useful in these methods are also described.

Flechner et al. (Am J Transplant 2004: 4 (9) 1475-1489) identifies several publications that employed DNA or microarrays to identify differential expression of various genes in subjects receiving kidney transplants, and also describes use of microarray analysis and RT-PCR to examine gene expression profile of peripheral blood lymphocytes and kidney biopsy samples from kidney transplant subjects, and identified over 60 genes that were differentially expressed.

Alakulppi et al, 2007 (Transplantation 83:791-798) discloses the diagnosis of acute renal allograft rejection using RT-PCT for eight nucleic acid markers. Further investigations by Alakulppi et al. (2008, Transplantation 86:1222-8) were unable to identify a robust whole blood gene expression nucleic acid marker for subclinical rejection.

Sarwal et al. 2003 (N. Engl. J. Med 349:125) reported that genes associated with apoptosis were increased in renal biopsies during acute rejection and found transcript groups indicating lymphocyte infiltration and activation driven by NF-kappaB and IFNγ.

Mueller et al., 2007. Am J. Transplant 7:2712 identified transcripts in the kidney tissue associated with cytotoxic T-lymphocytes, IFNγ signaling, and epithelial cell injury in both mouse and human.

Mehra et al., 2008 suggests that pathways regulating T-cell homeostatis and corticosteroid sensitivity may be associated with future acute rejection of cardiac transplants, but offers no comment with respect to kidney transplantation. Expression of ITGAX is one of the 33 genes addressed.

A review by Fildes et al 2008 (Transplant Immunology 19:1-11) discusses the role of cell types in immune processes following lung transplantation, and discloses that AICL (CLEC2B) interaction with NK cell proteins may have a role in acute and chronic rejection.

Integration of multiple platforms (proteomics, genomics) has been suggested for diagnosis and monitoring of various cancers, however discordance between protein and mRNA expression is identified in the field (Chen et al., 2002. Mol Cell Proteomics 1:304-313; Nishizuka et al., 2003 Cancer Research 63:5243-5250). Previous studies have reported low correlations between genomic and proteomic data (Gygi S P et al. 1999. Mol Cell Biol. 19:1720-1730; Huber et al., 2004 Mol Cell Proteomics 3:43-55).

Several studies have been done looking at the urine proteome of kidney transplant recipients (reviewed in Schaub et al., 2008. Contrib. Nephrol 160:65-75.

Bottelli et al., 2008 (J. Am Soc Nephrol 19:1904-18) teaches that macrophage stimulating protein (MSP) is upregulated during regeneration of injured tubule cells, and suggests that it may aid recovery from acute kidney injury. Gorgi et al. (2009 Transplantation Proceedings 41:660-662) investigated the association between acute kidney transplant rejection, and a polymorphism of the MBL gene, and concluded that the polymorphism could be involved in susceptibility to acute allograft rejection in the study population. Fiane et al., 2005 (Eur Heart J 26:1660-5) disclosed that a low MBL level was related to the development of acute rejection in cardiac transplant recipients. Fildes 2008 (J. Heart Lung Transplant 27:1353-1356) teaches that heart transplant recipients with MBL deficiency had fewer rejection episodes. Neither Fiane nor Fildes offers comment with respect to kidney transplants.

Berger et al., 2005 (Am J. Transplant 5:1361-1366) teaches that higher MBL (Mannose-binding lectin) may be associated with a more severe form of rejection in kidney transplant recipients, and suggests that pre-transplantation MBL levels may be useful for risk stratification prior to kidney transplantation.

Methods of assessing or diagnosing allograft rejection that are less invasive, repeatable and more robust (less susceptible to sampling and interpretation errors) are greatly desirable.

### SUMMARY OF THE INVENTION

The present invention relates to methods of diagnosing rejection of a kidney allograft using genomic expression profiling or proteomic expression profiling of one or more biological samples obtained from a subject.

The biological sample may be a blood or a plasma sample; use of such samples in the methods described herein provides an advantage over biopsy-based assessment and/or monitoring of kidney allograft rejection (including acute rejection) as such samples may be obtained in a minimally invasive manner (a peripheral blood sample, for example), with no requirement for biopsy of the allograft. Use of a blood or plasma sample provides a further advantage, in that it may reduce sampling error, and detection of proteomic or nucleic acid markers may be less subject to interpretation—the marker is present or it is not, or it is increased or decreased relative to a baseline, control or the like as described herein.

Some current surveillance techniques that do employ blood sampling (e.g. serum creatine levels) may not be specific for rejection; the nucleic acid or proteomic markers described herein, when obtained from a blood or plasma sample are specific for acute kidney allograft rejection, thus provide a further advantage of specificity.

The complex pathobiology of acute kidney allograft rejection is reflected in the heterogeneity of markers identified herein. Markers identified herein distribute over a range of biological processes: immune signal transduction, cytoskeletal reorganization, apoptosis, T-cell activation and proliferation, cellular and humoral immune responses, acute phase inflammatory pathways, and the like.

In accordance with another aspect of the invention, there is provided a method of determining the acute allograft rejection status of a subject, the method comprising the steps of: a) determining the nucleic acid expression profile of one or more than one nucleic acid markers in a biological sample from the subject, the nucleic acid markers selected from the group comprising TncRNA, FKSG49, ZNF438, 1558448_a_at, CAMKK2, LMAN2, 237442_at, FKSG49/LOC730444, JUNB, PRO1073 and ITGAX; b) comparing the expression profile of the one or more than one nucleic acid markers to a control profile; and c) determining whether the expression level of the one or more than one nucleic acid markers is increased relative to the control profile; wherein the increase of the one or more than one nucleic acid markers is indicative of the acute rejection status of the subject.

In some aspects the biological sample is blood or plasma.

In some aspects, the group of nucleic acid markers further comprises one or more than one of SFRS16, NFYC, NCOA3, PGS1, NEDD9, LIMK2, NASP, 240057_at, LOC730399/LOC731974, FKBP1A, HLA-G, RBMS1 and SLC6A6.

In some aspects, the control profile is obtained from a non-rejecting, allograft recipient subject or a non-allograft recipient subject.

In some aspects, the method further comprises obtaining a value for one or more clinical variables.

In some aspects, the method further comprises at step a) determining the expression profile of one or more than one of the nucleic acid markers selected from Table 2.

In some aspects, the nucleic acid expression profile of the one or more than one nucleic acid markers is determined by detecting an RNA sequence corresponding to one or more than one markers.

In some aspects, the nucleic acid expression profile of the one or more than one nucleic acid markers is determined by PCR.

In some aspects, the nucleic acid expression profile of the one or more than one nucleic acid markers is determined by hybridization. The hybridization may be to an oligonucleotide.

In some aspects the control is an autologous control.

In accordance with another aspect of the invention, there is provided a method of determining acute allograft rejection status of a subject, the method comprising the steps of a) determining a proteomic expression profile of proteomic markers in a biological sample from the subject, the proteomic markers including a polypeptide encoded by one or more than one of KNG1, AFM, TTN, MSTP9/MST1, PI16, C2, MBL2, SERPINA10, F9 and UBR4; b) comparing the expression profile of the proteomic markers to a control profile; and c) determining whether the expression level of the one or more than one proteomics markers is increased or decreased relative to the control profile; wherein the increase or decrease of the five or more proteomic markers is indicative of the acute rejection status of the subject.

In some aspects the biological sample is blood or plasma.

In some aspects, the level of polypeptides encoded by one or more than one of KNG1 and AFM are decreased relative to a control, and the level of polypeptides encoded by one or more than one of TTN, MSTP9, MST1, PI16, C2, MBL2, SERPINA10, F9 and UBR4 are increased relative to a control profile.

In some aspects the control profile is obtained from a non rejecting, allograft recipient subject or a non-allograft recipient subject.

In some aspects, the method further comprises obtaining a value for one or more clinical variables.

In some aspects, the proteomic expression profile is determined by an immunologic assay.

In some aspects, the proteomic expression profile is determined by ELISA.

In some aspects the proteomic expression profile is determined by mass spectrometry.

In some aspects the proteomic expression profile is determined by an isobaric or isotope tagging method.

In some aspects the proteomic markers further include a polypeptide encoded by one or more than one of LBP, VASN, ARNTL2, PI16, SERPINA5, CFD, USH1C, C9, LCAT, B2M, SHBG and C1S.

In some aspects the control is an autologous control.

In accordance with another aspect of the invention, there is provided a method of determining acute allograft rejection status of a subject, the method comprising the steps of: a. determining a proteomic expression profile of proteomic markers in a biological sample from the subject, the proteomic markers including a polypeptide included in one or more than one of protein group codes 111, 224, 23, 18, 100, 116, 38, 135, 125; b. comparing the expression profile of the proteomic markers to a control profile; and c. determining whether the expression level of the one or more than one proteomics markers is increased or decreased relative to the control profile; wherein the increase or decrease of the five or more proteomic markers is indicative of the acute rejection status of the subject.

In some aspects the protein group codes further includes one or more than one of groups 18, 108, 222, 97, 104, 26, 230, 103, 69 or 29.

In some aspects the biological sample is blood or plasma.

In some aspects, the level of polypeptides encoded by one or more than one of KNG1 and AFM are decreased relative to a control, and the level of polypeptides encoded by one or more than one of TTN, MSTP9, MST1, PI16, C2, MBL2, SERPINA10, F9 and UBR4 are increased relative to a control profile.

In some aspects the control profile is obtained from a non rejecting, allograft recipient subject or a non-allograft recipient subject.

In some aspects, the method further comprises obtaining a value for one or more clinical variables.

In some aspects, the proteomic expression profile is determined by an immunologic assay.

In some aspects, the proteomic expression profile is determined by ELISA.

In some aspects the proteomic expression profile is determined by mass spectrometry.

In some aspects the proteomic expression profile is determined by an isobaric or isotope tagging method.

In some aspects the proteomic markers further include a polypeptide encoded by one or more than one of LBP, VASN, ARNTL2, PI16, SERPINA5, CFD, USH1C, C9, LCAT, B2M, SHBG and C1S.

In some aspects the control is an autologous control.

In accordance with another aspect of the invention, there is provided an array comprising one or more probe sets for one or more than one of the nucleic acid markers TncRNA, FKSG49, ZNF438, 1558448_a_at, CAMKK2, LMAN2, 237442_at, FKSG49/LOC730444, JUNB, PRO1073, ITGAX.

In some aspects, the array further comprises one or more additional probe sets for one or more than one of the nucleic acid markers, SFRS16, NFYC, NCOA3, PGS1, NEDD9, LIMK2, NASP, 240057 at, LOC730399/LOC731974, FKBP1A, HLA-G, RBMS1 and SLC6A6.

In some aspects, the array further comprises one or more additional probe sets for the nucleic acid markers of Table 2.

In accordance with another aspect of the invention, there is provided an array comprising one or more detection reagents for one or more than one of the proteomic markers KNG1, AFM, TTN, MSTP9, MST1, PI16, C2, MBL2, SERPINA10, F9 and UBR4.

In some aspects, the array further comprises one or more additional detection reagents for one or more than one of LBP, VASN, ARNTL2, PI16, SERPINA5, CFD, USH1C, C9, LCAT, B2M, SHBG and C1S.

In accordance with another aspect of the invention, there is provided a method of assessing, monitoring or diagnosing kidney allograft rejection in a subject, the method comprising: a) determining the expression profile of at least one or more nucleic acid markers presented in Table 2 in a biological sample from the subject; b) comparing the expression profile of the at least one or more markers to a non-rejector profile; and c) determining whether the expression level of the at least one or more markers is up-regulated (increased) or down-regulated (decreased) relative to the control profile, wherein up-regulation or down-regulation of the at least one or more markers is indicative of the rejection status.

In some embodiments, the method further comprises obtaining a value for one or more clinical variables and comparing the one or more clinical variables to a control. The control is a non-rejection, allograft recipient subject or a non-allograft recipient subject. In some embodiments, the rejection is acute rejection. In some embodiments, the one or more nucleic acid markers includes 2, 3, 4, 5, 6, 7, 8, 9, 10, 11, 12, 13, 14, 15, 16, 17, 18, 19, 20, 21, 22, 23 or 24 nucleic acid markers selected from those presented in Table 2. In some embodiments, the nucleic acid markers may include one or more than one of the nucleic acid markers presented in Table 5.

In accordance with another aspect of the invention, there is provided a kit for assessing or diagnosing kidney allograft rejection in a subject, the kit comprising reagents for specific and quantitative detection of at least one or more markers presented in Table 2, along with instructions for the use of such reagents and methods for analyzing the resulting data. The kit may further comprise one or more oligonucleotides for selective hybridization to one or more of a gene, transcript or sequence unit representing one or more of the markers. Instructions or other information useful to combine the kit results with those of other assays to provide a non-rejection cutoff index or control for the diagnosis of a subject's rejection status may also be provided in the kit.

In some embodiments, the kit may further comprise instructions or materials for obtaining a value for one or more clinical variables and comparing the one or more clinical variables to a control. The control is a non-rejection, allograft recipient subject or a non-allograft recipient subject. In some embodiments, the rejection is acute rejection. In some embodiments, the one or more nucleic acid markers includes 2, 3, 4, 5, 6, 7, 8, 9, 10, 11, 12, 13, 14, 15, 16, 17, 18, 19, 20, 21, 22, 23 or 24 nucleic acid markers selected from those presented in Table 2. In some embodiments, the nucleic acid markers may include one or more than one of the nucleic acid markers presented in Table 5.

This summary of the invention does not necessarily describe all features of the invention. Other aspects, features and advantages of the present invention will become apparent to those of ordinary skill in the art upon review of the following description of specific embodiments of the invention.

### BRIEF DESCRIPTION OF THE DRAWINGS

These and other features of the invention will become more apparent from the following description in which reference is made to the appended drawings wherein:

FIG. 1 shows the results of a subject classification using a panel of 24 nucleic acid biomarkers (presented in Table 5). Subjects were determined to have. A) 24-probe-set classifier; B) Zoomed-in view of A) to more clearly illustrate the Gaussian peaks and samples below. For A and B, acute rejection—solid circle; no rejection—open circle. C) The same dataset as in A and B, displaying the data in the same format as for FIG. 2. Acute rejection (solid diamond) or no rejection (solid circle)

FIG. 2 shows the result of a subject classification using only clinical parameters (serum creatinine, GFR, BUN). Subjects were determined to have acute rejection (solid diamond) or no rejection (solid circle).

FIG. 3: Differential expression of probe-sets between subjects with and without BCAR detected by micro-array analysis. Points in grey indicate the probe-sets identified by LIMMA alone, while those in black indicate the 183 probe-sets identified by the intersection of LIMMA, robust LIMMA and SAM. Circles indicate the 24 probe-sets included in the primary classifier.

FIG. 4: Principal component analysis showing separation of same subject groups, demonstrating that the centroids of all groups are clearly separated. AR—acute rejector; NR—non-rejector; N—normal control (20 non-recipient subjects). The percentage variance as explained by the principal components are provide on the X axis (56%) and Y axis (12%).

FIG. 5. Gene ontologies and network analysis of 183 probe sets differentially expressed in BCAR. The x-axis shows −log 10 (p-values). A Most significantly enriched Gene ontology categories (“Biological processes”), sorted by increasing p-value. B Most significantly enriched Gene ontology categories (GeneGO MetaCore Biological Categories), sorted by increasing p-value.

FIG. 6. Performance of classifier. (A) Incremental classification accuracy demonstrating step-wise inclusion of 11 common most highly predictive probe-sets. Y-axis—classification accuracy; X-axis, biomarkers. (B) Linear discriminant analysis showing performance of 11 probe-set classifiers in distinguishing cases with (•, solid line) and without (°, stippled line) BCAR (biopsy-confirmed acute rejection). (C) Change in classifier score post-transplant relative to individual pre-transplant (baseline) value. The difference between cohorts is significant only at the time of rejection (week 1) (p=0.0001). Y axis—change from baseline (mean+/−2 se); X axis BL-baseline; W1-W12, week 1-week 12. “START” indicates the beginning of the tsep-wise analysis where there are no probe-set classifiers.

FIG. 7: Volcano plot showing all 144 protein group codes that were found in at least two thirds of the BCAR positive samples and two thirds of the BCAR negative samples. Circled points indicate the 18 protein groups whose plasma concentration differed significantly (p<0.05) between subjects with or without BCAR.

FIG. 8: Linear discriminant analysis showing separation of patients with or without BCAR based upon plasma protein biomarkers. Solid line/“X”—BCAR subjects; stippled line/“|”—control (non-rejector) subjects.

FIG. 9: Estimated classification accuracy demonstrating step-wise inclusion of protein groups as chosen by forward-selection stepwise discriminant analysis (SDA). Y Axis—classification accuracy; X axis—PGC codes. “START” is as for FIG. 6.

FIG. 10 shows target sequences of (SEQ ID NO: 1-183) of nucleic acid markers useful for diagnosis of acute kidney allograft rejection, listed in Table 2.

### DETAILED DESCRIPTION

The present invention provides for methods of diagnosing rejection in a subject that has received a tissue or organ allograft, specifically a kidney allograft.

The present invention provides genomic and proteomic expression profiles related to the assessment, prediction or diagnosis of allograft rejection in a subject. While several of the elements in the genomic or proteomic expression profiles may be individually known in the existing art, the specific combination of the altered expression levels (increased or decreased relative to a control) of specific sets of genomic, T-cell, proteomic or metabolite markers comprise a novel combination useful for assessment or diagnosis or allograft rejection in a subject.

An allograft is an organ or tissue transplanted between two genetically different subjects of the same species. The subject receiving the allograft is the ‘recipient’, while the subject providing the allograft is the ‘donor’. A tissue or organ allograft may alternately be referred to as a ‘transplant’, a ‘graft’, an ‘allograft’, a ‘donor tissue’ or ‘donor organ’, or similar terms. A transplant between two subjects of different species is a xenograft.

Subjects may present with a variety of symptoms or clinical variables well-known in the literature as an aid for monitoring allograft rejection. A myriad of clinical variables may be used in assessing a subject having, or suspected of having, allograft rejection, in addition to biopsy of the allograft. The information from these clinical variables is then used by a clinician, physician, veterinarian or other practitioner in a clinical field in attempts to determine if rejection is occurring, and how rapidly it progresses, to allow for modification of the immunosuppressive drug therapy of the subject. Examples of clinical variables are presented in Table 1.

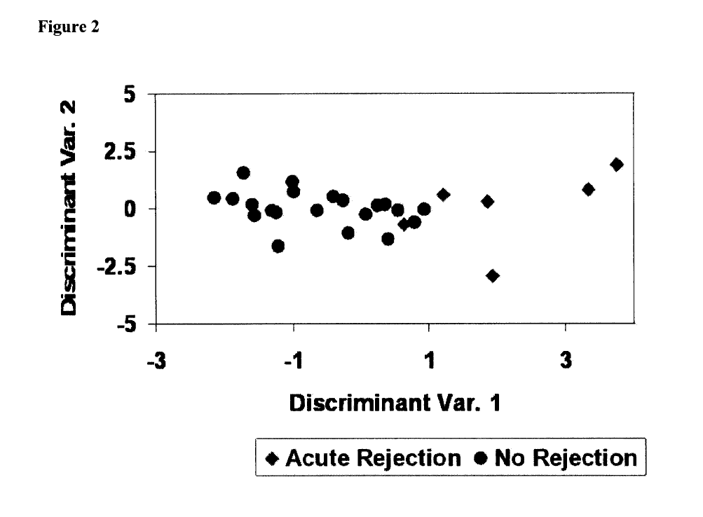

Clinical variables (optionally accompanied by biopsy), while currently the only practical tools available to a clinician in mainstream medical practice, are not always able to cleanly differentiate between rejecting and a non-rejecting subject, as is illustrated in FIG. 2. While the extreme left and right subjects are correctly classified as rejecting or non-rejecting, the bulk of the subjects are represented in the middle range and their status is unclear. This does not negate the value of the clinical variables in the assessment of allograft rejection, but instead indicates their limitation when used in the absence of other methods.

TABLE 1
Clinical variables for possible use in assessment of allograft rejection.

| | Renal/Heart/ | |
|---|---|---|
| Clinical Variable Name | Liver/All | Variable Explanation |
| Primary Diagnosis | All | Diagnosis leading to transplant |
| Secondary Diagnosis | All | Diagnosis leading to transplant |
| “Transplant Procedure - Living related, | | |
| Living unrelated, or cadaveric” | | |
| Blood Type | All | Blood Type |
| Blood Rh | All | Blood Rh |
| Height (cm) | All | Height (cm) |
| Weight (kg) | All | Weight (kg) |
| BMI | All | Calculation: Weight/(Height)2 |
| Liver Ascites | All | |
| HLA A1 | All | |
| HLA A2 | All | |
| HLA B1 | All | |
| HLA B2 | All | |
| HLA DR1 | All | |
| HLA DR2 | All | |
| CMV | All | Viral Status |
| CMV Date | All | Date of viral status |
| HIV | All | Viral Status |
| HBV | All | Viral Status |
| HBV Date | All | Date of viral status |
| HbsAb | All | Viral Status |
| HbcAb (Total) | All | Viral Status |
| HBvDNA | All | Viral Status |
| HCV | All | Viral Status |
| HCV Genotype | All | Hepatitis C genotype |
| HCV Genotype Sub | All | “Hepatitis C genotype, subtype” |
| EBV | All | Viral Status |
| Zoster | All | Viral Status |
| Dialysis Start Date | All | Dialysis Start Date |
| Dialysis Type | All | Dialysis Type |
| Cytoxicity Current Level | All | |
| Cytoxicity Current Date | All | |
| Cytoxicity Peak Level | All | |
| Cytoxicity Peak Date | All | |
| Flush Soln | All | Type of Flush Solution used at transplant |
| Cold Time 1 | All | |
| Cold Time 2 | All | |
| Re-Warm Time 1 | All | |
| Re-Warm Time 2 | All | |
| HTLV 1 | All | |
| HTLV 2 | All | |
| HCV RNA | All | |
| 24 hr Urine | All | 24 Hour urine output |
| Systolic Blood Pressure | All | Blood Pressure reading |
| Diastolic Blood Pressure | All | Blood Pressure reading |
| 24 Hr Urine | All | 24 hour urine |
| Sodium | All | Blood test |
| Potassium | All | Blood test |
| Chloride | All | Blood test |
| Total CO2 | All | Blood test |
| Albumin | All | Blood test |
| Protein | All | Blood test |
| Calcium | All | Blood test |
| Inorganic Phosphate | All | Blood test |
| Magnesium | All | Blood test |
| Uric Acid | All | Blood test |
| Glucose | All | Blood test |
| Hemoglobin A1C | All | Blood test |
| CPK | All | Blood test |
| Parathyroid Hormone | All | Blood test |
| Homocysteine | All | Blood test |
| Urine Protein | All | Urine test |
| Creatinine | All | Blood test |
| BUN | All | Blood test |
| Hemoglobin | All | Blood test |
| Platelet Count | All | Blood test |
| WBC Count | All | Blood test |
| Prothrombin Time | All | Blood test |
| Partial Thromboplastin Time | All | Blood test |
| INR | All | Blood test |
| Gamma GT | All | Blood test |
| AST | All | Blood test |
| Alkaline Phosphatase | All | Blood test |
| Amylase | All | Blood test |
| Total Bilirubin | All | Blood test |
| Direct Bilirubin | All | Blood test |
| LDH | All | Blood test |
| ALT | All | Blood test |
| Triglycerides | All | Blood test |
| Cholesterol | All | Blood test |
| HDL Cholesterol | All | Blood test |
| LDL Cholesterol | All | Blood test |
| FEV1 | All | Lung function test |
| FVC | All | Lung function test |
| Total Ferritin | All | Blood test |
| TIBC | All | Blood test |
| Transferrin Saturated | All | Blood test |
| Ferritin | All | Blood test |
| Angiography | Heart | Heart function test |
| Intravascular ultrasound | Heart | Heart function test |
| Dobutamine Stress Echocardiography | Heart | Heart function test |
| Cyclosporine WB | All | Immunosuppressive levels |
| Cyclosporine 2 hr | All | Immunosuppressive levels |
| Tacrolimus WB | All | Immunosuppressive levels |
| Sirolimus WB | All | Immunosuppressive total daily dose |
| Solumedrol | All | Immunosuppressive total daily dose |
| Prednisone | All | Immunosuppressive total daily dose |
| Prednisone ALT | All | Immunosuppressive total daily dose |
| Tacrolimus | All | Immunosuppressive total daily dose |
| Cyclosporine | All | Immunosuppressive total daily dose |
| Imuran | All | Immunosuppressive total daily dose |
| Mycophonelate Mofetil | All | Immunosuppressive total daily dose |
| Sirolimus | All | Immunosuppressive total daily dose |
| OKT3 | All | Immunosuppressive total daily dose |
| ATG | All | Immunosuppressive total daily dose |
| ALG | All | Immunosuppressive total daily dose |
| Basiliximab | All | Immunosuppressive total daily dose |
| Daclizumab | All | Immunosuppressive total daily dose |
| Ganciclovir | All | Anti-viral total daily dose |
| Lamivudine | All | Anti-viral total daily dose |
| Riboviron | All | Anti-viral total daily dose |
| Interferon | All | Anti-viral total daily dose |
| Hepatitis C Virus RNA | All | test for presence of HCV values ( ) |
| CMV Antigenemia | All | Antiviral and Virus |
| Valganciclovir | All | Anti-viral total daily dose |
| Neutrophil Number | All | Blood test |
| C Peptide | All | Blood test |
| Peg Interferon | All | Anti-viral total daily dose |
| GFR | All | Glomerular Filtration Rate |
| Complication Events | All | Complication Type |
| Biopsy Scores | Renal | Borderline 1A, 1B, 2A, 2B, 3 |
| | | Hyperacute |
| Biopsy Scores | Liver | Portal inflammation, Bile duct |
| | | inflammation damage, Venous endothelial |
| | | inflammation, each scored from 1-2 |
| Donor Blood Type | All | Donor Blood Type |
| Donor Blood Rh | All | Donor Rh |
| Donor HLA A1 | All | Donor HLA A1 |
| Donor HLA A2 | All | Donor HLA A2 |
| Donor HLA B1 | All | Donor HLA B1 |
| Donor HLA B2 | All | Donor HLA B2 |
| Donor HLA DR1 | All | Donor HLA DR1 |
| Donor HLA DR2 | All | Donor HLA DR2 |
| Donor CMV | All | Donor CMV |
| Donor HIV | All | Donor HIV |
| Donor HBV | All | Donor HBV |
| Donor HbsAb | All | Donor HbsAb |
| Donor HbcAb (total) | All | Donor HbcAb (total) |
| Donor Hbdna | All | Donor Hbdna |
| Donor HCV | All | Donor HCV |
| Donor EBV | All | Donor EBV |

The multifactorial nature of allograft rejection prediction, diagnosis and assessment is considered in the art to exclude the possibility of a single biomarker that meets even one of the needs of prediction, diagnosis or assessment of allograft rejection. Strategies involving a plurality of markers may take into account this multifactorial nature. Alternately, a plurality of markers may be assessed in combination with clinical variables that are less invasive (e.g. a biopsy not required) to tailor the prediction, diagnosis and/or assessment of allograft rejection in a subject.

Regardless of the methods used for prediction, diagnosis and assessment of allograft rejection, earlier is better—from the viewpoint of preserving organ or tissue function and preventing more systemic detrimental effects. There is no ‘cure’ for allograft rejection, only maintenance of the subject at a suitably immunosuppressed state, or in some cases, replacement of the organ if rejection has progressed too rapidly or is too severe to correct with immunosuppressive drug intervention therapy.

Applying a plurality of mathematical and/or statistical analytical methods to a protein or polypeptide dataset, metabolite concentration data set, or nucleic acid expression dataset may indicate varying subsets of significant markers, leading to uncertainty as to which method is ‘best’ or ‘more accurate’. Regardless of the mathematics, the underlying biology is the same in a dataset. By applying a plurality of mathematical and/or statistical methods to a microarray dataset and assessing the statistically significant subsets of each for common markers, uncertainty may be reduced, and clinically relevant core group of markers may be identified.

“Markers”, “biological markers” or “biomarkers” may be used interchangeably and refer generally to detectable (and in some cases quantifiable) molecules or compounds in a biological sample. A marker may be down-regulated (decreased), up-regulated (increased) or effectively unchanged in a subject following transplantation of an allograft. Markers may include nucleic acids (DNA or RNA), a gene, or a transcript, or a portion or fragment of a transcript in reference to ‘genomic’ markers (alternately referred to as “nucleic acid markers”); polypeptides, peptides, proteins, isoforms, or fragments or portions thereof for ‘proteomic’ markers, or selected molecules, their precursors, intermediates or breakdown products (e.g. fatty acid, amino acid, sugars, hormones, or fragments or subunits thereof). In some usages, these terms may reference the level or quantity of a particular protein, peptide, nucleic acid or polynucleotide, or metabolite (in absolute terms or relative to another sample or standard value) or the ratio between the levels of two proteins, polynucleotides, peptides or metabolites, in a subject's biological sample. The level may be expressed as a concentration, for example micrograms per milliliter; as a colorimetric intensity, for example 0.0 being transparent and 1.0 being opaque at a particular wavelength of light, with the experimental sample ranked accordingly and receiving a numerical score based on transmission or absorption of light at a particular wavelength; or as relevant for other means for quantifying a marker, such as are known in the art. In some examples, a ratio may be expressed as a unitless value. A “marker” may also reference to a ratio, or a net value following subtraction of a baseline value. A marker may also be represented as a ‘fold-change’, with or without an indicator of directionality (increase or decrease/up or down). The increase or decrease in expression of a marker may also be referred to as ‘down-regulation’ or ‘up-regulation’, or similar indicators of an increase or decrease in response to a stimulus, physiological event, or condition of the subject. A marker may be present in a first biological sample, and absent in a second biological sample; alternately the marker may be present in both, with a statistically significant difference between the two. Expression of the presence, absence or relative levels of a marker in a biological sample may be dependent on the nature of the assay used to quantify or assess the marker, and the manner of such expression will be familiar to those skilled in the art.

A marker may be described as being differentially expressed when the level of expression in a subject who is rejecting an allograft is significantly different from that of a subject or sample taken from a non-rejecting subject. A differentially expressed marker may be overexpressed or underexpressed as compared to the expression level of a normal or control sample.

A “profile” is a set of one or more markers and their presence, absence, relative level or abundance (relative to one or more controls). For example, a metabolite profile is a dataset of the presence, absence, relative level or abundance of metabolic markers. A proteomic profile is a dataset of the presence, absence, relative level or abundance of proteomic markers. A genomic or nucleic acid profile a dataset of the presence, absence, relative level or abundance of expressed nucleic acids (e.g. transcripts, mRNA, EST or the like). A profile may alternately be referred to as an expression profile.

The increase or decrease, or quantification of the markers in the biological sample may be determined by any of several methods known in the art for measuring the presence and/or relative abundance of a gene product or transcript, or a nucleic acid molecule comprising a particular sequence, polypeptide or protein, metabolite or the like. The level of the markers may be determined as an absolute value, or relative to a baseline value, and the level of the subject's markers compared to a cutoff index (e.g. a non-rejection cutoff index). Alternately, the relative abundance of the marker may be determined relative to a control. The control may be a clinically normal subject (e.g. one who has not received an allograft) or may be an allograft recipient that has not or is not demonstrating rejection.

In some embodiments, the control may be an autologous control, for example a sample or profile obtained from the subject before undergoing allograft transplantation. In some embodiments, the profile obtained at one time point (before, after or before and after transplantation) may be compared to one or more than one profiles obtained previously from the same subject. By repeatedly sampling the same biological sample from the same subject over time, a composite profile, illustrating marker level or expression over time may be provided. Sequential samples can also be obtained from the subject and a profile obtained for each, to allow the course of increase or decrease in one or more markers to be followed over time For example, an initial sample or samples may be taken before the transplantation, with subsequent samples being taken weekly, biweekly, monthly, bimonthly or at another suitable, regular interval and compared with profiles from samples taken previously. Samples may also be taken before, during and after administration of a course of a drug, for example an immunosuppressive drug.

Techniques, methods, tools, algorithms, reagents and other necessary aspects of assays that may be employed to detect and/or quantify a particular marker or set of markers are varied. Of significance is not so much the particular method used to detect the marker or set of markers, but what markers to detect. As is reflected in the literature, tremendous variation is possible. Once the marker or set of markers to be detected or quantified is identified, any of several techniques may be well suited, with the provision of appropriate reagents. One of skill in the art, when provided with the set of markers to be identified, will be capable of selecting the appropriate assay (for example, a PCR based or a microarray based assay for nucleic acid markers, an ELISA, protein or antibody microarray or similar immunologic assay, or in some examples, use of an iTRAQ, iCAT or SELDI proteomic mass spectrometric based method) for performing the methods disclosed herein.

The present invention provides nucleic acid expression profiles and proteomic expression profiles related to the assessment or diagnosis of allograft rejection in a subject. While several of the elements in the genomic or T-cell expression profiles or proteomic expression profiles may be individually known in the existing art, the specific combination of the altered expression levels (increased or decreased relative to a control) of specific sets of genomic or proteomic markers comprise a novel combination useful for assessment or diagnosis of allograft rejection in a subject.

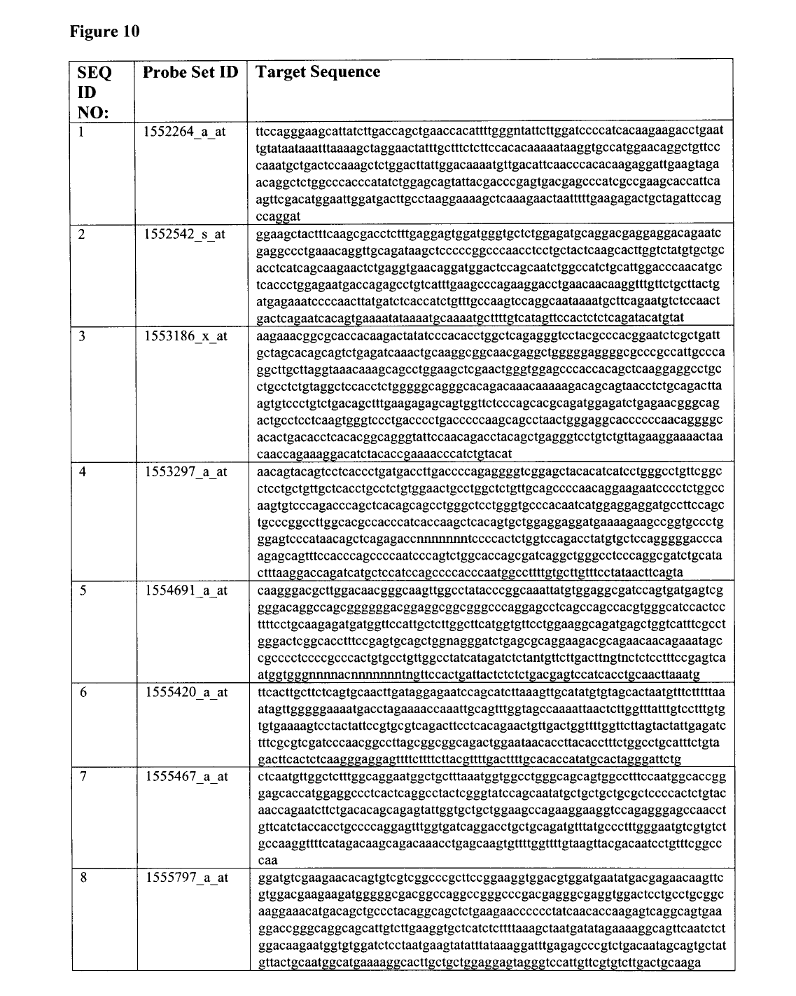

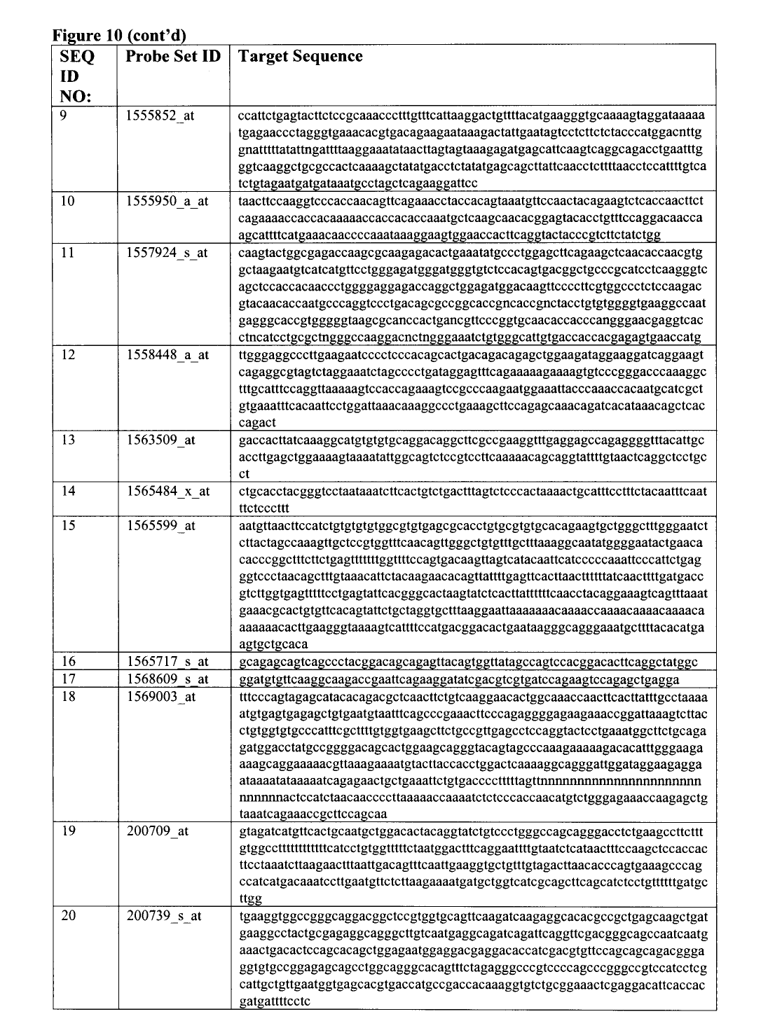

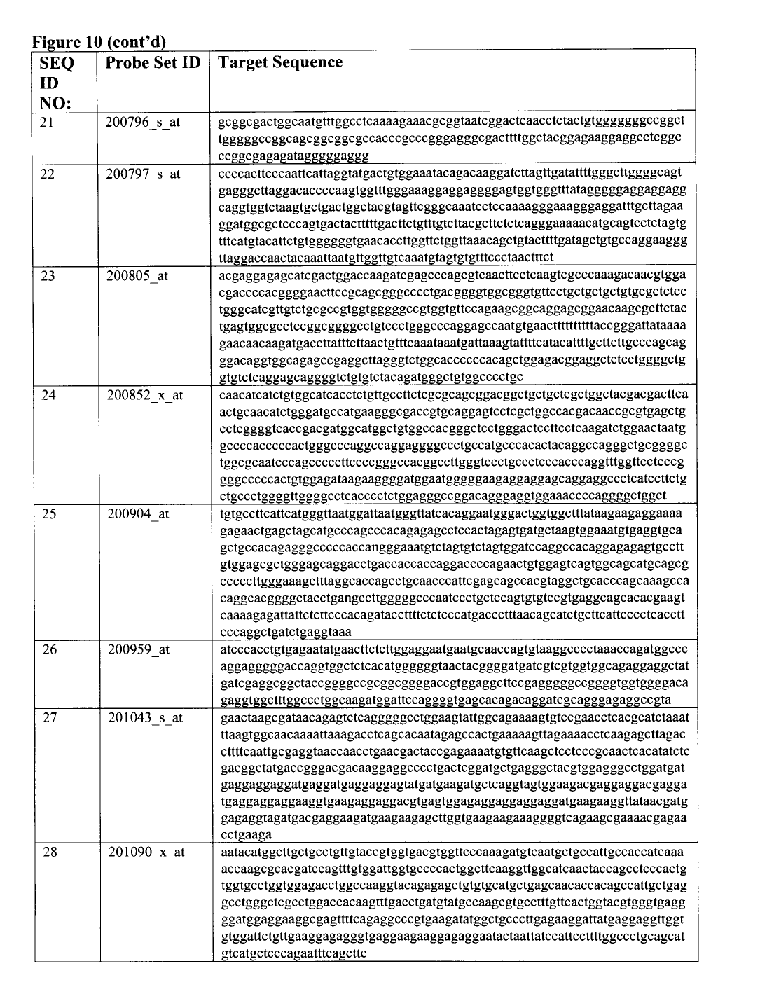

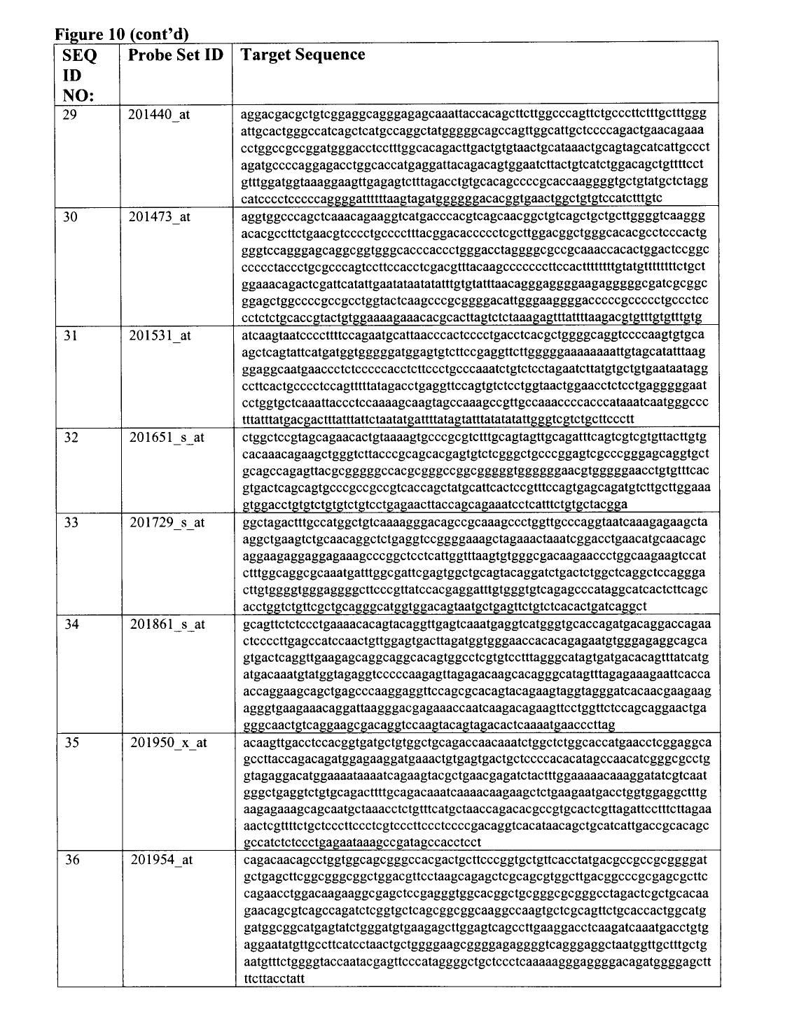

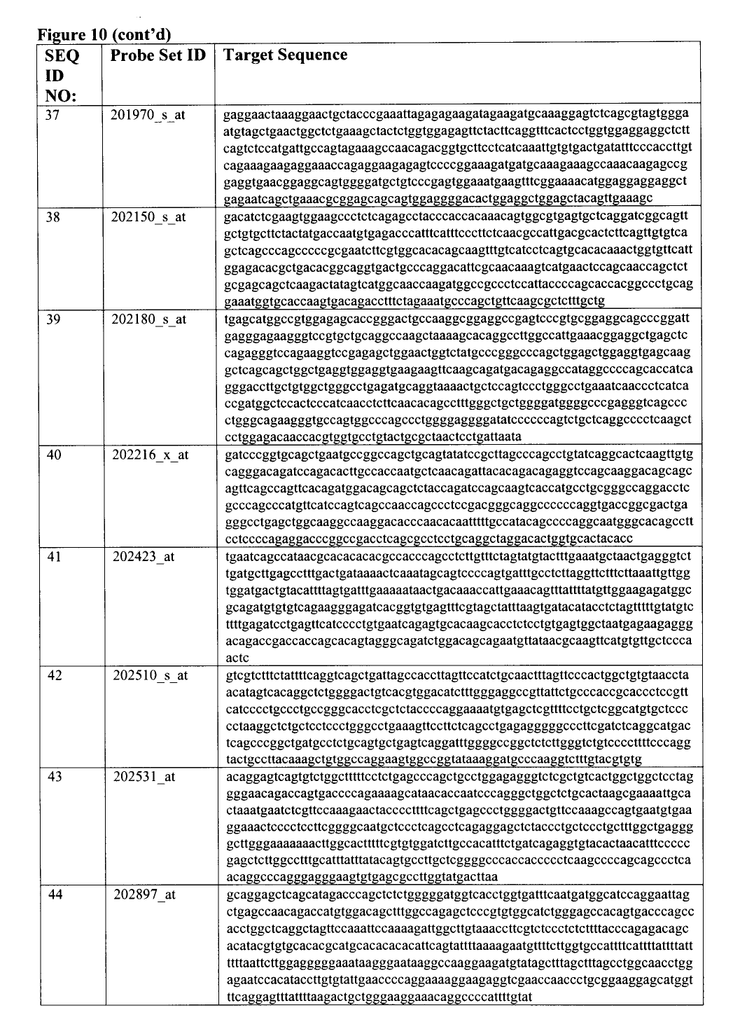

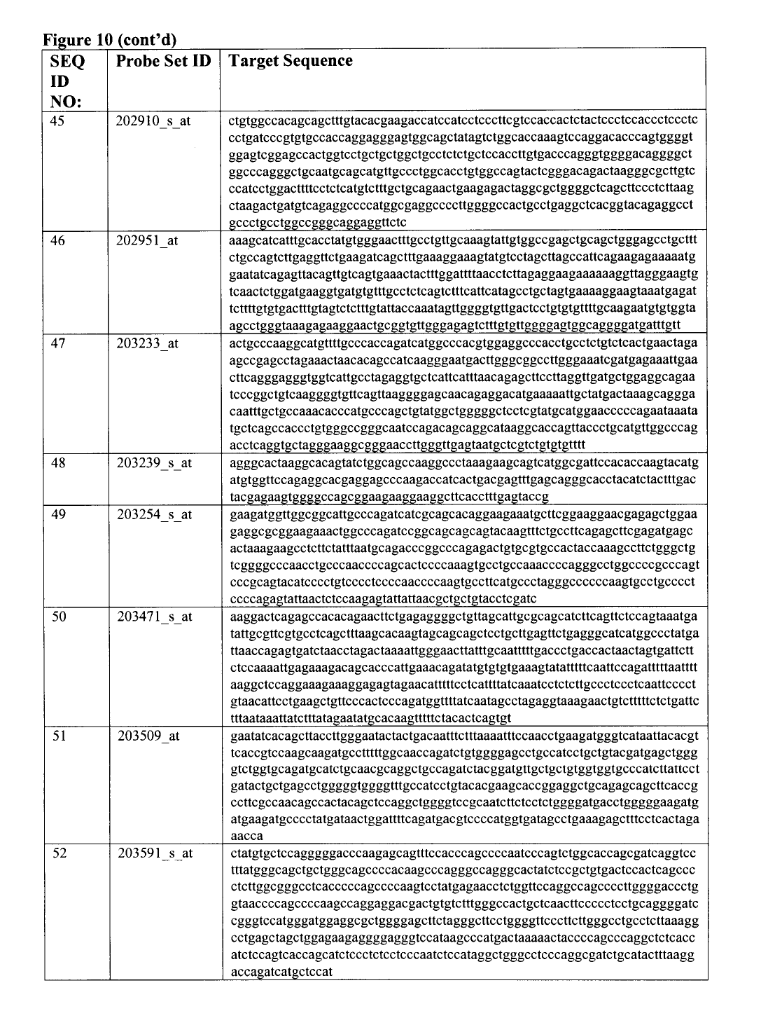

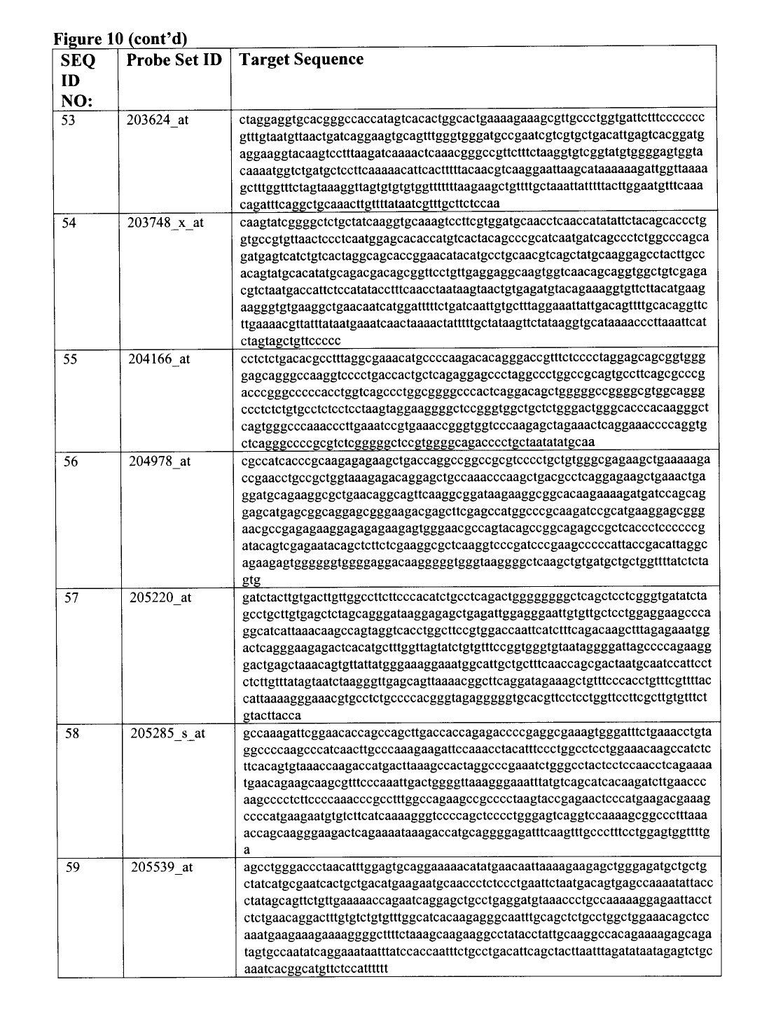

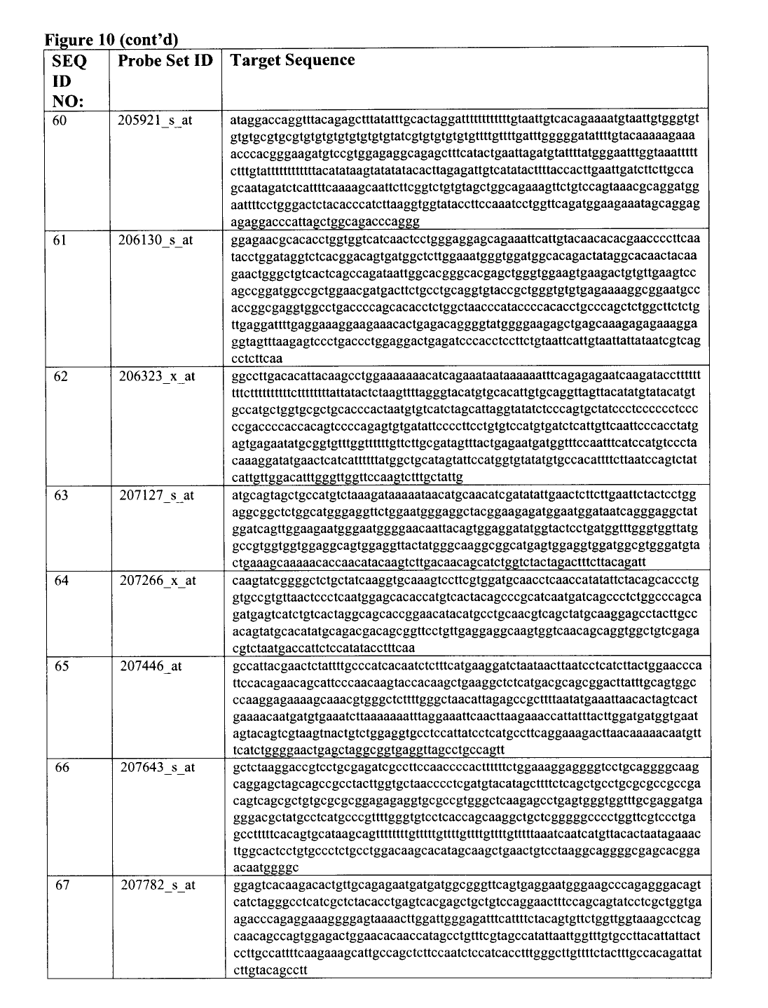

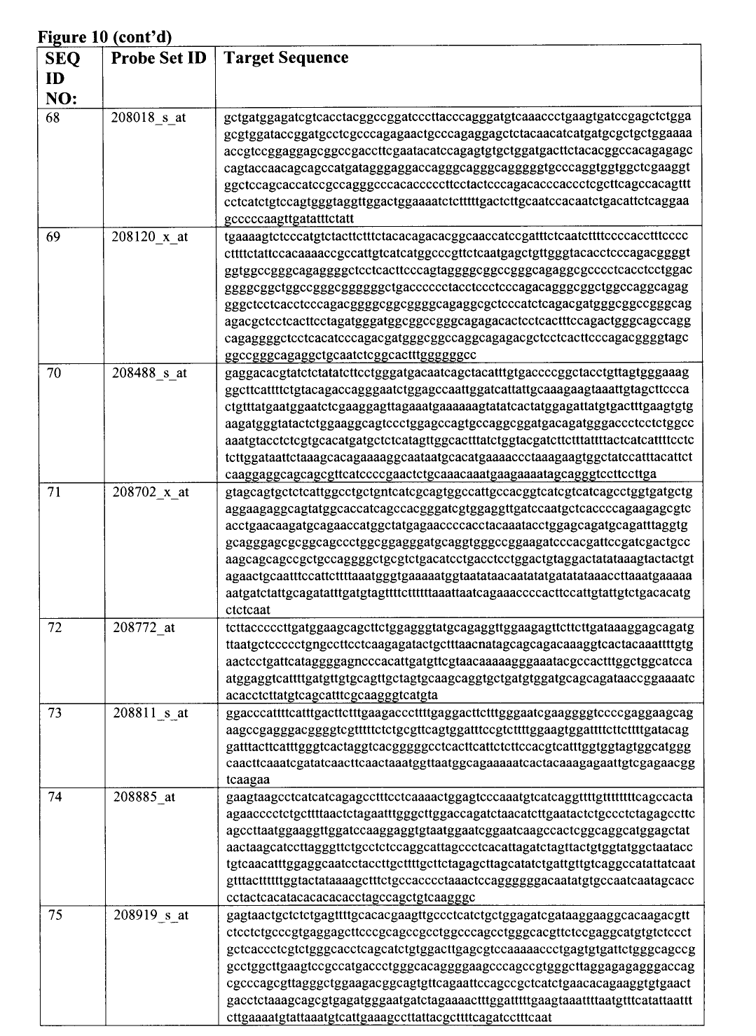

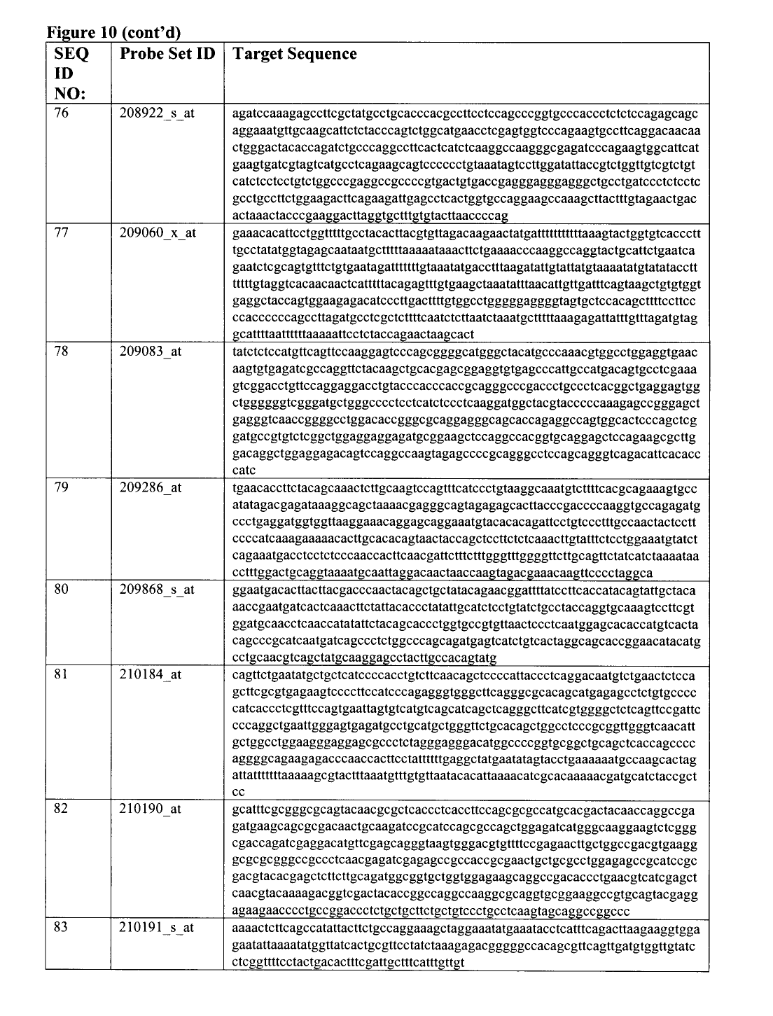

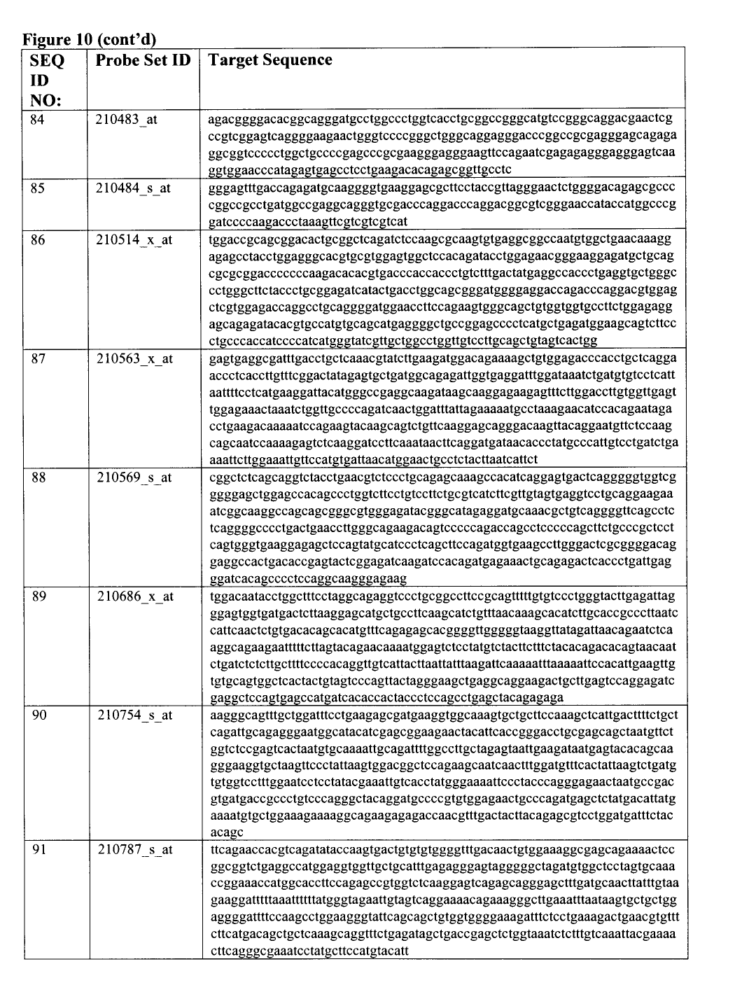

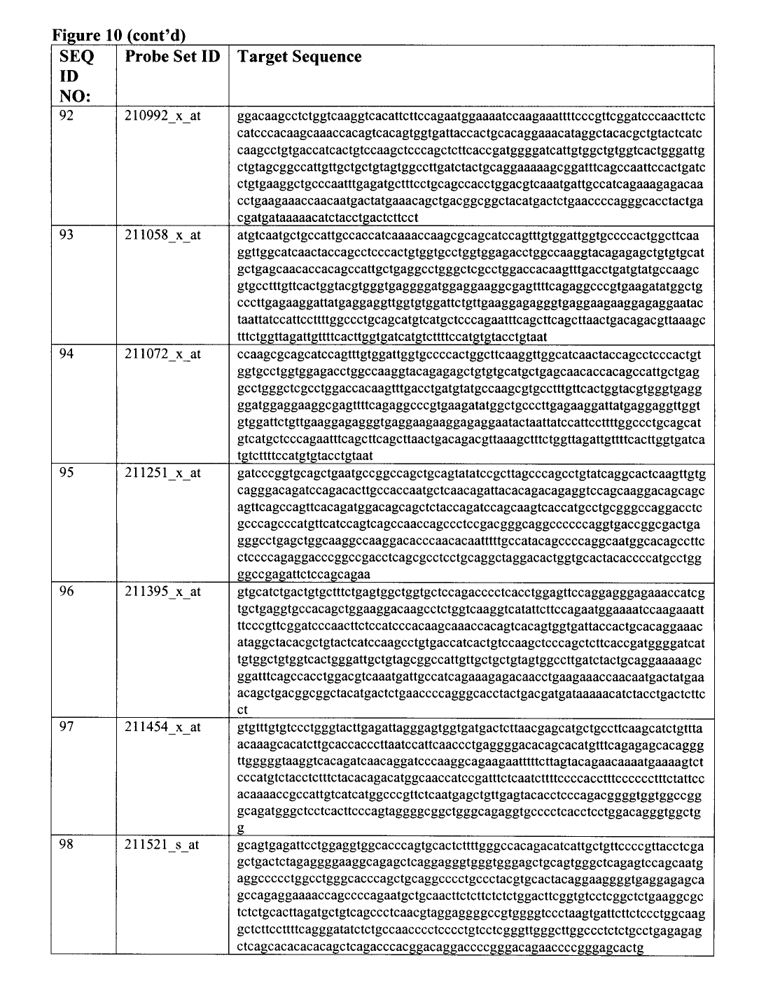

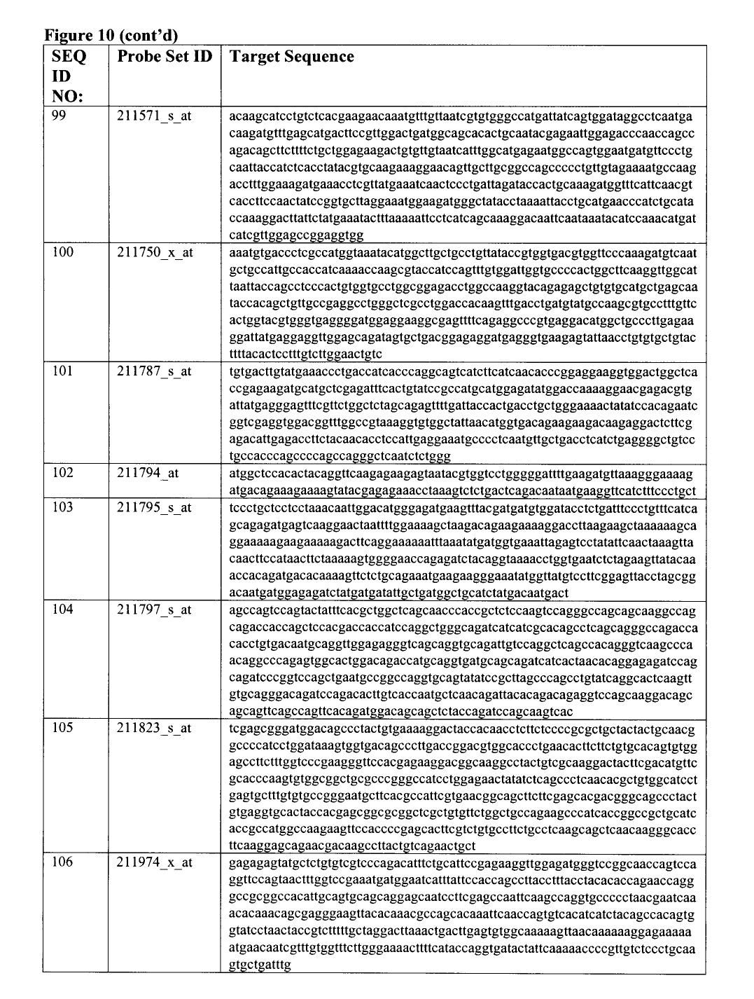

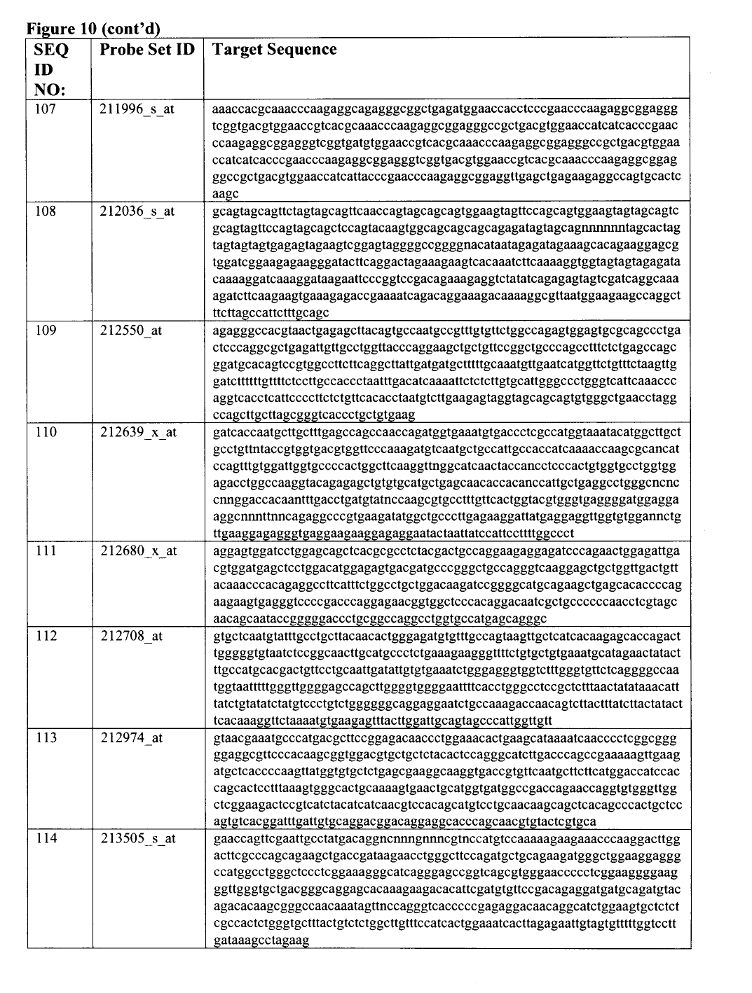

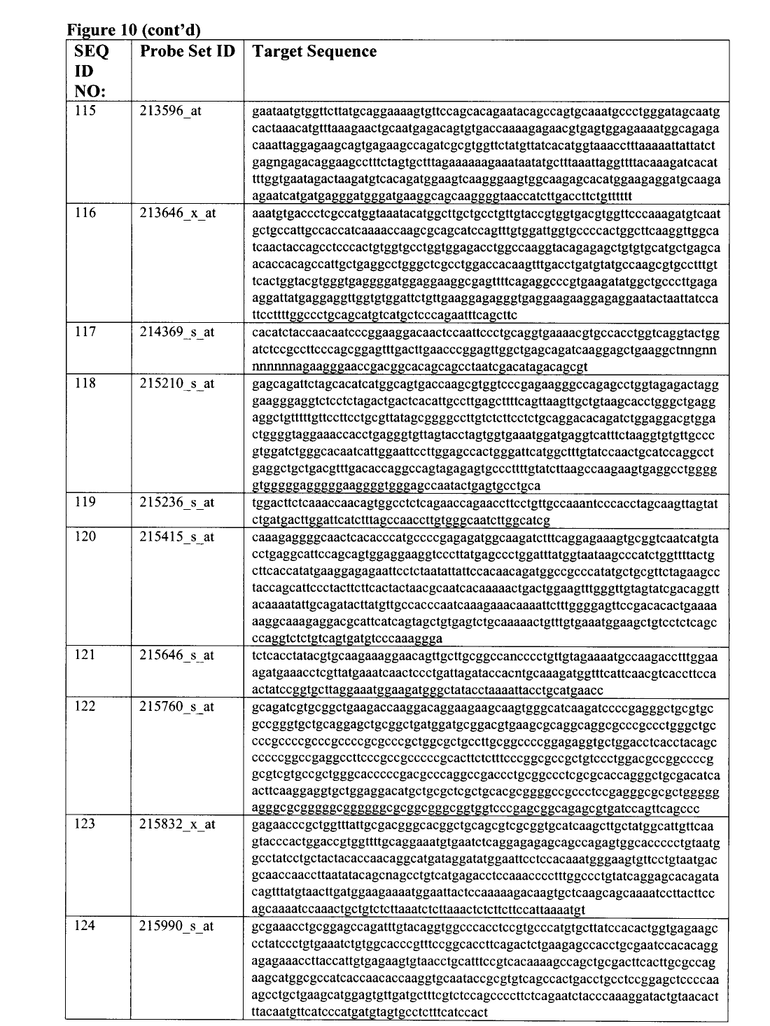

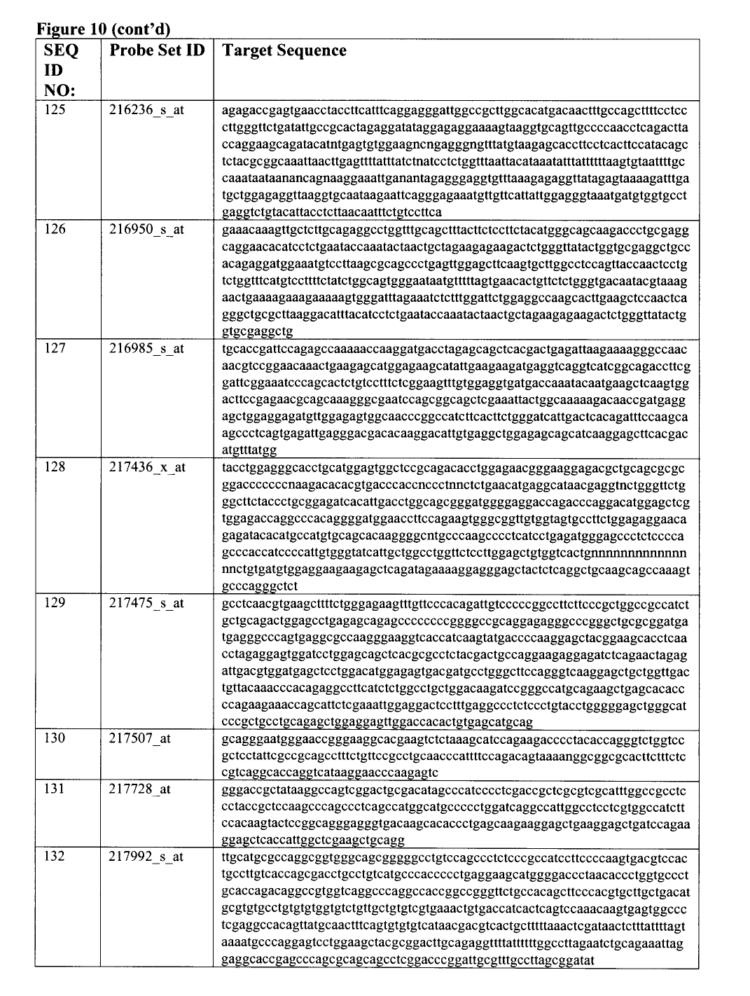

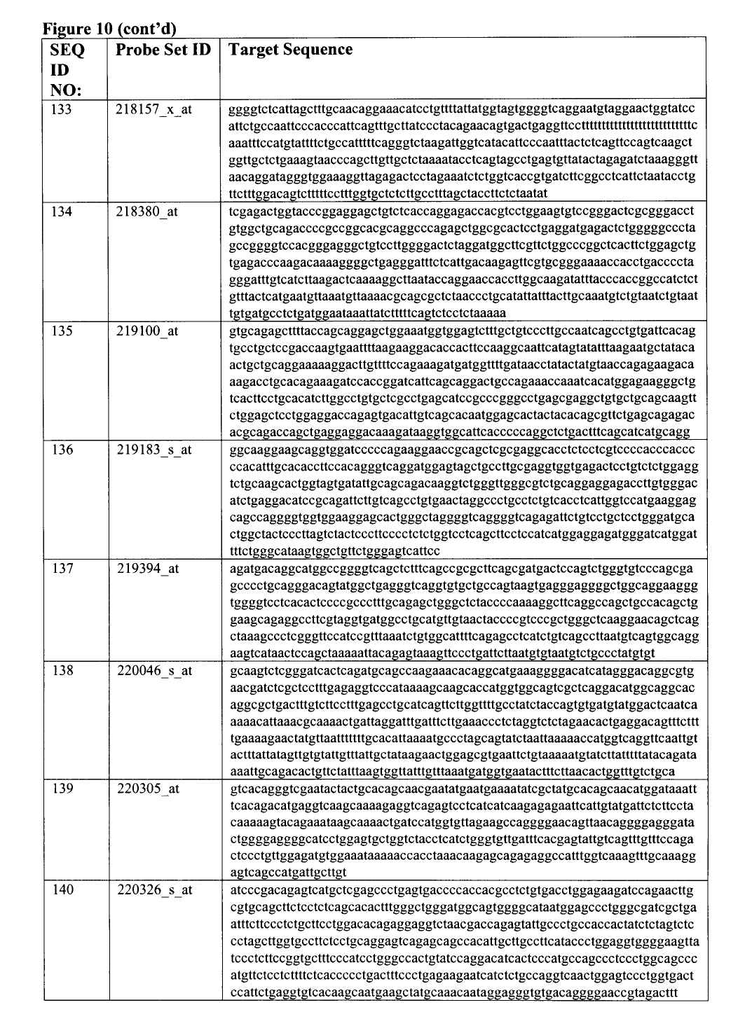

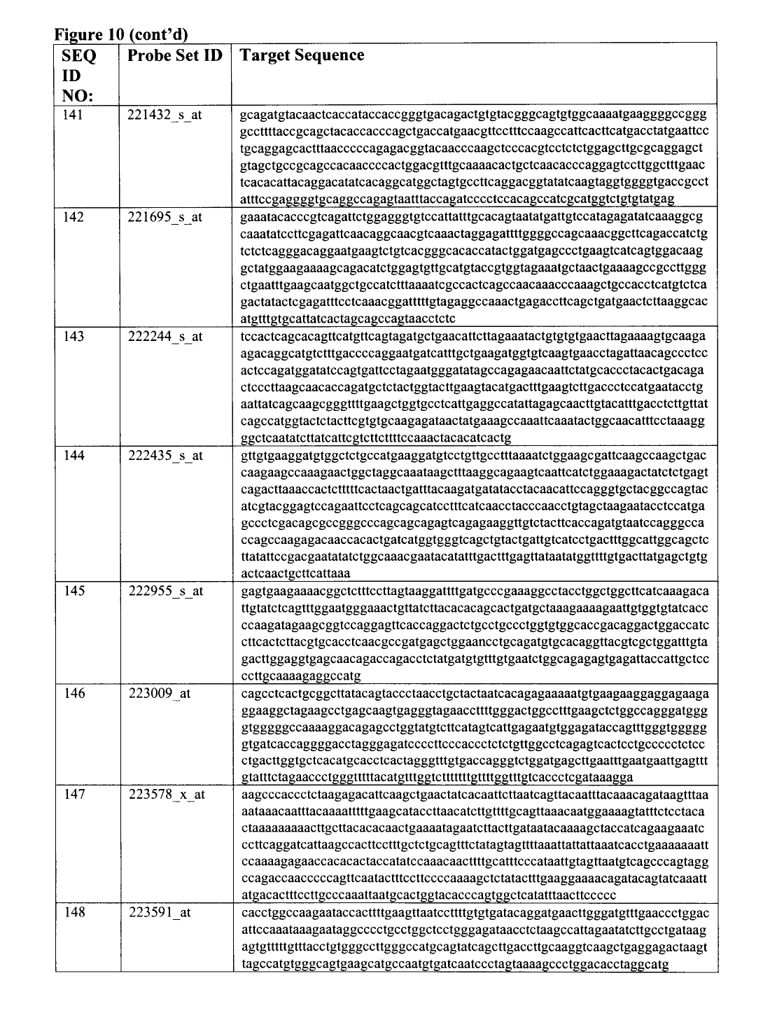

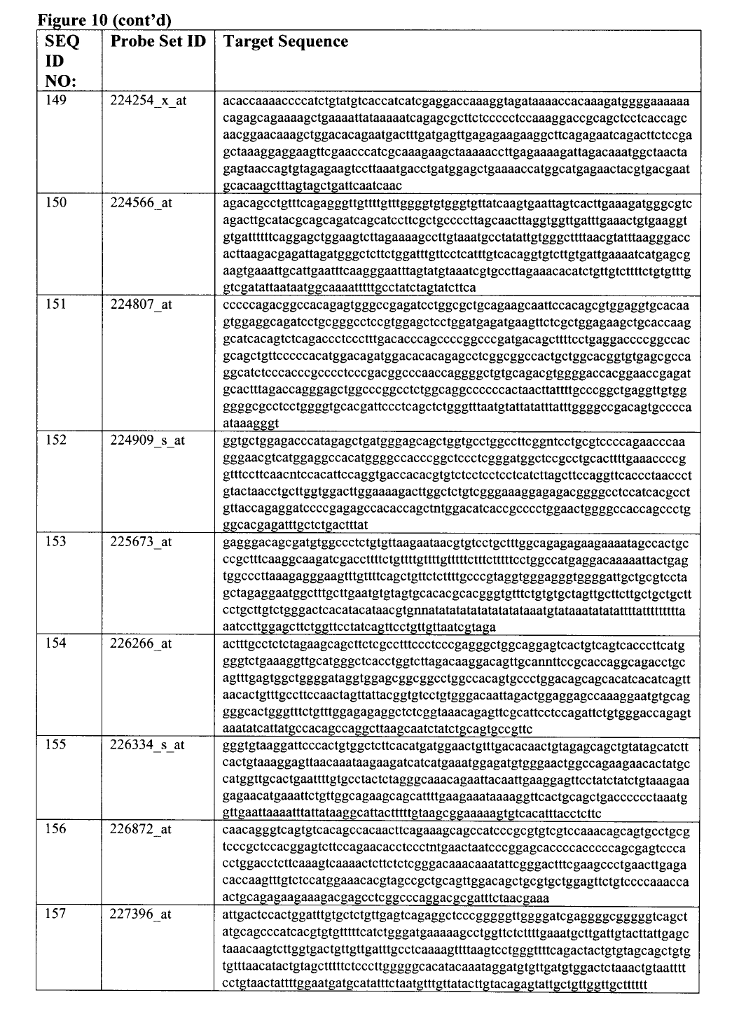

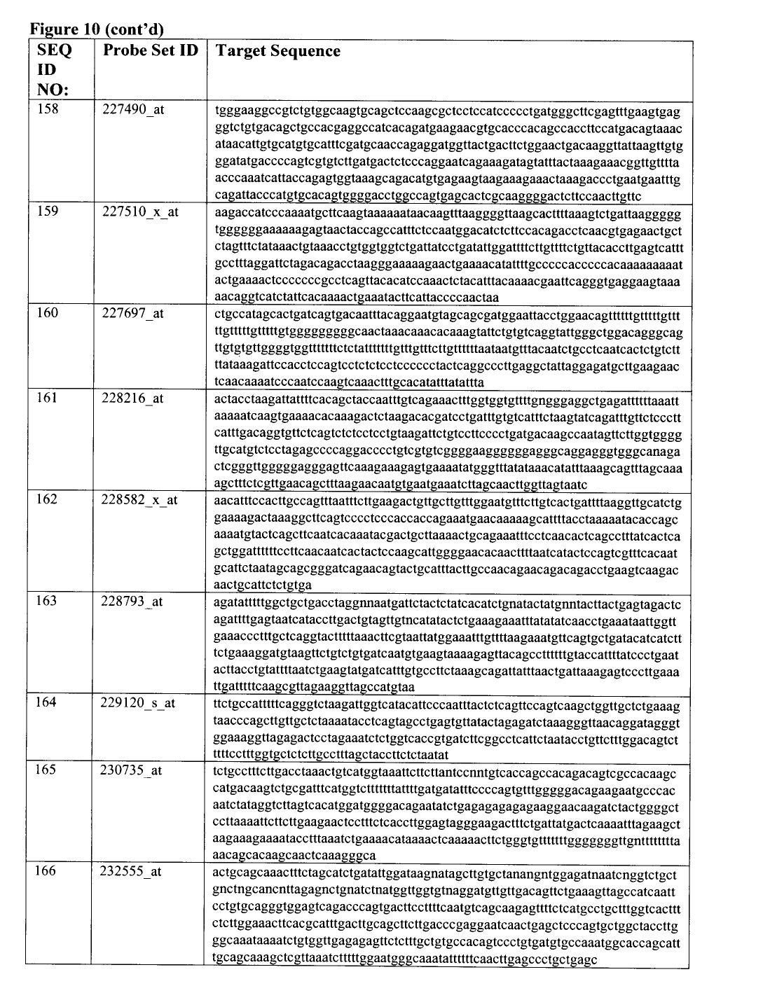

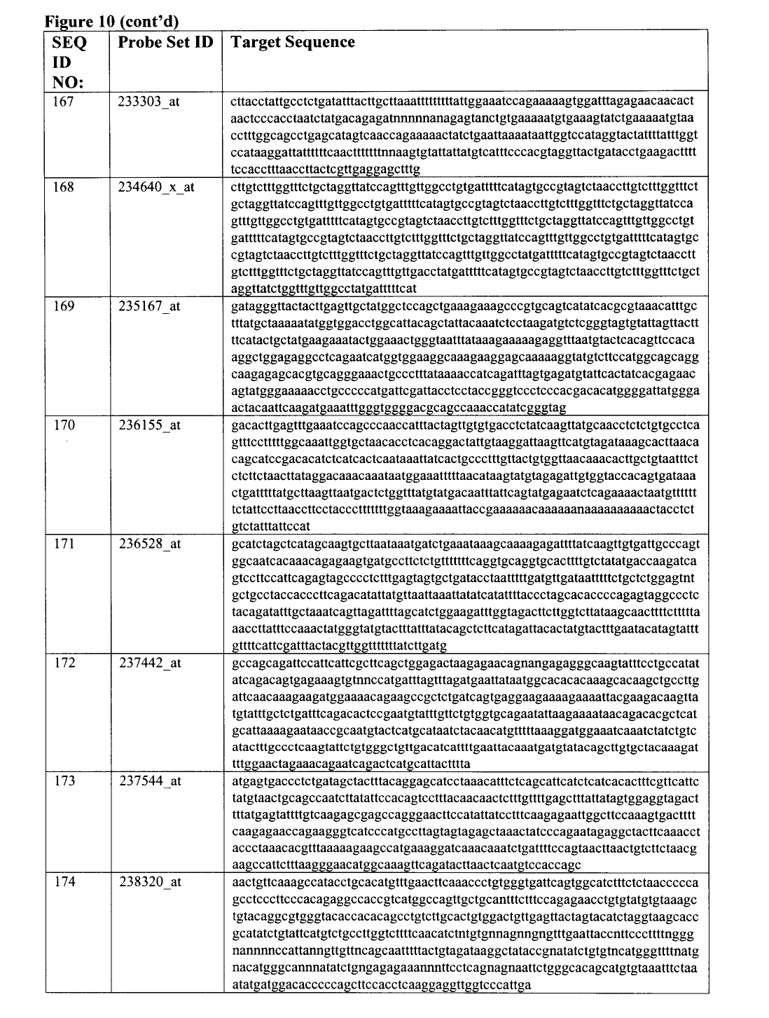

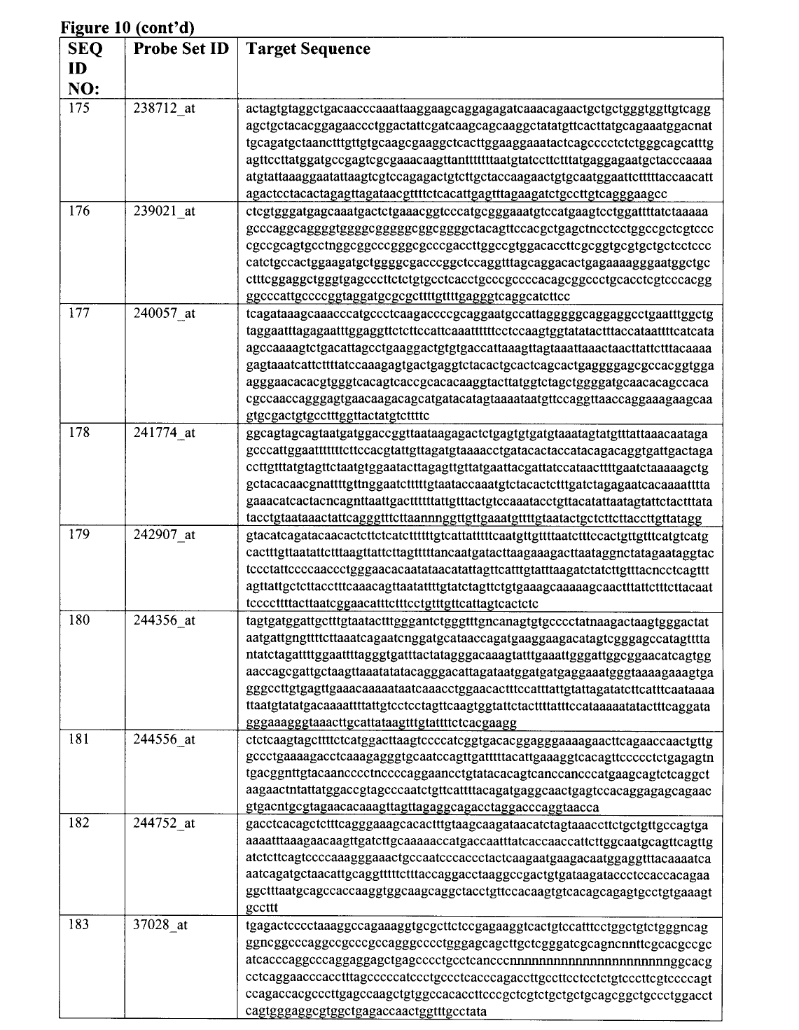

183 probe sets were found to specifically detect (by hybridization and detection of a label) and allow for quantitation of the expression level of the expressed nucleic acids. Of this set of 183 (listed in Table 2), representing 183 individual expressed transcripts or nucleic acids, a subset of 24 probe sets (Table 5) were detected, quantified and found to demonstrate a statistically significant fold change in the AR samples relative to non-rejecting transplant (NR). FIG. 10 provides nucleic acid sequence information of a portion of the nucleic acid identified by the probe sets listed in Tables 2 and 5. Sequences in FIG. 10 (SEQ ID NO: 1-183) may be useful as probes for specific hybridization to the indicated gene (e.g. in a microarray, blot, or other hybridization based assay), or for the design of a primer or primers for specific amplification of the indicated gene (e.g. by PCR, RT-PCR or other amplification-based assay).

18 significant protein group codes were found to have differential relative levels (relative to a reference sample) in AR and NR subjects, using a multiplexed iTRAQ methodology (Table 7). These protein group codes included proteomic markers encoded by one or more than one of TTN, KNG1, LBP, VASN, ARNTL2, AFM, MSTP9, MST1, PI16, SERPINA5, CFD, USH1C, C2, MBL2, SERPINA10, C9, LCAT, B2M, SHBG, C1S, UBR4 and F9. As described below, accession numbers providing specific reference to the nucleic acid sequences encoding these polypeptides, and the amino acid sequences of these polypeptides are provided herein. Unique identifiers (International Protein Index accession numbers) for each member of the indicated protein group codes are found in Table 7. Polypeptides comprising a portion of one or more of these sequences may be useful for the preparation of antibodies that specifically detect one or more of the proteomic markers, alternately, the sequences may be used to identify one or more proteomic markers in a sample subjected to tryptic digest and analysis by mass spectroscopy by comparison of the peptide fragments generated to the sequences, or to a database comprising such sequences.

Detection or determination, and in some cases quantification, of a nucleic acid may be accomplished by any one of a number methods or assays employing recombinant DNA technologies known in the art, including but not limited to, sequence-specific hybridization, polymerase chain reaction (PCR), RT-PCR, microarrays and the like. Such assays may include sequence-specific hybridization, primer extension, or invasive cleavage. Furthermore, there are numerous methods for analyzing/detecting the products of each type of reaction (for example, fluorescence, luminescence, mass measurement, electrophoresis, etc.). Furthermore, reactions can occur in solution or on a solid support such as a glass slide, a chip, a bead, or the like.

Methods of designing and selecting probes for use in microarrays or biochips, or for selecting or designing primers for use in PCR-based assays are known in the art. Once the marker or markers are identified and the sequence of the nucleic acid determined by, for example, querying a database comprising such sequences, or by having an appropriate sequence provided (for example, a sequence listing as provided herein), one of skill in the art will be able to use such information to select appropriate probes or primers and perform the selected assay.

Standard reference works setting forth the general principles of recombinant DNA technologies known to those of skill in the art include, for example: Ausubel et al, Current Protocols In Molecular Biology, John Wiley & Sons, New York (1998 and Supplements to 2001); Sambrook et al, Molecular Cloning: A Laboratory Manual, 2d Ed., Cold Spring Harbor Laboratory Press, Plainview, N.Y. (1989); Kaufman et al, Eds., Handbook Of Molecular And Cellular Methods In Biology And Medicine, CRC Press, Boca Raton (1995); McPherson, Ed., Directed Mutagenesis: A Practical Approach, IRL Press, Oxford (1991).

Proteins, protein complexes or proteomic markers may be specifically identified and/or quantified by a variety of methods known in the art and may be used alone or in combination. Immunologic- or antibody-based techniques include enzyme-linked immunosorbent assay (ELISA), radioimmunoassay (RIA), western blotting, immunofluorescence, microarrays, some chromatographic techniques (i.e. immunoaffinity chromatography), flow cytometry, immunoprecipitation and the like. Such methods are based on the specificity of an antibody or antibodies for a particular epitope or combination of epitopes associated with the protein or protein complex of interest. Non-immunologic methods include those based on physical characteristics of the protein or protein complex itself. Examples of such methods include electrophoresis, some chromatographic techniques (e.g. high performance liquid chromatography (HPLC), fast protein liquid chromatography (FPLC), affinity chromatography, ion exchange chromatography, size exclusion chromatography and the like), mass spectrometry, sequencing, protease digests, and the like. Such methods are based on the mass, charge, hydrophobicity or hydrophilicity, which is derived from the amino acid complement of the protein or protein complex, and the specific sequence of the amino acids. Exemplary methods include those described in, for example, PCT Publication WO 2004/019000, WO 2000/00208, U.S. Pat. No. 6,670,194. Immunologic and non-immunologic methods may be combined to identify or characterize a protein or protein complex. Furthermore, there are numerous methods for analyzing/detecting the products of each type of reaction (for example, fluorescence, luminescence, mass measurement, electrophoresis, etc.). Furthermore, reactions can occur in solution or on a solid support such as a glass slide, a chip, a bead, or the like.

Methods of producing antibodies for use in protein or antibody arrays, or other immunology based assays are known in the art. Once the marker or markers are identified and the amino acid sequence of the protein or polypeptide is identified, either by querying of a database or by having an appropriate sequence provided (for example, a sequence listing as provide herein), one of skill in the art will be able to use such information to prepare one or more appropriate antibodies and perform the selected assay.

For preparation of monoclonal antibodies directed towards a biomarker, any technique that provides for the production of antibody molecules may be used. Such techniques include, but are not limited to, hybridomas or triomas (e.g. Kohler and Milstein 1975, Nature 256:495-497; Gustafsson et al., 1991, Hum. Antibodies Hybridomas 2:26-32), human B-cell hybridoma or EBV hybridomas e.g. (Kozbor et al., 1983, Immunology Today 4:72; Cole et al., 1985, In: Monoclonal Antibodies and Cancer Therapy, Alan R. Liss, Inc., pp. 77-96). Human, or humanized antibodies may be used and can be obtained by using human hybridomas (Cote et al., 1983, Proc. Natl. Acad. Sci. USA 80:2026-2030) or by transforming human B cells with EBV virus in vitro (Cole et al., 1985, In: Monoclonal Antibodies and Cancer Therapy, Alan R. Liss, Inc., pp. 77-96). Techniques developed for the production of “chimeric antibodies” (Morrison et al, 1984, Proc. Natl. Acad. Sci. USA 81:6851-6855; Neuberger et al, 1984, Nature 312:604-608; Takeda et al, 1985, Nature 314:452-454) by splicing a sequence encoding a mouse antibody molecule specific for a particular biomarker together with a sequence encoding a human antibody molecule of appropriate biological activity may be used; such antibodies are within the scope of this invention. Techniques described for the production of single chain antibodies (U.S. Pat. No. 4,946,778) may be adapted to produce a biomarker-specific antibodies. An additional embodiment of the invention utilizes the techniques described for the construction of Fab expression libraries (Huse et al, 1989, Science 246:1275-1281) to allow rapid and easy identification of monoclonal Fab fragments with the desired specificity for a biomarker proteins. Non-human antibodies can be “humanized” by known methods (e.g., U.S. Pat. No. 5,225,539).

Antibody fragments that contain an idiotype of a biomarker can be generated by techniques known in the art. For example, such fragments include, but are not limited to, the F(ab′)2 fragment which can be produced by pepsin digestion of the antibody molecule; the Fab′ fragment that can be generated by reducing the disulfide bridges of the F(ab′)2 fragment; the Fab fragment that can be generated by treating the antibody molecular with papain and a reducing agent; and Fv fragments. Synthetic antibodies, e.g., antibodies produced by chemical synthesis, may also be useful in the present invention.

Standard reference works described herein and known to those skilled in the relevant art describe both immunologic and non-immunologic techniques, their suitability for particular sample types, antibodies, proteins or analyses. Standard reference works setting forth the general principles of immunology and assays employing immunologic methods known to those of skill in the art include, for example: Harlow and Lane, Antibodies: A Laboratory Manual, 2d Ed., Cold Spring Harbor Laboratory Press, Cold Spring Harbor, N.Y. (1999); Harlow and Lane, Using Antibodies: A Laboratory Manual. Cold Spring Harbor Laboratory Press, New York; Coligan et al. eds. Current Protocols in Immunology, John Wiley & Sons, New York, N.Y. (1992-2006); and Roitt et al., Immunology, 3d Ed., Mosby-Year Book Europe Limited, London (1993). Standard reference works setting forth the general principles of peptide synthesis technology and methods known to those of skill in the art include, for example: Chan et al., Fmoc Solid Phase Peptide Synthesis, Oxford University Press, Oxford, United Kingdom, 2005; Peptide and Protein Drug Analysis, ed. Reid, R., Marcel Dekker, Inc., 2000; Epitope Mapping, ed. Westwood et al., Oxford University Press, Oxford, United Kingdom, 2000; Sambrook et al., Molecular Cloning: A Laboratory Manual, 3^{rd} ed., Cold Spring Harbor Press, Cold Spring Harbor, N.Y. 2001; and Ausubel et al., Current Protocols in Molecular Biology, Greene Publishing Associates and John Wiley & Sons, NY, 1994).

A subject's rejection status may be described as “rejector” (R or “acute rejector” or AR) or as a “non-rejector” (NR) and is determined by comparison of the concentration of the markers to that of a non-rejector cutoff index. A “non-rejector cutoff index” is a numerical value or score, beyond or outside of which a subject is categorized as having rejector status. The non-rejector cutoff index may be alternately referred to as a ‘control value’, a ‘control index’, or simply as a ‘control’. A non-rejector cutoff-index may be the concentration of individual markers in a control subject population and considered separately for each marker measured; alternately the non-rejector cutoff index may be a combination of the concentration of the markers, and compared to a combination of the concentration of the markers in the subject's sample provided for diagnosing. The control subject population may be a normal or healthy control population, or may be an allograft recipient population that has not, or is not, rejecting the allograft. A control, or pool of controls, may be constant e.g. represented by a static value, or may be cumulative, in that the sample population used to obtain it may change from site to site, or over time and incorporate additional data points. For example, a central data repository, such as a centralized healthcare information system, may receive and store data obtained at various sites (hospitals, clinical laboratories or the like) and provide this cumulative data set for use with the methods of the invention at a single hospital, community clinic, for access by an end user (i.e. an individual medical practitioner, medical clinic or center, or the like). In some embodiments the cutoff index may be further characterized as being a genomic cutoff index (for genomic expression profiling of subjects), a proteomic cutoff index (for proteomic profiling of subjects), or the like.

A “biological sample” refers generally to body fluid or tissue or organ sample from a subject. For example, the biological sample may be a body fluid such as blood, serum, plasma, lymph fluid, urine or saliva. A tissue or organ sample, such as a non-liquid tissue sample may be digested, extracted or otherwise rendered to a liquid form—examples of such tissues or organs include cultured cells, blood cells, skin, liver, heart, kidney, pancreas, islets of Langerhans, bone marrow, blood, blood vessels, heart valve, lung, intestine, bowel, spleen, bladder, penis, face, hand, bone, muscle, fat, cornea or the like. A plurality of biological samples may be collected at any one time. A biological sample or samples may be taken from a subject at any time, including before allograft transplantation, at the time of transplantation or at anytime following transplantation. A biological sample may comprise “nucleic acid”, such as ‘deoxyribonucleic acid’ (also ‘DNA’) or ‘ribonucleic acid’ (also ‘RNA’ or ‘mRNA’), or a combination thereof, in either single or double-stranded form. A nucleic acid may also be referred to as a ‘transcript’.

The methods described herein may be employed before a subject receives an allograft, or at any time following receipt of an allograft to determine whether or not the allogaft is being rejected. For example, a sample obtained from a subject at any time following the receipt of the allogaft may be assessed for the presence of altered levels (increased or decreased) of one or more than one nucleic acid marker or proteomic marker listed in Tables 2 or 7. In some cases, a sample can be obtained from the subject 1, 2, 3, 4, 5, 6, 7, 8, or more hours after the allograft is received. In some cases, a sample can be obtained from the subject one or more days (e.g., 2, 3, 4, 5, 6, 7, 8, 9, 10, 15, 20, 25, 30, 40, or more days) after the allograft is received. In some examples, a sample can be obtained from 2 to 7 days (e.g., 5 to 7 days) after receipt of the to allograft and assessed for the presence of nucleic acid markers or proteomic markers listed in Tables 2 or 7.

The term “subject” or “patient” generally refers to mammals and other animals including humans and other primates, companion animals, zoo, and farm animals, including, but not limited to, cats, dogs, rodents, rats, mice, hamsters, rabbits, horses, cows, sheep, pigs, goats, poultry, etc. A subject includes one who is to be tested, or has been tested for prediction, assessment or diagnosis of allograft rejection. The subject may have been previously assessed or diagnosed using other methods, such as those described herein or those in current clinical practice, or may be selected as part of a general population (a control subject).

A fold-change of a marker in a subject, relative to a control may be at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0 or more, or any amount there between. The fold change may represent a decrease, or an increase, compared to the control value. One or more than one includes 1, 2, 3, 4, 5, 6, 7, 8, 9, 10, 11, 12, 13, 14, 15, 16, 17, 18, 19, 20, 21, 22, 23, 24 or more.

“Down-regulation” or ‘down-regulated’ may be used interchangeably and refer to a decrease in the level of a marker, such as a gene, nucleic acid, metabolite, transcript, protein or polypeptide. “Up-regulation” or “up-regulated” may be used interchangeably and refer to an increase in the level of a marker, such as a gene, nucleic acid, metabolite, transcript, protein or polypeptide. Also, a pathway, such as a signal transduction or metabolic pathway may be up- or down-regulated.

Once a subject is identified as an acute rejector, or at risk for becoming an acute rejector by any method (genomic or proteomic, or a combination thereof), therapeutic measures may be implemented to alter the subject's immune response to the allograft. The subject may undergo additional monitoring of clinical values more frequently, or using more sensitive monitoring methods. Additionally the subject may be administered immunosuppressive medicaments to decrease or increase the subject's immune response. Even though a subject's immune response needs to be suppressed to prevent rejection of the allograft, a suitable level of immune function is also needed to protect against opportunistic infection. Various medicaments that may be administered to a subject are known; see for example, Goodman and Gilman's The Pharmacological Basis of Therapeutics 11^{th} edition. Ch 52, pp 1405-1431 and references therein; L L Brunton, J S Lazo, K L Parker editors. Standard reference works setting forth the general principles of medical physiology and pharmacology known to those of skill in the art include: Fauci et al., Eds., Harrison's Principles Of Internal Medicine, 14th Ed., McGraw-Hill Companies, Inc. (1998). Other preventative and therapeutic strategies are reviewed in the medical literature—see, for example Djamali et al., 2006. Clin J Am Soc Nephrol 1:623-630.

Genomic Nucleic Acid Expression Profiling

A method of diagnosing acute allograft rejection in a subject as provided by the present invention comprises 1) determining the expression profile of at least one or more markers in a biological sample from the subject, the markers selected from the group presented in Table 2; 2) comparing the expression profile of the at least one or more markers to a non-rejector profile; and 3) determining whether the expression level of the at least one or more markers is up-regulated (increased) or down-regulated (decreased) relative to the control profile, wherein up-regulation or down-regulation of the at least one or more markers is indicative of the rejection status.

The invention also provides for a method of predicting, assessing or diagnosing kidney allograft rejection in a subject as provided by the present invention comprising 1) measuring the increase or decrease of at least one or more markers selected from the group presented in Table 2; and 2) determining the ‘rejection status’ of the subject, wherein the determination of ‘rejection status’ of the subject is based on comparison of the subject's marker expression profile to a control marker expression profile.

The phrase “gene expression data”, “gene expression profile” or “marker expression profile” as used herein refers to information regarding the relative or absolute level of expression of a gene or set of genes in a biological sample. The level of expression of a gene may be determined based on the level of RNA, such as mRNA, encoded by the gene. Alternatively, the level of expression may be determined based on the level of a polypeptide or fragment thereof encoded by the gene.

A ‘polynucleotide’, ‘oligonucleotide’, ‘nucleic acid’ or ‘nucleotide polymer’ as used herein may include synthetic or mixed polymers of nucleic acids, including RNA, DNA or both RNA and DNA, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog, internucleotide modifications such as uncharged linkages (e.g., methyl phosphonates, phosphotriesters, phosphoamidates, carbamates, etc.), charged linkages (e.g., phosphorothioates, phosphorodithioates, etc.), pendent moieties (e.g., polypeptides), and modified linkages (e.g., alpha anomeric polynucleotides, etc.). Also included are synthetic molecules that mimic polynucleotides in their ability to bind to a designated sequence via hydrogen bonding and other chemical interactions.

An oligonucleotide includes variable length nucleic acids, which may be useful as probes, primers and in the manufacture of microarrays (arrays) for the detection and/or amplification of specific nucleic acids. Oligonucleotides may comprise DNA, RNA, PNA or other polynucleotide moieties as described in, for example, U.S. Pat. No. 5,948,902. Such DNA or RNA strands may be synthesized by the sequential addition (5′-3′ or 3′-5′) of activated monomers to a growing chain which may be linked to an insoluble support. Numerous methods are known in the art for synthesizing oligonucleotides for subsequent individual use or as a part of the insoluble support, for example in arrays (Lashkari D A. et al. PNAS (1995) 92(17):7912-5; McGall G. et al. PNAS (1996) 93(24):13555-60; Albert T J. et al. Nucleic Acid Res. (2003) 31(7):e35; Gao X. et al. Biopolymers (2004) 73(5):579-96; and Moorcroft M J. et al. Nucleic Acid Res. (2005) 33(8):e75 and references therein). In general, oligonucleotides are synthesized through the stepwise addition of activated and protected monomers under a variety of conditions depending on the method being used. Subsequently, specific protecting groups may be removed to allow for further elongation and subsequently and once synthesis is complete all the protecting groups may be removed and the oligonucleotides removed from their solid supports for purification of the complete chains if so desired.

A “gene” is an ordered sequence of nucleotides located in a particular position on a particular chromosome that encodes a specific functional product and may include untranslated and untranscribed sequences in proximity to the coding regions (5′ and 3′ to the coding sequence), as well as exons and/or introns. Such non-coding sequences may contain regulatory sequences needed for transcription and translation of the sequence or splicing of introns, for example, or may as yet to have any function attributed to them beyond the occurrence of the mutation of interest. A gene may also include one or more promoters, enhancers, transcription factor binding sites, termination signals or other regulatory elements.

The term “microarray,” “array,” or “chip” refers to a plurality of defined nucleic acid probes coupled to the surface of a substrate in defined locations. The substrate may be a solid substrate. Microarrays have been generally described in the art in, for example, U.S. Pat. Nos. 5,143,854 (Pirrung); 5,424,186, 5,445,934, 5,744,305 and 5,800,992 to Fodor, 5,677,195 and 6,040,193 to Winkler, and Fodor et al. 1991 (Science, 251:767-777). Each of these references is incorporated by reference herein in their entirety.

“Hybridization” includes a reaction in which one or more polynucleotides and/or oligonucleotides interact in an ordered manner (sequence-specific) to form a complex that is stabilized by hydrogen bonding—also referred to as ‘Watson-Crick’ base pairing. Variant base-pairing may also occur through non-canonical hydrogen bonding includes Hoogsteen base pairing. Under some thermodynamic, ionic or pH conditions, triple helices may occur, particularly with ribonucleic acids. These and other variant hydrogen bonding or base-pairing are known in the art, and may be found in, for example, Lehninger—Principles of Biochemistry, 3^{rd} edition (Nelson and Cox, eds. Worth Publishers, New York.), herein incorporated by reference.

Hybridization reactions can be performed under conditions of different “stringency”. The stringency of a hybridization reaction can determine the ease or difficulty with which any two nucleic acid molecules will hybridize to one another. Stringency may be increased, for example, by increasing the temperature at which hybridization occurs, by decreasing the ionic (salt) concentration at which hybridization occurs, or a combination thereof. Under stringent conditions, nucleic acid molecules at least 60%, 65%, 70%, 75% or more identical to each other remain hybridized to each other, whereas molecules with low percent identity generally do not remain hybridized. An example of stringent hybridization conditions are hybridization in 6× sodium chloride/sodium citrate (SSC) at about 44-45° C., followed by one or more washes in 0.2×SSC, 0.1% SDS at 50° C., 55° C., 60° C., 65° C., or at a temperature there between.

Hybridization between two nucleic acids may occur in an antiparallel configuration—this is referred to as ‘annealing’, and the paired nucleic acids are described as complementary. A double-stranded polynucleotide may be “complementary”, if hybridization can occur between one of the strands of the first polynucleotide and the second. The degree of which one polynucleotide is complementary with another is referred to as homology, and is quantifiable in terms of the proportion of bases in opposing strands that are expected to hydrogen bond with each other, according to generally accepted base-pairing rules.

In general, sequence-specific hybridization involves a hybridization probe, which is capable of specifically hybridizing to a defined sequence. Such probes may be designed to differentiate between sequences varying in only one or a few nucleotides, thus providing a high degree of specificity. A strategy which couples detection and sequence discrimination is the use of a “molecular beacon”, whereby the hybridization probe (molecular beacon) has 3′ and/or 5′ reporter and quencher molecules and 3′ and 5′ sequences which are complementary such that absent an adequate binding target for the intervening sequence the probe will form a hairpin loop. The hairpin loop keeps the reporter and quencher in close proximity resulting in quenching of the fluorophor (reporter) which reduces fluorescence emissions. However, when the molecular beacon hybridizes to the target the fluorophor and the quencher are sufficiently separated to allow fluorescence to be emitted from the fluorophor.

Probes used in hybridization may include double-stranded DNA, single-stranded DNA and RNA oligonucleotides, and peptide nucleic acids. Hybridization conditions and methods for identifying markers that hybridize to a specific probe are described in the art—see, for example, Brown, T. “Hybridization Analysis of DNA Blots” in Current Protocols in Molecular Biology. F M Ausubel et al, editors. Wiley & Sons, 2003. doi: 10.1002/0471142727.mb0210s21. Suitable hybridization probes for use in accordance with the invention include oligonucleotides, polynucleotides or modified nucleic acids from about 10 to about 400 nucleotides, alternatively from about 20 to about 200 nucleotides, or from about 30 to about 100 nucleotides in length.

Specific sequences may be identified by hybridization with a primer or a probe, and this hybridization subsequently detected.

A “primer” includes a short polynucleotide, generally with a free 3′-OH group that binds to a target or “template” present in a sample of interest by hybridizing with the target, and thereafter promoting polymerization of a polynucleotide complementary to the target. A “polymerase chain reaction” (“PCR”) is a reaction in which replicate copies are made of a target polynucleotide using a “pair of primers” or “set of primers” consisting of “upstream” and a “downstream” primer, and a catalyst of polymerization, such as a DNA polymerase, and typically a thermally-stable polymerase enzyme. Methods for PCR are well known in the art, and are taught, for example, in Beverly, S M. Enzymatic Amplification of RNA by PCR (RT-PCR) in Current Protocols in Molecular Biology. F M Ausubel et al, editors. Wiley & Sons, 2003. doi: 10.1002/0471142727.mb1505s56. Synthesis of the replicate copies may include incorporation of a nucleotide having a label or tag, for example, a fluorescent molecule, biotin, or a radioactive molecule. The replicate copies may subsequently be detected via these tags, using conventional methods.

A primer may also be used as a probe in hybridization reactions, such as Southern or Northern blot analyses (see, e.g., Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989).

A “probe set” (or sometimes ‘primer set’) as used herein refers to a group of oligonucleotides that may be used to detect the presence of a nucleic acid molecule (a nucleic acid marker) in a sample; the detection may be quantitative, or semi-quantitative. Detection may be, for example, through amplification as in PCR and RT-PCR, or through hybridization, as on a microarray, or through selective destruction and protection, as in assays based on the selective enzymatic degradation of single or double stranded nucleic acids. Probes in a probe set may be labeled with one or more fluorescent, radioactive or other detectable moieties (including enzymes). Probes may be any size so long as the probe is sufficiently large to selectively detect the desired gene—generally a size range from about 15 to about 25, or to about 30 nucleotides is of sufficient size. A probe set may be in solution, e.g. for use in multiplex PCR. Alternately, a probe set may be adhered to a solid surface, as in an array or microarray. A probe set may detect the expression level of a full-length gene, a splice-variant of a full-length gene, a transcriptional unit, or a fragment of a gene or transcriptional unit. A probe set identifies a nucleic acid marker that is present in the sample.

In some embodiments of the invention, a probe set for detection of nucleic acids expressed by a set of nucleic acid markers comprising one or more than one of TncRNA, FKSG49, ZNF438, SFRS16, 1558448_a_at, CAMKK2, NFYC, NCOA3, LMAN2, PGS1, NEDD9, 237442_at, FKSG49/LOC730444, LIMK2, UNB, NASP, PRO1073, 240057_at, ITGAX, LOC730399/LOC731974, FKBP1A, HLA-G, RBMS1 and SLC6A6 is provided. Such a probe set may be useful for determining the rejection status of a subject. The probe set may comprise one or more pairs of primers for specific amplification (e.g. PCR, or RT-PCR) of nucleic acid sequences corresponding to one or more than one of TncRNA, FKSG49, ZNF438, SFRS16, 1558448_a_at, CAMKK2, NFYC, NCOA3, LMAN2, PGS1, NEDD9, 237442_at, FKSG49/LOC730444, LIMK2, UNB, NASP, PRO1073, 240057_at, ITGAX, LOC730399/LOC731974, FKBP1A, HLA-G, RBMS1 and SLC6A6. In another embodiment of the invention, the probe set is part of a microarray. In another embodiment of the invention, the nucleic acid markers include one or more than one of TncRNA, FKSG49, ZNF438, 1558448_a_at, CAMKK2, LMAN2, 237442_at, FKSG49/LOC730444, JUNB, PRO1073 and ITGAX. The markers are described in further detail below.

It will be appreciated that numerous other methods for sequence discrimination and detection are known in the art and some of which are described in further detail below. It will also be appreciated that reactions such as arrayed primer extension mini sequencing, tag microarrays and sequence-specific extension could be performed on a microarray. One such array based genotyping platform is the microsphere based tag-it high throughput array (Bortolin S. et al. 2004 Clinical Chemistry 50: 2028-36). This method amplifies genomic DNA by PCR followed by sequence-specific primer extension with universally tagged primers. The products are then sorted on a Tag-It array and detected using the Luminex xMAP system.

It will be appreciated by a person of skill in the art that any numerical designations of nucleotides within a sequence are relative to the specific sequence. Also, the same positions may be assigned different numerical designations depending on the way in which the sequence is numbered and the sequence chosen. Furthermore, sequence variations such as insertions or deletions, may change the relative position and subsequently the numerical designations of particular nucleotides at and around a mutational site. For example, the sequences represented by accession numbers e.g. AC124566, AF211864, AI035495, AI326085, AK089167, AK131133, AK155816, AK170432, BC042840 and BC057200 all represent human ITGAX nucleotide sequences, but may have some sequence differences, and numbering differences between them. As another example, the sequences represented by accession numbers NP_{—}115925, NP_{—}444509, P20702, NP_{—}776169, NP_{—}000878, NP_{—}001706, NP_{—}04223, AAA59180, AAA51620 all represent human ITGAX polypeptide sequences, but may have some sequence differences, and numbering differences between them. Other nucleic acid markers may demonstrate variants, and are described below.

Selection and/or design of probes, primers or probe sets for specific detection of expression of any gene of interest, including any of the above genes is within the ability of one of skill in the relevant art, when provided with one or more nucleic acid sequences of the gene of interest. Further, any of several probes, primers or probe sets, or a plurality of probes, primers or probe sets may be used to detect a gene of interest, for example, an array may include multiple probes for a single gene transcript—the aspects of the invention as described herein are not limited to any specific probes exemplified.

Sequence identity or sequence similarity may be determined using a nucleotide sequence comparison program (for DNA or RNA sequences, or fragments or portions thereof) or an amino acid sequence comparison program (for protein, polypeptide or peptide sequences, or fragments or portions thereof), such as that provided within DNASIS (for example, but not limited to, using the following parameters: GAP penalty 5, # of top diagonals 5, fixed GAP penalty 10, k-tuple 2, floating gap 10, and window size 5). However, other methods of alignment of sequences for comparison are well-known in the art for example the algorithms of Smith & Waterman (1981, Adv. Appl. Math. 2:482), Needleman & Wunsch (J. Mol. Biol. 48:443, 1970), Pearson & Lipman (1988, Proc. Nat'l. Acad. Sci. USA 85:2444), and by computerized implementations of these algorithms (e.g. GAP, BESTFIT, FASTA, and BLAST), or by manual alignment and visual inspection.

If a nucleic acid or gene, polypeptide or sequence of interest is identified and a portion or fragment of the sequence (or sequence of the gene polypeptide or the like) is provided, other sequences that are similar, or substantially similar may be identified using the programs exemplified above. For example, when constructing a microarray or probe sequences, the sequence and location are known, such that if a microarray experiment identifies a ‘hit’ (the probe at a particular location hybridizes with one or more nucleic acids in a sample, the sequence of the probe will be known (either by the manufacturer or producer of the microarray, or from a database provided by the manufacturer—for example the NetAffx databases of Affymetrix, the manufacturer of the Human Genome U133 Plus 2.0 Array). If the identity of the sequence source is not provided, it may be determined by using the sequence of the probe in a sequence-based search of one or more databases. For peptide or peptide fragments identified by proteomics assays, for example iTRAQ, the sequence of the peptide or fragment may be used to query databases of amino acid sequences as described above. Examples of such a database include those maintained by the National Centre for Biotechnology Information, or those maintained by the Swiss Institute of Bioinformatics, the Sanger Centre, or the European Bioinformatics Institute, such as the International Protein Index (IPI).

A protein or polypeptide, nucleic acid or fragment or portion thereof may be considered to be specifically identified when its sequence may be differentiated from others found in the same phylogenetic Species, Genus, Family or Order. Such differentiation may be identified by comparison of sequences. Comparisons of a sequence or sequences may be done using a BLAST algorithm (Altschul et al. 1009. J. Mol. Biol 215:403-410). A BLAST search allows for comparison of a query sequence with a specific sequence or group of sequences, or with a larger library or database (e.g. GenBank or GenPept) of sequences, and identify not only sequences that exhibit 100% identity, but also those with lesser degrees of identity. For example, regarding a protein with multiple isoforms (either resulting from, for example, separate genes or variant splicing of the nucleic acid transcript from the gene, or post translational processing), an isoform may be specifically identified when it is differentiated from other isoforms from the same or a different species, by specific detection of a structure, sequence or motif that is present on one isoform and is absent, or not detectable on one or more other isoforms.

Access to the methods of the invention may be provided to an end user by, for example, a clinical laboratory or other testing facility performing the individual marker tests—the biological samples are provided to the facility where the individual tests and analyses are performed and the predictive method applied; alternately, a medical practitioner may receive the marker values from a clinical laboratory and use a local implementation or an internet-based implementation to access the predictive methods of the invention.

Determination of statistical parameters such as multiples of the median, standard error, standard deviation and the like, as well as other statistical analyses as described herein are known and within the skill of one versed in the relevant art. Use of a particular coefficient, value or index is exemplary only and is not intended to constrain the limits of the various aspects of the invention as disclosed herein.

Interpretation of the large body of gene expression data obtained from, for example, microarray experiments, or complex RT-PCR experiments may be a formidable task, but is greatly facilitated through use of algorithms and statistical tools designed to organize the data in a way that highlights systematic features. Visualization tools are also of value to represent differential expression by, for example, varying intensity and hue of colour (Eisen et al. 1998. Proc Natl Acad Sci 95:14863-14868). The algorithm and statistical tools available have increased in sophistication with the increase in complexity of arrays and the resulting datasets, and with the increase in processing speed, computer memory, and the relative decrease in cost of these.

Mathematical and statistical analysis of nucleic acid or protein expression profiles may accomplish several things—identification of groups of genes that demonstrate coordinate regulation in a pathway or a domain of a biological system, identification of similarities and differences between two or more biological samples, identification of features of a gene expression profile that differentiate between specific events or processes in a subject, or the like. This may include assessing the efficacy of a therapeutic regimen or a change in a therapeutic regimen, monitoring or detecting the development of a particular pathology, differentiating between two otherwise clinically similar (or almost identical) pathologies, or the like.

Clustering methods are known and have been applied to microarray datasets, for example, hierarchical clustering, self-organizing maps, k-means or deterministic annealing. (Eisen et al, 1998 Proc Natl Acad Sci USA 95:14863-14868; Tamayo, P., et al. 1999. Proc Natl Acad Sci USA 96:2907-2912; Tavazoie, S., et al. 1999. Nat Genet. 22:281-285; Alon, U., et al. 1999. Proc Natl Acad Sci USA 96:6745-6750). Such methods may be useful to identify groups of genes in a gene expression profile that demonstrate coordinate regulation, and also useful for the identification of novel genes of otherwise unknown function that are likely to participate in the same pathway or system as the others demonstrating coordinate regulation.

The pattern of nucleic acid or proteomic expression in a biological sample may also provide a distinctive and accessible molecular picture of its functional state and identity. Two different samples that have related gene expression patterns are may be biologically and functionally similar to one another; conversely two samples that demonstrate significant differences in the pattern of nucleic acid or proteomic expression may not only be differentiated by the complex expression pattern displayed, but may indicate a diagnostic subset of gene products or transcripts that are indicative of a specific pathological state or other physiological condition, such as allograft rejection.

Applying a plurality of mathematical and/or statistical analytical methods to a microarray dataset may indicate varying subsets of significant markers, leading to uncertainty as to which method is ‘best’ or ‘more accurate’. Regardless of the mathematics, the underlying biology is the same in a dataset. By applying a plurality of mathematical and/or statistical methods to a microarray dataset and assessing the statistically significant subsets of each for common markers to all, the uncertainty is reduced, and clinically relevant core group of markers is identified.

Genomic Expression Profiling Markers

The present invention provides for a core group of nucleic acid markers useful for the assessment or diagnosis of allograft rejection, including acute kidney allograft rejection, comprising one or more than one of the nucleic acid markers presented in Table 2, and may include one or more than one of TncRNA, FKSG49, ZNF438, SFRS16, 1558448_a_at, CAMKK2, NFYC, NCOA3, LMAN2, PGS1, NEDD9, 237442_at, FKSG49/LOC730444, LIMK2, UNB, NASP, PRO1073, 240057_at, ITGAX, LOC730399/LOC731974, FKBP1A, HLA-G, RBMS1 and SLC6A6.

183 probe sets were detected, quantified and found to demonstrate a statistically significant discrimination, with a false discovery rate (FDR) below 1%, comparing the rejection (AR) samples and non-rejecting transplant (NR) controls in all of the three moderated t-tests applied, and may represent an increase/up-regulation or decrease/down-regulation of the gene or transcript in question. These probe sets specifically detect (by hybridization and detection of a label) and allow for quantitation of the expression level of the expressed nucleic acids. Of this set of 183 (listed in Table 2), representing 183 individual expressed transcripts or nucleic acids, a subset of 24 probe sets (Table 5) were detected, quantified and found to demonstrate a statistically significant fold change in the AR samples relative to non-rejecting transplant (NR) controls in all of the three moderated t-tests applied, and may represent an increase/up-regulation or decrease/down-regulation of the gene or transcript in question. Of these 24 probe sets, at least 18 detect specific genes (known, or known but not described) genes or transcripts. FIG. 10 provides nucleic acid sequence information of a portion of the nucleic acid identified by the probe sets listed in Tables 2 and 5.

In some embodiments, the present invention provides a method for the assessment, monitoring, prediction or diagnosis of allograft rejection, including acute kidney allograft rejection, comprising measuring the expression level of at least one or more of the markers or probe sets selected from the group listed in Table 2, and referred to by the indicated gene symbol. These probe sets are associated with and may specifically measure the expression level individual and unique genes or gene fragments referenced by the gene symbol.

The genes or markers indicated in Tables 2 or 5 may have a biological role in the allograft rejection process, and represent a therapeutic target.

In another embodiment, the present invention provides for a group of nucleic acid markers, useful for the assessment or diagnosis of acute allograft rejection, including kidney allograft rejection, comprising one or more than one of TncRNA, FKSG49, ZNF438, SFRS16, 1558448_a_at, CAMKK2, NFYC, NCOA3, LMAN2, PGS1, NEDD9, 237442_at, FKSG49/LOC730444, LIMK2, UNB, NASP, PRO1073, 240057_at, ITGAX, LOC730399/LOC731974, FKBP1A, HLA-G, RBMS1 and SLC6A6.

In another embodiment, the present invention provides for a subset of markers selected from the group of 24, that may be useful for the assessment, monitoring, prediction or diagnosis of allograft rejection, including acute kidney allograft rejection, comprising one or more than one of TncRNA, FKSG49, ZNF438, 1558448_a_at, CAMKK2, LMAN2, 237442_at, FKSG49/LOC730444, JUNB, PRO1073 and ITGAX.

In another embodiment, the present invention provides for a subset of markers selected from the group of 24, that may be useful for the assessment, monitoring, prediction or diagnosis of allograft rejection, including acute kidney allograft rejection, comprising TncRNA, FKSG49, ZNF438, 1558448_a_at, CAMKK2, LMAN2, 237442_at, FKSG49/LOC730444, JUNB, PRO1073 and ITGAX and one or more than one of SFRS16, NFYC, NCOA3, PGS1, NEDD9, LIMK2, NASP, 240057_at, LOC730399/LOC731974, FKBP1A, HLA-G, RBMS1 and SLC6A6. One or more than one includes 1, 2, 3, 4, 5, 6, 7, 8, 9, 10, 11, 12, 13 or more.

The results of Examples 1-3 illustrate the above embodiments—a 24 nucleic acid classifer set (TncRNA, FKSG49, ZNF438, 1558448_a_at, CAMKK2, LMAN2, 237442_at, FKSG49/LOC730444, JUNB, PRO1073, ITGAX; SFRS16, NFYC, NCOA3, PGS1, NEDD9, LIMK2, NASP, 240057_at, LOC730399/LOC731974, FKBP1A, HLA-G, RBMS1 and SLC6A6) are useful for discerning acute rejecting subjects from non-rejecting subjects. Any combination of one or more than one of the set of 24 may also be useful for discerning acute rejecting subjects from non-rejecting subjects. The intersecting set of 11 nucleic acid markers (TncRNA, FKSG49, ZNF438, 1558448_a_at, CAMKK2, LMAN2, 237442_at, FKSG49/LOC730444, JUNB, PRO1073 and ITGAX) may also be useful for discerning acute rejecting subjects from non-rejecting subjects.

TABLE 2
Differentially expressed probe sets, exhibiting about a 1.39 to 1.4-fold difference (or greater) between AR and NR subject.
The target sequence is the portion of the consensus or exemplar sequencer from which the probe sequences were selected (Affymetrix ™
NetAffx ™ Annotation database Update Release 25, Mar. 2008). The consensus or exemplary sequence is the sequence used at the time of
design of the array to represent the transcript that the GeneChip U133 2.0 probe set measures. A consensus sequence results from base-
calling algorithms that align and combine sequence data into groups. An exemplar sequence is a representative cDNA sequence for each
gene.

| | | | | log2 | | | | Representative |
|---|---|---|---|---|---|---|---|---|
| Affymetrix | | | RefSeq | (Fold | Fold | Direction | | sequence (SEQ |
| Probe Set ID | Gene Symbol | Gene Title | Accession No. | Change) | Change | (AR vs NR) | FDR value | ID NO:) |
| 1552264_a_at | MAPK1 | mitogen-activated | NM_002745 | 0.73 | 1.66 | up | 0.00749 | 1 |
| | | protein kinase 1 | NM_138957 | | | | | |
| 1552542_s_at | TAGAP | T-cell activation | NM_054114 | 0.95 | 1.93 | up | 0.00996 | 2 |
| | | GTPase activating | NM_138810 | | | | | |
| | | protein | NM_152133 | | | | | |
| 1553186_x_at | RASEF | RAS and EF-hand | NM_152573 | 1.12 | 2.18 | up | 0.00760 | 3 |
| | | domain containing | | | | | | |
| 1553297_a_at | CSF3R | colony stimulating factor | NM_000760 | 0.81 | 1.76 | up | 0.00574 | 4 |
| | | 3 receptor (granulocyte) | NM_156038 | | | | | |
| | | | NM_156039 | | | | | |
| | | | NM_172313 | | | | | |
| 1554691_a_at | PACSIN2 | protein kinase C and | NM_007229 | 0.74 | 1.67 | up | 0.00574 | 5 |
| | | casein kinase substrate | | | | | | |
| | | in neurons 2 | | | | | | |
| 1555420_a_at | KLF7 | Kruppel-like factor 7 | NM_003709 | 1.08 | 2.11 | up | 0.00638 | 6 |
| | | (ubiquitous) | | | | | | |
| 1555467_a_at | CUGBP1 | CUG triplet repeat, | NM_001025596 | 0.79 | 1.73 | up | 0.00363 | 7 |
| | | RNA binding protein 1 | NM_006560 | | | | | |
| | | | NM_198700 | | | | | |
| 1555797_a_at | ARPC5 | actin related protein ⅔ | NM_005717 | 0.76 | 1.70 | up | 0.00711 | 8 |
| | | complex, subunit 5. | | | | | | |
| | | 16 kDa | | | | | | |
| 1555852_at | 1555852_at | — | — | 0.90 | 1.87 | up | 0.00721 | 9 |
| 1555950_a_at | CD55 | CD55 molecule, decay | NM_000574 | 0.85 | 1.80 | up | 0.00703 | 10 |
| | | accelerating factor for | | | | | | |
| | | complement (Cromer | | | | | | |
| | | blood group) | | | | | | |
| 1557924_s_at | ALPL | alkaline phosphatase, | NM_000478 | 0.77 | 1.71 | up | 0.00758 | 11 |
| | | liver/bone/kidney | | | | | | |
| 1558448_a_at | 1558448_a_at | CDNA FLJ35687 fis, | — | 0.79 | 1.73 | up | 0.00677 | 12 |
| | | clone SPLEN2019349 | | | | | | |
| 1563509_at | 1563509_at | MRNA; cDNA | — | 0.84 | 1.79 | up | 0.00219 | 13 |
| | | DKFZp313O229 (from | | | | | | |
| | | clone DKEZp313O229) | | | | | | |
| 1565484_x_at | EGFR | epidermal growth factor | NM_005228 | −1.23 | 2.35 | down | 0.00562 | 14 |
| | | receptor (erythroblastic | NM_201282 | | | | | |
| | | leukemia viral (v-erb-b) | NM_201283 | | | | | |
| | | oncogene homolog, | NM_201284 | | | | | |
| | | avian) | | | | | | |
| 1565599_at | 1565599_at | Clone 23712 mRNA | — | 0.49 | 1.40 | up | 0.00760 | 15 |
| | | sequence | | | | | | |
| 1565717_s_at | FUS | fusion (involved in | NM_001010850 | 1.04 | 2.06 | up | 0.00375 | 16 |
| | | t(12; 16) in malignant | NM_004960 | | | | | |
| | | liposarcoma) | | | | | | |
| 1568609_s_at | FAM91A2/ | family with sequence | NM_207400 | 0.87 | 1.83 | up | 0.00703 | 17 |
| | FLJ39739/ | similarity 91, member | XM_001125827 | | | | | |
| | LOC727820 | A2 | XM_934503 | | | | | |
| | | hypothetical FLJ39739 | | | | | | |
| | | hypothetical protein | | | | | | |
| | | LOC727820 | | | | | | |
| 1569003_at | TMEM49 | transmembrane protein | NM_030938 | 0.95 | 1.94 | up | 0.00769 | 18 |
| | | 49 | | | | | | |
| 200709_at | FKBP1A | FK506 binding protein | NM_000801 | 0.59 | 1.50 | up | 0.00775 | 19 |
| | | 1A, 12 kDa | NM_054014 | | | | | |
| 200739_s_at | SUMO3 | SMT3 suppressor of mif | NM_006936 | 0.59 | 1.50 | up | 0.00711 | 20 |
| | | two 3 homolog 3 (S. cerevisiae) | | | | | | |
| 200796_s_at | MCL1 | myeloid cell leukemia | NM_021960 | 1.32 | 2.50 | up | 0.00269 | 21 |
| | | sequence 1 (BCL2- | NM_182763 | | | | | |
| | | related) | | | | | | |
| 200797_s_at | MCL1 | myeloid cell leukemia | NM_021960 | 0.60 | 1.52 | up | 0.00872 | 22 |
| | | sequence 1 (BCL2- | NM_182763 | | | | | |
| | | related) | | | | | | |
| 200805_at | LMAN2 | lectin, mannose-binding 2 | NM_006816 | 0.72 | 1.64 | up | 0.00265 | 23 |
| 200852_x_at | GNB2 | guanine nucleotide | NM_005273 | 0.53 | 1.44 | up | 0.00872 | 24 |
| | | binding protein (G | | | | | | |
| | | protein), beta | | | | | | |
| | | polypeptide 2 | | | | | | |
| 200904_at | HLA-E | major histocompatibility | NM_005516 | 0.66 | 1.58 | up | 0.00760 | 25 |
| | | complex, class I, E | | | | | | |
| 200959_at | FUS | fusion (involved in | NM_001010850 | 0.74 | 1.67 | up | 0.00254 | 26 |
| | | t(12; 16) in malignant | NM_004960 | | | | | |
| | | liposarcoma) | | | | | | |
| 201043_s_at | ANP32A | acidic (leucine-rich) | NM_006305 | 0.81 | 1.75 | up | 0.00087 | 27 |
| | | nuclear phosphoprotein | | | | | | |
| | | 32 family, member A | | | | | | |
| 201090_x_at | TUBA1B | tubulin, alpha 1b | NM_006082 | 0.63 | 1.55 | up | 0.00468 | 28 |
| 201440_at | DDX23 | DEAD (Asp-Glu-Ala- | NM_004818 | 0.54 | 1.46 | up | 0.00826 | 29 |
| | | Asp) box polypeptide | | | | | | |
| | | 23 | | | | | | |
| 201473_at | JUNB | jun B proto-oncogene | NM_002229 | 0.76 | 1.69 | up | 0.00638 | 30 |
| 201531_at | ZFP36 | zinc finger protein 36, | NM_003407 | 0.86 | 1.81 | up | 0.00265 | 31 |
| | | C3H type, homolog | | | | | | |
| | | (mouse) | | | | | | |
| 201651_s_at | PACSIN2 | protein kinase C and | NM_007229 | 0.68 | 1.60 | up | 0.00891 | 32 |
| | | casein kinase substrate | | | | | | |
| | | in neurons 2 | | | | | | |
| 201729_s_at | KIAA0100 | KIAA0100 | NM_014680 | 0.59 | 1.51 | up | 0.00711 | 33 |
| 201861_s_at | LRRFIP1 | leucine rich repeat (in | NM_004735 | 1.11 | 2.16 | up | 0.00222 | 34 |
| | | FLII) interacting protein 1 | | | | | | |
| 201950_x_at | CAPZB | capping protein (actin | NM_004930 | 0.70 | 1.62 | up | 0.00872 | 35 |
| | | filament) muscle Z-line, | | | | | | |
| | | beta | | | | | | |
| 201954_at | ARPC1B/ | actin related protein ⅔ | NM_005720 | 0.68 | 1.60 | up | 0.00219 | 36 |
| | LOC653888 | complex, subunit 1B, | XM_936251 | | | | | |
| | | 41 kDa | | | | | | |
| | | similar to Actin-related | | | | | | |
| | | protein 2/3 complex | | | | | | |
| | | subunit 1B (ARP2/3 | | | | | | |
| | | complex 41 kDa | | | | | | |
| | | subunit) (p41-ARC) | | | | | | |
| 201970_s_at | NASP | nuclear autoantigenic | NM_002482 | 0.52 | 1.43 | up | 0.00917 | 37 |
| | | sperm protein (histone- | NM_152298 | | | | | |
| | | binding) | NM_172164 | | | | | |
| 202150_s_at | NEDD9 | neural precursor cell | NM_006403 | 0.49 | 1.40 | up | 0.00879 | 38 |
| | | expressed, | NM_182966 | | | | | |
| | | developmentally down- | | | | | | |
| | | regulated 9 | | | | | | |
| 202180_s_at | MVP | major vault protein | NM_005115 | 0.82 | 1.77 | up | 0.00703 | 39 |
| | | | NM_017458 | | | | | |
| 202216_x_at | NFYC | nuclear transcription | NM_014223 | 0.55 | 1.46 | up | 0.00775 | 40 |
| | | factor Y, gamma | | | | | | |
| 202423_at | MYST3 | MYST histone | NM_001099412 | 0.52 | 1.43 | up | 0.00798 | 41 |
| | | acetyltransferase | NM_001099413 | | | | | |
| | | (monocytic leukemia) 3 | NM_006766 | | | | | |
| 202510_s_at | TNFAIP2 | tumor necrosis factor, | NM_006291 | 0.86 | 1.81 | up | 0.00465 | 42 |
| | | alpha-induced protein 2 | | | | | | |
| 202531_at | IRF1 | interferon regulatory | NM_002198 | 0.85 | 1.81 | up | 0.00998 | 43 |
| | | factor 1 | | | | | | |
| 202897_at | SIRPA | signal-regulatory protein | NM_001040022 | 0.99 | 1.99 | up | 0.00219 | 44 |
| | | alpha | NM_001040023 | | | | | |
| | | | NM_080792 | | | | | |
| 202910_s_at | CD97 | CD97 molecule | NM_001025160 | 0.93 | 1.90 | up | 0.00760 | 45 |
| | | | NM_001784 | | | | | |
| | | | NM_078481 | | | | | |
| 202951_at | STK38 | serine/threonine kinase | NM_007271 | 0.78 | 1.71 | up | 0.00879 | 46 |
| | | 38 | | | | | | |
| 203233_at | IL4R | interleukin 4 receptor | NM_000418 | 0.92 | 1.90 | up | 0.00912 | 47 |
| | | | NM_001008699 | | | | | |
| 203239_s_at | CNOT3 | CCR4-NOT | NM_014516 | 0.69 | 1.61 | up | 0.00857 | 48 |
| | | transcription complex, | | | | | | |
| | | subunit 3 | | | | | | |
| 203254_s_at | TLN1 | talin 1 | NM_006289 | 0.83 | 1.78 | up | 0.00434 | 49 |
| 203471_s_at | PLEK | pleckstrin | NM_002664 | 0.80 | 1.74 | up | 0.00879 | 50 |
| 203509_at | SORL1 | sortilin-related receptor, | NM_003105 | 0.68 | 1.60 | up | 0.00652 | 51 |
| | | L(DLR class) A repeats- | | | | | | |
| | | containing | | | | | | |
| 203591_s_at | CSF3R | colony stimulating factor | NM_000760 | 0.88 | 1.85 | up | 0.00638 | 52 |
| | | 3 receptor (granulocyte) | NM_156038 | | | | | |
| | | | NM_156039 | | | | | |
| | | | NM_172313 | | | | | |
| 203624_at | SFRS17A | splicing factor, | NM_005088 | 0.72 | 1.65 | up | 0.00972 | 53 |
| | | arginine/serine-rich 17A | | | | | | |
| 203748_x_at | RBMS1 | RNA binding motif, | NM_002897 | 0.80 | 1.74 | up | 0.00219 | 54 |
| | | single stranded | NM_016836 | | | | | |
| | | interacting protein 1 | NM_016839 | | | | | |
| 204166_at | SBNO2 | strawberry notch | NM_001100122 | 0.80 | 1.74 | up | 0.00314 | 55 |
| | | homolog 2 (Drosophila) | NM_014963 | | | | | |
| 204978_at | SFRS16 | splicing factor, | NM_007056 | 0.76 | 1.70 | up | 0.00552 | 56 |
| | | arginine/serine-rich 16 | | | | | | |
| 205220_at | GPR109B | G protein-coupled | NM_006018 | 1.15 | 2.22 | up | 0.00552 | 57 |
| | | receptor 109B | XM_001134375 | | | | | |
| 205285_s_at | FYB | FYN binding protein | NM_001465 | 0.62 | 1.53 | up | 0.00872 | 58 |
| | | (FYB-120/130) | NM_199335 | | | | | |
| 205539_at | AVIL | advillin | NM_006576 | 0.92 | 1.89 | up | 0.00087 | 59 |
| 205921_s_at | SLC6A6 | solute carrier family 6 | NM_003043 | 0.65 | 1.57 | up | 0.00756 | 60 |
| | | (neurotransmitter | | | | | | |
| | | transporter, taurine). | | | | | | |
| | | member 6 | | | | | | |
| 206130_s_at | ASGR2 | asialoglycoprotein | NM_001181 | 0.83 | 1.78 | up | 0.00419 | 61 |
| | | receptor 2 | NM_080912 | | | | | |
| | | | NM_080913 | | | | | |
| | | | NM_080914 | | | | | |
| 206323_x_at | OPHN1 | oligophrenin 1 | NM_002547 | 0.57 | 1.48 | up | 0.00922 | 62 |
| 207127_s_at | HNRPH3 | heterogeneous nuclear | NM_012207 | 0.64 | 1.56 | up | 0.00749 | 63 |
| | | ribonucleoprotein H3 | NM_021644 | | | | | |
| | | (2H9) | | | | | | |
| 207266_x_at | RBMS1 | RNA binding motif, | NM_002897 | 0.92 | 1.89 | up | 0.00222 | 64 |
| | | single stranded | NM_016836 | | | | | |
| | | interacting protein 1 | NM_016839 | | | | | |
| 207446_at | TLR6 | toll-like receptor 6 | NM_006068 | 0.78 | 1.72 | up | 0.00470 | 65 |
| 207643_s_at | TNFRSF1A | tumor necrosis factor | NM_001065 | 0.77 | 1.71 | up | 0.00288 | 66 |
| | | receptor superfamily, | | | | | | |
| | | member 1A | | | | | | |
| 207782_s_at | PSEN1 | presenilin 1 (Alzheimer | NM_000021 | 0.70 | 1.63 | up | 0.00971 | 67 |
| | | disease 3) | | | | | | |
| 208018_s_at | HCK | hemopoietic cell kinase | NM_002110 | 0.87 | 1.83 | up | 0.00930 | 68 |
| 208120_x_at | FKSG49/ | FKSG49 hypothetical | XM_001125803 | 0.56 | 1.47 | up | 0.00356 | 69 |
| | LOC730444 | protein LOC730444 | | | | | | |
| 208488_s_at | CR1 | complement component | NM_000573 | 0.78 | 1.72 | up | 0.00749 | 70 |
| | | (3b/4b) receptor 1 | NM_000651 | | | | | |
| | | (Knops blood group) | XM_001126036 | | | | | |
| 208702_x_at | APLP2 | amyloid beta (A4) | NM_001642 | 0.76 | 1.69 | up | 0.00870 | 71 |
| | | precursor-like protein 2 | | | | | | |
| 208772_at | ANKHD1/ | ankyrin repeat and KH | NM_017747 | 0.85 | 1.81 | up | 0.00775 | 72 |
| | MASK-BP3 | domain containing 1 | NM_017978 | | | | | |
| | | MASK-4E-BP3 | NM_020690 | | | | | |
| | | alternate reading frame | NM_024668 | | | | | |
| | | gene | | | | | | |
| 208811_s_at | DNAJB6 | DnaJ (Hsp40) homolog, | NM_005494 | 0.82 | 1.77 | up | 0.00777 | 73 |
| | | subfamily B, member 6 | NM_058246 | | | | | |
| 208885_at | LCP1 | lymphocyte cytosolic | NM_002298 | 0.77 | 1.70 | up | 0.00912 | 74 |
| | | protein 1 (L-plastin) | | | | | | |
| 208919_s_at | NADK | NAD kinase | NM_023018 | 0.81 | 1.76 | up | 0.00872 | 75 |
| 208922_s_at | NXF1 | nuclear RNA export | NM_001081491 | 0.64 | 1.56 | up | 0.00703 | 76 |
| | | factor 1 | NM_006362 | | | | | |
| 209060_x_at | NCOA3 | nuclear receptor | NM_006534 | 0.83 | 1.77 | up | 0.00652 | 77 |
| | | coactivator 3 | NM_181659 | | | | | |
| 209083_at | CORO1A | coronin, actin binding | NM_007074 | 0.82 | 1.77 | up | 0.00760 | 78 |
| | | protein, 1A | | | | | | |
| 209286_at | CDC42EP3 | CDC42 effector protein | NM_006449 | 0.91 | 1.88 | up | 0.00222 | 79 |
| | | (Rho GTPase binding) 3 | | | | | | |
| 209868_s_at | RBMS1 | RNA binding motif, | NM_002897 | 0.88 | 1.84 | up | 0.00337 | 80 |
| | | single stranded | NM_016836 | | | | | |
| | | interacting protein 1 | NM_016839 | | | | | |
| 210184_at | ITGAX | integrin, alpha X | NM_000887 | 0.65 | 1.57 | up | 0.00392 | 81 |
| | | (complement | XM_001127869 | | | | | |
| | | component 3 receptor 4 | | | | | | |
| | | subunit) | | | | | | |
| 210190_at | STX11 | syntaxin 11 | NM_003764 | 1.00 | 1.99 | up | 0.00684 | 82 |
| 210191_s_at | PHTF1 | putative homeodomain | NM_006608 | 0.90 | 1.87 | up | 0.00972 | 83 |
| | | transcription factor 1 | | | | | | |
| 210483_at | MGC31957 | hypothetical protein | — | 0.59 | 1.51 | up | 0.00652 | 84 |
| | | MGC31957 | | | | | | |
| 210484_s_at | MGC31957/ | tumor necrosis factor | NM_003841 | 0.89 | 1.85 | up | 0.00262 | 85 |
| | TNFRSF10C | receptor superfamily, | | | | | | |
| | | member 10c, decoy | | | | | | |
| | | without an intracellular | | | | | | |
| | | domain | | | | | | |
| | | hypothetical protein | | | | | | |
| | | MGC31957 | | | | | | |
| 210514_x_at | HLA-G | HLA-G | NM_002127 | 0.58 | 1.50 | up | 0.00875 | 86 |
| | | histocompatibility | | | | | | |
| | | antigen, class I, G | | | | | | |
| 210563_x_at | CFLAR | CASP8 and FADD-like | NM_003879 | 0.83 | 1.78 | up | 0.00722 | 87 |
| | | apoptosis regulator | | | | | | |
| 210569_s_at | SIGLEC9 | sialic acid binding Ig- | NM_014441 | 0.67 | 1.59 | up | 0.00087 | 88 |
| | | like lectin 9 | | | | | | |
| 210686_x_at | SLC25A16 | solute carrier family 25 | NM_152707 | 0.74 | 1.67 | up | 0.00179 | 89 |
| | | (mitochondrial carrier; | | | | | | |
| | | Graves disease | | | | | | |
| | | autoantigen), member | | | | | | |
| | | 16 | | | | | | |
| 210754_s_at | LYN | v-yes-1 Yamaguchi | NM_002350 | 0.71 | 1.64 | up | 0.00758 | 90 |
| | | sarcoma viral related | | | | | | |
| | | oncogene homolog | | | | | | |
| 210787_s_at | CAMKK2 | calcium/calmodulin- | NM_006549 | 0.62 | 1.54 | up | 0.00560 | 91 |
| | | dependent protein | NM_153499 | | | | | |
| | | kinase kinase 2, beta | NM_153500 | | | | | |
| | | | NM_172214 | | | | | |
| | | | NM_172215 | | | | | |
| | | | NM_172216 | | | | | |
| | | | NM_172226 | | | | | |
| 210992_x_at | FCGR2C | Fc fragment of IgG, low | NM_001005410 | 0.83 | 1.77 | up | 0.00522 | 92 |
| | | affinity IIc, receptor for | NM_001005411 | | | | | |
| | | (CD32) | NM_001005412 | | | | | |
| | | | NM_201563 | | | | | |
| 211058_x_at | TUBA1B | tubulin, alpha 1b | NM_006082 | 0.57 | 1.48 | up | 0.00761 | 93 |
| 211072_x_at | TUBA1B | tubulin, alpha 1b | NM_006082 | 0.60 | 1.51 | up | 0.00409 | 94 |
| 211251_x_at | NFYC | nuclear transcription | NM_014223 | 0.49 | 1.40 | up | 0.00888 | 95 |
| | | factor Y, gamma | | | | | | |
| 211395_x_at | FCGR2C | Fc fragment of IgG, low | NM_001005410 | 0.84 | 1.79 | up | 0.00562 | 96 |
| | | affinity IIc, receptor for | NM_001005411 | | | | | |
| | | (CD32) | NM_001005412 | | | | | |
| | | | NM_201563 | | | | | |
| 211454_x_at | FKSG49 | FKSG49 | — | 0.75 | 1.69 | up | 0.00087 | 97 |
| 211521_s_at | PSCD4 | pleckstrin homology, | NM_013385 | 0.69 | 1.61 | up | 0.00219 | 98 |
| | | Sec7 and coiled-coil | | | | | | |
| | | domains 4 | | | | | | |
| 211571_s_at | VCAN | versican | NM_004385 | 1.47 | 2.77 | up | 0.00470 | 99 |
| 211750_x_at | TUBA1C | tubulin, alpha 1c | NM_032704 | 0.61 | 1.52 | up | 0.00840 | 100 |
| 211787_s_at | EIF4A1 | eukaryotic translation | NM_001416 | 0.58 | 1.50 | up | 0.00715 | 101 |
| | | initiation factor 4A, | | | | | | |
| | | isoform 1 | | | | | | |
| 211794_at | FYB | FYN binding protein | NM_001465 | 0.79 | 1.73 | up | 0.00711 | 102 |
| | | (FYB-120/130) | NM_199335 | | | | | |
| 211795_s_at | FYB | FYN binding protein | NM_001465 | 1.02 | 2.03 | up | 0.00756 | 103 |
| | | (FYB-120/130) | NM_199335 | | | | | |
| 211797_s_at | NFYC | nuclear transcription | NM_014223 | 0.81 | 1.76 | up | 0.00760 | 104 |
| | | factor Y, gamma | | | | | | |
| 211823_s_at | PXN | paxillin | NM_002859 | 0.65 | 1.57 | up | 0.00356 | 105 |
| | | | XM_001132665 | | | | | |
| 211974_x_at | RBPJ | recombination signal | NM_005349 | 0.70 | 1.63 | up | 0.00179 | 106 |
| | | binding protein for | NM_015874 | | | | | |
| | | immunoglobulin kappa J | NM_203283 | | | | | |
| | | region | NM_203284 | | | | | |
| 211996_s_at | DKFZp547E087/ | KIAA0220-like protein | NR_002555 | 0.88 | 1.84 | up | 0.00711 | 107 |
| | LOC23117/ | hypothetical gene | NR_002603 | | | | | |
| | LOC440345/ | LOC283846 | XM_496136 | | | | | |
| | LOC440353/ | hypothetical protein | XM_931802 | | | | | |
| | LOC613037/ | LOC440345 | XM_931808 | | | | | |
| | LOC728888 | nuclear pore complex | XM_931814 | | | | | |
| | | interacting protein | XM_931818 | | | | | |
| | | pseudogene similar to | XM_931827 | | | | | |
| | | Protein KIAA0220 | XM_931837 | | | | | |
| | | | XM_931840 | | | | | |
| | | | XM_933834 | | | | | |
| | | | XM_933869 | | | | | |
| | | | XR_015786 | | | | | |
| | | | XR_015889 | | | | | |
| 212036_s_at | PNN | pinin, desmosome | NM_002687 | 1.34 | 2.53 | up | 0.00688 | 108 |
| | | associated protein | | | | | | |
| 212550_at | STAT5B | signal transducer and | NM_012448 | 0.66 | 1.58 | up | 0.00760 | 109 |
| | | activator of transcription | | | | | | |
| | | 5B | | | | | | |
| 212639_x_at | TUBA1B | tubulin, alpha 1b | NM_006082 | 0.65 | 1.56 | up | 0.00369 | 110 |
| 212680_x_at | PPP1R14B | protein phosphatase 1, | NM_138689 | 0.77 | 1.71 | up | 0.00519 | 111 |
| | | regulatory (inhibitor) | | | | | | |
| | | subunit 14B | | | | | | |
| 212708_at | MSL-1 | male-specific lethal-1 | NM_001012241 | 0.80 | 1.74 | up | 0.00826 | 112 |
| | | homolog | XM_932082 | | | | | |
| | | | XM_932097 | | | | | |
| | | | XM_932107 | | | | | |
| | | | XM_943695 | | | | | |
| | | | XM_943702 | | | | | |
| 212974_at | DENND3 | DENN/MADD domain | NM_014957 | 0.91 | 1.87 | up | 0.00564 | 113 |
| | | containing 3 | | | | | | |
| 213505_s_at | SFRS14 | splicing factor, | NM_001017392 | 0.55 | 1.46 | up | 0.00998 | 114 |
| | | arginine/serine-rich 14 | NM_014884 | | | | | |
| 213596_at | CASP4 | caspase 4, apoptosis- | NM_001225 | 1.00 | 2.00 | up | 0.00996 | 115 |
| | | related cysteine | NM_033306 | | | | | |
| | | peptidase | NM_033307 | | | | | |
| 213646_x_at | TUBA1B | tubulin, alpha 1b | NM_006082 | 0.63 | 1.55 | up | 0.00468 | 116 |
| 214369_s_at | RASGRP2 | RAS guanyl releasing | NM_001098670 | 0.67 | 1.59 | up | 0.00703 | 117 |
| | | protein 2 (calcium and | NM_001098671 | | | | | |
| | | DAG-regulated) | NM_005825 | | | | | |
| | | | NM_153819 | | | | | |
| 215210_s_at | DLST/DLSTP | dihydrolipoamide S- | NM_001933 | 0.48 | 1.39 | up | 0.00826 | 118 |
| | | succinyltransferase (E2 | | | | | | |
| | | component of 2-oxo- | | | | | | |
| | | glutarate complex) | | | | | | |
| | | dihydrolipoamide S- | | | | | | |
| | | succinyltransferase | | | | | | |
| | | pseudogene (E2 | | | | | | |
| | | component of 2-oxo- | | | | | | |
| | | glutarate complex) | | | | | | |
| 215236_s_at | PICALM | phosphatidylinositol | NM_001008660 | 1.21 | 2.31 | up | 0.00869 | 119 |
| | | binding clathrin | NM_007166 | | | | | |
| | | assembly protein | | | | | | |
| 215415_s_at | LYST | lysosomal trafficking | NM_000081 | 0.74 | 1.67 | up | 0.00369 | 120 |
| | | regulator | NM_001005736 | | | | | |
| 215646_s_at | VCAN | versican | NM_004385 | 1.39 | 2.61 | up | 0.00761 | 121 |
| 215760_s_at | SBNO2 | strawberry notch | NM_001100122 | 0.80 | 1.74 | up | 0.00339 | 122 |
| | | homolog 2 (Drosophila) | NM_014963 | | | | | |
| 215832_x_at | PICALM | phosphatidylinositol | NM_001008660 | 0.71 | 1.63 | up | 0.00879 | 123 |
| | | binding clathrin | NM_007166 | | | | | |
| | | assembly protein | | | | | | |
| 215990_s_at | BCL6 | B-cell CLL/lymphoma 6 | NM_001706 | 1.64 | 3.13 | up | 0.00439 | 124 |
| | | (zinc finger protein 51) | NM_138931 | | | | | |
| 216236_s_at | SLC2A14/ | solute carrier family 2 | NM_006931 | 0.90 | 1.86 | up | 0.00959 | 125 |
| | SLC2A3 | (facilitated glucose | NM_153449 | | | | | |
| | | transporter), member 3 | | | | | | |
| | | solute carrier family 2 | | | | | | |
| | | (facilitated glucose | | | | | | |
| | | transporter), member 14 | | | | | | |
| 216950_s_at | FCGR1A | Fc fragment of IgG, high | NM_000566 | 1.44 | 2.70 | up | 0.00653 | 126 |
| | | affinity Ia, receptor | | | | | | |
| | | (CD64) | | | | | | |
| 216985_s_at | STX3 | syntaxin 3 | NM_004177 | 0.92 | 1.89 | up | 0.00996 | 127 |
| 217436_x_at | LOC730399/ | hypothetical protein | XR_015561 | 0.60 | 1.51 | up | 0.00777 | 128 |
| | LOC731974 | LOC730399 | XR_015670 | | | | | |
| | | hypothetical protein | | | | | | |
| | | LOC731974 | | | | | | |
| 217475_s_at | LIMK2 | LIM domain kinase 2 | NM_001031801 | 0.78 | 1.71 | up | 0.00468 | 129 |
| | | | NM_005569 | | | | | |
| | | | NM_016733 | | | | | |
| 217507_at | SLC11A1 | solute carrier family 11 | NM_000578 | 1.09 | 2.13 | up | 0.00864 | 130 |
| | | (proton-coupled divalent | | | | | | |
| | | metal ion transporters), | | | | | | |
| | | member 1 | | | | | | |
| 217728_at | S100A6 | S100 calcium binding | NM_014624 | 0.84 | 1.79 | up | 0.00745 | 131 |
| | | protein A6 | | | | | | |
| 217992_s_at | EFHD2 | EF-hand domain family, | NM_024329 | 0.77 | 1.70 | up | 0.00879 | 132 |
| | | member D2 | | | | | | |
| 218157_x_at | CDC42SE1 | CDC42 small effector 1 | NM_001038707 | 0.72 | 1.65 | up | 0.00591 | 133 |
| | | | NM_020239 | | | | | |
| 218380_at | NLRP1 | NLR family, pyrin | NM_001033053 | 0.75 | 1.68 | up | 0.00333 | 134 |
| | | domain containing 1 | NM_014922 | | | | | |
| | | | NM_033004 | | | | | |
| | | | NM_033006 | | | | | |
| | | | NM_033007 | | | | | |
| 219100_at | OBFC1 | oligonucleotide/oligosaccharide- | NM_024928 | 0.69 | 1.62 | up | 0.00998 | 135 |
| | | binding fold | | | | | | |
| | | containing 1 | | | | | | |
| 219183_s_at | PSCD4 | pleckstrin homology, | NM_013385 | 0.80 | 1.74 | up | 0.00574 | 136 |
| | | Sec7 and coiled-coil | | | | | | |
| | | domains 4 | | | | | | |
| 219394_at | PGS1 | phosphatidylglycerophosphate | NM_024419 | 1.02 | 2.03 | up | 0.00293 | 137 |
| | | synthase 1 | | | | | | |
| 220046_s_at | CCNL1 | cyclin L1 | NM_020307 | 1.05 | 2.08 | up | 0.00919 | 138 |
| 220305_at | MGC3260 | hypothetical protein | NM_024030 | 0.97 | 1.96 | up | 0.00219 | 139 |
| | | MGC3260 | | | | | | |
| 220326_s_at | FLJ10357 | hypothetical protein | NM_018071 | 0.91 | 1.88 | up | 0.00909 | 140 |
| | | FLJ10357 | | | | | | |
| 221432_s_at | SLC25A28 | solute carrier family 25, | NM_031212 | 0.56 | 1.47 | up | 0.00671 | 141 |
| | | member 28 | | | | | | |
| 221695_s_at | MAP3K2 | mitogen-activated | NM_006609 | 0.89 | 1.85 | up | 0.00995 | 142 |
| | | protein kinase kinase | XM_001128799 | | | | | |
| | | kinase 2 | | | | | | |
| 222244_s_at | TUG1 | taurine upregulated gene 1 | NR_002323 | 0.57 | 1.48 | up | 0.00711 | 143 |
| 222435_s_at | UBE2J1 | ubiquitin-conjugating | NM_016021 | 0.97 | 1.96 | up | 0.00658 | 144 |
| | | enzyme E2, J1 (UBC6 | | | | | | |
| | | homolog, yeast) | | | | | | |
| 222955_s_at | FAM45A/ | family with sequence | NM_018472 | 0.66 | 1.58 | up | 0.00814 | 145 |
| | FAM45B/ | similarity 45, member B | NM_207009 | | | | | |
| | LOC731832 | family with sequence | XM_001130983 | | | | | |
| | | similarity 45, member A | | | | | | |
| | | similar to family with | | | | | | |
| | | sequence similarity 45, | | | | | | |
| | | member A | | | | | | |
| 223009_at | C11orf59 | chromosome 11 open | NM_017907 | 0.63 | 1.55 | up | 0.00760 | 146 |
| | | reading frame 59 | | | | | | |
| 223578_x_at | PRO1073 | PRO1073 protein | — | 0.84 | 1.79 | up | 0.00991 | 147 |
| 223591_at | RNF135 | ring finger protein 135 | NM_032322 | 0.72 | 1.64 | up | 0.00879 | 148 |
| | | | NM_197939 | | | | | |
| 224254_x_at | 224254_x_at | — | — | 0.88 | 1.84 | up | 0.00499 | 149 |
| 224566_at | TncRNA | trophoblast-derived | NR_002802 | 1.33 | 2.52 | up | 0.00536 | 150 |
| | | noncoding RNA | | | | | | |
| 224807_at | GRAMD1A | GRAM domain | NM_020895 | 0.74 | 1.67 | up | 0.00219 | 151 |
| | | containing 1A | | | | | | |
| 224909_s_at | PREX1 | phosphatidylinositol | NM_020820 | 0.94 | 1.91 | up | 0.00468 | 152 |
| | | 3,4,5-trisphosphate- | | | | | | |
| | | dependent RAC | | | | | | |
| | | exchanger 1 | | | | | | |
| 225673_at | MYADM | myeloid-associated | NM_001020818 | 0.88 | 1.84 | up | 0.00749 | 153 |
| | | differentiation marker | NM_001020819 | | | | | |
| | | | NM_001020820 | | | | | |
| | | | NM_001020821 | | | | | |
| | | | NM_138373 | | | | | |
| 226266_at | PGS1 | phosphatidylglycerophosphate | NM_024419 | 0.91 | 1.88 | up | 0.00552 | 154 |
| | | synthase 1 | | | | | | |
| 226334_s_at | AHSA2 | AHA1, activator of heat | NM_152392 | 1.01 | 2.01 | up | 0.00468 | 155 |
| | | shock 90 kDa protein | | | | | | |
| | | ATPase homolog 2 | | | | | | |
| | | (yeast) | | | | | | |
| 226872_at | RFX2 | regulatory factor X, 2 | NM_000635 | 0.81 | 1.76 | up | 0.00879 | 156 |
| | | (influences HLA class II | NM_134433 | | | | | |
| | | expression) | | | | | | |
| 227396_at | PTPRJ | protein tyrosine | NM_001098503 | 0.86 | 1.82 | up | 0.00814 | 157 |
| | | phosphatase, receptor | NM_002843 | | | | | |
| | | type, J | | | | | | |
| 227490_at | WDFY2 | WD repeat and FYVE | NM_052950 | 0.51 | 1.43 | up | 0.00342 | 158 |
| | | domain containing 2 | | | | | | |
| 227510_x_at | PRO1073 | PRO1073 protein | — | 1.16 | 2.24 | up | 0.00265 | 159 |
| 227697_at | SOCS3 | suppressor of cytokine | NM_003955 | 1.12 | 2.18 | up | 0.00470 | 160 |
| | | signaling 3 | | | | | | |
| 228216_at | 228216_at | Transcribed locus | — | 0.91 | 1.88 | up | 0.00982 | 161 |
| 228582_x_at | 228582_x_at | Transcribed locus | — | 1.20 | 2.30 | up | 0.00591 | 162 |
| 228793_at | JMJD1C | jumonji domain | NM_004241 | 1.31 | 2.48 | up | 0.00870 | 163 |
| | | containing 1C | NM_032776 | | | | | |
| 229120_s_at | CDC42SE1 | CDC42 small effector 1 | NM_001038707 | 0.92 | 1.89 | up | 0.00362 | 164 |
| | | | NM_020239 | | | | | |
| 230735_at | 230735_at | Transcribed locus | — | 0.93 | 1.91 | up | 0.00652 | 165 |
| 232555_at | 232555_at | CDNA FLJ11431 fis, | — | 0.89 | 1.86 | up | 0.00362 | 166 |
| | | clone HEMBA1001094 | | | | | | |
| 233303_at | 233303_at | Homo sapiens, clone | — | 1.20 | 2.30 | up | 0.00872 | 167 |
| | | IMAGE: 4295366, | | | | | | |
| | | mRNA | | | | | | |
| 234640_x_at | 234640_x_at | CDNA: FLJ22614 fis, | — | 1.86 | 3.64 | up | 0.00425 | 168 |
| | | clone HSI05089 | | | | | | |
| 235167_at | DKFZp547E087 | hypothetical gene | XM_496136 | 1.56 | 2.94 | up | 0.00326 | 169 |
| | | LOC283846 | XM_931802 | | | | | |
| | | | XM_931808 | | | | | |
| | | | XM_931814 | | | | | |
| | | | XM_931818 | | | | | |
| | | | XM_931827 | | | | | |
| | | | XM_931837 | | | | | |
| | | | XM_931840 | | | | | |
| 236155_at | ZCCHC6 | Zinc finger, CCHC | NM_024617 | 1.07 | 2.10 | up | 0.00823 | 170 |
| | | domain containing 6 | | | | | | |
| 236528_at | 236528_at | Transcribed locus | — | 1.14 | 2.20 | up | 0.00677 | 171 |
| 237442_at | 237442_at | — | — | 1.03 | 2.05 | up | 0.00562 | 172 |
| 237544_at | 237544_at | Transcribed locus | — | 1.07 | 2.10 | up | 0.00711 | 173 |
| 238320_at | TncRNA | trophoblast-derived | NR_002802 | 1.34 | 2.54 | up | 0.00023 | 174 |
| | | noncoding RNA | | | | | | |
| 238712_at | 238712_at | Transcribed locus | — | 0.78 | 1.71 | up | 0.00917 | 175 |
| 239021_at | 239021_at | Transcribed locus, | — | 1.19 | 2.28 | up | 0.00891 | 176 |
| | | moderately similar to | | | | | | |
| | | XP_530714.1 | | | | | | |
| | | hypothetical protein | | | | | | |
| | | XP_530714 [Pan | | | | | | |
| | | troglodytes] | | | | | | |
| 240057_at | 240057_at | Transcribed locus | — | 0.52 | 1.43 | up | 0.00703 | 177 |
| 241774_at | 241774_at | Transcribed locus | — | 1.20 | 2.29 | up | 0.00891 | 178 |
| 242907_at | 242907_at | — | — | 1.16 | 2.23 | up | 0.00494 | 179 |
| 244356_at | 244356_at | Transcribed locus | — | 1.17 | 2.25 | up | 0.00254 | 180 |
| 244556_at | LCP2 | Lymphocyte cytosolic | NM_005565 | 0.87 | 1.83 | up | 0.00777 | 181 |
| | | protein 2 (SH2 domain | | | | | | |
| | | containing leukocyte | | | | | | |
| | | protein of 76 kDa) | | | | | | |
| 244752_at | ZNF438 | zinc finger protein 438 | NM_182755 | 0.67 | 1.59 | up | 0.00468 | 182 |
| 37028_at | PPP1R15A | protein phosphatase 1, | NM_014330 | 0.52 | 1.43 | up | 0.00470 | 183 |
| | | regulatory (inhibitor) | | | | | | |
| | | subunit 15A | | | | | | |

The Representative sequence indicated in Table 6 refers to the target sequences for the corresponding probe set. The target sequence comprises a portion of the expressed nucleic acid marker found to be differentially expressed in the AR and NR subject samples. A target sequence may be used to obtain a sequence of the full gene or expressed nucleic acid marker by, for example, use of a BLAST search at a suitable database, such as is described herein.

Biological Pathways Associated with Genomic Biomarkers of the Invention

Large scale gene expression analysis methods, such as microarrays have indicated that groups of genes that have an interaction (often with two or more degrees of separation) are expressed together and may have common regulatory elements. Other examples of such coordinate regulation are known in the art, see, for example, the diauxic shift of yeast (DiRisi et al 1997 Science 278:680-686; Eisen et al. 1998. Proc Natl Acad Sci 95:14863-14868).

Microarray analysis using peripheral blood samples may be used to document the biological processes invoked during graft rejection; identification of nucleic acid markers of BCAR has also been demonstrated in the preceding examples. These markers have been demonstrated to correctly classify samples with high cross-validation specificity. The biological functions of the genes differentially expressed during rejection (Table 2) encompass three major biological categories of processes related to immune signal transduction, cytoskeletal reorganization, and apoptosis, and emphasize the participation of the cytokine-activated Jak-Stat pathway, interferon signaling, and lymphocyte activation, proliferation, chemotaxis and adhesion.

Upregulation of 4 mammalian Jak family kinases was identified in the rejecting subjects, as well as STAT3, STATS and STAT6 in patients with BCAR—the Jak tyrosine kinase-Stat transcription factor pathway is known to be involved in immune cell development, proliferation and function While acute rejection may be classically ascribed to cytotoxic T cell mediated events, these data demonstrate that Th2/STAT6 processes are also important. Genes involved in interferon (IFN) signaling are also upregulated in BCAR, including interferon-inducible guanylate-binding protein (GBP), the interferon-response factor 1 (IRF1) and STAT1. Two MHC class I genes, HLA-E and HLA-G are known to have immunomodulatory functions and are increased in AR subjects.

T cell activation and proliferation are known to involve actin remodeling. On MHC-peptide/TCR engagement, the actin cytoskeleton is bundled at the site of engagement and is essential to forming the immune synapse; this bundling is known to be mediated by structural proteins like SLP-76, and ADAP, CDC42EP, and the actin bundling protein LCP-2. The actin cytoskeleton is remodeled to link to the integrin-receptor complex through proteins like talin and paxillin. The genes encoding these proteins are upregulated in AR subjects. AVIL (Advillin) was one of the most highly differentially expressed genes, and codes for known to be a Ca²⁺ regulated actin-binding protein and a member of the gelsolin/villin family of actin regulatory proteins.

Apoptotic cell death, another central theme detected in this dataset, was represented by caspase 4, presenilin 1, NACHT leucine rich repeat and PYD containing 1 (NLRP1), and tumor necrosis factor receptor 1 (TNF-R1). ANP32A (Acidic nuclearphosphoprotein 32 family, member a), was a highly differentially expressed nucleic acid marker and this gene encodes a protein known to have pro-apoptotic function and as illustrated in this dataset, is linked to acute rejection in AR subjects. The apoptotic signature detected in peripheral blood samples of AR subjects may thus represent a combination of T cell activation (TNF-R1 is a T cell co-receptor) and activation induced cell death (AICD) of cells which have transited from the organ. Interestingly, SIGLEC-9 (Sialic-acid binding Ig-like lectin 9), another of the most highly differentially-expressed genes, encodes a cell-adhesion molecule expressed on blood leukocytes which is upregulated during inflammation and is known to negatively regulate T cell and other leukocytes through induction of apoptosis.

A product of the CAMKK2 (calcium/calmodulin-dependent protein kinase kinase 2, beta) gene encodes a protein which belongs to the Serine/Threonine protein kinase family, and plays a role in calcium-mediated signaling. Seven transcript variants encoding six distinct isoforms have been identified for this gene. CAMKK2 beta is ubiquitously expressed and known to regulate activation of the transcription factor NfkappaB. Additional splice variants have been described but their full-length nature has not been determined. The identified isoforms undergo autophosphorylation and also phosphorylate other kinases. Nucleotide sequences of human CAMKK2 are known (e.g. GenBank Accession No. AB018081, CH473973).

A product of the FKBP1A (FK506 binding protein 1A, 12 kDa) gene encodes a protein which is a member of the immunophilin protein family, which play a role in immunoregulation and basic cellular processes involving protein folding and trafficking. Nucleotide sequences of human FKBP1A are known (e.g. AB241120, AB241121, AB241122, AF483488, AF483489, AI847849, AK002777, AK010693, AK019362, AK085599, AK141261, AK145400, AK145986, AK151047, AK154751, AK168333, AK169186, AK169242, AL928719, BC004671, BG074872, BY065108, CH466551, U65098, U65099, U65100, X60203).

A product of the HLA-G (HLA-G histocompatibility antigen, class I, G) gene encodes a protein which belongs to the HLA class I heavy chain paralogues and is a heterodimer consisting of a heavy chain and a light chain. Nucleotide sequences of human HLA-G are known (e.g. AB088083, AB103589).

A product of the ITGAX (integrin, alpha X (complement component 3 receptor 4 subunit) gene encodes a heterodimeric integral membrane protein composed of an alpha chain and a beta chain. Nucleotide sequences of human ITGAX are known (e.g. AC124566, AF211864, A1035495, AI326085, AK089167, AK131133, AK155816, AK170432, BC042840, BC057200).

A product of the JUNB (jun B proto-oncogene) gene encodes a. Nucleotide sequences of human JUNB are known (e.g. BC053234, BX548032, EC268690).

A product of the LIMK2 (LIM domain kinase 2) gene encodes a protein which belongs to the LIM-domain containing family of proteins. LIMK2 is involved in regulation of actin cytoskeleton. Nucleotide sequences of human LIMK2 are known (e.g. NC_{—}000022.9 NT_{—}011520.11).

A product of the LMAN2 (lectin, mannose-binding 2) gene encodes an intracellular lectin which is known to function as a chaperone protein and transmembrane cargo receptor in the endoplasmic reticulum and golgi apparatus. Nucleotide sequences of human LMAN2 are known (e.g. X76392).

A product of the NASP (nuclear autoantigenic sperm protein (histone-binding)) gene encodes a protein which is involved in transporting histones into the nucleus of dividing cells. Multiple isoforms are encoded by transcript variants of this genes. The nucleotide sequence of the human NASP are known (e.g. BC081913, CH474008).

A product of the NCOA3 (nuclear receptor coactivator 3) gene encodes a nuclear receptor coactivator that interacts with nuclear hormone receptors to enhance their transcriptional activator functions. Nucleotide sequences of the human NCOA3 are known (e.g. AF322224, BC088343, CH474005).

A product of the NEDD9 (neural precursor cell expressed, developmentally down-regulated 9) gene encodes a docking protein which plays a central coordinating role for tyrosine-kinase-based signaling related to cell adhesion. Nucleotide sequences of the human NEDD9 are known (e.g. AC167669, AF009366, AK030985, AK033729, AK046357, AK054179, AK083374, BB458177, BC004696, BC053713, CH466546, CT025639, D10919).

A product of the NFYC (nuclear transcription factor Y, gamma) gene encodes one subunit of a trimeric complex, forming a highly conserved transcription factor that binds with high specificity to CCAAT motifs in the promoter regions in a variety of genes. Nucleotide sequences of human MFYC are known (e.g. BC045364, BC065645, BC155102, CR388024, CT027763).

A product of the PGS1 (phosphatidylglycerophosphate synthase 1) gene encodes a protein which is a phosphatidyltransferase and participates in metabolic pathways. Nucleotide sequences of human PGS1 are known (e.g. AC061992, AK024529, AK225030, AL359590, BC008903, BC015570, BC025951, BC035662, BC108732, CH471099, CR594011, CR749720, DQ892813, DQ896059).

A product of the RBMS1 (RNA binding motif, single stranded interacting protein 1) gene encodes a protein which is a member of a small family of proteins which bind single stranded DNA/RNA. Nucleotide sequences of human RBMS1 are known (e.g. AB009975).

A product of the SFRS16 (splicing factor, arginine/serine-rich 16) gene encodes a protein which may participate in processes such as mRNA processing or RNA splicing. Nucleotide sequences for human SFRS16 are known (e.g. AC011489, AF042800, AF042802, AF042803, AF042804, AF042805, AF042806, AF042807, AF042808, AF042809, AF042810, AK074590, AK094681, AL080189, AY358944, BC013178, BC080554, BC131496, CH471126, CR604154).

A product of the SLC6A6 (solute carrier family 8 (neurotransmitter transporter, taurine) member 6) gene encodes a protein which may have a role in amino acid transport or neurotransmitter transport. Nucleotide sequences of human SLC6A6 are known (e.g. NC_{—}006602, NW_{—}876271).

A short noncoding RNA, designated TncRNA (trophoblast-derived ncRNA), originates from the 3-prime end of NEAT1 and is expressed exclusively in trophoblasts. TncRNA is known to suppress MHC class II expression in mice through inhibition of CIITApIII activity, and may be a target for TP53 (p53), suggesting involvement in apoptosis or cell cycle control Nucleotide sequences of human TncRNA are known (e.g. AF001892, AF001893, AF080092, AF508303, AK027191, AP000769, AP000944, CR611820, CR618687, U60873).

A product of the ZNF438 (zinc finger protein 438) gene encodes a protein which belongs to the family of zinc-finger motif containing proteins and may play a role in regulation of DNA-dependent transcription of immunoglobulins. Nucleotide sequences of human ZNF438 are known (e.g. AF428258, AF440405, AK057323, AK131357, AK292730, AL359532, AL591707, AL596113, AL833056, BC101622, BC104757, CH471072, DQ356011, DQ356012).

A product of the PRO1073 gene (MALAT1, metastasis associated lung adenocarcinoma transcript 1) encodes a protein which may be involved in cell cycle progression. Nucleotide sequences of human PRO1073 are known (e.g. AE017126, NP_{—}875465).

Probe set 1558448_a_at is unannotated in the Affymetrix™ NetAffx™ Annotation database, but the target sequence is part of the IMAGE clone 5215251, according to NCBI Blast. IMAGE clone 5215251 is uncharacterized. A nucleotide sequence of IMAGE clone 5215251 is known (e.g. GenBank Accession No. BC0324515.1).

Probe set 208120_x_at is unannotated in the Affymetrix™ NetAffx™ Annotation database, but the target sequence is part of the gene FKSG63, according to NCBI Blast. FKSG63 is uncharacterized. A nucleotide sequence of FKSG63 is known (e.g. GenBank Accession No. AF338192).

Probe set 237442_is unannotated in the Affymetrix™ NetAffx™ Annotation database identifies a nucleic acid marker that includes sequences on chromosome 10 and may be part of the gene APBB1IP (amyloid beta (A4) precursor protein-binding family B member 1 interacting protein). Nucleotide sequence of APBB1IP is known (e.g. GenBank Accession No. A160287.18).

Probe set 240057_at is unannotated in the Affymetrix™ NetAffx™ Annotation database, and is part of an EST, according to NCBI Blast. Nucleotide sequence of the human EST is known (e.g. GenBank Accession No. AP000763.5).

Probe set 217436_x_at is annotated as coding for a “hypothetical protein” in the Affymetrix™ NetAffx™ Annotation database, but was found to be part of Homo sapiens major histocompatibility complex, class I, G, mRNA (cDNA clone IMAGE:4694038), partial cds in NCBI Blast. Nucleotide sequences of human HLA-I, G, are known (e.g. GenBank Accession No. BC020891.1)

FKSG49 is unannotated in the Affymetrix™ NetAffx™ Annotation database. Nucleotide sequence of the human FKSG49 is known (e.g. GenBank Accession No. AC113404.3).

While the specific biological roles of FKSG49, FKSG49/LOC730444, and 1558448_a_at are as yet unknown, their identification and upregulation in AR samples is indicative of their suitability as nucleic acid markers of acute rejection.

Proteomic Profiling for Diagnosing Allograft Rejection

Proteomic profiling may also be used for diagnosing allograft rejection. Proteomic profiling may be used alone, or in combination with genomic expression profiling or metabolite profiling.

In some embodiments, the invention provides for a method of assessing or diagnosing allograft rejection, including acute kidney allograft rejection in a subject comprising 1) determining the expression profile of one or more than one proteomic markers in a biological sample from the subject, the proteomic markers selected from the group comprising a polypeptide encoded by TTN, KNG1, LBP, VASN, ARNTL2, AFM, MSTP9, MST1, PI16, SERPINA5, CFD, USH1C, C2, MBL2, SERPINA10, C9, LCAT, B2M, SHBG, C1S, UBR4 and F9; 2) comparing the expression profile of the one or more than one proteomic markers to a non-rejector profile; and 3) determining whether the expression level of the one or more than one proteomic markers is increased or decreased relative to the control profile, wherein increase or decrease of the one or more than one proteomic markers is indicative of the acute rejection status. These markers are described in further detail below.

The invention also provides for a method of assessing or diagnosing allograft rejection, including acute kidney allograft rejection, in a subject as provided by the present invention comprises 1) measuring the increase or decrease of one or more than one proteomic markers selected from the group comprising a polypeptide encoded by TTN, KNG1, LBP, VASN, ARNTL2, AFM, MSTP9, MST1, PI16, SERPINA5, CFD, USH1C, C2, MBL2, SERPINA10, C9, LCAT, B2M, SHBG, CIS, UBR4, and F9; and 2) determining the ‘rejection status’ of the subject, wherein the determination of ‘rejection status’ of the subject is based on comparison of the subject's proteomic marker expression profile to a control proteomic marker expression profile.

In some embodiments, the one or more than one proteomic markers are KNG1, AFM, TTN, MSTP9/MST1, PI16, C2, MBL2, SERPINA10 and UBR4.

A myriad of methods for protein identification and quantitation are currently available, such as glycopeptide capture (Zhang et al., 2005. Mol Cell Proteomics 4:144-155), multidimensional protein identification technology (Mud-PIT) Washburn et al., 2001 Nature Biotechnology (19:242-247), and surface-enhanced laser desorption ionization (SELDI-TOF) (Hutches et al., 1993. Rapid Commun Mass Spec 7:576-580). In addition, several isotope labelling methods which allow quantification of multiple protein samples, such as isobaric tags for relative and absolute protein quantification (iTRAQ) (Ross et al, 2004 Mol Cell Proteomics 3:1154-1169); isotope coded affinity tags (ICAT) (Gygi et al., 1999 Nature Biotechnology 17:994-999), isotope coded protein labelling (ICPL) (Schmidt et al., 2004. Proteomics 5:4-15), and N-terminal isotope tagging (NIT) (Fedjaev et al., 2007 Rapid Commun Mass Spectrom 21:2671-2679; Nam et al., 2005. J Chromatogr B Analyt Technol Biomed Life Sci. 826:91-107), provide a format suitable for high-throughput performance, a trait particularly useful in biomarker screening/identification studies.

A multiplexed iTRAQ methodology was employed for identification of plasma proteomic markers in allograft recipients. iTRAQ was first described by Ross et al, 2004 (Mol Cell Proteomics 3:1154-1169). Briefly, subject plasma samples (control and allograft recipient) were depleted of the 14 most abundant proteins and quantitatively analyzed by iTRAQ-MALDI-TOF/TOF, resulting in the identification of 460 protein group codes in at least one BCAR positive and BCAR negative sample. 144 protein group codes were detected in at least 8 out of 11 BCAR positive samples, and in at least 14 of 21 controls. Table 7 presents the 18 significant protein group codes identified.

Thus, while a single candidate biomarkers may not clearly differentiate AR and NR subjects, together, a set of proteomic markers comprising KNG1, AFM, TTN, MSTP9/MST1, PI16, C2, MBL2, SERPINA10 and UBR4 achieved a satisfactory classification (63% sensitivity and 86% specificity). As described below and in the accompanying examples, amino acids sequences of the isoforms of the proteomic markers identified as members of the protein group codes are known, and may be specifically identified by the accession numbers described herein (e.g. GenBank, GenPept, IPI or the like).

While iTRAQ was one exemplary method used to detect the peptides, other methods described herein, for example immunological based methods such as ELISA may also be useful. Alternately, specific antibodies may be raised against the one or more proteins, isoforms, precursors, polypeptides, peptides, or portions or fragments thereof, and the specific antibody used to detect the presence of the one or more proteomic marker in the sample. Methods of selecting suitable peptides, immunizing animals (e.g. mice, rabbits or the like) for the production of antisera and/or production and screening of hybridomas for production of monoclonal antibodies are known in the art, and described in the references disclosed herein.

Proteomic Expression Profiling Markers (“Proteomic Markers”)

One or more precursors, splice variants, isoforms may be encoded by a single gene Examples of genes and the isoforms, precursors and variants encoded are provided in Table 7, under the respective Protein Group Code (PGC).

A polypeptide encoded by TTN (Titin, Connectin, TMD, CMH9, CMD1G, CMPD4, EOMFC, HMERF, LGMD2J, FLJ26020, FLJ26409, FLJ32040, FLJ34413, FLJ39564, FLJ43066, DKFZp451N061) is a muscle protein expressed in regions of cardiac and skeletal muscle. Nucleotide sequences encoding TTN are known (e.g. GenBank Accession Nos. AC009948.3, AF321609.2, NM_{—}133437.2, NM_{—}133432.2, NM_{—}003319.3, NM_{—}133378.3, NM133379.2,). Amino acid sequences for TTN are known (e.g. GenPept Accession Nos. NP_{—}597676.2, NP_{—}596870.2, NP_{—}597681.2NP_{—}003310.3, NP_{—}596869.3, Q4ZG20, Q8WZ50, Q6ZP81, Q8WZ42.2).

A polypeptide encoded by KNG1 (Kininogen 1, BDK) may have a role in assembly of plasma kallikrein, and has high and low molecular weight isoforms, generated by alternate splicing. Nucleotide sequences encoding KNG1 are known (e.g. GenBank Accession Nos. NM_{—}000893.2, NM001102416.1, AC109780.7, AI133186.1, BC060039.1,). Amino acid sequences for KNG1 are known (e.g. GenPept Accession Nos. NP_{—}000884.1, NP_{—}001095886.1, AAH600396.1, P01042.2, Q05CF8).

A polypeptide encoded by LBP (lipopolysaccharide binding protein) may have a role in an acute-phase immunologic response to a bacterial infection. Nucleotide sequences encoding LBP are known (e.g. GenBank Accession Nos. NM_{—}004139.2, AF013512.1, AF106067/1, M35533.1, DQ891394.2). Amino acid sequences for LBP are known (e.g. GenPept Accession Nos. NP_{—}004130.2, AAC39547.1, AAD21962.1, AAA59493.1, ABM85360.1, P18428.3, Q8TCF0).

A polypeptide encoded by VASN (vasorin) is a TGF-beta binding protein found in vascular smooth muscle cells. Nucleotide sequences encoding VASN are known (e.g. GenBank Accession Nos. NM_{—}138440.2, CH471112.2, AY166584.1). Amino acid sequences for VASN are known (e.g. GenPept Accession Nos. NP_{—}612449.2, EAW85311.1, Q6EMK4.1, AA027704.1).

A polypeptide encoded by ARNTL2 (aryl hydrocarbon receptor nuclear translocator-like-2, BMAL2, MOP9) is a member of the basic helix-loop-helix family of transcription factors, which may have roles in various physiological processes including circadian rhythms. Nucleotide sequences encoding ARNTL2 are known (e.g. GenBank Accession Nos. NM_{—}020183.3, AC068794.25, AB03992.1). Amino acid sequences for ARNTL2 are known (e.g. GenPept Accession Nos. NP_{—}064568.3, Q8WYA1.2, BAB01485.4).

A polypeptide encoded by AFM (afamin, ALB2, ALBA, ALF, MGC125338, MGC125339, AFM) is a serum transport protein of the albumin gene family. Nucleotide sequences encoding AFM are known (e.g. GenBank Accession Nos. NM_{—}001133.2, AC108157.3, AK290556.1). Amino acid sequences for AFM are known (e.g. GenPept Accession Nos. NP_{—}001124.1, BAF83245.1, P43652.1, Q4W5C5).

A polypeptide encoded by MSTP9 is a putative macrophage-stimulating protein (brain rescue factor 1), and a homolog of hepatocyte growth factor-like protein. Nucleotide sequences encoding MSTP9 are known (e.g. GenBank Accession Nos. AF083416.1, AF116647.1, AY192149.1, U28055.1). Amino acid sequences for MSTP9 are known (e.g. GenPept Accession Nos. Q2TV78.2, AAP20103.12, AAC35412.1).

A polypeptide encoded by MST1 (macrophage stimulating 1, MSP, HGFL, NF15S2, D3F15S2) may have a role in inflammatory bowel disease. Nucleotide sequences encoding MST1 are known (e.g. GenBank Accession Nos. NM020998.3, AC099668.2, AK222893.1, M74178.1). Amino acid sequences for MST1 are known (e.g. GenPept Accession Nos. NP_{—}066278.3, P26928.2, Q13208, Q49A61, Q53GN8, BAD96613.1, AAA50165.1).

A polypeptide encoded by PI16 (Peptidase inhibitor 16, PSPBP, CRISP9, MSMBBP, MGC45378, DKFZp586B1817) is a blood protein that may interact with prostate secretory proteins. Nucleotide sequences encoding PI16 are known (e.g. GenBank Accession Nos. NM_{—}153370.2, AL122034.29, AK075470.1, AK124589.1, AK302193.1, AK312785.1, BC022399.1). Amino acid sequences for PI16 are known (e.g. GenPept Accession Nos. NP_{—}699201.2, Q6UXB8.1, BAC11640.1, BAG35648.1, AAH22399.2).

A polypeptide encoded by SERPINA5 (serpin peptidase inhibitor, clade A member 5, PAI3, PCI, PROCI, protein C inhibitor) is a plasma protein inhibitor of activated protein C. Nucleotide sequences encoding SERPINA5 are known (e.g. GenBank Accession Nos. NM_{—}000624.4, AF361796.1, AK096131.1, BC018915.2, U35464.1). Amino acid sequences for SERPINA5 are known (e.g. GenPept Accession Nos. NP_{—}000615.3, P05154.2AAB60386.1, AAH08915.1, BAG53218.1).

A polypeptide encoded by CFD (complement factor D, adipsin) is a member of the trypsin factor of peptidases. Nucleotide sequences encoding CFD are known (e.g. GenBank Accession Nos. NM_{—}001928.2, AC112706.2, AJ313463.1, BC034529.1, BC057807.1, M84526.1). Amino acid sequences for CFD are known (e.g. GenPept Accession Nos. NP_{—}001919.2, P00746.5, Q6FHW3, AAA35527.1, AAH570807.1, CAC48304.1).

A polypeptide encoded by USH1C is a scaffold protein that functions in the assembly of Usher protein complexes. Nucleotide sequences encoding USH1C are known (e.g. GenBank Accession Nos. NM_{—}005709.3, NM_{—}153676.3, kAC124799.5, AB006955.1, AF039699.1, AK000936.1, BK000147.1). Amino acid sequences for USH1C are known (e.g. GenPept Accession Nos. NP_{—}005700.2, NP_{—}710142.1, AAC18049.1, BAG62565.1, DAA00086.1, Q7RTU8, Q9H758, Q9Y6N9.3).

A polypeptide encoded by C2 (complement component 2, CO2, DKFZp779M0311) is a serum glycoprotein having a role in the classical complement pathway. Nucleotide sequences encoding C2 are known (e.g. GenBank Accession Nos. NM_{—}000063.4, NM_{—}001145903.1, AF019413.1, AK096258.1, BC029781.1, BX537504.1, M26301.1, X04481.1). Amino acid sequences for C2 are known (e.g. GenPept Accession Nos. NP_{—}000054.2, NP_{—}001139375.1, AAA35604.1, CAA28169.1, CAD97767.1).

A polypeptide encoded by MBL2 (mannose binding lectin 2, MBL, MBP, MBP1, COLEC1, HSMBPC, MGC116832, MGC116833) is a soluble mannose-binding lectin found in serum. Nucleotide sequences encoding MBL2 are known (e.g. GenBank Accession Nos. NM_{—}000242.2, AB025350.1, AF360991.1, BC096181.2). Amino acid sequences for MBL2 are known (e.g. GenPept Accession Nos. NP_{—}000233.1, BAB17020.1, AAK52907.1, AAH96182.3, P11226.2, Q5SQS3, Q9HCS8).

A polypeptide encoded by SERPINA10 (serpin peptidase inhibitor clade A ember 10, ZPI, PDI) is a serpin that inhibits the activated coagulation factors X and XI. Nucleotide sequences encoding SERPINA10 are known (e.g. GenBank Accession Nos. NM_{—}001100607.1, NM_{—}016186.2, CH471061.1, AF181467.1, BC022261.1, CR606434.1). Amino acid sequences for SERPINA10 are known (e.g. GenPept Accession Nos. NP_{—}001094077.1, NP_{—}057270.1, EAW81564.1, AAD53962.1, CAD62339.1, Q9UK55.1).

A polypeptide encoded by LCAT (lecithin-cholesterol acetyltransferase) is an extracellular cholesterol esterifying enzyme, affecting cholesterol transport. Nucleotide sequences encoding LCAT are known (e.g. GenBank Accession Nos. NM_{—}000229.1, AC040162.5, BC014781.1, X06537.1). Amino acid sequences for LCAT are known (e.g. GenPept Accession Nos. NP_{—}000299.1, P04180.1, Q53XQ3, Q9Y5N3, AAH14781.1, CAB56610.1).

A polypeptide encoded by B2M (Beta-2-Microglobulin) is a serum protein found in association with the major histocompatibility complex (MHC) class 1 heavy chain on the surface of most nucleated cells. Nucleotide sequences encoding B2M are known (e.g. GenBank Accession No. NM_{—}004048, BU658737.1, BC032589.1 and AI686916.1). Amino acid sequences for B2M are known (e.g. GenPept Accession No. P61769, AAA51811, CAA23830).

A polypeptide encoded by SHBG (Sex-hormone binding globulin, androgen-binding protein, ABP, testosterone-binding beta-globulin, TEBG) is a plasma glycoprotein that binds sex steroids. Nucleotide sequences encoding SHBG are known (e.g. GenBank Accession No. AK302603.1, NM_{—}001040.2). Amino acid sequences for SHBG are known (e.g. GenPept Accession No. P04728.2, CAA34400.1, NP001031.2).

A polypeptide encoded by C1S (complement component 1, S subcomponent) is a serine protease and a component of the human complement C1. Nucleotide sequences encoding C1S are known (e.g. GenBank Accession Nos. NM_{—}001734.3, NM_{—}201442.2, AB009076.1, AK025309.1, J04080.1, M18767.1). Amino acid sequences for C1S are known (e.g. GenPept Accession Nos. NP_{—}001725.1, NP_{—}958850.1, BAA86864.1, AAA51852.1, AAA51853.1).

A polypeptide encoded by UBR4 (ubiquitin protein ligase D3 component n-recognin 4, p600; ZUBR1; RBAF600; FLJ41863; KIAA0462; KIAA1307; RP5-1126H10.1) may have a role in regulation of anchorage-independent growth associated with some oncogenic viruses. Nucleotide sequences encoding UBR4 are known (e.g. GenBank Accession Nos. NM_{—}020765.2, AL137127.7, AA748129.1, AB007931.1, BC096758.1). Amino acid sequences for UBR4 are known (e.g. GenPept Accession Nos. NP_{—}065816.2, CAI19268.1, BAA32307.1, AAH96758.1, Q5T4S7.1, Q6ZUC7, Q96HY5).

A polypeptide encoded by F9 (coagulation factor XI) is a vitamin K-dependent coagulation factor found in the blood as an active zymogen. Nucleotide sequences encoding F9 are known (e.g. GenBank Accession Nos. NM_{—}000133.3, A01819.1, AB186358.1, A13997.1, M11390.1). Amino acid sequences for F9 are known (e.g. GenPept Accession Nos. NP_{—}1000124.1, CAA00205.1, BAD89383.1, P00740.2, Q14316, CAA01140.1, AAA52023.1).

Table 7 and the IPI accession numbers provided therein further indicate database records where the amino acid sequence information of specific isoforms of the indicated protein group code members may be obtained.

Interpretation of the large body of expression data obtained from, for example, iTRAQ protein or proteomic experiments, but is greatly facilitated through use of algorithms and statistical tools designed to organize the data in a way that highlights systematic features. Visualization tools are also of value to represent differential expression by, for example, varying intensity and hue of colour. The algorithm and statistical tools available have increased in sophistication with the increase in complexity of arrays and the resulting datasets, and with the increase in processing speed, computer memory, and the relative decrease in cost of these.

Mathematical and statistical analysis of protein or polypeptide expression profiles may accomplish several things—identification of groups of genes that demonstrate coordinate regulation in a pathway or a domain of a biological system, identification of similarities and differences between two or more biological samples, identification of features of a gene expression profile that differentiate between specific events or processes in a subject, or the like. This may include assessing the efficacy of a therapeutic regimen or a change in a therapeutic regimen, monitoring or detecting the development of a particular pathology, differentiating between two otherwise clinically similar (or almost identical) pathologies, or the like.

Methods for selecting and manufacturing such antibodies, as well as their inclusion on a ‘chip’ or an array, or in an assay, and methods of using such chips, arrays or assays are referenced or described herein.

### Other Embodiments

Nucleic acid profiling may also be used in combination with metabolite (“metabolomics”) or proteomic profiling. Minor alterations in a subject's genome, such as a single nucleotide change or polymorphism, or expression of the genome (e.g. differential gene expression) may result in rapid response in the subject's small molecule metabolite profile. Small molecule metabolites may also be rapidly responsive to environmental alterations, with significant metabolite changes becoming evident within seconds to minutes of the environmental alteration—in contrast, protein or gene expression alterations may take hours or days to become evident. The list of clinical variables includes, for example, cholesterol, homocysteine, glucose, uric acid, malondialdehyde and ketone bodies. Other non-limiting examples of small molecule metabolites are listed in Table 3.

TABLE 3
Metabolites identified and quantified in NMR spectra of serum samples
obtained from subject population.

| Compound Name | |
|---|---|
| Glucose | Lactate |
| Glutamine | Alanine |
| Glycine | Proline |
| Glycerol | Valine |
| Taurine | Lysine |
| Citrate | Serine |
| Leucine | Ornithine |
| Creatinine | Tyrosine |
| Phenylalanine | Pyruvate |
| Histidine | Carnitine |
| Glutamate | Acetate |
| Isoleucine | Asparagine |
| Betaine | 3-Hydroxybutyrate |
| Creatine | Propylene glycol |
| 2-Hydroxybutyrate | Formate |
| Methionine | Choline |
| Acetone | |

Various techniques and methods may be used for obtaining a metabolite profile of a subject. The particulars of sample preparation may vary with the method used, and also on the metabolites of interest—for example, to obtain a metabolite profile of amino acids and small, generally water soluble molecules in the sample may involve filtration of the sample with a low molecular weight cutoff of 2-10 kDa, while obtaining a metabolite profile of lipids, fatty acids and other generally poorly-water soluble molecules may involve one or more steps of extraction with an organic solvent and/or drying and resolubilization of the residues. While some exemplary methods of detecting and/or quantifying markers have been indicated herein, others will be known to those skilled in the art and readily usable in the methods and uses described in this application.

Some examples of techniques and methods that may be used (either singly or in combination) to obtain a metabolite profile of a subject include, but are not limited to, nuclear magnetic resonance (NMR), gas chromatography (GC), gas chromatography in combination with mass spectroscopy (GC-MS), mass spectroscopy, Fourier transform MS (FT-MS), high performance liquid chromatography or the like. Exemplary methods for sample preparation and techniques for obtaining a metabolite profile may be found at, for example, the Human Metabolome Project website (Wishart D S et al., 2007. Nucleic Acids Research 35:D521-6).

Standard reference works setting forth the general principles of such methods useful in metabolite profiling as would be known to those of skill in the art include, for example, Handbook of Pharmaceutical Biotechnology, (ed. S C Gad) John Wiley & Sons, Inc., Hoboken, N.J., (2007), Chromatographic Methods in Clinical Chemistry and Toxicology (R Bertholf and R. Winecker, eds.) John Wiley & Sons, Inc., Hoboken, N.J., (2007), Basic One-and Two-Dimensional NMR Spectroscopy by H., Friebolin. Wiley-VCH 4^{th} Edition (2005).

Access to the methods of the invention may be provided to an end user by, for example, a clinical laboratory or other testing facility performing the individual marker tests—the biological samples are provided to the facility where the individual tests and analyses are performed and the predictive method applied; alternately, a medical practitioner may receive the marker values from a clinical laboratory and use a local implementation or an internet-based implementation to access the predictive methods of the invention.

Kits

The invention also provides for a kit for use in assessing or diagnosing a subject's rejection status. The kit may comprise reagents for specific and quantitative detection of one or more nucleic acid markers, selected from the group comprising TncRNA, FKSG49, ZNF438, SFRS16, 1558448_a_at, CAMKK2, NFYC, NCOA3, LMAN2, PGS1, NEDD9, 237442_at, FKSG49/LOC730444, LIMK2, UNB, NASP, PRO1073, 240057_at, ITGAX, LOC730399/LOC731974, FKBP1A, HLA-G, RBMS1 and SLC6A6, along with instructions for the use of such reagents and methods for analyzing the resulting data. In some embodiments, the nucleic acid markers are TncRNA, FKSG49, ZNF438, 1558448_a_at, CAMKK2, LMAN2, 237442_at, FKSG49/LOC730444, JUNB, PRO1073 and ITGAX. The kit may be used alone for predicting or diagnosing a subject's rejection status, or it may be used in conjunction with other methods for determining clinical variables, or other assays that may be deemed appropriate. The kit may include, for example, a labelled oligonucleotide capable of selectively hybridizing to the marker. The kit may further include, for example, an oligonucleotide operable to amplify a region of the marker (e.g. by PCR). Instructions or other information useful to combine the kit results with those of other assays to provide a non-rejection cutoff index for the prediction or diagnosis of a subject's rejection status may also be provided.

The invention also provides for a nucleic acid array. The array may be a two-dimensional array, and may contain at least 10 different nucleic acid molecules (e.g., at least 20, at least 30, at least 50, at least 100, or at least 200 different nucleic acid molecules). Each nucleic acid molecule may have any length sufficient to specifically identify a nucleic acid marker by hybridization. For example, each nucleic acid molecule may be between 10 and 250 nucleotides (e.g., between 12 and 200, 14 and 175, 15 and 150, 16 and 125, 18 and 100, 20 and 75, or 25 and 50 nucleotides, or any amount therebetween) in length. For example, the nucleic acid molecules of the arrays provided herein may comprise sequences that hybridize with and specifically identify one or more than one of the nucleic acid markers presented in Table 2. Examples of such sequences include SEQ ID NO: 1-183.

The invention also provides for a kit for use in assessing or diagnosing a subject's rejection status. The kit may comprise reagents for specific and quantitative detection of one or more than one proteomic markers selected from the group comprising TTN, KNG1, LBP, VASN, ARNTL2, AFM, MSTP9, MST1, PI16, SERPINA5, CFD, USH1C, C2, MBL2, SERPINA10, C9, LCAT, B2M, SHBG, C1S, UBR4 and F9, along with instructions for the use of such reagents and methods for analyzing the resulting data. In some embodiments, the one or more than one proteomic markers are KNG1, AFM, TTN, MSTP9, MST1, PI16, C2, MBL2, SERPINA10, F9 and UBR4. For example, the kit may comprise antibodies or fragments thereof, specific for the proteomic markers (primary antibodies), along with one or more secondary antibodies that may incorporate a detectable label; such antibodies may be used in an assay such as an ELISA. Alternately, the antibodies or fragments thereof may be fixed to a solid surface, e.g. an antibody array. The kit may be used alone for predicting or diagnosing a subject's rejection status, or it may be used in conjunction with other methods for determining clinical variables, or other assays that may be deemed appropriate. Instructions or other information useful to combine the kit results with those of other assays to provide a non-rejection cutoff index for the prediction or diagnosis of a subject's rejection status may also be provided.

The invention also provides for computer-readable storage medium configured with instructions for causing a programmable processor to determine whether an allograft is being rejected. Methods for determining whether an allograft is being rejected (rejection status of the subject) are described herein, and the processor comprises instructions to receive a signal (e.g. light emission, a change in intensity or frequence of fluorescence, or the like, representative of the relative quantity of the nucleic acid or proteomic marker present in the sample) and assess the level of a nucleic acid or proteomic marker relative to a control and determine if the level is increased or decreased. The processor may be further provided with instructions to interpret the pattern of increase and/or decrease of the indicated nucleic acid or proteomic marker, and provide information to a user (for example a physician) on the rejection status of the subject. Instruction and information for removal of baseline noise or other aberrant signals from the detected signals may also be included. The instructions may be provided on a computer-readable storage medium and may be implemented in a high level procedural or object oriented programming language to communicate with a computer system. Alternatively, such instructions can be implemented in assembly or machine language. The language further can be compiled or interpreted language.

The nucleic acid detection signals can be obtained using an apparatus (e.g., a chip or an array reader) and a determination of tissue rejection can be generated using a separate processor (e.g., a computer). Alternatively, a single apparatus having a programmable processor may combine these and/or other functions and obtain the detection signals and process the signals to generate a determination of the rejection status of the subject. The processing step may be performed simultaneously with the step of collecting the detection signals (e.g., “real-time”).

Methods for selecting and manufacturing such antibodies, as well as their inclusion on a ‘chip’ or an array, or in an assay, and methods of using such chips, arrays or assays are referenced or described herein.

Methods

### Subjects and Specimens

All subjects in this study received a renal transplant between 2005 and 2007 at St. Paul's Hospital or Vancouver General Hospital, Vancouver, UBC, Canada, and appropriate consent was obtained. Immunosuppression was mainly based on Mycophenolate Mofetil (MMF) in combination with Tacrolimus and/or Prednisolone. Age, gender, ethnicity and primary disease of the subjects are summarized in Table 4, below. Whole blood was drawn using PAXgene™ tubes pre-transplant (baseline) and post-transplant at 0.5, 1, 2, 3, 4, 8, 12, and 26 weeks, every 6 months through year 3, and at the time of suspected rejection. Urine samples were obtained for the same time points. PAXgene™ whole blood samples were also taken from a cohort of control subjects with no disease using representative ages and sexes from the transplant patients. All samples were stored at −80° C. until selection for analysis. 33 subjects were included in the genomic marker study, and 32 of these 33 were included in the proteomic marker study.

TABLE 4
Kidney transplant subject demographics.

| | Subjects | | Subjects | |
|---|---|---|---|---|
| | with AR | | without AR | |
| | (n = 11) | | (n = 22) | |
| Mean Age (standard deviation) | 41.85 | (11.98) | 48.97 | (10.57) |
| Gender (n, % male) | 8 | (72.73%) | 14 | (63.64%) |
| Ethnicity (n, %) | | | | |
| Caucasian | 9 | (81.82%) | 15 | (68.18%) |
| North American Indian | 1 | (9.09%) | 2 | (9.09%) |
| Asian | 0 | (0%) | 2 | (9.09%) |
| Indian Sub-continent | 1 | (9.09%) | 2 | (9.09%) |
| Other | 0 | (0%) | 1 | (4.55%) |
| Primary Disease (n, %) | | | | |
| Chronic renal failure, aetiology uncertain | 4 | (36.36%) | 2 | (9.09%) |
| Cortical or tubular necrosis | 1 | (9.09%) | 1 | (4.55%) |
| Diabetic nephropathy associated with Type II | 1 | (9.09%) | 1 | (4.55%) |
| Focal glomerulosclerosis - adults | 3 | (27.27%) | 4 | (18.18%) |
| Polycystic kidneys, adult type (dominant) | 0 | (0%) | 5 | (22.73%) |
| IgA Nephropathy (proven by immunofluorescence) | 2 | (18.18%) | 1 | (4.55%) |
| Other | 0 | (0%) | 8 | 36.36%) |
| Donor | | | | |
| Living | 3 | (27.3%) | 8 | (38.1%) |
| Deceased | 8 | (72.7%) | 13 | (61-9%) |

All kidney transplant subject clinical data was reviewed. Samples were selected from subjects with acute rejection, borderline rejection or no rejection who had no significant co-morbidities (infections, disease recurrence, or other co-morbid events). To ensure homogeneous phenotypes and to minimize biological variability for this analysis, patients were considered eligible if they were less than 75 years of age; were not receiving immunosuppression prior to transplantation; had not received pre-transplant immunological desensitization; had received a kidney transplant from a deceased or non-HLA-identical living donor; had a negative AHG-CDC anti-donor T-cell cross-match; had not received depleting antibody induction therapy with ATG or OKT3; were able to receive oral medication, had immediate graft function, and had no clinical or laboratory evidence of infections, disease recurrence, and other major co-morbid events. Biopsies were diagnosed and recorded using the Banff criteria (Solez et al 2008 Am J Transplant 8: 753; Table 1). The cohort for this study consisted of 11 acute rejection (AR) subjects within the first week, and 22 non-rejection (NR) subjects within the first week (biopsy-confirmed acute rejection, BCAR). For all NR subjects data was available at weeks 1, 2, 3, 4 and baseline (BL). One AR subject did not have a baseline sample, and three subjects did not have a week 1, week 2 and week 4 sample, respectively. Several subjects had data for additional time points at weeks 8 and 12. Two AR patients had their rejection at day 3. For the analysis, these rejections were considered in the week 1 group. 20 normal samples from 20 healthy individuals are included to calculate results relative-to-normal. Thus, the analysis includes samples from 53 individuals, 33 of which were patients who provided samples at different time points during the 3-month post-transplant period

The study employed a closed cohort case-control design to compare differential gene expression in subjects with or without BCAR during the first 3 months post-transplant. Patients with BCAR (cases) diagnosed during the first 12 weeks post-transplant were matched 1:2 with those who did not have evidence of clinical or BCAR (controls) during the same period of observation. All rejection episodes were diagnosed by conventional clinical and laboratory parameters, were confirmed by biopsy, and graded according to the Banff criteria for working classification of renal allograft pathology. Banff categories 2 and 4 (antibody-mediated or acute/active cellular rejection) were considered significant. Subjects with borderline changes (Category 3) were analyzed separately. All baseline demographic and follow-up data were recorded in the transplant program electronic database and there was no loss to follow-up during the period of study.

Immunosuppression: Immunosuppression consisted of basiliximab at 20 mg i.v. on days 0 and 4, with tacrolimus 0.075 mg/kg b.i.d and mycophenolate 1000 mg b.i.d. Drug concentrations were measured by tandem mass spectrometry; the tacrolimus dose was adjusted to achieve 12-hour trough levels of 8-12 ng/mL for the first month post-transplant, 6-9 ng/ml for the second month, then 4-8 ng/ml thereafter. First graft and non-sensitized subjects received methylprednisolone 125 mg iv on the day of transplantation, and oral prednisone of 1 mg/kg on day 1, declining to zero by day 3 post-transplant. For recipients of a second or subsequent graft, the prednisone dose was reduced slowly and in a stepwise fashion to a maintenance dose of 10 mg on alternate days after three months. Rejection episodes were treated with methylprednisolone 500 mg i.v. daily for 3-5 days. Steroid resistant rejections were treated with OKT3 5 mg i.v. or ALG 15 mg/kg i.v daily for 7-10 days.

Plasma collection and depletion: Whole blood samples from transplant recipients, taken at the scheduled time-points and at the time of suspected rejection, and similar blood samples from normal disease-free controls of comparable ages and sexes, were drawn into EDTA tubes, stored on ice before processing. Plasma was separated and stored at −80° C. within 2 hours then transferred to liquid nitrogen until selected for analysis. Plasma samples were then thawed to room temperature, diluted 5 times with 10 mM phosphate buffered saline (PBS) at pH 7.6, and filtered with spin-X centrifuge tube filters. Diluted plasma was injected via a 325 μL sample loop onto a 5 mL avian antibody affinity column (Genway Biotech; San Diego, Calif.) capable of removing the 14 most abundant plasma proteins: HAS, IgG, fibrinogen, transferring, IgA, IgM, haptoglobin, α2-macroglobulin, α1-acid glycoprotein, α1-antitrypsin, Apoliprotein-I, Apoliprotein-II, Complement C3 and low density lipoproteins (mainly Apoliprotein B). Flow-through fractions were collected and precipitated by adding TCA to a final concentration of 10% and incubated at 4° C. for 16-18 hours. The protein precipitate was recovered by centrifugation at 3200 g at 4° C. for 1 hour, washed three times with ice cold acetone (EMD; Gibbstown, N.J.) and re-hydrated with 200-300 μL iTRAQ buffer consisting of 45:45:10 saturated urea (J. T. Baker; Phillipsburg, N.J.), 0.05 M TEAB buffer (Sigma-Aldrich; St Louis, Mo.), and 0.2% SDS (Sigma-Aldrich; St Louis, Mo.). Each sample was then stored at −80° C.

### RNA Extraction and Microarray Analysis

RNA extraction was performed on thawed samples using the PAXgene™ Blood RNA Kit [Cat #762134] to isolate total RNA. Between 4 and 10 μg of RNA was routinely isolated from 2.5 ml whole blood and the RNA quality confirmed using the Agilent BioAnalyzer. Samples with 1.5 μg of RNA, an RIN (RNA integrity number) >5, and A240/A280>1.9 were packaged on dry ice and shipped by overnight courier to the Microarray Core (MAC) Laboratory, Children's Hospital, Los Angeles, Calif. for Affymetrix microarray analysis. The microarray analysis was performed by a single technician at the CAP/CLIA accredited MAC laboratory. Nascent RNA was used for double stranded cDNA synthesis. The cDNA was then labeled using the Affymetrix cDNA Synthesis Kit (Affymetrix Inc., Santa Clara, Calif.), fragmented, mixed with hybridization cocktail and hybridized onto GeneChip Human Genome U133 Plus 2.0 Arrays. The arrays were scanned with the Affymetrix System in batches of 48 with an internal RNA control made from pooled normal whole blood. Microarrays were checked for quality issues using Affymetrix version 1.16.0 and affyPLM version 1.14.0 BioConductor packages (Bolstad, B., Low Level Analysis of High-density Oligonucleotide Array Data: Background, Normalization and Summarization. 2004, University of California, Berkeley; Irizarry et al. 2003. Biostatistics 4(2): 249-64). The arrays with lower quality were repeated with a different RNA aliquot from the same time point. The Affymetrix™ NetAffx™ Annotation database Update Release 25 (March 2008) was used for identification and analysis of microarray results.

### Gene Expression Analysis

The microarray analysis produced one Cel file per sample with 54,000 probe sets that analyzes over 47,000 transcripts and variants from over 38,500 well-substantiated human genes. All Cel files were pre-processed before the final analysis. The pre-processing steps were: (1) quality control of gene chip results, (2) adjustment of background intensities, (3) normalization of all data together, (4) summarization of probe-level data into probe-set intensity values, and (5) filtering of probe-sets to removed probe-sets that did not show a high enough intensity across samples.

Quality control was performed using issues using Affy version 1.16.0 and affyPLM version 1.14.0 BioConductor packages. Samples with low quality were repeated. Cel files were RMA normalized (Bolstad, et al. Bioinformatics, 2003. 19(2): p. 185-93) and log 2-transformed with the Affy BioConductor package version 1.16.0 (Bolstad, 2004, supra). A raw expression filter left 21,771 probe sets with a signal intensity of 2⁶=64 in at least 3 of 416 samples. The filtering step was then used to include probe-sets with a log 2-expression value of at least 6 in at least 3 samples over all 416 samples that were used in the normalization. The overall number of samples included in the pre-processing steps was 416; 33 of these were from transplant subject samples were used in the final analysis.

Trypsin Digest and iTRAQ labeling: Total protein concentration was determined using the bicinchoninic acid assay (BCA) (Sigma-Aldrich, St Louis, Mo. USA) were used to obtain 100 μg of total protein from each sample. Each sample was then precipitated by the addition of 10 volumes of HPLC grade acetone at −20° C. (Sigma-Aldrich, Seelze, Germany) and incubated for 16-18 hours at −20° C. The protein precipitate was recovered by centrifugation at 16,110 g for 10 min and dissolved in 50 mM TEAB buffer (Sigma-Aldrich; St Louis, Mo.) and 0.2% electrophoresis grade SDS (Fisher Scientific; Fair Lawn, N.J.). Proteins in each sample were reduced with TCEP (Sigma-Aldrich; St Louis, Mo.) at 3.3 mM and incubated at 60° C. for 60 min. Cysteines were blocked with methyl methane thiosulfonate at a final concentration of 6.7 mM and incubated at room temperature for 10 min.

Reduced and blocked samples were then digested with sequencing grade modified trypsin (Promega; Madison, Wis.) and incubated at 37° C. for 16-18 hours. Trypsin digested peptide samples were then dried in a speed vacuum (Thermo Savant; Holbrook, N.Y.) and labeled with iTRAQ reagent according to the manufacturer's protocol (Applied Biosystems; Foster City, Calif.). Labeled samples were pooled and acidified to pH 2.5-3.0 with concentrated phosphoric acid (ACP Chemicals Inc; Montreal, QC, Canada).

2D-LC Chromatography: iTRAQ labeled peptides were separated by strong cation exchange chromatography (SCX) using a 4.6 mm internal diameter (ID) and 100 mm in length polysulphoethyl A column packed with 5 μm beads with 300 Å pores (PolyLC Inc., Columbia, Md. USA) on a VISION workstation (Applied Biosystems; Foster City, Calif.). Mobile phases used were Buffer A composed of 10 mM monobasic potassium phosphate (Sigma-Aldrich; St Louis, Mo.) and 25% acetonitrile (EMD Chemicals; Gibbstown, N.J.) pH 2.7, and Buffer B that was the same as A except for the addition of 0.5 M potassium chloride (Sigma-Aldrich St Louis, Mo., USA). Fractions of 500 μL were collected over an 80 minute gradient divided into two linear profiles: 1) 0-30 min with 5% to 35% of Buffer B, and 2) 30-80 min with 35% to 100% of Buffer B. The 20 to 30 fractions with the most peptides detected by UV trace were selected and their volumes were reduced to 150 μL in preparation for nano reverse phase chromatography. Peptides were desalted by loading fractions onto a C18 PepMap guard column (300 μm ID×5 mm, 5 μm, 100 Å, LC Packings, Amsterdam) and washing for 15 min at 50 μL/min with mobile phase A consisting of water/acetonitrile/TFA 98:2:0.1 (v/v). The trapping column was then switched into the nano flow stream at 200 mL/min where peptides were loaded onto a Magic C18 nano LC column (15 cm, 5 μm pore size, 100 Å, Michrom Bioresources Inc., Auburn Calif., USA) for high resolution chromatography. Peptides were eluted by the following gradient: 0-45 min with 5% to 15% B (acetonitrile/water/TFA 98:2:0.1, v/v); 45-100 min with 15% to 40% B, and 100-105 min with 40% to 75% B. The eluent was spotted directly onto MALDI ABI 4800 plates using a Probot microfraction collector (LC Packings, Amsterdam, Netherlands). Matrix solution, 3 mg/mL α-cyano-4-hydroxycinnamic acid (Sigma-Aldrich, St Louis, Mo. USA) in 50% ACN, 0.1% TFA, was then added at 0.75 μL per spot.

Proteomic Methodology: Proteomic analysis was performed using iTRAQ-MALDI-TOF/TOF methodology. The multiplexing capability of iTRAQ technology allows simultaneous processing of four samples per experimental run. To ensure interpretable results across different experimental runs, a reference sample was processed together with 3 patient samples in all iTRAQ runs. The reference sample consisted of a pool of plasma from 16 healthy individuals and was consistently labeled with iTRAQ reagent 114. Patient samples were randomly labeled between reagents 115, 116 and 117. Each iTRAQ run enabled the identification and quantitation of proteins of 3 patient samples relative to the reference sample.

Mass Spectrometry and Data Processing: For each experiment, peptides spotted on MALDI plates and analyzed using the 4800 MALDI TOF/TOF analyzer (Applied Biosystems; Foster City, Calif.) controlled using 4000 series Explorer version 3.5 software. The mass spectrometer was set in the positive ion mode with an MS/MS collision energy of 1 keV. A maximum of 1400 shots/spectrum were collected for each MS/MS run causing the total mass time to range from 35 to 40 hours. Peptide identification and quantitation was carried out by ProteinPilot™ Software v2.0 (Applied Biosystems/MDS Sciex, Foster City, Calif. USA) with the integrated Paragon™ Search Algorithm (Applied Biosystems) and Pro Group™ Algorithm. Database searching was performed against the International protein index (IPI HUMAN v3.39) (Kersey et al., 2004. Proteomics 4:1985-8) to identify the polypeptides present in the samples. The precursor tolerance was set to 150 ppm and the iTRAQ fragment tolerance was set to 0.2 Da. Identification parameters were set for trypsin cleavages, cysteine alkylation by MMTS, with special factors set at urea denaturation and an ID focus on biological modifications. The detected protein threshold was set at the 85% confidence interval.

Pro Group™ Algorithm (Applied Biosystems) assembled the peptide evidence from the Paragon™ Algorithm into a comprehensive summary of proteins in the sample. The set of identified proteins from each iTRAQ run were organized into protein groups to avoid redundancies. Relative protein levels (levels of labels 115, 116 and 117 relative to 114, respectively) were estimated for each protein group by Protein Pilot based on a weighted average of the log ratios of the individual peptides for each protein. The weight of each log ratio is the inverse of the Error Factor, an estimate of the error in the quantitation, calculated by Pro Group Algorithm. These weighted averages were then converted back into the linear space and corrected for experimental bias using the Auto Bias correction option in Pro Group Algorithm. Peptide ratios coming from the following cases are excluded from the calculation of the corresponding average protein ratios: shared peptides (i.e., the same peptide sequence is claimed by more than one protein), peptides with a precursor overlap (i.e., the spectrum yielding the identified peptide is also claimed by a different protein but with an unrelated peptide sequence), peptides with a low confidence (i.e., peptide ID confidence <1.0%), peptides that do not have an iTRAQ modification, peptides with only one member of the reagent pair identified, and peptide ratios where the sum of the signal-to-noise ratio for all of the peak pairs is less than 9. When all (non-blank) peptide ratios are 0 or 9999 (indicating that only one member of the reagent pair was identified), the average ratio for the corresponding protein is shown as 0 or 9999. Further information on these and other quantitative measures assigned to each protein and on the bias correction are given in ProteinPilot Software documentation.

Although each protein group in an iTRAQ experiment may consist of more than one identified protein, a single set of three iTRAQ ratios was assigned for the entire group based on its corresponding list of identified peptides. An in-house algorithm, called the Protein Group Code Algorithm (PGCA) was employed to link protein groups across all iTRAQ experiments. PGCA assigns an identification code to all the protein groups within each iTRAQ run and a common code to similar protein groups across runs. The latter code, also referred to as the protein group code (PGC), was then used to match proteins across different iTRAQ runs. This process ensures common identifier nomenclature for related proteins and protein families across all experimental runs.

Statistical Analysis

The statistical analysis for the microarray experiments was performed using SAS version 9.1, R version 2.6.1 and BioConductor version 2.1 (Gentleman, R., et al., Genome Biology, 2004. 5: p. R80). Robust Multi-array Average (RMA) (Bolstad, 2003, supra) technique was used for background correction, normalization and summarization as available in the Affy BioConductor package. A noise minimization was then performed; probe sets with expression values consistently lower than 50 across at least 3 samples were considered as noise and eliminated from further analysis. The remaining probe sets were analyzed using three different moderated T-tests. Two of the methods are available in the Linear Models for Microarray data (limma) BioConductor package—robust fit combined with eBayes and least square fit combined with eBayes. The third statistical analysis method, Statistical Analysis of Microarrays (SAM), is available in the same BioConductor package. A gene was considered statistically significant if it had a false discovery rate (FDR) <0.01 in all three methods (Smyth, G., Limma: linear models for microarray data, in Bioinformatics and Computational Biology Solutions using R and Bioconductor, R. Gentleman, et al., Editors. 2005, Springer: N.Y.). The fold-change and maximum FDR value [the highest FDR from the 3 methods] are presented in Table 2.

The nucleic acid markers were identified by applying Stepwise Discriminant Analysis (SDA) with forward selection on the statistically significant probe sets. Linear Discriminant Analysis (LDA) was used to train and test the biomarker panel as a ‘classifier marker’ to generate a minimal or small subset of markers with optimal diagnostic qualities. An 11-fold cross-validation of the entire process of classifier construction was used to evaluate the performance of the principal classifier based on the biomarker panel. Samples were randomly divided into 11 disjoint sets, each consisting of one sample from subjects with and two without BCAR, mirroring the one-to-two distribution in the overall study cohort. For each of the 11 disjoint sets, a new classifier was constructed in the same manner as the principal classifier: identification of a list of differentially expressed probe sets based on 3 moderated t-tests, followed by forward selection discriminant analysis. The classification accuracy (sensitivity and specificity) of each of the 11 classifiers was then determined based on the 3 samples left out at each fold. Sensitivity and specificity for the principal classifier were estimated by averaging the performance across the 11-fold cross-validation samples.

Statistical analysis for proteomics: A one-protein at a time evaluation of differential relative levels was performed using a robust moderated t-test (empirical Bayes, eBayes; Smyth et al., 2004 Stat Appl Genet Mol Biol 3: Article 3) on a set of proteins that have been detected, using the assigned protein group code by PGCA, in at least two thirds within each analyzed group). Using the eBayes approach decreases the number of false positives caused by artificially low sample variance estimates when the sample size in the study is small. In addition, its robust version assigns less analytical weight to protein levels that are statistical outliers. This makes the procedure less sensitive to observations deviating from the bulk of the data than classical, non-robust tests. Protein group codes with mean relative concentrations (relative to pool control's level) differing significantly between BCAR positive and negative (i.e., with p-value <0.05) were identified as potential markers.

The proteomic biomarker panel proteins were then determined using a forward selection stepwise discriminant analysis (SDA) based on the identified list of potential markers. The SDA algorithm incorporates one protein group code at a time from the list of potential markers. In the first step it identifies the protein group code that best classifies samples based on leave-one-out cross validation. In the second step it identifies the second protein group code that, together with the previously identified code, best classify samples in a leave-one-out cross validation. This procedure is repeated until all protein group codes are sequentially incorporated or until (n−2) steps are performed, where n is the number of available samples. The proteomic biomarker panel is defined by the first k protein group codes selected by the SDA algorithm, where k=k₀+kₘ is the step at which the maximum cross-validation accuracy is reached for the first time (k₀) and maintained for kₘ additional steps. In each cross-validation, sample classification is performed using a linear discriminant analysis (LDA) with prior probabilities for each group set to 0.5. In LDA, the relative concentration for each protein undetected in patient sample(s) and/or pooled control was imputed using the average relative concentration calculated from remaining training samples in each group (BCAR positive and negative).

Internal validation (proteomics data): Statistical validation was performed by a leave-one-out cross-validation of the entire process of biomarker panel selection. More specifically, at each step of the leave-one-out cross-validation one sample is left out for classification (test set) and the remaining samples are used to build a classifier (training set). The entire biomarker selection process is then performed on the training set, i.e., from the selection of protein group codes detected in at least ⅔ of the samples in each group through the biomarker panel selection by SDA. A classifier based on the resulting proteomic biomarker panel is built using LDA and tested on the test set (priors and missing values have been treated as explained above). This process is repeated until all samples are used as test set once. The overall specificity and sensitivity are estimated based on the classification accuracy of each run. All statistical analyses were implemented using R version 2.7.0 (The R Project for Statistical Computing).

Technical validation: 2 proteins from a panel of 9 proteomic biomarkers were selected for validation by Enzyme-Linked ImmunoSorbent Assay (ELISA) using commercially available kits, following manufacturer's directions: Hepatocyte growth factor-like protein homolog (R&D DHG00) and E3 ubiquitin-protein ligase UBR4 (DiaPharma—DPGRO32A).

The present invention will be further illustrated in the following examples. However it is to be understood that these examples are for illustrative purposes only, and should not be used to limit the scope of the present invention in any manner.

### Example 1

### Comparison of Biomarkers with Clinical Diagnosis

A total of 33 subjects were included in the study, comprising 11 patients with an acute rejection within the first week of transplantation, and 22 patients who were free of rejection for at least 6 months following transplantation. The 33 transplanted patients were clinically stable 3 months following renal transplantation. A total of 183 probe sets representing 160 genes were found to be statistically significantly and consistently differentially expressed between AR and NR subjects (Table 2). The sequences that the probe sets represent are presented in FIG. 10. Samples from subjects with acute rejection within the first week after transplantation clustered together, separately from samples from non-rejection patients.

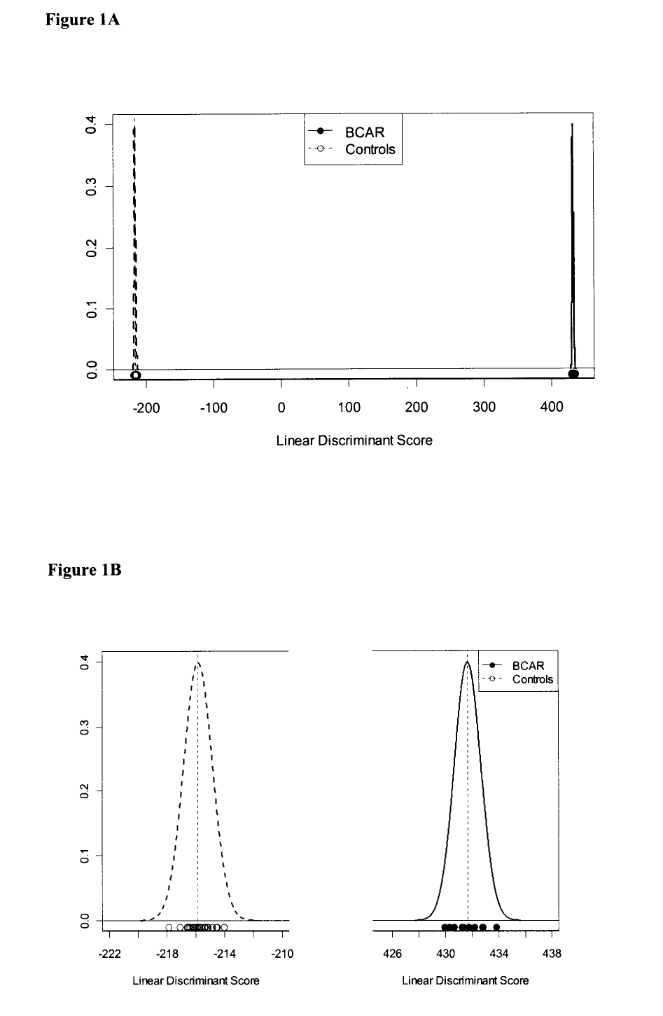

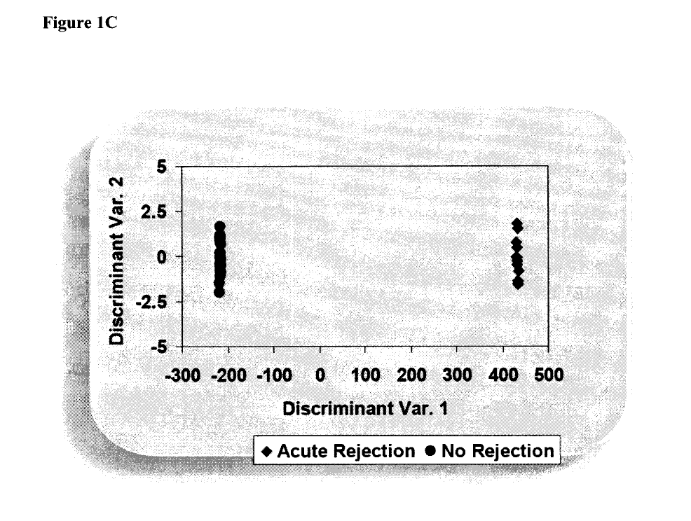

Classifying the test subjects using the panel of nucleic acid markers listed in Table 5 divided the subjects into rejectors (AR) or non-rejectors (NR) (FIG. 1A-C).

As a comparison, an independent classification of a set of subjects using only clinical parameters did allow for separation of AR and NR subject, however the boundary between the two groups was not as clear as demonstrated for the set of subjects illustrated in FIGS. 1A-C, as some overlap of AR subjects and NR subjects was observed (FIG. 2).

TABLE 5
Primary classifier (24 nucleic acid markers) associated with acute graph
rejection.

| | | | | | Direction | |
|---|---|---|---|---|---|---|
| Affymetrix | | | log2 | | (AR | |
| Probe Set | | | (Fold | Fold | versus | SEQ ID |
| ID | Gene Symbol | Gene Title | Change) | Change | NR) | NO: |
| 238320_at | ++TncRNA | trophoblast-derived | 1.34 | 2.54 | up | 150 |
| | | noncoding RNA | | | | |
| 211454_x_at | ++FKSG49 | FKSG49 | 0.75 | 1.69 | up | 69 |
| 244752_at | ++ZNF438 | zinc finger protein 438 | 0.67 | 1.59 | up | 182 |
| 204978_at | SFRS16 | splicing factor, | 0.76 | 1.70 | up | 56 |
| | | arginine/serine-rich 16 | | | | |
| 1558448_a_at | ++1558448_a_at | CDNA FLJ35687 fis, | 0.79 | 1.73 | up | 12 |
| | | clone SPLEN2019349 | | | | |
| 210787_s_at | ++CAMKK2 | calcium/calmodulin- | 0.62 | 1.54 | up | 91 |
| | | dependent protein | | | | |
| | | kinase kinase 2, beta | | | | |
| 211251_x_at | NFYC | nuclear transcription | 0.49 | 1.40 | up | 40 |
| | | factor Y, gamma | | | | |
| 209060_x_at | NCOA3 | nuclear receptor | 0.83 | 1.77 | up | 77 |
| | | coactivator 3 | | | | |
| 200805_at | ++LMAN2 | lectin, mannose-binding 2 | 0.72 | 1.64 | up | 23 |
| 226266_at | PGS1 | phosphatidylglycerophosphate | 0.91 | 1.88 | up | 137 |
| | | synthase 1 | | | | |
| 202150_s_at | NEDD9 | neural precursor cell | 0.49 | 1.40 | up | 38 |
| | | expressed, | | | | |
| | | developmentally down- | | | | |
| | | regulated 9 | | | | |
| 237442_at | ++237442_at | — | 1.03 | 2.05 | up | 172 |
| 208120_x_at | ++FKSG49/LOC730444 | FKSG49 hypothetical | 0.56 | 1.47 | up | 69 |
| | | protein LOC730444 | | | | |
| 217475_s_at | LIMK2 | LIM domain kinase 2 | 0.78 | 1.71 | up | 129 |
| 201473_at | ++JUNB | jun B proto-oncogene | 0.76 | 1.69 | up | 30 |
| 201970_s_at | NASP | nuclear autoantigenic | 0.52 | 1.43 | up | 37 |
| | | sperm protein (histone- | | | | |
| | | binding) | | | | |
| 227510_x_at | ++PRO1073 | PRO1073 protein | 1.16 | 2.24 | up | 147 |
| 240057_at | 240057_at | Transcribed locus | 0.52 | 1.43 | up | 177 |
| 210184_at | ++ITGAX | integrin, alpha X | 0.65 | 1.57 | up | 81 |
| | | (complement | | | | |
| | | component 3 receptor 4 | | | | |
| | | subunit) | | | | |
| 217436_x_at | LOC730399/LOC731974 | hypothetical protein | 0.60 | 1.51 | up | 128 |
| | | LOC730399 | | | | |
| | | hypothetical protein | | | | |
| | | LOC731974 | | | | |
| 200709_at | FKBP1A | FK506 binding protein | 0.59 | 1.50 | up | 19 |
| | | 1A, 12 kDa | | | | |
| 210514_x_at | HLA-G | HLA-G | 0.58 | 1.50 | up | 86 |
| | | histocompatibility | | | | |
| | | antigen, class I, G | | | | |
| 203748_x_at | RBMS1 | RNA binding motif, | 0.80 | 1.74 | up | 54 |
| | | single stranded | | | | |
| | | interacting protein 1 | | | | |
| 205921_s_at | SLC6A6 | solute carrier family 6 | 0.65 | 1.57 | up | 60 |
| | | (neurotransmitter | | | | |
| | | transporter, taurine), | | | | |
| | | member 6 | | | | |

++intersection of the 11 probe sets identified in the cross-validation process to estimate out-of-sample performance

### Example 2

Subjects: Of the 305 subjects who received a renal transplant during the period of observation, 27 (8.9%) developed BCAR with a Banff grade of ≧1 a during the first 3 months post-transplant, while a further 24 (7.9%) had only borderline changes. A total of 11/27 (40.74%) subjects with grade ≧1a rejection on biopsy (range: 3-10 days, mean: 6 days) fulfilled the case selection criteria with immediate graft function, and absence of infection or other confounding co-morbid events, as did 5/24 (20.83%) subjects with borderline changes on biopsy (range: 5-7 days, mean: 6 days). A further 22 subjects who had immediate graft function, with no clinical or BCAR for at least 6 months following transplantation, and no confounding clinical co-morbid events, were selected as matched controls, and 20 normal control subjects served as a comparator group. Demographic details are shown in Table 4. Graft function was significantly inferior in cases with BCAR at the first week post-transplant (27±10 vs. 42±13 ml/min/1.73M², P=0.004), but was comparable in both cases and controls by month 3 (48±11 vs. 51±8 ml/min/1.73M², P=0.359) and remained clinically stable with good allograft function throughout the 12 months period of observation (54±13 vs. 53±15 ml/min/1.73M² at month 12, P=0.859).

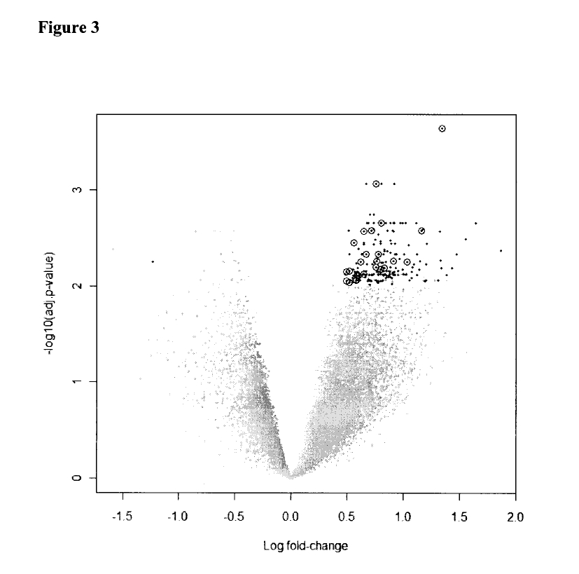

Micro-array expression: Peripheral blood samples were selected from each of the cases with BCAR at the time of biopsy for acute rejection, and from the respective controls without BCAR at a time-point identical to the respective case, and were compared with samples from normal comparators. Microarray analysis of the samples from patients with or without BCAR at an FDR <0.01 identified a total of 239 probe-sets that were differentially expressed using LIMMA, 575 probe-sets with robust LIMMA and 2677 probe-sets using SAM. The intersection of the three methods found a more restricted set of 183 probe sets which were differentially expressed between cases (BCAR) and controls (no BCAR) for all three analytical methods. Of the 183 significantly differentially expressed probe sets, 182 were over-expressed in subjects with BCAR while one (1565484_x_at coding for the epidermal-growth factor receptor; EGFR) was under-expressed (FIG. 3).

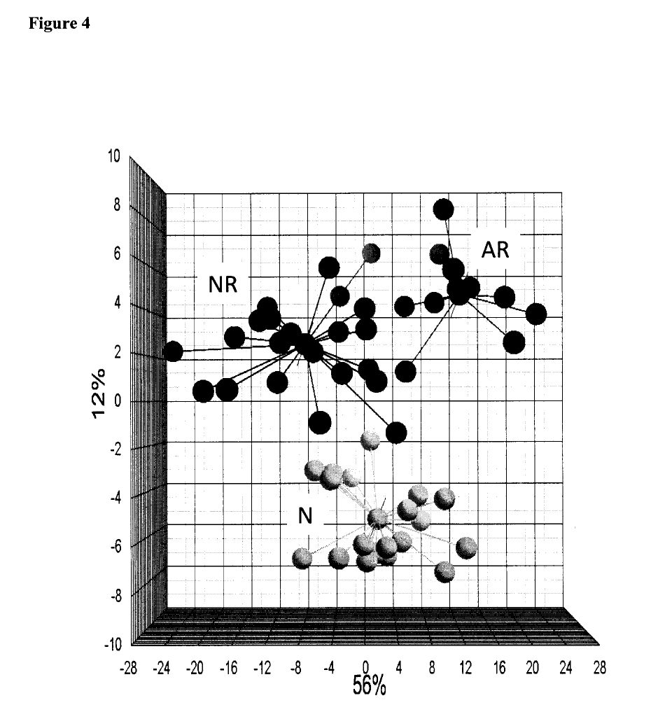

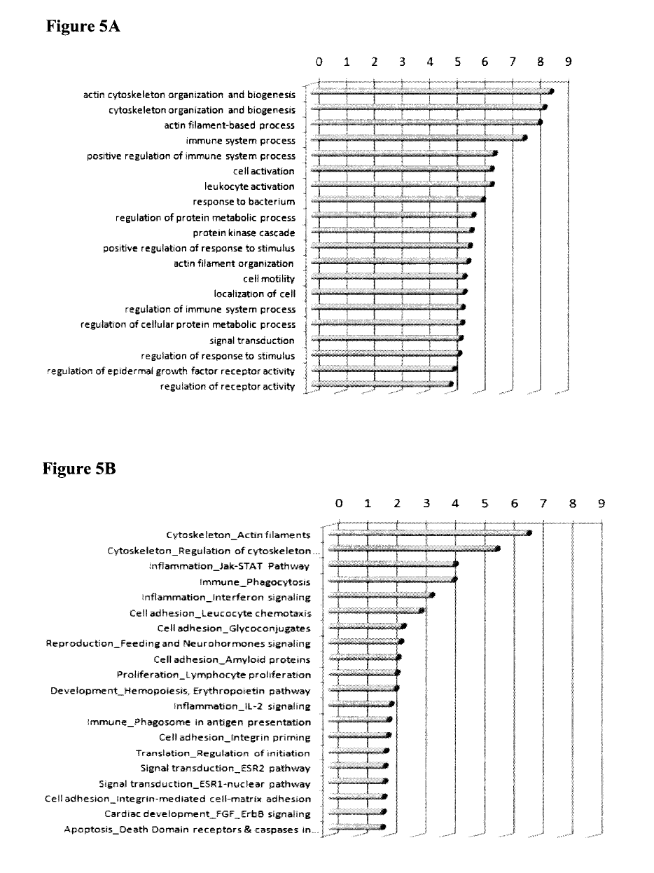

Unsupervised two-way hierarchical clustering and principal component analysis based upon these probe-sets showed discrete separation between normal subjects, patients with BCAR and those without BCAR. A principle component analysis (FIG. 4) illustrates the separation of the subject groups (AR, NR and N), demonstraing that the centroids of all groups are clearly separated. When samples from subjects with borderline changes were introduced, they were distributed heterogeneously among the cases and controls with and without BCAR. The biological processes encompassed by the 183 differentially expressed probe sets, representing approximately 160 genes, are shown in FIG. 5. Combination of overlapping networks in which probe-sets were shared identified three major biological categories implying involvement of processes related to immune responses, signal transduction, and cytoskeletal reorganization. Analysis of gene-gene and protein-protein networks (Ekins et al., 2007. Methods Mol Biol 356:319-50) revealed that the cytokine-activated Jak-Stat pathway, interferon signaling, lymphocyte activation, proliferation, chemotaxis, and apoptosis were prominently represented among the 183 differentially expressed probe-sets.

Classifier selection: Although many genes were highly associated with BCAR, co-linearity implied that not all were necessary to develop a classifier for this event. Forward selection discriminant analysis was therefore employed to identify a linear discriminant function consisting of a more parsimonious classifier from among the 183 differentially expressed probe-sets initially documented. The principal 24 probe-sets identified within this classifier, and their respective genes, are shown in Table 5.

### Example 3

### Cross Validation of Nucleic Acid Biomarkers

Cross-validation of the entire gene set using the same reductive process was employed to enhance the robustness of this classifier and to estimate the out-of-sample performance. An 11 nucleic acid marker set lists produced by this process contained a mean of 103 probe-sets, and the six most significantly differentially expressed of the original 183 probe-sets (TncRNA, FKSG49, AVIL, SIGLEC9, ANP32A, SLC25A16) were present in each list. Forward selection discriminant analysis identified a group of 11 classifiers with a union of 87 probe-sets. Eleven of these probe-sets, depicted in Table 5, were contained within the original 24 probe-set classifier. Cross-validation yielded an overall mean sensitivity of 73% and specificity of 91% for the identification of samples with or without BCAR.

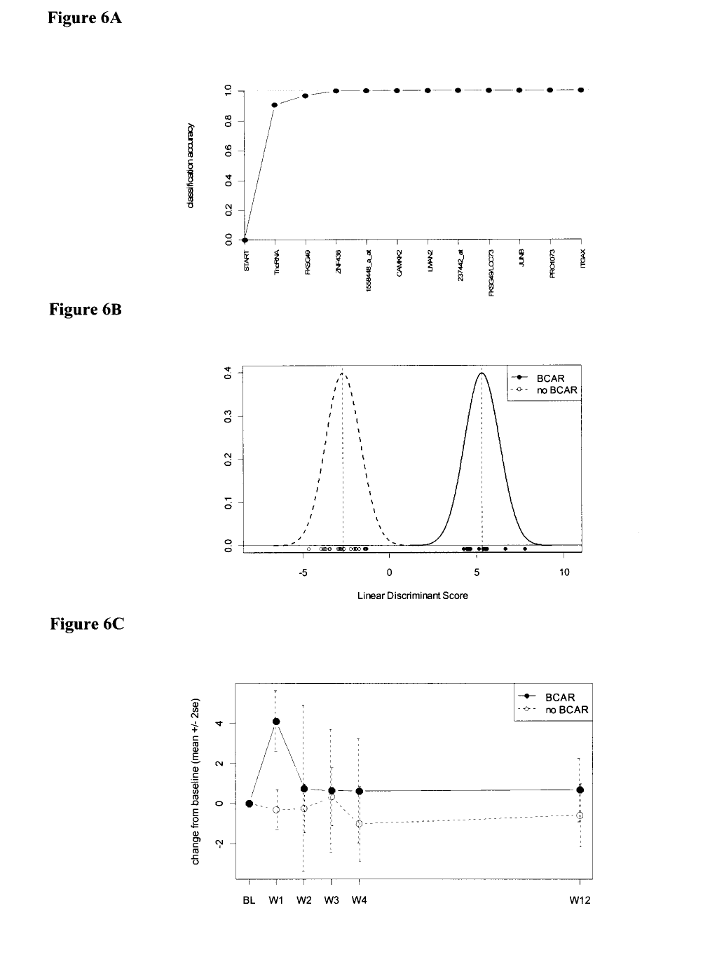

Performance of the final 11 probe-set (nucleic acid marker) classifier is shown in FIG. 6. The set of 11 nucleic acid markers included TncRNA, FKSG49, ZNF438, 1558448_a_at, CAMKK2, LMAN2, 237442_at, FKSG49/LOC730444, JUNB, PRO1073 and ITGAX.

Diagnostic accuracy improved rapidly with addition of sequential probe-sets (FIG. 6A), and the linear discriminant scores for the full 11 probe-set classifier showed clear separation of the samples with and without BCAR (FIG. 6B). Finally, longitudinal monitoring over the first 3 months post-transplant showed a significant increase in classifier score at the time of BCAR (p=0.001), with a subsequent return to the baseline value following treatment and resolution of the rejection episode. No comparable increase occurred in subjects who did not experience BCAR and there was no significant difference between these curves at any other time post-transplant (FIG. 6C).

An 11 cross-validation analysis demonstrated an average prediction accuracy of 72.7% (sensitivity) for AR and 90.9% (specificity) for NR (Table 6) and is an estimate of the prediction accuracy of the panel of 24 biomarkers listed presented in Table 5. The “++” designation in Table 5 indicates the nucleic acid markers in the intersecting set of the 11 probe sets identified in the cross-validation process to estimate out-of-sample performance.

TABLE 6
Sensitivity and specificity outcome of cross-validation analysis of nucleic
acid markers.

| | Sensitivity | Specificity |
|---|---|---|
| Fold 1 | 100% | 100% |
| Fold 2 | 0% | 100% |
| Fold 3 | 100% | 100% |
| Fold 4 | 100% | 100% |
| Fold 5 | 100% | 100% |
| Fold 6 | 100% | 100% |
| Fold 7 | 100% | 100% |
| Fold 8 | 100% | 100% |
| Fold 9 | 0% | 100% |
| Fold 10 | 100% | 50% |
| Fold 11 | 0% | 50% |

### Example 4

### Proteomics Biomarker Identification and Validation

A total of 305 subjects received a renal transplant during the period of observation, of whom 27 (8.8%) developed BCAR ≧1a during the first 3 months post-transplant. Eleven of these fulfilled the case selection criteria, with immediate graft function, BCAR ≧1a within the first 4 weeks post transplant (range: 3-10 days, mean: 6 days), and no infection or other confounding co-morbid events. A further 21 subjects who had immediate graft function, with no clinical or BCAR for at least 6 months following transplantation, and no confounding clinical co-morbid events, were selected as controls; for a total of 32 transplanted subjects. Except for the incidence of BCAR, all patients were otherwise clinically stable, with good allograft function throughout the 12-month period of observation. Six additional BCAR negative samples were selected for an internal validation, one each from three patients without BCAR included in the discovery study, and three from new patients.

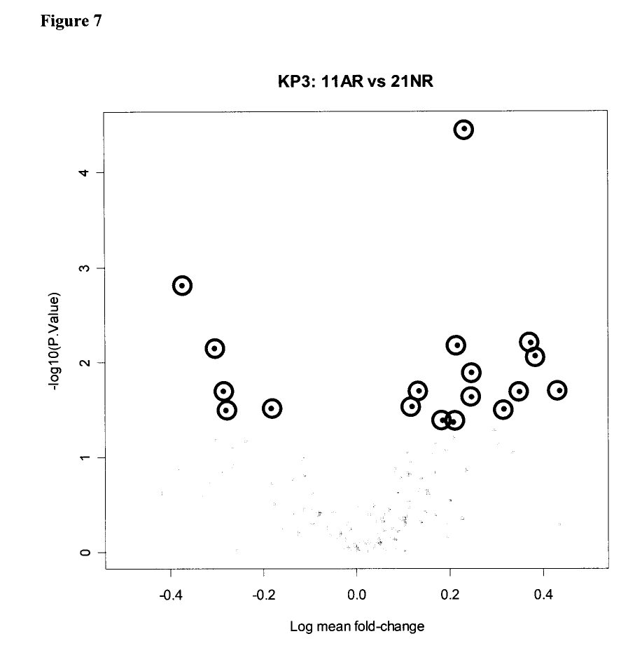

After depletion of the 14 most abundant proteins (albumin, fibrinogen, transferin, IgG, IgA, IgM, haptoglobin, α2-macroglobulin, α1-acid glycoprotein, α1-antitrypsin, Apoliprotein-I, Apoliprotein-II, complement C3 and Apoliprotein B) by immuno-affinity chromatography (Genway Biotech; San Diego, Calif.), less than 5% of the total protein mass remained. The remaining protein was trypsin digested with sequencing grade modified trypsin (Promega; Madison, Wis.) and labelled with iTRAQ reagents according to manufacturer's (Applied Biosystems; Foster City, Calif.) protocol and was examined to identify plasma proteomic markers of renal acute rejection. A total of 460 protein group codes were identified in at least one BCAR positive sample and one BCAR negative sample, among which 144 protein group codes were detected in at least 8 out of 11 BCAR positive samples and in at least 14 out of 21 controls, passing the two-thirds selection criteria per group. Analysis of the 144 protein group codes with the robust eBayes identified a total of 18 protein group codes whose concentrations differed significantly (p<0.05) between the two groups (FIG. 7). The results for the 18 significant protein group codes are shown in Table 7.

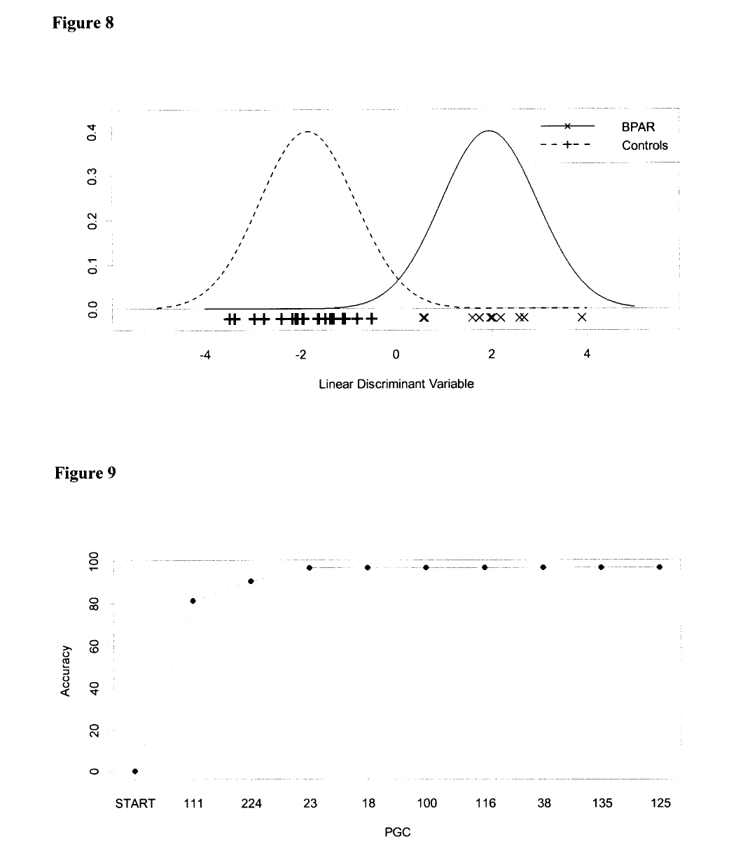

Forward selection stepwise discriminant analysis (SDA) identified a subset of 9 protein group codes that constitutes the proteomic biomarker panel (blue bold font in Table 7). Seven of the biomarker panel PGCs were up-regulated (TTN, MSTP9, PI16, C2, MBL2, SERPINA10, UBR4) and two were down-regulated (KNG1 and AFM) in patients with compared to those without BCAR. FIG. 8 illustrates the marginal classification performance achieved by protein group codes at each step of the forward selection. The x-axis shows the protein group code selected at each step to join the protein group codes selected in previous steps. The y-axis shows the classification accuracy achieved by each successively larger panel. The marginal gain in prediction accuracy quickly stabilized as protein group codes were added into the panel, and even three proteins were sufficient to achieve maximum accuracy (FIG. 8).

TABLE 7
Plasma proteins with differential relative concentrations at p-value < 0.05.
Protein group identified in the column AR vs NR constitute the plasma proteomic biomarker
panel. The column “PGC” contains the code assigned by the PGCA. Accession numbers and
protein names of all proteins in each group, corresponding genes, p-values calculated by the
robust-eBayes test, fold changes and their directions (up- or down-regulated) in BCAR positive
relative to negative are given in the remaining columns.

| | | | | | Adj. | | AR |
|---|---|---|---|---|---|---|---|
| | | Gene | | P- | P- | Fold | vs |
| Accession # | PGC | Symbol | Protein Name | Value | Value | Change | NR |
| IPI00759754.1 | **111 | TTN | Isoform 1 of Titin | 0.00003 | 0.0045 | 1.21 | up* |
| IPI00749039.2 | | TTN | titin isoform N2-A | | | | |
| IPI00179357.2 | | TTN | Isoform 7 of Titin | | | | |
| IPI00023283.3 | | TTN | Isoform 2 of Titin | | | | |
| IPI00759542.1 | | TTN | Isoform 8 of Titin | | | | |
| IPI00759637.1 | | TTN | Isoform 4 of Titin | | | | |
| IPI00759613.1 | | TTN | Isoform 5 of Titin | | | | |
| IPI00375499.2 | | TTN | titin isoform novex-2 | | | | |
| IPI00375498.2 | | TTN | titin isoform novex-1 | | | | |
| IPI00455173.4 | | TTN | Isoform 3 of Titin | | | | |
| IPI00412307.8 | | TTN | 2268 kDa protein | | | | |
| IPI00436021.3 | | TTN | Titin (Fragment) | | | | |
| IPI00884109.1 | | — | Cellular titin isoform | | | | |
| | | | PEVK variant 3 | | | | |
| | | | (Fragment) | | | | |
| IPI00789376.1 | **18 | KNG1 | KNG1 protein | 0.00149 | 0.1108 | 1.18 | down |
| IPI00797833.3 | | KNG1 | Kininogen 1 | | | | |
| IPI00032328.2 | | KNG1 | Isoform HMW of | | | | |
| | | | Kininogen-1 precursor | | | | |
| IPI00215894.1 | | KNG1 | Isoform LMW of | | | | |
| | | | Kininogen-1 precursor | | | | |
| IPI00032311.4 | 108 | LBP | Lipopolysaccharide- | 0.00641 | 0.2024 | 1.22 | up |
| | | | binding protein precursor | | | | |
| IPI00395488.2 | 222 | VASN | Vasorin precursor | 0.00666 | 0.2024 | 1.14 | up |
| IPI00827866.1 | | ARNTL2 | Isoform 7 of Aryl | | | | |
| | | | hydrocarbon receptor | | | | |
| | | | nuclear translocator-like | | | | |
| | | | protein 2 | | | | |
| IPI00142781.3 | | ARNTL2 | Isoform 1 of Aryl | | | | |
| | | | hydrocarbon receptor | | | | |
| | | | nuclear translocator-like | | | | |
| | | | protein 2 | | | | |
| IPI00163662.3 | | ARNTL2 | Isoform 2 of Aryl | | | | |
| | | | hydrocarbon receptor | | | | nuclear translocator-like |
| | | | protein 2 | | | | |
| IPI00465306.3 | | ARNTL2 | Isoform 5 of Aryl | | | | |
| | | | hydrocarbon receptor | | | | |
| | | | nuclear translocator-like | | | | |
| | | | protein 2 | | | | |
| IPI00788724.2 | | ARNTL2 | Isoform 6 of Aryl | | | | |
| | | | hydrocarbon receptor | | | | |
| | | | nuclear translocator-like | | | | |
| | | | protein 2 | | | | |
| IPI00789255.2 | | ARNTL2 | Isoform 3 of Aryl | | | | |
| | | | hydrocarbon receptor | | | | |
| | | | nuclear translocator-like | | | | |
| | | | protein 2 | | | | |
| IPI00795339.2 | | ARNTL2 | Isoform 4 of Aryl | | | | |
| | | | hydrocarbon receptor | | | | |
| | | | nuclear translocator-like | | | | |
| | | | protein 2 | | | | |
| IPI00827897.1 | | ARNTL2 | Isoform 8 of Aryl | | | | |
| | | | hydrocarbon receptor | | | | |
| | | | nuclear translocator-like | | | | |
| | | | protein 2 | | | | |
| IPI00019943.1 | **23 | AFM | Afamin precursor | 0.00679 | 0.2024 | 1.29 | down |
| IPI00873854.1 | **224 | MSTP9 | 64 kDa protein | 0.00863 | 0.2143 | 1.09 | up |
| IPI00292218.4 | | MST1 | Hepatocyte growth factor- | | | | |
| | | | like protein precursor | | | | |
| IPI00384647.1 | | MST1 | Hepatocyte growth factor- | | | | |
| | | | like protein homolog | | | | |
| IPI00718805.1 | | MSTP9 | Brain-rescue-factor-1 | | | | |
| IPI00816378.1 | | — | 21 kDa protein | | | | |
| IPI00847702.2 | | MST1 | 14 kDa protein | | | | |
| IPI00301143.5 | **135 | PI16 | Isoform 1 of Peptidase | 0.01286 | 0.2738 | 1.25 | up |
| | | | inhibitor 16 precursor | | | | |
| IPI00845506.1 | | PI16 | Isoform 2 of Peptidase | | | | |
| | | | inhibitor 16 precursor | | | | |
| IPI00007221.1 | 97 | SERPINA5 | Plasma serine protease | 0.01925 | 0.2870 | 1.22 | down |
| | | | inhibitor precursor | | | | |
| IPI00165972.3 | 104 | CFD | Complement factor D | 0.02044 | 0.2870 | 1.43 | up |
| | | | preproprotein | | | | |
| IPI00218195.3 | | USH1C | Harmonin (Usher | | | | |
| | | | syndrome type-1C | | | | |
| | | | protein) (Autoimmune | | | | |
| | | | enteropathy- related | | | | |
| | | | antigen AIE-75) (Antigen | | | | |
| | | | NY-CO-38/NY-CO-37) | | | | |
| | | | (PDZ-73 protein) (Renal | | | | |
| | | | carcinoma antigen NY- | | | | |
| | | | REN-3). Isoform 3 | | | | |
| IPI00412105.2 | | USH1C | harmonin isoform b3 | | | | |
| IPI00478105.4 | | USH1C | Isoform 4 of Harmonin | | | | |
| IPI00478519.3 | | USH1C | Isoform 3 of Harmonin | | | | |
| IPI00790818.1 | | USH1C | 29 kDa protein | | | | |
| IPI00872537.1 | | USH1C | 60 kDa protein | | | | |
| IPI00303963.1 | **38 | C2 | Complement C2 precursor | 0.01939 | 0.2870 | 1.09 | up |
| | | | (Fragment) | | | | |
| IPI00643506.3 | | C2 | Complement component 2 | | | | |
| IPI00004373.1 | **116 | MBL2 | Mannose-binding protein | 0.02119 | 0.2870 | 1.3678 | up |
| | | | C precursor | | | | |
| IPI00007199.4 | **125 | SERPINA10 | Protein Z-dependent | 0.02335 | 0.2899 | 1.2317 | up |
| | | | protease inhibitor | | | | |
| | | | precursor | | | | |
| IPI00022395.1 | 26 | C9 | Complement component | 0.02917 | 0.2962 | 1.1321 | up |
| | | | C9 precursor | | | | |
| IPI00022331.1 | 230 | LCAT | Phosphatidylcholine- | 0.03031 | 0.2962 | 1.1822 | down |
| | | | sterol acyltransferase | | | | |
| | | | precursor | | | | |
| IPI00868938.1 | 103 | — | Beta-2-microglobulin | 0.03179 | 0.2962 | 1.2735 | up |
| IPI00796379.1 | | B2M | B2M protein | | | | |
| IPI00004656.2 | | B2M | Beta-2-microglobulin | | | | |
| IPI00219583.1 | 69 | SHBG | Isoform 2 of Sex | 0.03180 | 0.2962 | 1.1924 | down |
| | | | hormone-binding globulin | | | | |
| | | | precursor | | | | |
| IPI00023019.1 | | SHBG | Isoform 1 of Sex | | | | |
| | | | hormone-binding globulin | | | | |
| | | | precursor | | | | |
| IPI00749179.2 | 29 | C1S | Uncharacterized protein | 0.04030 | 0.3532 | 1.0817 | up |
| | | | C1S | | | | |
| IPI00017696.1 | | C1S | Complement C1s | | | | |
| | | | subcomponent precursor | | | | |
| | | | Putative uncharacterized | | | | |
| | | | protein | | | | |
| IPI00385294.2 | | C1S | DKFZp686M10257 | | | | |
| IPI00791987.1 | | C1S | 17 kDa protein | | | | |
| IPI00877989.1 | | C1S | Protein | | | | |
| IPI00878772.1 | | C1S | 19 kDa protein | | | | |
| IPI00843999.2 | **100 | UBR4 | Isoform 1 of E3 ubiquitin- | 0.04317 | 0.3574 | 1.0943 | up |
| | | | protein ligase UBR4 | | | | |
| IPI00640981.3 | | UBR4 | Isoform 4 of E3 ubiquitin- | | | | |
| | | | protein ligase UBR4 | | | | |
| IPI00296176.2 | | F9 | Coagulation factor IX | | | | |
| | | | precursor | | | | |
| IPI00180305.7 | | UBR4 | Isoform 5 of E3 ubiquitin- | | | | |
| | | | protein ligase UBR4 | | | | |
| IPI00646605.3 | | UBR4 | Isoform 3 of E3 ubiquitin- | | | | |
| | | | protein ligase UBR4 | | | | |
| IPI00746934.2 | | UBR4 | Isoform 2 of E3 ubiquitin- | | | | |
| | | | protein ligase UBR4 | | | | |
| IPI00816532.1 | | F9 | Coagulation factor IX | | | | |
| | | | (Fragment) | | | | |

*“Up” with respect to “AR vs NR” indicates that one or more members of the specified protein group code are increased in the AR subjects, relative to the NR subjects. “Down” with respect to “AR vs NR” indicates that one or more members of the specified protein group code are decreased in the AR subjects, relative to the NR subjects.
**Indicates the protein group codes selected by SDA. One or more of the members of the indicated protein group code are increased or decreased (as indicated in the right-most column) in the AR subject, relative to the NR subject.

The Accession # is the International Protein Index (IPI) accession number; the amino acid sequence of the corresponding polypeptide is available from the IPI database as indicated in the methods section.

In an internal validation, two approaches were taken to estimate the ability of the proteomic biomarker panel to classify new samples. First, a leave-one-out cross-validation using LDA estimated a sensitivity of 63% and a specificity of 86% associated with the outlined discovery strategy. Second, a classifier based on the 9 protein group codes in the biomarker panel was built using LDA and was tested on 6 new NR samples. Four out of these 6 samples were correctly classified.

All citations are herein incorporated by reference, as if each individual publication was specifically and individually indicated to be incorporated by reference herein and as though it were fully set forth herein. Citation of references herein is not to be construed nor considered as an admission that such references are prior art to the present invention.

One or more currently preferred embodiments of the invention have been described by way of example. The invention includes all embodiments, modifications and variations substantially as hereinbefore described and with reference to the examples and figures. It will be apparent to persons skilled in the art that a number of variations and modifications can be made without departing from the scope of the invention as defined in the claims. Examples of such modifications include the substitution of known equivalents for any aspect of the invention in order to achieve the same result in substantially the same way.

What is claimed is:

1. A method of determining the acute allograft rejection status of a subject, the method comprising the steps of:
a. determining the nucleic acid expression profile of one or more than one nucleic acid markers in a biological sample from the subject, the nucleic acid markers selected from the group comprising TncRNA, FKSG49, ZNF438, 1558448_a_at, CAMKK2, LMAN2, 237442_at, FKSG49/LOC730444, JUNB, PRO1073 and ITGAX;
b. comparing the expression profile of the one or more than one nucleic acid markers to a control profile; and
c. determining whether the expression level of the one or more than one nucleic acid markers is increased relative to the control profile;
wherein the increase of the one or more than one nucleic acid markers is indicative of the acute rejection status of the subject.

2. The method of claim 1 wherein the group of nucleic acid markers further comprises one or more than one of SFRS16, NFYC, NCOA3, PGS1, NEDD9, LIMK2, NASP, 240057_at, LOC730399/LOC731974, FKBP1A, HLA-G, RBMS1 and SLC6A6.

3. The method of claim 1 wherein the control profile is obtained from a non-rejecting, allograft recipient subject or a non-allograft recipient subject.

4. The method of claim 1, further comprising obtaining a value for one or more clinical variables.

5. The method of claim 1, further comprising at step a) determining the expression profile of one or more than one of the nucleic acid markers selected from Table 2.

6. The method of claim 1, wherein the nucleic acid expression profile of the one or more than one nucleic acid markers is determined by detecting an RNA sequence corresponding to one or more than one markers.

7. The method of claim 1, wherein the nucleic acid expression profile of the one or more than one nucleic acid markers is determined by PCR.

8. The method of claim 1, wherein the nucleic acid expression profile of the one or more than one nucleic acid markers is determined by hybridization.

9. The method of claim 9, wherein the hybridization is to an oligonucleotide.

10. A method of determining acute allograft rejection status of a subject, the method comprising the steps of
a. determining a proteomic expression profile of proteomic markers in a biological sample from the subject, the proteomic markers including a polypeptide encoded by KNG1, AFM, TTN, MSTP9/MST1, PI16, C2, MBL2, SERPINA10, F9 and UBR4;
b. comparing the expression profile of the proteomic markers to a control profile; and
c. determining whether the expression level of the one or more than one proteomics markers is increased or decreased relative to the control profile;
wherein the increase or decrease of the five or more proteomic markers is indicative of the acute rejection status of the subject.

11. The method of claim 10 wherein the level of polypeptides encoded by KNG1 or AFM are decreased relative to a control, and the level of polypeptides encoded by TTN, MSTP9/MST1, PI16, C2, MBL2, SERPINA10, F9, or UBR4 are increased relative to a control profile.

12. The method of claim 10 wherein the control profile is obtained from a non rejecting, allograft recipient subject or a non-allograft recipient subject.

13. The method of claim 10 further comprising obtaining a value for one or more clinical variables.

14. The method of claim 10, wherein the proteomic expression profile is determined by an immunologic assay.

15. The method of claim 10, wherein the proteomic expression profile is determined by ELISA.

16. The method of claim 10, wherein the proteomic expression profile is determined by mass spectrometry.

17. The method of claim 10, wherein the proteomic expression profile is determined by an isobaric or isotope tagging method.

18. The method of claim 10 wherein the proteomic markers further include a polypeptide encoded by one or more than one of LBP, VASN, ARNTL2, PI16, SERPINA5, CFD, USH1C, C9, LCAT, B2M, SHBG and C1S.

19. The method of claim 1 wherein the control is an autologous control.

20. The method of claim 10 wherein the control is an autologous control.

21. The method of claim 1 wherein the biological sample is blood or plasma.

22. The method of claim 10 wherein the biological sample is blood or plasma.
